SEQUENCE LISTING (38 sequences)

SEQ ID NO: 1 - DNA, 1071 bp, Artificial Sequence
  Synthetic construct
  misc_feature (1)...(1071): XhoI-SacI GAL1p-MS fragment
ctcgaggtcgacggtatcgataagcttgatatcgaattccttgaattttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatccactagttctagagaacaaaagttgatctctgaagaagacttgaacggtactagaaggttataaaacttattgtcttttttatttttttcaaaagccattctaaagggctttagcaacgagtgacgaatgtaaaactttatgatttcaaagaataacctccaaaccattgaaaatgtatttttatttttattttctcccgaccccagttacctggaatttgttctttatgtactttatataagtataattctcttaaaaatttttactactttgcaatagacatcattttttcacgtaataaacccacaatcgtaatgtagttgccttacactactaggatggaccgagctc

SEQ ID NO: 2 - DNA, 593 bp, Artificial Sequence
  Synthetic construct
  misc_feature (1)...(593): Human Bax alpha BglII-XbaI fragment synthesised using yeast-biased codons
atagatctaatggacggttccggtgaacaaccaagaggtgggggtccaacctcctctgaacaaatcatgaagactggtgccttgttgcttcaaggtttcatccaagacagagctggtagaatgggtggtgaagctccagaattggccttggacccagttccacaagacgcttccaccaagaagttgtctgaatgtttgaagagaatcggtgacgaattggactctaacatggaattgcaaagaatgattgccgctgtcgacaccgactccccaagagaagtcttcttcagagtcgccgctgacatgttctctgacggtaacttcaactggggtagagttgtcgccttgttctacttcgcctctaagttggtcttgaaggccttgtgtaccaaggtcccagaattgatcagaaccatcatgggttggactttggacttcttgagagaaagattgttgggttggatccaagaccaaggtggttgggacggtttgttgtcctacttcggtactccaacttggcaaaccgttaccatcttcgttgccggtgtcttgaccgcctctttgaccatctggaagaagatgggttctagaat

SEQ ID NO: 3 - protein, 197 aa, Artificial Sequence
  Synthetic construct
  DOMAIN (1)...(197): Polypeptide sequence encoded by human Bax alpha BglII-XbaI fragment
IDLMDGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKALCTKVPELIRTIMGWTLDFLRERLLGWIQNQGGWDGLLSYFGTPTWQTVTIFVAGVLTASLTIWKKMGSR

SEQ ID NO: 4 - skipped

SEQ ID NO: 5 - DNA, 423 bp, Homo sapiens
atggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaa

SEQ ID NO: 6 - DNA, 429 bp, Homo sapiens
atgaggctgagctggttccgggtcctgacagtactgtccatctgcctgagcgccgtggccacggccacgggggccgagggcaaaaggaagctgcagatcggggtcaagaagcgggtggaccactgtcccatcaaatcgcgcaaaggggatgtcctgcacatgcactacacggggaagctggaagatgggacagagtttgacagcagcctgccccagaaccagccctttgtcttctcccttggcacaggccaggtcatcaagggctgggaccaggggctgctggggatgtgtgagggggaaaagcgcaagctggtgatcccatccgagctagggtatggagagcggggagctcccccaaagattccaggcggtgcaaccctggtgttcgaggtggagctgctcaaaatagagcgacgaactgagctgtaa

SEQ ID NO: 7 - DNA, 1389 bp, Homo sapiens
atgggaaaggaaaagactcatatcaacattgtcgtcattggacacgtagattcgggcaagtccaccactactggccatctgatctataaatgcggtggcatcgacaaaagaaccattgaaaaatttgagaaggaggctgctgagatgggaaagggctccttcaagtatgcctgggtcttggataaactgaaagctgagcgtgaacgtggtatcaccattgatatctccttgtggaaatttgagaccagcaagtactatgtgactatcattgatgccccaggacacagagactttatcaaaaacatgattacagggacatctcaggctgactgtgctgtcctgattgttgctgctggtgttggtgaatttgaagctggtatctccaagaatgggcagacccgagagcatgcccttctggcttacacactgggtgtgaaacaactaattgtcggtgttaacaaaatggattccactgagccaccctacagccagaagagatatgaggaaattgttaaggaagtcagcacttacattaagaaaattggctacaaccccgacacagtagcatttgtgccaatttctggttggaatggtgacaacatgctggagccaagtgctaacatgccttggttcaagggatggaaagtcacccgtaaggatggcaatgccagtggaaccacgctgcttgaggctctggactgcatcctaccaccaactcgtccaactgacaagcccttgcgcctgcctctccaggatgtctacaaaattggtggtattggtactgttcctgttggccgagtggagactggtgttctcaaacccggtatggtggtcacctttgctccagtcaacgttacaacggaagtaaaatctgtcgaaatgcaccatgaagctttgagtgaagctcttcctggggacaatgtgggcttcaatgtcaagaatgtgtctgtcaaggatgttcgtcgtggcaacgttgctggtgacagcaaaaatgacccaccaatggaagcagctggcttcactgctcaggtgattatcctgaaccatccaggccaaataagcgccggctatgcccctgtattggattgccacacggctcacattgcatgcaagtttgctgagctgaaggaaaagattgatcgccgttctggtaaaaagctggaagatggccctaaattcttgaagtctggtgatgctgccattgttgatatggttcctggcaagcccatgtgtgttgagagcttctcagactatccacctttgggtcgctttgctgttcgtgatatgagacagacagttgcggtgggtgtcatcaaagcagtggacaagaaggctgctggagctggcaaggtcaccaagtctgcccagaaagctcagaaggctaaatga

SEQ ID NO: 8 - DNA, 303 bp, Homo sapiens
atgtctacaggtccaactgctgccactggcagtaatcgaagacttcagcagacacaaaatcaagtagatgaggtggtggacataatgcgagttaacgtggacaaggttctggaaagagaccagaagctctctgagttagacgaccgtgcagacgcactgcaggcaggcgcttctcaatttgaaacgagcgcagccaagttgaagaggaaatattggtggaagaattgcaagatgtgggcaatcgggattactgttctggttatcttcatcatcatcatcatcgtgtgggttgtctcttcatga

SEQ ID NO: 9 - DNA, 621 bp, Homo sapiens
atggccgaagacgcagacatgcgcaatgagctggaggagatgcagcgaagggctgaccagttggctgatgagtcgctggaaagcacccgtcgtatgctgcaactggttgaagagagtaaagatgctggtatcaggactttggttatgttggatgaacaaggagaacaactcgatcgtgtcgaagaaggcatgaaccatatcaaccaagacatgaaggaggctgagaaaaatttaaaagatttagggaaatgctgtggccttttcatatgtccttgtaacaagcttaaatcaagtgatgcttacaaaaaagcctggggcaataatcaggacggagtggtggccagccagcctgctcgtgtagtggacgaacgggagcagatggccatcagtggcggcttcatccgcagggtaacaaatgatgcccgagaaaatgaaatggatgaaaacctagagcaggtgagcggcatcatcgggaacctccgtcacatggccctggatatgggcaatgagatcgatacacagaatcgccagatcgacaggatcatggagaaggctgattccaacaaaaccagaattgatgaggccaaccaacgtgcaacaaagatgctgggaagtggttaa

SEQ ID NO: 10 - DNA, 927 bp, Homo sapiens
atgtttaacggggagccaggtcctgcctcatctggggcctccaggaatgtggtgcggagctccagcattagcggtgaaatctgcggatcccagcaagccgggggcggggctgggaccaccaccgccaagaagcggcggagcagcctgggtgccaagatggtggccatcgtgggcctgactcagtggagcaagagcacactccagcttccgcagcctgaaggggccaccaagaagctgcgcagcaacatccgccggagcacggagacaggcatcgcggtggagatgcggagccgggtcacacgccagggcagccgggagtccaccgatgggagcaccaacagcaacagctccgacggcacgttcatcttccccactacccggctaggggctgaaagccagttcagcgatttcctggatgggctgggaccagctcagattgtggggcgacagacactggcaacaccacccatgggagatgtgcacattgccatcatggaccggagtggccagctggaggtggaagtgattgaagctcggggcctgacccccaaaccaggctccaaatccctcccagccacctatatcaaggtttacctgctggagaatggggcctgcttggccaagaagaagacaaagatgaccaagaagacctgtgatcccctgtaccagcaggctctgctctttgacgagggaccccagggcaaggtgctgcaggtgatcgtctggggagactatggccgcatggaccacaagtgcttcatgggcatggcccagatcatgctggacgagctggacctcagcgccgcggtcaccggctggtacaaactcttccccacctcctcagtggcagactccacactcggatccctcaccaggcgcctgtcccagtcttccctggagagtgccaccagcccctcatgctcttaa

SEQ ID NO: 11 - DNA, 324 bp, Homo sapiens
atgccgcagcagcacgatcctggccagctccgtgaacgactctgtgatgttgaaattgcacagagggctgacctcaaagaaggtcacgcccaggcgctcggcgtaggcctgggcctgctccgtgggcacctgccgcttgaacgccaggtgcaggcggttccccaccaggatcttggggactccgggggcatgctcatcgatctccttaatccatcgatcaatgccgtcaaaagaccagcggttcgcaatgtcatagaccaggatcacaccctgtgcgccccgggagtaggagcggaatatggtacaaaatcttccctggcctga

SEQ ID NO: 12 - DNA, 1563 bp, Homo sapiens
atgcctggatcacttcctttgaatgcagaagcttgctggccaaaagatgtgggaattgttgcccttgagatctattttccttctcaatatgttgatcaagcagagttggaaaaatatgatggtgtagatgctggaaagtataccattggcttgggccaggccaagatgggcttctgcacagatagagaagatattaactctctttgcatgactgtggttcagaatcttatggagagaaataacctttcctatgattgcattgggcggctggaagttggaacagagacaatcatcgacaaatcaaagtctgtgaagactaatttgatgcagctgtttgaagagtctgggaatacagatatagaaggaatcgacacaactaatgcatgctatggaggcacagctgctgtcttcaatgctgttaactggattgagtccagctcttgggatggacggtatgccctggtagttgcaggagatattgctgtatatgccacaggaaatgctagacctacaggtggagttggagcagtagctctgctaattgggccaaatgctcctttaatttttgaacgagggcttcgtgggacacatatgcaacatgcctatgatttttacaagcctgatatgctatctgaatatcctatagtagatggaaaactctccatacagtgctacctcagtgcattagaccgctgctactctgtctactgcaaaaagatccatgcccagtggcagaaagagggaaatgataaagattttaccttgaatgattttggcttcatgatctttcactcaccatattgtaaactggttcagaaatctctagctcggatgttgctgaatgacttccttaatgaccagaatagagataaaaatagtatctatagtggcctggaagcctttggggatgttaaattagaagacacctactttgatagagatgtggagaaggcatttatgaaggctagctctgaactcttcagtcagaaaacaaaggcatctttacttgtatcaaatcaaaatggaaatatgtacacatcttcagtatatggttcccttgcatctgttctagcacagtactcacctcagcaattagcagggaagagaattggagtgttttcttatggttctggtttggctgccactctgtactctcttaaagtcacacaagatgctacaccggggtctgctcttgataaaataacagcaagtttatgtgatcttaaatcaaggcttgattcaagaactggtgtggcaccagatgtcttcgctgaaaacatgaagctcagagaggacacccatcatttggtcaactatattccccagggttcaatagattcactctttgaaggaacgtggtacttagttagggtggatgaaaagcacagaagaacttacgctcggcgtcccactccaaatgatgacactttggatgaaggagtaggacttgtgcattcaaacatagcaactgagcatattccaagccctgccaagaaagtaccaagactccctgccacagcagcagaacctgaagcagctgtcattagtaatggggaacattaa

SEQ ID NO: 13 - DNA, 399 bp, Homo sapiens
atgccaggcccggccaccgacgcggggaagatccctttctgcgacgccaaggaagaaatccgtgccgggctcgaaagctctgagggcggcggcggcccggagaggccaggcgcgcgcgggcagcggcagaacatcgtctggaggaatgtcgtcctgatgagcttgctccacttgggggccgtgtactccctggtgctcatccccaaagccaagccactcactctgctctgggcctacttctgcttcctcctggccgctctgggtgtgacagctggtgcccatcgcttgtggagccacaggtcctaccgggccaagctgcctctgaggatatttctggctgtcgccaactccatggctttccaggcagcagcagaagtggtcctgacccagatgccctga

SEQ ID NO: 14 - DNA, 1050 bp, Homo sapiens
atggagaacgcgcacaccaagacggtggaggaggtgctgggccacttcggcgtcaacgagagtacggggctgagcctggaacaggtcaagaagcttaaggagagatggggctccaacgagttaccggctgaagaaggaaaaaccttgctggaacttgtgattgagcagtttgaagacttgctagttaggattttattactggcagcatgtatatcttttgttttggcttggtttgaagaaggtgaagaaacaattacagcctttgtagaaccttttgtaattttactcatattagtagccaatgcaattgtgggtgtatggcaggaaagaaatgctgaaaatgccatcgaagcccttaaggaatatgagcctgaaatgggcaaagtgtatcgacaggacagaaagagtgtgcagcggattaaagctaaagacatagttcctggtgatattgtagaaattgctgttggtgacaaagttcctgctgatataaggttaacttccatcaaatctaccacactaagagttgaccagtcaattctcacaggtgaatctgtctctgtcatcaagcacactgatcccgtccctgacccacgagctgtcaaccaagataaaaagaacatgctgttttctggtacaaacattgctgctgggaaagctatgggagtggtggtagcaactggagttaacaccgaaattggcaagatccgggatgaaatggtggcaacagaacaggagagaacaccccttcagcaaaaactagatgaatttggggaacagctttccaaagtcatctcccttatttgcattgcagtctggatcataaatattgggcacttcaatgacccggttcatggagggtcctggatcagaggtgctatttactactttaaaattgcagtggccctggctgtagcagccattcctgaaggtctgcctgcagtcatcaccacctgcctggctcttggaactcgcagaatggcaaagaaaaatgccattgttcgaagcctcccgtctgtggaaacccttggttgtacttctgttatctgctca

SEQ ID NO: 15 - DNA, 1302 bp, Homo sapiens
atggcaacatcaggtgactgtcccagaagtgaatcgcagggagaagagcctgctgagtgcagtgaggcgggtctcctgcaggagggagtacagccagaggagtttgtggccatcgcggactacgctgccaccgatgagacccagctcagttttttgagaggagaaaaaattcttatcctgagacaaaccactgcagattggtggtggggtgagcgtgcgggctgctgtgggtacattccggcaaaccatgtggggaagcacgtggatgagtacgaccccgaggacacgtggcaggatgaagagtacttcggcagctatggaactctgaaactccacttggagatgttggcagaccagccacgaacaactaaataccacagtgtcatcctgcagaataaagaatccctgacggataaagtcatcctggacgtgggctgtgggactgggatcatcagtctcttctgtgcacactatgcgcggcctagagcggtgtacgcggtggaggccagtgagatggcacagcacacggggcagctggtcctgcagaacggctttgctgacatcatcaccgtgtaccagcagaaggtggaggatgtggtgctgcccgagaaggtggacgtgctggtgtctgagtggatggggacctgcctgctgtttgagttcatgatcgagtccatcctgtatgcccgggatgcctggctgaaggaggacggggtcatttggcccaccatggctgcgttgcaccttgtgccctgcagtgctgataaggattatcgtagcaaggtgctcttctgggacaacgcgtacgagttcaacctcagcgctctgaaatctttagcagttaaggagtttttttcaaagcccaagtataaccacattttgaaaccagaagactgtctctctgaaccgtgcactatattgcagttggacatgagaaccgtgcaaatttctgatctagagaccctgaggggcgagctgcgcttcgacatcaggaaggcggggaccctgcacggcttcacggcctggtttagcgtccacttccagagcctgcaggaggggcagccgccgcaggtgctcagcaccgggcccttccaccccaccacacactggaagcagacgctgttcatgatggacgacccagtccctgtccatacaggagacgtggtcacgggttcagttgtgttgcagagaaacccagtgtggagaaggcacatgtctgtggctctgagctgggctgtcacttccagacaagaccccacatctcaaaaagttggagaaaaagtcttccccatctggagatga

SEQ ID NO: 16 - DNA, 621 bp, Homo sapiens
gtgcatgtgaaatgctgatcccattcttaccaattggaagcacatgagaaaaaatattcacagaattgcctgacaagtgttacacattctagtatatatgttgcgaaatttgggcttggttgcattcgtgcttgagaaattgtttttggaggatgaaagtgctgttcatgtgattttacaggtgatgtggcatcttttctttaaactaaaaatgtaactaatgtgctgcatgtcttaaagaacatttcttcactgtcgtatgtgtcaggaatttaaaaatgtttcttactggtgtgtttgtgctctttttcaggaatccacattttcagtattccagctaactgcccccttcccgagttgacactgatttctattctataggcatgtcttgctataagtacagggaaaaaaattacaatctctttttctactagagttcaacatgatctttgaaaaaaatatatatttccaatgaggagagatacatcttgttatcagtcagcactctaaaaatgatcacttgtgcaaataggaacctactgctatgcatctttcttcttgctctgcctatatagctcatttcaaggtcatttggcatttgttcaaaaaataggatttgat

SEQ ID NO: 17 - DNA, 702 bp, Homo sapiens
caaggtgaaaataaaggctcggaaggggagcaatcttgaaaacatgtcatcccatggtggtgggaagtccctggagaagatcaggggaaacacagtcataggctgcaagtctataagataattccattggggagggagcccatttgtcatgcatggctgcaaggggcagatacaagtgtggagtaagcttgcaagagctgatcctggtcccagagagggaaaaatatgccttggtgggtaatgaaccttttgttcccagaggcagaaggattgggactaggccaacatagagattggcgatggttgtgagattctaagagtgtgtgtgcatcttgacaatattagaggaggctgagcccaagcaggcacattctcttcgacccctccctcattcagtctgctttggagtctactgaacatcaagcttgctatgagcaggatcttagagctgaggaattggcctcccaatccgaacaggtgttataatcctttcttaataggttgtgctgtggacccaatgtgagggctgtgctggtgtaaatggtgacatattgagctggggggatgctttcggggtggggggactggttccattccatcaaaggccctcttgagagtctatccagggacccattgttttactttaacagaccagaaaagatgtttgttttccatgtcattacccccaggggataccgaatg

SEQ ID NO: 18 - DNA, 652 bp, Homo sapiens
ttataacaaaccctgagaaccaaaatgaacgaaaatctgttcgcttcattcattgcccccacaatcctaggcctacccgccacagtactgatcattctatttccccctctattgatccccacctccaaatatctcatcaacaaccgactaatcaccacccaacaatgactaatcaaactaacctcaaaacaaatgataaccatacacaacactaaaggacgaacctgatctcttatactagtatccttaatcatttttattgccacaactaacctcctcggactcctgcctcactcatttacaccaaccacccaactatctataaacctagccatggccatccccttatgagcgggcgcagtgattataggctttcgctctaagattaaaaatgccctagcccacttcttaccacaaggcacacctacaccccttatccccatactagttattatcgaaaccatcagcctactcattcaaccaatagccctggccgtacgcctaaccgctaacattactgcaggccacctactcatgcacctaattggaagcgccaccctagcaatatcaaccattaaccttccctctacacttatcatcttcacaattctaattctactgactatcctagaaatcgctgtcgccttaatcc

SEQ ID NO: 19 - DNA, 567 bp, Homo sapiens
aacactcacaacaaaactaactaatactaacatctcagacgctcaggaaatagaaaccgtctgaactatcctgcccgccatcatcctagtcctcatcgccctcccatccctacgcatcctttacataacagacgaggtcaacgatccctcccttaccatcaaatcaattggccaccaatggtactgaacctacgagtacaccgactacggcggactaatcttcaactcctacatacttcccccattattcctagaaccaggcgacctgcgactccttgacgttgacaatcgagtagtactcccgattgaagcccccattcgtataataattacatcacaagacgtcttgcactcatgagctgtccccacattaggcttaaaaacagatgcaattcccggacgtctaaaccaaaccactttcaccgctacacgaccgggggtatactacggtcaatgctctgaaatctgtggagcaaaccacagtttcatgcccatcgtcctagaattaattcccctaaaaatctttgaaatagggcccgtatttaccctatagcaccccctctagag

SEQ ID NO: 20 - protein, 39 aa, Homo sapiens
MLRNLGLVAFVLEKLFLEDESAVHVILQVMWHLFFKLKM

SEQ ID NO: 21 - protein, 33 aa, Homo sapiens
MCQEFKNVSYWCVCALFQESTFSVFQLTAPFPS

SEQ ID NO: 22 - protein, 462 aa, Homo sapiens
MGKEKTHINIVVIGHVDSGKSTTTGHLIYKCGGIDKRTIEKFEKEAAEMGKGSFKYAWVLDKLKAERERGITIDISLWKFETSKYYVTIIDAPGHRDFIKNMITGTSQADCAVLIVAAGVGEFEAGISKNGQTREHALLAYTLGVKQLIVGVNKMDSTEPPYSQKRYEEIVKEVSTYIKKIGYNPDTVAFVPISGWNGDNMLEPSANMPWFKGWKVTRKDGNASGTTLLEALDCILPPTRPTDKPLRLPLQDVYKIGGIGTVPVGRVETGVLKPGMVVTFAPVNVTTEVKSVEMHHEALSEALPGDNVGFNVKNVSVKDVRRGNVAGDSKNDPPMEAAGFTAQVIILNHPGQISAGYAPVLDCHTAHIACKFAELKEKIDRRSGKKLEDGPKFLKSGDAAIVDMVPGKPMCVESFSDYPPLGRFAVRDMRQTVAVGVIKAVDKKAAGAGKVTKSAQKAQKAK

SEQ ID NO: 23 - protein, 463 aa, Homo sapiens
MGKEKTHINIVVIGHVDSGKSTTTGHLIYKCGGIDKRTIEKFEKEAAEMGKGSFKYAWVLDKLKAERERGITIDISLWKFETTKYYITIIDAPGHRDFIKNMITGTSQADCAVLIVAAGVGEFEAGISKNGQTREHALLAYTLGVKQLIVGVNKMDSTEPAYSEKRYDEIVKEVSAYIKKIGYNPATVPFVPISGWHGDNMLEPSPNMPWFKGWKVERKEGNASGVSLLEALDTILPPTRPTDKPLRLPLQDVYKIGGIGTVPVGRVETGILRPGMVVTFAPVNITTEVKSVEMHHEALSEALPGDNVGFNVKNVSVKDIRRGNVCGDSKSDPPQEAAQFTSQVIILNHPGQISAGYSPVIDCHTAHIACKFAELKEKIDRRSGKKLEDNPKSLKSGDAAIVEMVPGKPMCVESFSQYPPLGRFAVRDMRQTVAVGVIKNVEKKSGGAGKVTKSAQKAQKAGK

SEQ ID NO: 24 - DNA, 30 bp, Artificial Sequence
  Synthetic construct
  misc_feature (1)...(30): 5′ Bax PCR primer
ggaagatctatggacggttccggtgaacaa

SEQ ID NO: 25 - DNA, 32 bp, Artificial Sequence
  Synthetic construct
  misc_feature (1)...(32): 3′ Bax PCR primer
ctagtctagaacccatcttcttccagatggtc

SEQ ID NO: 26 - DNA, 24 bp, Artificial Sequence
  Synthetic construct
  misc_feature (1)...(24): 5′ pSYE224 sequencing PCR primer
cctctatactttaacgtcaaggag

SEQ ID NO: 27 - DNA, 23 bp, Artificial Sequence
  Synthetic construct
  misc_feature (1)...(23): 3′ pSYE224 sequencing PCR primer
cgtgaatgtaagcgtgacataac

SEQ ID NO: 28 - DNA, 34 bp, Artificial Sequence
  Synthetic construct
  misc_feature (1)...(34): 5′ Bcl2A1 PCR primer
ggactagtgccaccatgacagactgtgaatttgg

SEQ ID NO: 29 - DNA, 29 bp, Artificial Sequence
  Synthetic construct
  misc_feature (1)...(29): 3′ Bcl2A1 PCR primer
ccgctcgagacagtattgcttcaggagag

SEQ ID NO: 30 - DNA, 39 bp, Artificial Sequence
  Synthetic construct
  misc_feature (1)...(39): 5′ FKBP2 PCR primer
ggactagtgccaccatgaggctgagctggttccgggtcc

SEQ ID NO: 31 - DNA, 37 bp, Artificial Sequence
  Synthetic construct
  misc_feature (1)...(37): 3′ FKBP2 PCR primer
ccgctcgagcagctcagttcgtcgctctattttgagc

SEQ ID NO: 32 - DNA, 40 bp, Artificial Sequence
  Synthetic construct
  misc_feature (1)...(40): 5′ alpha synuclein PCR primer
ggactagtgccaccatggatgtattcatgaaaggactttc

SEQ ID NO: 33 - DNA, 36 bp, Artificial Sequence
  Synthetic construct
  misc_feature (1)...(36): 3′ alpha synuclein PCR primer
ccgctcgagggcttcaggttcgtagtcttgataccc

SEQ ID NO: 34 - DNA, 40 bp, Artificial Sequence
  Synthetic construct
  misc_feature (1)...(40): 5′ VAMP3 PCR primer
ggactagtgccaccatgtctacaggtccaactgctgccac

SEQ ID NO: 35 - DNA, 34 bp, Artificial Sequence
  Synthetic construct
  misc_feature (1)...(34): 3′ VAMP3 PCR primer
ccgctcgagtgaagagacaacccacacgatgatg

SEQ ID NO: 36 - DNA, 45 bp, Artificial Sequence
  Synthetic construct
  misc_feature (1)...(45): 5′ ETEF1A1 PCR primer
ggactagtgccaccatgggaaaggaaaagactcatatcaacattg

SEQ ID NO: 37 - DNA, 35 bp, Artificial Sequence
  Synthetic construct
  misc_feature (1)...(35): 3′ ETEF1A1 PCR primer
ccgctcgagtttagccttctgagctttctgggcag

SEQ ID NO: 38 - DNA, 979 bp, Homo sapiens
tgctatgggctctggcactgcggtggctaaaaccgcctctgagatggtcctggcggatgacaacttctccaccattgtggctgccgttgaggaggggcgggcaatctacaacaacatgaaacagttcatccgctacctcatctcgtccaacgtcggggaagttgtctgtattttcctgacagcagcccttggatttcccgaggctttgattcctgttcagctgctctgggtcaatctggtgacagatggcctgcctgccactgcactggggttcaaccctcctgatctggacatcatgaataaacctccccggaacccaaaggaaccattgatcagcgggtggctctttttccgttacttggctattggctgttacgtcggcgctgctaccgtgggtgctgctgcatggtggttcattgctgctgacggtggtccaagagtgtccttctaccagctgagtcatttcctacagtgtaaagaggacaacccggactttgaaggcgtggattgtgcaatctttgaatccccatacccgatgacaatggcgctctctgttctagtaactatagaaatgtgtaacgccctcaacagcttgtccgaaaaccagtccttgctgaggatgcccccctgggagaacatctggctcgtgggctccatctgcctgtccatgtcactccacttcctgatcctctatgtcgaacccttgccactcatcttccagatcacaccgctgaacgtgacccagtggctgatggtgctgaaaatctccttgcccgtgattctcatggatgagacgctcaagtttgtggcccgcaactacctggaacctggtaaagagtgtgtgcagcctgccaccaaatcctgctcgttctcggcatgcaccgatgggatttcctggccgtttgtgctgctcataatgcccctggtgatctgggtctatagcacagacactaactttagcgatatgttctggtcttga

(19) United States
(12) Patent Application Publication
(10) Pub. No.: US 2009/0258794 A1
(43) Pub. Date: Oct. 15, 2009

(54) APOPTOSIS METHODS, GENES AND PROTEINS

(76) Inventor: Bhabatosh Chaudhuri, Leicester (GB)

Correspondence Address: MORRISON & FOERSTER LLP, 12531 HIGH BLUFF DRIVE, SUITE 100, SAN DIEGO, CA, 92130-2040, US

(21) Appl. No.: 12/279,642

(22) PCT Filed: Feb. 15, 2007
(86) PCT No.: PCT/GB2007/000540

§ 371 Date: Aug. 15, 2008

(30) Foreign Application Priority Data
Feb. 15, 2006 (GB) 0602992.0

(51) Int. Cl. C40B 30/06; C12N 1/19; C40B 40/02

(52) U.S. Cl. 506/10; 435/254.21; 506/14

(57) ABSTRACT

A W303a Saccharomyces cerevisiae yeast cell which contains a polynucleotide that encodes a functional Bax polypeptide under the control of a galactose-inducible promoter that is integrated at the LEU2 chromosomal locus. A kit of parts comprising the yeast cells and a yeast plasmid vector suitable for transforming a cDNA library into the yeast cells. Use of the yeast cell for screening a cDNA library for a polynucleotide that is or encodes an inhibitor of Bax-mediated apoptosis. Genes and polypeptides that inhibit Bax-mediated apoptosis and which were identified from a human hippocampus cDNA library screened in the yeast cells. A method of combating Bax-mediated apoptosis in a cell using an inhibitor of Bax-mediated apoptosis which was identified from a human hippocampus cDNA library screened in the yeast cells. A method of promoting Bax-mediated apoptosis in a cell using an inhibitor or antagonist of the anti-apoptotic polypeptides identified from a human hippocampus cDNA library screened in the yeast cells.

The present invention relates to the identification of genes and proteins that regulate apoptosis. In particular, the invention relates to a yeast strain that is useful in a method of identifying regulators of apoptosis, screening methods employing the yeast strain, and genes and proteins thus identified. The invention further relates to medical uses of the identified genes and proteins.

Aberrant expression of apoptosis-regulatory proteins is often the cause of diverse diseases such as cancer, rheumatoid arthritis, neurodegeneration and cardiovascular disease. Accumulating evidence strongly suggests that apoptosis contributes to neuronal cell death in a variety of neurodegenerative contexts. Apoptosis plays a central role in human neurodegenerative disease as observed in stroke, spinal cord trauma, head injury, spinal muscular atrophy (SMA), amyotrophic lateral sclerosis (ALS), Alzheimer's disease (AD), Parkinson's disease (PD) and Huntington's disease (HD).

The pro-apoptotic molecule Bax is required for death of sympathetic and motor neurons in the setting of trophic factor deprivation. Furthermore, adult Bax-deficient transgenic mice have more motor neurons than do their wild-type counterparts. These findings suggest that Bax controls naturally occurring cell death during development in many neuronal populations. It is also been observed that Bax is a critical mediator of naturally occurring death of peripheral and CNS neurons during embryonic life (Davies, 2000).

Under certain experimental conditions, the known anti-apoptotic proteins Bcl-2 and Bcl-xL counteract the activity of Bax. It is assumed that apoptotic events are stimulated when concentrations of pro-apoptotic proteins exceed those of anti-apoptotic proteins. Such apoptotic events include changes in mitochondria which ultimately lead to the activation of a family of cysteine proteases called caspases. This results in the digestion of the dying cell from within, a hallmark of apoptosis (Cory et al, 2003).

A better understanding of the genes and proteins that regulate apoptosis, and especially of those that negatively-control (i.e. inhibit) apoptosis, can lead to the design of new treatments that would prevent the inappropriate activation of apoptosis or arrest the apoptotic process once started. Discovery of these anti-apoptotic genes and proteins will be beneficial for developing new practical therapeutic approaches for diseases characterised by inappropriate apoptosis, including both acute and chronic neurodegenerative conditions.

### The Role of Apoptosis in Neurodegeneration

Stroke, AD, PD, HD, SMA, and motor neuron disease including ALS have all been associated with apoptosis. Unlike necrosis, which involves cell swelling, plasma membrane lysis and massive cell death, apoptosis involves individual cell death with caspase activation, oxidative stress, perturbed calcium homeostasis and mitochondrial dysfunction. Some survival signals protect against this by suppressing oxy-radicals and stabilizing calcium homeostasis and mitochondrial function. Mitochondria in many forms of apoptosis show increased oxy-radical production, opening of pores in their membranes and release of cytochrome c. As evidence of this, manganese superoxide dismutase and cyclosporin A, which suppress oxidative stress and membrane pore formation, also prevent neuronal death in experimental models (Pong, 2003; Sullivan et al, 2005).

The B-cell lymphoma-2 (Bcl-2) family of proteins includes both pro- and anti-apoptotic members. Anti-apoptotic members in neurons include Bcl-2 and Bcl-xL; pro-apoptotic members include Bcl-2-associated X-protein (Bax) and Bcl-associated death promoter (Bad). For example, the over-expression of Bcl-2 in cell cultures and transgenic mice increases resistance of neurons to death induced by excitotoxic, metabolic and oxidative insults. Neurons lacking Bax are protected against apoptosis. Bcl-2 proteins may control cell death by interacting with proteins associated with the mitochondria, causing a change in ions across mitochondrial membranes (Soane & Fiskum, 2005; Kirkland et al, 2002).

Caspases are cysteine proteases. Caspase-8 is activated in response to death receptors (e.g. Fas, p75 neurotrophin receptor). These upstream caspases activate effector caspases (e.g. caspase-3) and are able to elicit apoptosis independent of mitochondrial alterations (Davies, 2000). Effector caspases are also activated in response to mitochondrial changes and cytochrome c release and then activate a DNase. Caspases can also cleave various proteins e.g. AMPA, actin etc. Levels of Par-4 increase rapidly (Mundle, 2005). A leucine zipper domain in the carboxyl terminus of Par-4 is essential for its pro-apoptotic function and its interactions with other proteins, including protein kinase C and Bcl-2 (Leroy et al, 2005).

In the initiation phase of apoptosis, the death signal activates an intracellular cascade of events that may involve increases in levels of oxy-radicals and Ca²⁺, production of Par-4 and translocation of pro-apoptotic Bcl-2 family members (Bax and Bad) to the mitochondrial membrane. Certain caspases (e.g. caspase-8) can act early in the cell death process before, or independently of, mitochondrial changes.

The effector phase of apoptosis involves increased mitochondrial Ca²⁺ and oxy-radical levels, the formation of permeability transition pores in the mitochondrial membrane, and release of cytochrome c into the cytosol. Cytochrome c forms a complex with apoptotic protease-activating factor 1 (Apaf-1) and caspase-9 (Hajra & Liu, 2004).

In the degradation phase of apoptosis, activated caspase-9 activates caspase-3, leading to cleavage of caspase and other enzyme substrates; changes in the plasma membrane occur (blebbing and exposure of phosphatidylserine on the cell surface); signals are released that stimulate cell phagocytosis by macrophages/microglia; and the nuclear chromatin becomes condensed and fragmented (Budihardjo et al, 1999).

Mitochondrial changes are probably pivotal in the cell death decision in many cases.

Signals which are known to trigger apoptosis in neurons include:

[1] Lack of neurotrophic factor support. Bax is required for the apoptotic death of sympathetic neurons deprived of NGF. After NGF withdrawal, Bax translocates from the cytoplasm to the mitochondria of these cells and induces release of cytochrome c. Withdrawing NGF from sympathetic neurons causes an increase of mitochondria-derived reactive oxygen species (ROS). Suppressing these ROS inhibits apoptosis. Bax deletion blocks death and prevents the ROS burst, thus Bax lies upstream from increased ROS (Heaton et al, 2003).
[2] Over-activation of glutamate receptors, for example through calcium influx or excitotoxicity (Johnston, 2005).
[3] Increased oxidative stress, for example free radicals (e.g. superoxide anion radical, hydroxyl radical) damage cellular lipids, proteins and nucleic acids (Halliwell & Whiteman, 2004).
[4] Metabolic stress, e.g. after a stroke or during ageing, levels of glucose, oxygen and other molecules required for ATP production are decreased (Poon et al, 2004).
[5] Environmental toxins (Valko et al, 2005; Savolainen et al, 1998).

Factors which are known to be anti-apoptotic triggers, include:

[1] Telomerase consists of a catalytic reverse-transcriptase subunit (TERT), an RNA template and regulatory proteins. Telomerase activity is increased during development, and then downregulated. Telomerase activity and TERT are associated with increased resistance of neurons to apoptosis in experimental models of developmental neuronal death and neurodegenerative disorders. The anti-apoptotic action of TERT in neurons is exerted at an early step before mitochondrial alterations and caspase activation (Sung et al, 2005).
[2] Stress can induce the expression of neurotrophic factors and heat-shock proteins. The neurotrophic factors, in turn, act in an autocrine or paracrine manner to activate cell surface receptor-mediated kinase signalling pathways that induce expression of survival-promoting genes coding for proteins such as antioxidant enzymes. Neurotrophic factors (brain-derived neurotrophic factor (BDNF), nerve growth factor (NGF) basic fibroblast growth factor (bFGF)) and cytokines (tumour necrosis factor (TNF)-α, ciliary neurotrophic factor (CNTF) and leukaemia inhibitory factor (LIF) can prevent neuronal death in experimental models of neuronal death (Zweifel et al, 2005).
[3] Heat-shock proteins act as chaperones for many proteins, maintaining protein stability. They may also interact directly with caspases, inhibiting their activation (Sreedhar & Csermely, 2004).
[4] Calcium, as well as promoting neuronal death, can also activate four distinct survival pathways (Distelhorst & Shore, 2004):
(a) Activation of protein kinase B (PKB/Akt) by calcium/calmodulin-dependent protein kinase (Yano et al, 2005; Chong et al, 2005).
(b) Regulation of cellular responses to stress, activating transcription through cyclic-AMP response element-binding protein (CREB), which can promote neuron survival in models of developmental cell death (Yano et al, 2005; Rouaux et al, 2004).
(c) Activation of actin-severing protein gelsolin which induces actin depolymerisation, resulting in suppression of calcium influx through membrane NMDA receptors and voltage-dependent calcium channels. This may occur through intermediary actin-binding proteins that interact with NMDA receptor and calcium channel proteins (Harms et al, 2004; Burtnick et al, 2004).
(d) Calcium and secreted amyloid precursor protein α, which increase cyclic GMP production, can induce activation of potassium channels and the transcription factor NF-κB, and increase resistance of neurons to excitotoxic apoptosis (Cardoso & Oliveira, 2003).

It is difficult to demonstrate apoptosis in the brains of patients suffering from neurodegenerative disease states. Apoptosis usually occurs rapidly (hours), so at any one time few cells will be showing classic features. Thus much of the evidence in support of apoptosis comes from animal and cell-culture models.

In AD, early changes are observed in the hippocampus, also later the cortex. There is some evidence of calcium-mediated proteolysis and oxidative stress. Increased DNA damage and caspase activity, and alterations in expression of apoptosis-related genes such as Bcl-2 family members, Par-4 and DNA damage response genes have been found in neurons associated with amyloid deposits in the brains of patients with AD. Expression-profile analysis of genes in brain tissue samples from AD patients show a marked decrease in expression of an anti-apoptotic gene called NCKAP1 (NCK-associated protein 1) (Yamamoto & Behl, 2001).

Mutations in the amyloid precursor protein (APP), Presenilin 1 (PS1) and Presenilin 2 (PS2) genes have been shown to cause early onset AD. Cleavage of APP by β-secretase (BACE) results in the production of the 40-42 peptide Aβ and a secreted product called sAPPβ. This only occurs 5-10% of the time. Usually α-secretase (ADAMs family of metalloproteases) cleaves APP and Aβ is not produced, and the neurite promoting sAPPα is secreted. Aβ exposure in cultured neurons can induce apoptosis directly, and increase vulnerability to death by oxidative stress. Aβ probably sensitizes neurons by membrane lipid peroxidation. This impairs the function of ATPases and glucose and glutamate transporters, resulting in membrane depolarization, ATP depletion, excessive calcium influx and mitochondrial dysfunction. Antioxidants that suppress lipid peroxidation and drugs that stabilize cellular calcium homeostasis can protect neurons against Aβ-induced apoptosis. Neurotrophic factors and cytokines can also protect against Aβ. Mutations in APP, PS1 and PS2 all cause an increase in Aβ production and in some cases may also cause an increase in its more toxic form, Aβ42.

APP is also a substrate for caspase-3. Caspase-mediated cleavage of APP can release a carboxy-terminal peptide called C31 that is a potent inducer of apoptosis. When mutant PS1 is expressed in cultured cells and in transgenic and knock-in mice, neurons become susceptible to death induced by various insults, including trophic-factor withdrawal, exposure to Aβ or glutamate, and energy deprivation. Mutant PS1 acts at an early step before Par-4 production, mitochondrial dysfunction and caspase activation. Calcium homeostasis in the endoplasmic reticulum is disturbed such that more calcium is released when neurons are exposed to potentially damaging oxidative and metabolic insults. Agents that suppress ER calcium release, including dantrolene and xestospongin, can counteract the effects of the mutations.

In PD, dopamine neurons degenerate in the substantia nigra. Environmental and genetic factors may sensitize dopamine neurons to age-related increases in oxidative stress and energy deficits. Environmental toxins have been implicated—monkeys and people exposed to the toxin 1-methyl-4-phenyl-1,2,3,6-tetrahydropyridine (MPTP) show Parlinson's-like symptoms. Brain tissue from patients with PD show apoptosis-related DNA damage and gene activation in the death of dopamine neurons. Levels of Par-4 are increased in dopamine neurons of the substantia nigra before their death, and suppression of Par-4 expression protects dopamine neurons against death. Caspase-1 inhibition, drugs that suppress macromolecular synthesis, and neurotrophic factors, such as glial cell-derived neurotrophic factor (GDNF), can protect dopamine neurons in PD models. In rare cases, mutations in α-synuclein, a component of Lewy bodies, cause Parkinson's disease cases. Expression of mutant α-synuclein in cultured cells promotes apoptosis. Normally parkin and ubiquitin are involved in the removal of synuclein via apoptosis. If this process goes awry, for instance with a defective parkin gene, then apoptosis fails to occur. If synuclein is not eliminated in these cells, it builds up and becomes toxic to dopamine. In such cases, synuclein accumulates in Lewy bodies.

Some patients with PD have a deficit of the mitochondrial complex I which may arise from, or contribute to, increased cellular oxidative stress. Chronic complex I inhibition caused by rotenone induces features of PD in rats, including selective nigrostriatal dopaminergic degeneration and Lewy bodies with α-synuclein-positive inclusions. In rotenone-induced cell death of dopaminergic SH-SY5Y cells, rotenone induces Bad dephosphorylation without changing the amount of Bad proteins. Rotenone also increases the amount of α-synuclein in cells showing morphological changes. Rotenone causes a decrease in Bad and an increase in α-synuclein binding to 14-3-3 proteins. Dephosphorylation by calcineurin activates Bad. The calcineurin inhibitor tacrolimus (FK506) suppresses rotenone-induced Bad dephosphorylation and apoptosis. Inhibition of caspase-9, which functions downstream from Bad, completely suppresses rotenone-induced apoptosis. MPP+ inhibits mitochondrial complex-1 and aconitase activities leading to enhanced H₂O₂ generation, TfR expression and α-synuclein expression and aggregation. Cells over-expressing α-synuclein exacerbate MPP+ toxicity whereas antisense α-synuclein treatment totally abrogated MPP+ induced apoptosis in neuroblastoma cells without affecting oxidant generation. The increased cytotoxic effects of α-synuclein in MPP+treated cells were attributed to inhibition of mitogen-activated protein kinase (MAPK) and proteasomal function (Kalivendi et al, 2004).

### Yeast as a Tool for Apoptosis Research

As described above, there are a few known regulators of apoptosis, some of which stimulate cell death while others prevent it. Human pro-apoptotic proteins expressed in the yeast Saccharomyces cerevisiae (S. cerevisiae) can block cell growth in a mitochondria-independent or dependent manner, and can cause cell death in the presence of functional mitochondria. Many of the hallmarks of mammalian apoptosis are manifested in dying yeast and known anti-apoptotic proteins can overcome mitochondria-dependent yeast death. Therefore, a yeast screening system for regulators of apoptosis provides a useful mimic of the human system and can allow exploration of areas not amenable to mammalian test systems (e.g. mitochondria-independent growth arrest pathways).

In GB 2 326 413 and Greenhalf et al (1996), the inventor previously described a method of screening cDNA for putative apoptosis inhibitors in the S. cerevisiae yeast strain HT444. A pRS305 yeast integrating vector containing a polynucleotide encoding the human Bax protein under the control of the GAL10 promoter, the SUC2 transcription terminator and the LEU2 selectable marker gene, was integrated into HT444 cells to obtain the yeast strain HT444_bax. Expression of the human Bax protein in the presence of galactose stopped growth of, and killed, the HT444_bax cells. When the HT444_bax cells were transformed with yeast plasmids containing polynucleotides encoding bcl-2 and bcl-xL, expression of these proteins was shown to restore yeast cell growth in the Bax-expressing cells. A human cerebellum cDNA library was screened using this system.

Greenhalf et al (1996) demonstrated that although Bax induction invariably prevents yeast cell growth under all circumstances, it does not lead to death in “petite” cells which cannot respire because they lack functional mitochondria. This indicated that Bax-mediated growth inhibition and cell death is linked to mitochondrial function and respiration. Unlike mammalian cells which do not survive in the absence of mitochondria, yeast has a viable alternative—the fermentation pathway. Galactose is a less efficient inhibitor of respiration in yeast than glucose. Thus the expression of Bax in yeast under a galactose-inducible promoter leads to respiration and more profound cell death than the expression of Bax under a glucose-inducible promoter (when yeast cells undergo fermentation).

Manon et al (1997) reported that Bax-induced growth arrest is related to a decrease in mitochondrial cytochtrome c oxidase levels, and an increase in cytochrome c release from the mitochondria to the cytosol.

Ligr et al (1998) later confirmed that mammalian Bax triggers apoptotic changes in yeast that strongly resemble the apoptotic changes in mammalian cells.

Xu & Reed (1998) identified the mammalian apoptosis suppressor, Bax Inhibitor-1 (BI-1) using a functional yeast screening system utilising Bax expression under a galactose inducible promoter in yeast strain BF264-15Dau.

US 2005/0148062 described the use of a Ty transposon based vector which espresses the mouse Bax-α protein for the analysis of differential gene expression upon Bax-induced cell death in S. cerevisiae strain INVSc1.

The use of yeast as a tool for apoptosis research was reviewed in 1999 by Matsuyama et al (1999), and yeast as a tool for the study of Bax/mitochondrial interactions in cell death was reviewed by Priault et al (2003).

The inventor has now developed a Saccharomyces cerevisiae yeast strain, W303baxleu, that is particularly useful in a functional screening system for identifying genes and proteins that regulate apoptosis. As described below, the yeast strain W303baxleu has superior properties to any of the strains previously described for screening for regulators of apoptosis. In particular, the false positive rate using W303baxleu is surprisingly low. Indeed, the inventor has found that yeast strain W303baxleu has a much lower false positive rate than otherwise equivalent W303 strains in which the BAX expression cassette has been integrated at the ADE2, HIS3 or TRP1 loci. Accordingly, the use of W303baxleu is expected to lead to the identification of several potential therapeutic targets more quickly and at a reduced cost. Indeed, using W303baxleu, the inventor has identified a number of potential new apoptosis regulators (see Table 1), some of which the inventor has independently confirmed as having activity as inhibitors of Bax-mediated apoptosis in mammalian cells.

A first aspect of the invention provides a Saccharomyces cerevisiae (S. cerevisiae) yeast cell which has the genotype MAT-a, ade2-1, trp1-1, leu2-3, leu2-112, his3-11, his3-15, ura3-1, can1-100, and which contains a polynucleotide that encodes a functional Bax polypeptide under the control of a galactose-inducible promoter integrated at the LEU2 chromosomal locus.

The S. cerevisiae CAN1 gene is an arginine permease. The can1-100 mutation is usually a silent mutation in strains with the usual leu, ura or ade markers, so it is possible not to be aware that the can1-100 mutation is present in such strains. However, the can1-100 mutation can pose a problem in His mutant cells (such as W303a). These cells have a slow growth phenotype if grown on exogenous histidine. This slow growth phenotype in the presence of exogenous histidine (which is absolutely essential for growth in a strain which is, for example, a His3 mutant) helps in the screening and eliminates false positives. Without wishing to be bound by theory, the inventor proposes that other His mutant yeast strains that bear the can1-100 mutation, together with an integrated copy of human BAX (preferably synthesised using yeast-biased codons, and optionally with a c-myc tag at the C-terminus), would be helpful in eliminating false positives in a Bax-mediated apoptosis screen, particularly in strains which are also Mat-a (as opposed to Mat-alpha).

A second aspect of the invention provides a S. cerevisiae yeast cell which has the genotype MAT-a, ade2-1, trp1-1, leu2-3, leu2-112, his3-11, his3-15, ura3-1, can1-100 and which contains a yeast integrating plasmid that comprises a polynucleotide that encodes a functional Bax polypeptide under the control of a galactose-inducible promoter, and which is suitable for integration at the LEU2 chromosomal locus.

By “suitable for integration at the LEU2 chromosomal locus” we mean that the plasmid is designed and constructed for targeted integration at the LEU2 locus.

Preferably, the S. cerevisiae yeast cell is W303 MATa (also known as W303-1A and W303a). W303 is a well known yeast strain. This strain was made diploid by transforming W301-18A (Rothstein, 1983, Meth. Enzymol. 101: 202-211) with an HO-containing plasmid. The diploid was dissected to obtain the isogenic MAlTa (W303-1A) and MATalpha (W303-1B) strains (Thomas & Rothstein, 1989, Cell 56: 619-630).

As described at http://www.yeastgenome.org/straintable.shtml#W303, W303 has the following genotype: leu2-3, 112; trp1-1; can1-100; ura3-1; ade2-1; his3-11,15; [phi+]. W303-1A possesses a ybp1-1 mutation (I7L, F328V, K343E, N571D) which abolishes Ybp1p function, increasing sensitivity to oxidative stress (Veal et al, 2003). W303 also contains a bud4 mutation that causes haploids to bud with a mixture of axial and bipolar budding patterns (Voth, et al, 2005). In addition, the original W303 strain contains the rad5-535 allele (a G to R change at position 535; see Fan et al, 1996, Genetics 142: 749). Bud4 and Rad5 are cell division cycle associated genes.

More detailed information on W303 is available at http://www.yeastgenome.org/community/W303.html, which is incorporated herein in its entirety by reference.

The amino acid sequence of a functional human Bax polypeptide is listed in SEQ ID No: 3 (FIG. 61). Other suitable functional Bax polypeptides are well known in the art and include those described in GB 2 326 413, Greenhalf et al (1996), Manon et al (1997), Ligr et al (1998) and Xu & Reed (1998), supra.

The functional Bax polypeptide may be the human Bax sequence, or a pro-apoptotic fragment or variant thereof. Bax is also known as BCL2-associated X protein.

By a “functional” Bax polypeptide we mean a polypeptide that has the ability to induce cell death in yeast cells under the experimental conditions described below. Suitable yeast cells include W303 cells.

It is preferred if the functional Bax polypeptide is also able to induce cell death in mammalian cells under the conditions described below. Suitable mammalian cells are HE 293, COS-1 and SH-SY5Y cells. The observations in yeast can be confirmed in a wide number of mammalian lines; HE 93 cells were chosen because they are claimed to have neuronal features.

By “a functional Bax polypeptide” we include the gene product of the human Bax gene and naturally occurring variants thereof. The mRNA sequence of the human Bax beta transcriptional variant, which encodes the longest isoform (beta), can be found in Accession No NM_{—}004324, and the corresponding polypeptide sequence of isoform beta can be found in Accession No NP_{—}004315.

The human Bax alpha transcriptional variant contains a distinct 3′ coding region and 3′ UTR when compared to variant beta. It encodes an isoform (alpha) that has a shorter and different C terminus, as compared to isoform beta. The mRNA sequence of the alpha variant, which encodes the alpha isoform, can be found in Accession No NM_{—}138761, and the corresponding polypeptide sequence of the alpha isoform can be found in Accession No NP_{—}620116. Human Bax isoform alpha is very similar to isoform psi.

It is preferred if the functional Bax polypeptide is the alpha isoform, which is known to exist in most cells. The Bax alpha form is more effective at killing cells than the beta and delta forms.

The human Bax gamma transcriptional variant lacks a segment within the coding region, which leads to a translation frameshift, when compared to variant beta. The resulting gamma isoform has a shorter and distinct C terminus, as compared to isoform beta. The mRNA sequence of the gamma variant, which encodes the gamma isoform, can be found in Accession No NM_{—}138762, and the corresponding polypeptide sequence of isoform gamma can be found in Accession No NP_{—}620117.

The human Bax delta transcriptional variant lacks a segment within the coding region, and contains a distinct 3′ coding region and 3′ UTR, when compared to variant beta. The translation remains in-frame, and results in an isoform (delta) that is missing an internal segment, and has a shorter and different C terminus, as compared to isoform beta. The mRNA sequence of the delta variant, which encodes the delta isoform, can be found in Accession No NM_{—}138763, and the corresponding polypeptide sequence of isoform delta can be found in Accession No NP_{—}620118.

The human Bax epsilon transcriptional variant contains an extra segment within the coding region, and has a distinct 3′ coding region and 3′ UTR, when compared to variant beta. The extra segment causes a translation frameshift, and thus results in an isoform (epsilon) that has a shorter and distinct C terminus, as compared to isoform beta. The mRNA sequence of the epsilon variant, which encodes the epsilon isoform, can be found in Accession No NM_{—}138764, and the corresponding polypeptide sequence of isoform epsilon can be found in Accession No NP_{—}620119.

The human Bax sigma transcriptional variant contains a distinct 3′ coding region and 3′ UTR when compared to variant beta. It encodes an isoform (sigma) that has a shorter and different C terminus, as compared to isoform beta. The mRNA sequence of the sigma variant, which encodes the sigma isoform, can be found in Accession No NM_{—}138765, and the corresponding polypeptide sequence of isoform sigma can be found in Accession No NP_{—}620120.

Polynucleotides that encode the functional Bax are typically made by recombinant DNA technology. Suitable techniques for cloning, manipulation, modification and expression of nucleic acids, and purification of expressed proteins, are well known in the art and are described for example in Sambrook et al (2001) “Molecular Cloning, a Laboratoiy Manual”, 3^{rd} edition, Sambrook et al (eds), Cold Spring Harbor Laboratory Press, Cold Spring Harbor, N.Y., USA.

A functional Bax polypeptide does not have to be the full-length polypeptide, nor does it have to have an identical sequence to a wild-type Bax polypeptide, as long as it retains at least some of the pro-apoptotic activity of wild-type Bax, i.e. the functional Bax polypeptide must retain at least some of the ability of wild-type Bax to stimulate apoptotic cell death, or to block cell growth, under the conditions described below.

Thus the functional Bax polypeptide may include a derivative of a full-length Bax polypeptide that retains at least some of the pro-apoptotic activity of wild-type Bax. Suitable derivatives include variants or fragments of a full-length Bax polypeptide, or a variant of the fragment of the full-length polypeptide, that retains at least some of the pro-apoptotic activity of wild-type Bax.

By a “fragment” of human Bax we mean any portion of the polypeptide that stimulates apoptotic cell death. This can be tested for using the methods described herein, and preferably, when tested in W303 cells. Typically, the fragment has at least 30% of the apoptotic activity of the functional Bax polypeptide of SEQ ID No: 3 (listed in FIG. 61). It is more preferred if the fragment has at least 50%, preferably at least 70% and more preferably at least 90% of the apoptotic activity of the functional Bax polypeptide of SEQ ID No: 3. Most preferably, the fragment has 100% or more of the apoptotic activity of the functional Bax polypeptide of SEQ ID No: 3.

Variants of full-length Bax, or of a fragment thereof, include amino acid insertions, deletions and substitutions, either conservative or non-conservative, at one or more positions. By “conservative substitutions” is intended combinations such as Gly, Ala; Val, Ile, Leu; Asp, Glu; Asn, Gln; Ser, Thr; Lys, Arg; and Phe, Tyr. Such modifications may be made using the methods of protein engineering and site-directed mutagenesis, as described in Sambrook et al 2001, supra. Preferably, the variant Bax or variant Bax fragment retains at least 90% sequence identity with full-length human Bax, or the respective Bax fragment. More preferably, the variant Bax or variant Bax fragment has at least 91%, 92%, 93%, 94% or 95% sequence identity, and yet more preferably at least 96%, 97%, 98% or 99% sequence identity with full-length Bax, or the respective Bax fragment. Preferably, the variant Bax or the variant Bax fragment retains at least 30% of the pro-apoptotic activity of the functional Bax polypeptide of SEQ ID No: 3. It is more preferred if the variant Bax or the variant Bax fragment has at least 50%, preferably at least 70% and more preferably at least 90% of the pro-apoptotic activity of the functional Bax polypeptide of SEQ ID No: 3. Most preferably, the variant Bax or the variant Bax fragment has 100% or more of the pro-apoptotic activity of the functional Bax polypeptide of SEQ ID No: 3 (listed in FIG. 61).

It is appreciated that functional Bax polynucleotides and polypeptides from mammalian species other than humans may be employed in this invention, and reference to fragments or variants of the full-length Bax polypeptide should be construed accordingly.

Preferably, the codons of the polynucleotide encoding the functional Bax polypeptide are optimised for yeast as is well known in the art (Bennetzen & Hall, 1982). SEQ ID No: 2 is an example of a polynucleotide encoding a functional Bax polypeptide with codons optimised for yeast, and the functional Bax polypeptide it encodes is shown in SEQ ID No: 3 (both sequences are listed in FIG. 61).

The galactose-inducible promoter may be the GAL1 or GAL10 promoter (Johnston, 1987).

It is preferred that the polynucleotide encoding the functional Bax polypeptide in the S. cerevisiae yeast cell is terminated by a SUC2 transcription terminator sequence (Reeder & Lang, 1994). Other suitable transcriptional terminators are known in the art and include PHO5 and ADH1.

For the purposes of this invention, expression of the functional Bax polypeptide in the S. cerevisiae yeast cell in the presence of galactose preferably results in cell death. However, for petite yeast cells, expression of the functional Bax polypeptide results in inhibition of cell growth but not death.

In a preferred embodiment of the invention, the S. cerevisiae yeast cell is strain W303baxleu as described below. The W303 MAT-a strain was used for production of W303baxleu. W303 MAT-a (W303-1A) is available from Open Systems as Catalogue No. YSC 1058.

As discussed in detail below, when the functional Bax polypeptide is expressed in the presence of galactose in the S. cerevisiae yeast cells of the first or second aspects of the invention, the apoptotic effect of the functional Bax polypeptide is sufficient to arrest growth, and kill, virtually all of the cells. Therefore, these yeast cells are particularly useful in a method of screening for inhibitors of Bax-mediated apoptosis because the yeast strain has an exceptionally low level of background, i.e. false positives (see FIG. 53). In such a screening method, a plurality of polynucleotides, typically a cDNA library, is introduced into the yeast cells, and expression of the polynucleotides is induced. Only cells that contain a putative inhibitor of apoptosis show growth, and every cell that shows growth contains a putative inhibitor of apoptosis. Cells that contain proteins that down-regulate transcription mediated by the GAL1/GAL10 promoters in the presence of galactose would be false positives. Other possible false positives include cells that contain a spontaneous “non-lethal” mutant of BAX (which is improbable) and cells that contain mutations in specific genes that cause the cells to be resistant to Bax's lethal effects (for example, mutations in the yeast UTH1 gene nullifies Bax's effects in yeast).

Thus the invention provides a kit of parts comprising the yeast cells of the first or second aspects of the invention, and other components useful for performing a method of screening for inhibitors of Bax-mediated apoptosis.

Typically, the kit of parts comprises a yeast plasmid vector suitable for transforming a library of polynucleotides into the yeast cells. Preferably, the yeast plasmid vector is suitable for expressing polynucleotides from the library under the control of an inducible promoter. Suitable yeast plasmid vectors include pYES2 (Stratagene). It is appreciated that any 2-micron (multi-copy) or centromere (single-copy) yeast shuttle vector known in the art can be employed.

The kit may further comprise an agent that induces expression of the polynucleotides from the library in the yeast cell. Many suitable inducible promoters, and their corresponding inducer, are known in the art of yeast genetics. For example, tetracycline-inducible promoters, methionine-inducible promoters and galactose-inducible promoters are all well known in the art. Other suitable promoters include the ADH2 alcohol dehydrogenase promoter (repressed in glucose, induced when glucose is exhausted and ethanol is made) and the CUP1 metallothionein promoter (induced in the presence of Cu²⁺, Zn²⁺).

It is preferred if the agent that induces expression of the polynucleotides in the yeast cell is galactose. In any event, it may be useful to include galactose in the kit since it is needed to induce expression of the functional Bax polypeptide.

The kit may further comprise instructions for performing a method of screening the library of polynucleotides, which is typically a cDNA library, for an inhibitor of Bax-mediated apoptosis.

As described below in the Examples, the inventor has used a yeast cell as defined in the first aspect of the invention to screen for inhibitors of Bax-mediated apoptosis. The polynucleotide encoding the functional Bax polypeptide in the yeast is under the control of a galactose-inducible promoter which is OFF in the presence of glucose, but which is ON in the presence of galactose. Thus, the functional Bax polypeptide is only expressed in the presence of galactose and kills all yeast cells in the presence of galactose. As a control for this screening method, co-expression of Bcl-2 and Bcl-xL with the functional Bax polypeptide was shown to prevent Bax-induced cell death in media containing galactose as the sole carbon source. Galactose permits respiratory (i.e. mitochondria-mediated) growth, whereas glucose permits both fermentative and respiratory growth. Therefore, the identified genes/proteins that rescue Bax-mediated death in this screen are ones that abrogate death via the mitochondria. In other words, these identified genes/proteins might be considered to protect mitochondrial function.

A human hippocampus cDNA library was amplified in a yeast expression vector to obtain 1.2×10⁶ individual clones and transformed into W303baxleu yeast cells which contain an integrated Bax-expression cassette and which give “no background” (see FIG. 53). The transformants were directly screened in plates containing galactose as the sole carbon source to avoid growth in glucose, and then replica-plated. (Replica plating is important since cells that grow directly on galactose are the ones that should contain the plasmid-borne anti-apoptotic genes of interest). 3.8×10¹⁰ individual yeast transformants were screened (calculated on the basis of transformants obtained on glucose plates in a control experiment to determine the number of cells obtained from each transformation). Plasmids were isolated from cells that grow in galactose, i.e. in the presence of the functional Bax polypeptide. Purified plasmids were retransformed into Bax-containing yeast cells and checked again for prevention of cell death. The polynucleotide inserts of those plasmids that definitely prevented Bax-mediated cell death in yeast, and which bear the anti-apoptotic polynucleotides of interest were sequenced.

As described in the Examples, performance of this screening method on a human hippocampus cDNA library resulted in the identification of sixteen polynucleotides (corresponding to genes or partial genes) that abrogate Bax-mediated apoptosis and cell death in the yeast. Of the sixteen polynucleotides thus identified, one was Bcl-2A1, a homologue of Bcl-2 which is a known inhibitor of apoptosis. The identification of Bcl-2A1 as a result of the screening method is clear proof-of-principle that the method is highly suitable and effective at screening for polynucleotides that are, or that encode, inhibitors of Bax-mediated apoptosis.

A fourth aspect of the invention thus provides a method of screening for a polynucleotide that is or that encodes an inhibitor of Bax-mediated apoptosis, the method comprising:
(a) providing a library of polynucleotides in yeast plasmid vectors;
(b) transforming the library of polynucleotides into yeast cells as defined in the first or second aspects of the invention;
(c) plating the transformed yeast under conditions that allow expression of the functional Bax polypeptide and of the polynucleotides in the yeast plasmid vectors; and
(d) identifying one or more yeast colonies that grow under the conditions of step (c),
   wherein growth of a yeast colony indicates that the polynucleotide in the yeast plasmid vector is, or encodes, an inhibitor of Bax-mediated apoptosis.

Preferably, the library of polynucleotides is a cDNA library, which may be generated from sources such as human brain tissue, a tissue or cells that are involved in diabetes, a tissue that is involved in rheumatoid arthritis, or from a cell line. The cDNA library may also be made from cancer tissue, heart tissue, muscle tissue, or a viral or bacterial genome.

Typically, the library of polynucleotides in the yeast plasmid vectors is under the control of an inducible promoter. Suitably, the inducible promoter can be a tetracycline-inducible promoter, a methionine-inducible promoter, a galactose-inducible promoter, the ADH2 alcohol dehydrogenase promoter (repressed in glucose, induced when glucose is exhausted and ethanol is made), or the CUP1 metallothionein promoter (induced in the presence of Cu²⁺, Zn²⁺). Typically, the galactose-inducible promoter is GAL1 or GAL10.

Many suitable yeast plasmid vectors are known in the art and include pYES2 (Stratagene), and other 2-micron (multi-copy) or centromere (single-copy) yeast shuttle vectors known in the art.

It is preferred if transforming step (b) is a high efficiency transformation.

Typically, plating step (c) comprises incubating the plated yeast cells at 30° C. in one of the known standard yeast growth media, with galactose as the sole carbon source, for at least 72 hours, and possibly up to 7 days. Clearly, if the polynucleotides in the yeast plasmid vectors are under the control of a promoter that is inducible by an agent other than galactose, that inducing agent is also included.

In an alternative embodiment, the Bax polypeptide may be under the control of the ADH2 promoter rather than a GAL promoter, for example, to screen for proteins that abrogate the Bax-mediated block of cell growth in petites (that grow without functional mitochondria).

As is immediately apparent to a person of skill in the art of yeast genetics, the method may further comprise one or more of the following steps, and typically all three.
(e) isolating yeast cells from a yeast colony identified in step (d);
(f) isolating the yeast plasmid vector from the yeast colony identified in step (d) or from the yeast cells isolated in step (e); and
(g) sequencing the polynucleotide (i.e. the insert) from the yeast plasmid vector isolated in step (f).

As described below in the examples, the method typically further comprises the step of:
(h) retesting the polynucleotide from the plasmid vector present in a yeast colony identified in step (d), or a polypeptide encoded by said polynucleotide, for the ability to inhibit Bax-mediated apoptosis in a model of apoptosis.

Additionally or alternatively, the method may further comprise the step of:
(i) modifying the polynucleotide from the plasmid vector present in a yeast colony identified in step (d), and testing the modified polynucleotide, or a polypeptide encoded by said modified polynucleotide, for the ability to inhibit Bax-mediated apoptosis in a model of apoptosis.

Further, the method may also comprise the step of:
j) identifying the polynucleotide from a yeast colony identified in step (d) based upon the sequence data obtained in step (g), and testing a polynucleotide that corresponds to the identified polynucleotide, or a polypeptide encoded by said corresponding polynucleotide, for the ability to inhibit Bax-mediated apoptosis in a model of apoptosis.

Since the polynucleotide obtained from a yeast colony identified in step (d) may not encode the full-length naturally occurring polypeptide, by a polynucleotide that “corresponds to” the identified polynucleotide, we include a full-length version of the identified polynucleotide that encodes the full-length naturally occurring polypeptide. Also, since the gene corresponding to the identified polynucleotide may encode several polypeptide isoforms, by a polynucleotide that “corresponds to” the identified polynucleotide, we include a polynucleotide that encodes a different isoform of the naturally occurring polypeptide. In addition, since the identified polynucleotide may possess sequence differences from the naturally-occurring polynucleotide sequence, typically if the cDNA library was obtained from a cell line, by a polynucleotide that “corresponds to” the identified polynucleotide, we include the naturally occurring polynucleotide. Further, since the polynucleotide may be isolated from a non-human cDNA library, by a polynucleotide that “corresponds to” the identified polynucleotide we include a homologue of the identified polynucleotide from another species, and preferably a human homologue.

Suitable models of apoptosis include yeast cell models, mammalian cell models, and in vivo models of apoptosis. The yeast cell model for testing whether a polynucleotide has the ability to inhibit Bax-mediated apoptosis in a cell may be one as described above in the first aspect of the invention, such as WT303baxleu. The mammalian cell model for testing whether a polynucleotide has the ability to inhibit Bax-mediated apoptosis in a cell may be almost any mammalian cell or cell line. As described below in the Examples, the HEK293 cell line was chosen since the anti-apoptotic genes identified from a hippocampal cDNA library are expected to be effective in neuronal cells, and HEK293 is suggested to have some neuronal features (at least, it is routinely used as a replacement of typical neuronal cells which are difficult to use).

For example, cells can be transfected with an indicator plasmid carrying a reporter gene encoding an indicator molecule, and the degree of cell death or apoptosis can be measured by detection of the expressed indicator molecule. For example, the degree of apoptosis in cells transfected with an indicator plasmid expressing the indicator E. coli β-galactosidase can be determined by a β-galactosidase ELISA (Boehringer Mannheim), in accordance with the manufacturer's recommendations. Also, the degree of apoptotic activity can be determined by visually scoring, under a microscope, blue cells expressing β-galactosidase, after staining them with X-gal. As another example, the degree of apoptosis in cells transfected with an indicator plasmid expressing Green Fluorescent Protein can be determined by measuring the fraction of fluorescent cells in the total cell population, using a flow cytometer (FACScan, Becton-Dickinson) or fluorescent microscope.

Also, DNA degradation, indicative of apoptosis, can be examined by exposing the cells to anti-Fas antibody in the presence of CHX. Thereafter, the DNA in the cells is extracted and purified using standard protocols. Any methods detecting cell death or apoptosis can be used such as those described below or in Sellers et al (1994); Telford et al (1994); and Poirier, Ed. (1997) Apoptosis Techniques and Protocols, Humana Press, Totowa, N.J., USA.

The time course of apoptosis can be analysed by measuring the level of expression of phosphatidylserine on the cell surface, as detected, for example, with FITC-labeled Annexin V, and/or by a dye-exclusion test using propidium iodide. These two tests can be performed using a commercially available kit, for example, the ApoAlert Annexin V Apoptosis kit (Clontech), in accordance with the manufacturer's recommendations, and using a flow cytometer (FACScan, Becton-Dickinson) or fluorescent microscope.

As an in vivo model of apoptosis in neurodegeneration, one uses chemicals (which results in PD or AD) to induce apoptosis in mouse or rat brain. As an in vivo model of apoptosis in cancer, one injects tumour cells that result in tumour formation and then determines if any agent would induce apoptosis (i.e. shrinkage of the tumour).

As would be appreciated by the person skilled in the art, the method may further comprise the step of formulating a polynucleotide or polypeptide which has the ability to inhibit Bax-mediated apoptosis in an in vivo model of apoptosis into a pharmaceutically acceptable composition.

As described herein, the above method of screening for a polynucleotide that is or that encodes an inhibitor of Bax-mediated apoptosis, was performed on a human hippocampus cDNA library. Sixteen polynucleotides (corresponding to genes or partial genes) that abrogate Bax-mediated cell death in the yeast system of apoptosis were identified and are listed in Table 1.

TABLE 1
“Bax antagonists” identified in the human hippocampus

| | Identified | | SEQ ID |
|---|---|---|---|
| # | Gene | Brief Description | No |
| 1 | Bcl-2A1 | Homologue of Bcl-2. Known to be expressed in the | 4 |
| | | hippocampus. | |
| 2 | α-Synuclein | Mutant α-Synuclein forms play a major role in PD and | 5 |
| | (SNCA) | AD. The role of the wild-type protein is unclear. | |
| 3 | FKBP2 | An endoplasmic reticulum resident FK506 binding | 6 |
| | (FKBP-13) | protein. Highly overproduced during protein | |
| | | misfolding in the ER. | |
| 4 | EEF1A1 | Eukaryotic translation elongation factor 1 α1. | 7 |
| | | Reported to be involved in oncogenic transformation. | |
| | | Acts as a dominant oncogene in prostate carcinoma | |
| 5 | VAMP3 | Vesicle-associated membrane protein 3 (cellubrevin) | 8 |
| 6 | SNAP25 | Synaptosomal-associated protein | 9 |
| 7 | RIMS3 | Regulates synaptic membrane exocytosis | 10 |
| 8 | RAB40B | A member of the RAS oncogene family | 11 |
| 9 | HMGCS1 | 3-hydroxy-3-methylglutaryl-coenzyme A synthase 1 | 12 |
| | | (soluble) | |
| 10 | SCD5 | Stearoyl-CoA desaturase 5 | 13 |
| 11 | Atp2a2 | ATPase. Ca2+ transporting, cardiac muscle, slow | 14 |
| | | twitch 2 | |
| 12 | HRMT1L1 | hnRNP methyltransferase-like 1. Has a homologue in | 15 |
| | | S. cerevisiae. | |
| 13 | Clone | A sequence from human chromosome 3p of unknown | 16 |
| | RP11-605M1 | function | |
| 14 | Clone | A sequence from human chromosome 22q11.22-12.2 | 17 |
| | CTA-373H7 | of unknown function | |
| 15 | Isolate | A sequence from the genome of human mitochondrial | 18 |
| | WH6967 | isolate WH6967 of unknown function | |
| 16 | Isolate | A sequence from the genome of human mitochondrial | 19 |
| | S1216 | isolate S1216 of unknown function | |

Bcl-2A1, SNCA, FKBP2 (FKBP-13), EEF1A1, and VAMP3 were tested in a mammalian system of apoptosis, and were each confirmed as having activity as an inhibitor of Bax-mediated apoptosis. Bcl-2A1 has previously been reported to be an inhibitor of apoptosis. It is therefore reasonable to conclude that the other eleven polynucleotides identified as a result of the screening method also are, or encode, inhibitors of Bax-mediated apoptosis in mammals.

A fifth aspect of the invention thus provides a method of combating Bax-mediated apoptosis in a cell, the method comprising administering to the cell a polypeptide selected from FKBP2, SNCA, EEF1A1, VAMP3, SNAP25, RIMS3, RAB40B, HMGCS1, SCD5, ATP2A2, HRMT1L1, and a polypeptide encoded by a polynucleotide comprising SEQ ID No: 16, SEQ ID No: 17, SEQ ID No: 18 or SEQ ID No: 19, or an anti-apoptotic derivative of any of these polypeptides, or a polynucleotide that encodes any of said polypeptides or derivatives.

Typically, the method is performed in vitro.

Typically, the cell is a mammalian cell, such as a human cell, although as is clear from the Examples, the cell may be from other species such as yeast. The cell may be from an established cell line, a primary cell culture, or a cell which is present in a tissue ex vivo.

Alternatively, the method may be performed in vivo.

The polypeptides, the anti-apoptotic derivatives thereof, and polynucleotides that encode any of said polypeptides or derivatives, have a clear utility as research tools in the study of apoptosis. They also have utility in methods of screening for further therapeutic agents that modulate apoptosis, as described below.

A sixth aspect of the invention provides the use of a polypeptide selected from FKBP2, SNCA, EEF1A1, VAMP3, SNAP25, RIMS3, RAB40B, HMGCS1, SCD5, ATP2A2, HRMT1L1, and a polypeptide encoded by a polynucleotide comprising SEQ ID No: 16, SEQ ID No: 17, SEQ ID No: 18 or SEQ ID No: 19, or an anti-apoptotic derivative of any of these polypeptides, or a polynucleotide that encodes any of said polypeptides or derivatives, in the preparation of a medicament for combating Bax-mediated apoptosis in a cell.

By “combating Bax-mediated apoptosis in a cell” we mean inhibiting or preventing Bax-mediated apoptosis in a cell.

It is appreciated that the polypeptides, derivatives and polynucleotides of the fifth aspect and the medicament of the sixth aspect of the invention may be suitable for combating any apoptosis where mitochondrial dysfunction occurs since the apoptotic effect of Bax could be mimicked by other proteins or by exogenous/endogenous chemicals. For example, staurospaurine is known to mimic the effects of Bax. Thus the polypeptides, derivatives and polynucleotides of the fifth aspect and the medicament of the sixth aspect of the invention may be suitable for combating staurospaurine induced cell death.

“Derivatives” of any given polypeptide may be made using protein chemistry techniques, for example using partial proteolysis (either exolytically or endolytically), or by de novo synthesis. Alternatively, the derivatives may be made by recombinant DNA technology. Suitable techniques for cloning, manipulation, modification and expression of nucleic acids, and purification of expressed proteins, are well known in the art and are described for example in Sambrook et al (2001) “Molecular Cloning, a Laboratory Manual”, 3^{rd} edition, Sambrook et al (eds), Cold Spring Harbor Laboratory Press, Cold Spring Harbor, N.Y., USA.

Suitable “anti-apoptotic derivatives” of these polypeptides include fragments thereof and modifications of the full-length polypeptide or fragment thereof, that have the ability to inhibit Bax-mediated apoptosis in a cell.

By “modifications” of a given polypeptide we include amino acid insertions, deletions and substitutions, either conservative or non-conservative, at one or more positions. Such modifications may be called analogues of the given polypeptide. By “conservative substitutions” is intended combinations such as Gly, Ala; Val, Ile, Leu; Asp, Glu; Asn, Gln; Ser, Thr; Lys, Arg; and Phe, Tyr. Such modifications may be made using the methods of protein engineering and site-directed mutagenesis, as described in Sambrook et al 2001, supra.

Further modifications of a given polypeptide include the addition of NH₂ or COOH terminal tags that would allow entry of proteins into cells.

The hippocampus is a region of the brain that is known to suffer from apoptosis in neurodegenerative disease. Since the anti-apoptotic polynucleotides were identified from a human hippocampus cDNA library, it is also reasonable to conclude that each of the identified anti-apoptotic polynucleotides, or the anti-apoptotic polypeptides that they encode, may have utility in combating neurodegenerative disease.

A seventh aspect of the invention thus provides a polypeptide selected from FKBP2, SNCA, VAMP3, SNAP25, RIMS3, RAB40B, HMGCS1, SCD5, HRMT1L1, and a polypeptide encoded by a polynucleotide comprising SEQ ID No: 16, SEQ ID No: 17, SEQ ID No: 18 or SEQ ID No: 19, or an anti-apoptotic derivative of any of these polypeptides, or a polynucleotide that encodes any of said polypeptides or derivatives, for use in medicine.

An eighth aspect of the invention provides a pharmaceutical composition comprising a polypeptide selected from FKBP2, SNCA, VAMP3, SNAP25, RIMS3, RAB40B, HMGCS1, SCD5, HRMT1L1, and a polypeptide encoded by a polynucleotide comprising SEQ ID No: 16, SEQ ID No: 17, SEQ ID No: 18 or SEQ ID No: 19, or an anti-apoptotic derivative of any of these polypeptides, or a polynucleotide that encodes any of said polypeptides or derivatives, and a pharmaceutically acceptable carrier or excipient.

In addition, a number of other disorders or conditions are associated with inappropriate Bax-mediated apoptosis in cells or tissues, such as cardiovascular cells in cardiovascular disease (Reeve et al, 2005), synovial cells in rheumatoid arthritis (Baier et al, 2003), and pancreatic B-cells in diabetes (Cnop et al, 2005; Millet et al, 2005).

A ninth aspect of the invention thus provides a method of combating a disease or condition in a patient selected from a neurodegenerative disease or condition, cardiovascular disease, rheumatoid arthritis and diabetes, the method comprising administering to the patient a polypeptide selected from FKBP2, SNCA, EEF1A1, VAMP3, SNAP25, RIMS3, RAB40B, HMGCS1, SCD5, ATP2A2, HRMT1L1, and a polypeptide encoded by a polynucleotide comprising SEQ ID No: 16, SEQ ID No: 17, SEQ ID No: 18 or SEQ ID No: 19, or an anti-apoptotic derivative of any of these polypeptides, or a polynucleotide that encodes any of said polypeptides or derivatives.

Cardiovascular disease is a leading cause of death worldwide. Loss of function or death of cardiomyocytes is a major contributing factor to cardiovascular disease which is a leading cause of death worldwide. Cell death in conditions such as heart failure and myocardial infarction is associated with apoptosis. Apoptotic pathways have been well studied in non-myocytes and it is thought that similar pathways exist in cardiomyocytes. These pathways include death initiated by ligation of membrane-bound death receptors, release of pro-apoptotic factors from mitochondria or stress at the endoplasmic reticulum. The key regulators of apoptosis include inhibitors of caspases (IAPs), the Bcl-2 family of proteins, growth factors, stress proteins, calcium and oxidants. The highly organized and predictive nature of apoptotic signalling means it is amenable to manipulation. A thorough understanding of the apoptotic process would facilitate intervention at the most suitable points, alleviating myocardium decline and dysfunction (Reeve et al, 2005).

By “combating” a disease, disorder or condition in a patient we mean treating, preventing, or ameliorating the symptoms of, that particular disorder or condition.

Neurodegenerative diseases or disorders that can be treated using the therapeutic methods and uses of the present invention include stroke, spinal cord trauma, head injury, spinal muscular atrophy (SMA), motor neuron disease including amyotrophic lateral sclerosis (ALS), Alzheimer's disease (AD), Parkinson's disease (PD) and Huntington's disease (HD).

A tenth aspect of the invention thus provides the use of a polypeptide selected from FKBP2, SNCA, EEF1A1, VAMP3, SNAP25, RIMS3, RAB40B, HMGCS1, SCD5, ATP2A2, HRMT1L1, and a polypeptide encoded by a polynucleotide comprising SEQ ID No: 16, SEQ ID No: 17, SEQ ID No: 18 or SEQ ID No: 19, or an anti-apoptotic derivative of any of these polypeptides, or a polynucleotide that encodes any of said polypeptides or derivatives, in the preparation of a medicament for combating a disease or condition in a patient selected from a neurodegenerative disease or condition, cardiovascular disease, rheumatoid arthritis and diabetes.

### FKBP2

FKBP2 (also known as FK506-Binding protein 2 and FKBP13) is a member of a family of proteins which bind the immunosuppressant drugs, FK506 and rapamycin. The FKBP2 gene is 3 kb in length and contains six exons and is located at human chromosome 11q13.1-q13.3 (DiLella et al, 1992). Partaledis & Berlin (1993) describe the FKB2 gene of S. cerevisiae which encodes a homologue of human FKBP-13 having 57% sequence identity with human FKBP-13, and suggest that FKB2/FKB13 plays a role in protein trafficking in the endoplasmic reticulum (ER).

According to Genbank Accession No NP_{—}476433, the protein encoded by the FKB2 gene is a member of the immunophilin protein family, which plays a role in immunoregulation and basic cellular processes involving protein folding and trafficking. The FKB2 encoded protein is a cis-trans prolyl isomerase that binds the immunosuppressants FK506 and rapamycin. It is thought to function as an ER chaperone and may also act as a component of membrane cytoskeletal scaffolds. This gene has two alternatively spliced transcript variants that encode the same isoform. Multiple polyadenylation sites have been described for this gene, but the full-length nature of this gene has not been determined. FKBP2 has a signal peptide at residues 1-21 (as defined in NP_{—}476433), and the mature polypeptide is at residues 22-142. FKBP2 is a peptidylprolyl isomerase (EC 5.2.1.8).

To the best of the inventor's knowledge, FKBP2 has not been associated with apoptosis.

Thus the invention includes combating Bax-mediated apoptosis in a cell by administering to the cell a FKBP2 polypeptide, or an anti-apoptotic derivative thereof, or a polynucleotide that encodes said polypeptide or derivative.

Similarly, to the best of the inventor's knowledge, FKBP2 has not been associated with any neurodegenerative disorder. Indeed, according to Online Mendelian Inheritance in Man (OMIM, Reference No. 186946) FKBP2 has not been associated with any disease state and, as far as the inventor is aware, FKBP2 has not been used therapeutically.

Thus the invention includes an FKBP2 polypeptide, or an anti-apoptotic derivative thereof, or a polynucleotide that encodes said polypeptide or derivative, for use in medicine.

The invention also includes combating a neurodegenerative disease in a patient by administering to the patient a FKBP2 polypeptide, or an anti-apoptotic derivative thereof, or a polynucleotide that encodes said polypeptide or derivative.

By “FKBP2 polypeptide” we include the meaning of a gene product of the human FKBP2 gene, including naturally occurring variants thereof. The cDNA sequence corresponding to a human FKBP2 mRNA is found in Genbank Accession No NM_{—}057092 (variant 2) and NM_{—}004470 (variant 1). Transcript variant 2 has a distinct exon at the 5′ UTR compared to variant 1, although the coding region is the same in both variants. Human FKBP2 polypeptide includes the amino acid sequence found in Genbank Accession No NP_{—}476433, and naturally occurring variants thereof.

Suitable “anti-apoptotic derivatives” of FKBP2 include anti-apoptotic fragments of FKBP2. By “an anti-apoptotic fragment” of FKBP2 we mean any portion of the FKBP2 polypeptide that has the ability to inhibit Bax-mediated apoptosis in a cell. Typically, the fragment has at least 30% of the anti-apoptotic activity of full-length human FKBP2. It is more preferred if the fragment has at least 50%, preferably at least 70% and more preferably at least 90% of the activity of full-length human FKBP2. Most preferably, the fragment has 100% or more of the anti-apoptotic activity of full-length human FKBP2.

Suitable “anti-apoptotic derivatives” of FKBP2 also include modifications of full-length FKBP2, or a fragment thereof, that have the ability to inhibit Bax-mediated apoptosis in a cell. Preferably, the modified FKBP2 or modified FKBP2 fragment retains at least 80%, or at least 85% or at least 90% sequence identity with full-length FKBP2, or the respective FKBP2 fragment. More preferably, the modified FKBP2 or modified FKBP2 fragment has at least 91%, or at least 92%, or at least 93%, or at least 94% or at least 95% sequence identity, and yet more preferably at least 96%, or at least 97%, or at least 98% or at least 99% sequence identity with full-length FKBP2, or the respective FKBP2 fragment. Preferably, the modified FKBP2 or modified FKBP2 fragment retains at least 30% of the anti-apoptotic activity of full-length human FKBP2. It is more preferred if the modified FKBP2 or FKBP2 derivative has at least 50%, preferably at least 70% and more preferably at least 90% of the activity of full-length human FKBP2. Most preferably, the modified FKBP2 or modified FKBP2 fragment has 100% or more of the anti-apoptotic activity of full-length human FKBP2.

By FKBP2 we also include a homologous gene product from FKBP2 genes from other species. By “homologous gene product” we include an FKBP2 polypeptide having at least 80% sequence identity with the human FKBP2 amino acid sequence in Genbank Accession No NP_{—}476433. More preferably, a homologous gene product includes an FKBP2 polypeptide having at least 85% or at least 90% sequence identity with human FKBP2. Yet more preferably, a homologous gene product includes an FKBP2 polypeptide having at least 91%, or at least 92%, or at least 93%, or at least 94%, or at least 95%, or at least 96%, or at least 97%, or at least 98% sequence identity with human FKBP2. Most preferably, a homologous gene product includes an FKBP2 polypeptide having at least 99% sequence identity with the human FKBP2 amino acid sequence. It is appreciated that for applications in which FKBP2 is administered to a non-human subject, the FKBP2 is preferably from the same species as the subject. If the FKBP2 is administered to a human subject, the FKBP2 is preferably human FKBP2, or an anti-apoptotic fragment or variant thereof.

Although there is only 47% sequence identity (perfect match) and 69% sequence homology (accepting conserved residues) between the FKBP2 proteins from humans and yeast, the human FKBP2 can complement inactivating FKBP2 mutations in yeast cells (data not included).

By a polynucleotide encoding FKBP2 we include the cDNA encoding the human FKBP2 polypeptide and naturally occurring variants thereof. cDNA sequences encoding a human FKBP2 mRNA are found in Genbank Accession Nos. NM_{—}057092 and NM_{—}004470. We also include other sequences which, by virtue of the degeneracy of the genetic code, also encode the FKBP2 polypeptide.

The polynucleotide (SEQ ID No: 6) encoding FKBP2 that was initially identified by the inventor was identified as a full-length fragment. This gene was re-cloned by PCR from a cDNA library and was C-terminally tagged for protein expression.

FKBP2 is a peptidylprolyl isomerase (EC 5.2.1.8; also known as Peptidyl-prolyl cis-trans isomerase). There are three distinct families of peptidylprolyl isomerases: the cyclophilins, the FKBPs, and another family that includes parvulin from E. coli. The three families are structurally unrelated and can be distinguished by being inhibited by cyclosporin A, FK-506 and 5-hydroxy-1,4-naphthoquinone, respectively (http://www.chem.qmul.ac.uk/iubmb/enzyme/EC5/2/1/8.html).

Without wishing to be bound by any theory, the inventor considers that since FKBP2 is a peptidylprolyl isomerase (EC 5.2.1.8; also known as peptidyl-prolyl cis-trans isomerase), it is possible that its peptidylprolyl isomerase activity contributes to its anti-apoptotic activity. Thus other peptidylprolyl isomerases, and especially other FKPBs, as well as polynucleotides encoding them, may also be useful in combating apoptosis. This is particularly unexpected since over-expression of cyclophilin D, a peptidylprolyl isomerase, is known to enhance the apoptotic process (Li et al, 2004). Moreover, inhibition of peptidylprolyl isomerases (also known as immunophilins) has been used as a target for prevention of neurodegeneration.

### α-Synuclein (SNCA)

SNCA is a member of the synuclein family, which also includes beta- and gamma-synuclein. Synucleins are abundantly expressed in the brain and alpha- and beta-synuclein inhibit phospholipase D2 selectively. SNCA may serve to integrate presynaptic signalling and membrane trafficking. Defects in SNCA have been implicated in the pathogenesis of Parkinson disease (PD). SNCA peptides are a major component of amyloid plaques in the brains of patients with Alzheimer's disease (AD). Two alternatively spliced transcripts of SNCA have been identified.

To the best of the inventor's knowledge, SNCA has not previously been shown to be an inhibitor of Bax-mediated apoptosis.

Thus the invention includes combating Bax-mediated apoptosis in a cell by administering to the cell a SNCA polypeptide, or an anti-apoptotic derivative thereof, or a polynucleotide that encodes said polypeptide or derivative.

Similarly, although SNCA has been associated with both AD and PD (OMIM Reference No. 163890), to the best of the inventor's knowledge, there has been no previous suggestion to use an SNCA polypeptide, or an anti-apoptotic derivative thereof, or a polynucleotide that encodes said polypeptide or derivative, in combating a neurodegenerative disease in a patient. Indeed, as far as the inventor is aware, SNCA has not been used therapeutically.

Thus the invention includes an SNCA polypeptide, or an anti-apoptotic derivative thereof, or a polynucleotide that encodes said polypeptide or derivative, for use in medicine.

The invention also includes combating a neurodegenerative disease in a patient by administering to the patient a SNCA polypeptide, or an anti-apoptotic derivative thereof, or a polynucleotide that encodes said polypeptide or derivative.

In an embodiment, the invention includes combating a neurodegenerative disease other than AD of PD in a patient by administering to the patient a SNCA polypeptide, or an anti-apoptotic derivative thereof, or a polynucleotide that encodes said polypeptide or derivative.

By “SNCA polypeptide” we include the meaning of a gene product of the human SNCA gene, including naturally occurring variants thereof. Two alternative transcript variants encoding different protein isoforms have been described for the human SNCA gene. A cDNA sequence corresponding to a human SNCA mRNA is found in Genbank Accession No NM_{—}000345 (isoform NACP140) and in Genbank Accession No NM_{—}007308 (isoform NACP112). The NACP140 transcript is the longer transcript and encodes the longer NACP140 isoform (Genbank Accession No NP_{—}000336). The NACP112 transcript lacks an alternate in-frame segment, compared to variant NACP140, resulting in a shorter protein (isoform NACP112; Genbank Accession No NP_{—}009292) that has a distinct C-terminus, compared to isoform NACP140. The amino acid sequence of human SNCA polypeptides includes the sequences found in Genbank Accession Nos NP_{—}000336 and NP_{—}009292, and naturally occurring variants thereof. The SNCA sequence identified in the screening experiments described herein was from the NACP140 variant.

Suitable “anti-apoptotic derivatives” of SNCA include anti-apoptotic fragments of SNCA. By “an anti-apoptotic fragment” of SNCA we mean any portion of a SNCA polypeptide that has the ability to inhibit Bax-mediated apoptosis in a cell. Typically, the fragment has at least 30% of the anti-apoptotic activity of the SNCA polypeptide of SEQ ID No: 5. It is more preferred if the fragment has at least 50%, preferably at least 70% and more preferably at least 90% of the activity of the SNCA polypeptide of SEQ ID No: 5. Most preferably, the fragment has 100% or more of the anti-apoptotic activity of the SNCA polypeptide of SEQ ID No: 5.

Suitable “anti-apoptotic derivatives” of SNCA also include modifications of full-length SNCA, or a fragment thereof, that have the ability to inhibit Bax-mediated apoptosis in a cell. Preferably, the modified SNCA or modified SNCA fragment retains at least 80%, or at least 85% or at least 90% sequence identity with full-length SNCA, or the respective SNCA fragment. More preferably, the modified SNCA or modified SNCA fragment has at least 91%, or at least 92%, or at least 93%, or at least 94% or at least 95% sequence identity, and yet more preferably at least 96%, or at least 97%, or at least 98% or at least 99% sequence identity with full-length SNCA, or the respective SNCA fragment. Preferably, the modified SNCA or modified SNCA fragment retains at least 30% of the anti-apoptotic activity of full-length human SNCA. It is more preferred if the modified SNCA or SNCA derivative has at least 50%, preferably at least 70% and more preferably at least 90% of the activity of full-length human SNCA. Most preferably, the modified SNCA or modified SNCA fragment has 100% or more of the anti-apoptotic activity of full-length human SNCA.

By SNCA we also include a homologous gene product from SNCA genes from other species. By “homologous gene product” we include an SNCA polypeptide having at least 80% sequence identity with the human SNCA amino acid sequences in Genbank Accession Nos NP_{—}000336 and NP_{—}009292. More preferably, a homologous gene product includes an SNCA polypeptide having at least 85% or at least 90% sequence identity with a human SNCA. Yet more preferably, a homologous gene product includes an SNCA polypeptide having at least 91%, or at least 92%, or at least 93%, or at least 94%, or at least 95%, or at least 96%, or at least 97%, or at least 98% sequence identity with human SNCA. Most preferably, a homologous gene product includes an SNCA polypeptide having at least 99% sequence identity with a human SNCA amino acid sequence in Genbank Accession No NP_{—}000336 or NP_{—}009292. It is appreciated that for applications in which SNCA is administered to a non-human subject, the SNCA is preferably from the same species as the subject. If the SNCA is administered to a human subject, the SNCA is preferably human SNCA, or an anti-apoptotic fragment or variant thereof.

By a polynucleotide encoding SNCA we include the cDNA encoding the human SNCA polypeptide and naturally occurring variants thereof. cDNA sequences encoding a human SNCA mRNA are found in Genbank Accession Nos. NM_{—}000345 and NM_{—}007308. We also include other sequences which, by virtue of the degeneracy of the genetic code, also encode the SNCA polypeptide. An example of a polynucleotide encoding an anti-apoptotic fragment of SNCA is listed in SEQ ID No: 5.

The polynucleotide (SEQ ID No: 5) encoding SNCA that was initially identified by the inventor is believed to be a full-length fragment: the insert was sequenced from the 3′ end and the last few nucleotides in the read were N's. A full-length transcript was subsequently cloned by PCR from the same hippocampal cDNA library used for the screening, and the Bax-rescuing ability of the initially isolated clone and of the full-length PCR fragment was the same.

### EEF1A

The eukaryotic elongation factor 1 A (eEF1A, formerly known as EF1alpha) is a key factor in protein synthesis, where it promotes the transfer of aminoacylated tRNAs to the A site of the ribosome. Two differentially expressed isoforms of eEF1A, designated eEF1A-1 and eEF1A-2, are found in mammals. In humans, the eEF1A-1 and eEF1A-2 isoforms have 92.6% sequence identity (see FIG. 67).

According to NP_{—}001393, the EEF1A1 gene encodes an isoform of the alpha subunit of the elongation factor-1 complex, which is responsible for the enzymatic delivery of aminoacyl tRNAs to the ribosome. This isoform (alpha 1) is expressed in brain, placenta, lung, liver, kidney, and pancreas, whereas the other isoform (alpha 2) is expressed in brain, heart and skeletal muscle. The alpha 1 isoform is identified as an autoantigen in 66% of patients with Felty syndrome. The eEF1A gene is located at human chromosome 6q14, but has been found to have multiple copies on many chromosomes, some of which, if not all, represent different pseudogenes. The alpha subunit of elongation factor-1 (EEF1A) is involved in the binding of aminoacyl-tRNAs to 80S ribosomes. During the process, GTP is hydrolyzed into GDP. To perform this function, EEF1A has domains that bind guanine nucleotides, 80S ribosomes, and aminoacyl-tRNAs. Also, EEF1A interacts with the beta subunit of EEF1 to exchange bound GDP for GTP. The primary structure of human EEF1A was determined by Brands et al (1986).

Ditzel et al (2000) identified EEF1A1 as an autoantibody in 66% of patients with Felty syndrome, a disorder characterized by the association of rheumatoid arthritis, splenomegaly, and peripheral destruction of neutrophils leading to neutropaenia.

According to the review by Lamberti et al (2004), EEF1A is involved in apoptosis (without specifying whether this is EEF1A1 or EEF1A2, or both). Lamberti et al also reports that EEF1A2 plays a role in the protection of caspase 3-mediated apoptosis. Based on the anti-apoptotic effects of EEF1A2, Thornton et al (2003) suggest that EEF1A1 may also inhibit apoptosis. Talapatra et al (2001) identified EEF1A1 in a screen for genes that protect against apoptosis caused by interleukin 3 withdrawal.

To the best of the inventor's knowledge, EEF1A1 has not been associated with Bax-mediated apoptosis.

Thus the invention includes combating Bax-mediated apoptosis in a cell by administering to the cell a EEF1A1 polypeptide, or an anti-apoptotic derivative thereof, or a polynucleotide that encodes said polypeptide or derivative.

Similarly, to the best of the inventor's knowledge, EEF1A1 has not been associated with any neurodegenerative disorder.

Thus the invention also includes combating a neurodegenerative disease in a patient by administering to the patient an EEF1A1 polypeptide, or an anti-apoptotic derivative thereof, or a polynucleotide that encodes said polypeptide or derivative.

By “EEF1A1 polypeptide” (eukaryotic translation elongation factor 1 α1) we include the meaning of a gene product of the human EEF1A1 gene, including naturally occurring variants thereof. A cDNA sequence corresponding to a human EEF1A1 mRNA is found in Genbank Accession No NM_{—}001402. Human EEF1A1 polypeptide includes the amino acid sequence found in Genbank Accession No NP_{—}001393, and naturally occurring variants thereof.

Suitable “anti-apoptotic derivatives” of EEF1A1 include anti-apoptotic fragments of EEF1A1. By “an anti-apoptotic fragment” of EEF1A1 we mean any portion of the EEF1A1 polypeptide that has the ability to inhibit Bax-mediated apoptosis in a cell. Typically, the fragment has at least 30% of the anti-apoptotic activity of full-length human EEF1A1. It is more preferred if the fragment has at least 50%, preferably at least 70% and more preferably at least 90% of the activity of full-length human EEF1A1. Most preferably, the fragment has 100% or more of the anti-apoptotic activity of full-length human EEF1A1.

Suitable “anti-apoptotic derivatives” of EEF1A1 also include fragments of full-length EEF1A1, or modifications of the full-length polypeptide of fragment thereof, that have the ability to inhibit Bax-mediated apoptosis in a cell. Preferably, the modified EEF1A1 or modified EEF1A1 fragment retains at least 93% sequence identity with full-length EEF1A1, or the respective EEF1A1 fragment. More preferably, the modified EEF1A1 or modified EEF1A1 fragment has at least 94% or at least 95% sequence identity, and yet more preferably at least 96%, or at least 97%, or at least 98% or at least 99% sequence identity with full-length EEF1A1, or the respective EEF1A1 fragment. Preferably, the modified EEF1A1 or modified EEF1A1 fragment retains at least 30% of the anti-apoptotic activity of full-length human EEF1A1. It is more preferred if the modified EEF1A1 or EEF1A1 derivative has at least 50%, preferably at least 70% and more preferably at least 90% of the activity of full-length human EEF1A1. Most preferably, the modified EEF1A1 or modified EEF1A1 fragment has 100% or more of the anti-apoptotic activity of full-length human EEF1A1.

By EEF1A1 we also include a homologous gene product from EEF1A1 genes from other species. By “homologous gene product” we include an EEF1A1 polypeptide having at least 93% sequence identity with the human EEF1A1 amino acid sequence in Genbank Accession No NP_{—}001393. More preferably, a homologous gene product includes an EEF1A1 polypeptide having at least 94%, or at least 95%, or at least 96%, or at least 97%, or at least 98% sequence identity with human EEF1A1. Most preferably, a homologous gene product includes an EEF1A1 polypeptide having at least 99% sequence identity with the human EEF1A1 amino acid sequence. It is appreciated that for applications in which EEF1A1 is administered to a non-human subject, the EEF1A1 is preferably from the same species as the subject. If the EEF1A1 is administered to a human subject, the EEF1A1 is preferably human EEF1A1, or an anti-apoptotic fragment or variant thereof.

By a polynucleotide encoding EEF1A1 we include the cDNA encoding the human EEF1A1 polypeptide and naturally occurring variants thereof. A cDNA sequence encoding a human EEF1A1 mRNA is found in Genbank Accession No NM_{—}001402. We also include other sequences which, by virtue of the degeneracy of the genetic code, also encode the EEF1A1 polypeptide. An example of a polynucleotide encoding an anti-apoptotic fragment of EEF1A1 is listed in SEQ ID No: 7 (and the full-length EEF1A1 was subsequently cloned for confirmation of its effects in yeast and in HEK293 cells).

### VAMP3

VAMP3 is a member of the vesicle-associated membrane protein (VAMP)/synaptobrevin family. VAMPs, together with syntaxins, and the 25-kD synaptosomal-associated protein (SNAP25), are the main components of a protein complex involved in the docking and/or fusion of synaptic vesicles with the presynaptic membrane. Because of VAMP3's high homology to other known VAMPs, its broad tissue distribution, and its subcellular localisation, the protein encoded by this gene was considered to be the human equivalent of the rodent cellubrevin. In platelets the protein resides on a compartment that is not mobilized to the plasma membrane on calcium or thrombin stimulation

To the best of the inventor's knowledge, VAMP3 has not been associated with apoptosis.

Thus the invention includes combating Bax-mediated apoptosis in a cell by administering to the cell a VAMP3 polypeptide, or an anti-apoptotic derivative thereof, or a polynucleotide that encodes said polypeptide or derivative. VAMP3 was found to be one of 25 genes within a locus on human chromosome 1p36 that is involved in early-onset recessive parkinsonism (van Duijn et al, 2001). However, VAMP3 has not been directly associated with any neurodegenerative disease or condition. Indeed, according to OMIM Reference No 603657, VAMP3 has not been directly associated with any disease state and, as far as the inventor is aware, VAMP3 has not been used therapeutically.

Thus the invention includes a VAMP3 polypeptide, or an anti-apoptotic derivative thereof, or a polynucleotide that encodes said polypeptide or derivative, for use in medicine.

Thus the invention also includes combating a neurodegenerative disease or condition in a patient by administering to the patient a VAMP3 polypeptide, or an anti-apoptotic derivative thereof, or a polynucleotide that encodes said polypeptide or derivative.

By VAMP3 polypeptide (vesicle-associated membrane protein 3) we include the meaning of a gene product of the human VAMP3 gene, including naturally occurring variants thereof. A cDNA sequence corresponding to a human VAMP3 mRNA is found in Genbank Accession No NM_{—}004781. Human VAMP3 polypeptide includes the amino acid sequence found in Genbank Accession No NP_{—}004772, and naturally occurring variants thereof.

Suitable “anti-apoptotic derivatives” of VAMP3 include anti-apoptotic fragments of VAMP3. By “an anti-apoptotic fragment” of VAMP3 we mean any portion of the VAMP3 polypeptide that has the ability to inhibit Bax-mediated apoptosis in a cell. Typically, the fragment has at least 30% of the anti-apoptotic activity of full-length human VAMP3. It is more preferred if the fragment has at least 50%, preferably at least 70% and more preferably at least 90% of the activity of full-length human VAMP3. Most preferably, the fragment has 100% or more of the anti-apoptotic activity of full-length human VAMP3.

Suitable “anti-apoptotic derivatives” of VAMP3 also include fragments of full-length VAMP3, or modifications of the full-length polypeptide of fragment thereof, that have the ability to inhibit Bax-mediated apoptosis in a cell. Preferably, the modified VAMP3 or modified VAMP3 fragment retains at least 90% sequence identity with full-length VAMP3, or the respective VAMP3 fragment. More preferably, the modified VAMP3 or modified VAMP3 fragment has at least 91%, 92%, 93%, 94% or 95% sequence identity, and yet more preferably at least 96%, 97%, 98% or 99% sequence identity with full-length VAMP3, or the respective VAMP3 fragment. Preferably, the modified VAMP3 or modified VAMP3 fragment retains at least 30% of the anti-apoptotic activity of full-length human VAMP3. It is more preferred if the modified VAMP3 or VAMP3 derivative has at least 50%, preferably at least 70% and more preferably at least 90% of the activity of full-length human VAMP3. Most preferably, the modified VAMP3 or modified VAMP3 fragment has 100% or more of the anti-apoptotic activity of full-length human VAMP3.

By VAMP3 we also include a homologous gene product from VAMP3 genes from other species. By “homologous gene product” we include an VAMP3 polypeptide having at least 90% sequence identity with the human VAMP3 amino acid sequence in Genbank Accession No NP_{—}004772. More preferably, a homologous gene product includes an VAMP3 polypeptide having at least 95%, or at least 96%, or at least 97%, or at least 98% sequence identity with human VAMP3. Most preferably, a homologous gene product includes an VAMP3 polypeptide having at least 99% sequence identity with the human VAMP3 amino acid sequence. It is appreciated that for applications in which VAMP3 is administered to a non-human subject, the VAMP3 is preferably from the same species as the subject. If the VAMP3 is administered to a human subject, the VAMP3 is preferably human VAMP3, or an anti-apoptotic fragment or variant thereof.

By a polynucleotide encoding VAMP3 we include the cDNA encoding the human VAMP3 polypeptide and naturally occurring variants thereof. A cDNA sequence encoding a human VAMP3 mRNA is found in Genbank Accession No NM_{—}004781. We also include other sequences which, by virtue of the degeneracy of the genetic code, also encode the VAMP3 polypeptide.

An example of a polynucleotide encoding an anti-apoptotic fragment of VAMP3 is listed in SEQ ID No: 8.

The polynucleotide (SEQ ID No: 8) encoding VAMP3 was isolated in the yeast screen as a full-length fragment.

### SNAP25

Synaptic vesicle membrane docking and fusion is mediated by SNAREs (soluble N-ethylmaleimide-sensitive factor attachment protein receptors) located on the vesicle membrane (v-SNAREs) and the target membrane (t-SNAREs). The assembled v-SNARE/t-SNARE complex consists of a bundle of four helices, one of which is supplied by v-SNARE and the other three by t-SNARE. For t-SNAREs on the plasma membrane, the protein syntaxin supplies one helix and the protein encoded by SNAP25 contributes the other two. Thus the gene product of SNAP25 is a presynaptic plasma membrane protein involved in the regulation of neurotransmitter release.

To the best of the inventor's knowledge, SNAP25 has not been associated with apoptosis.

Thus the invention includes combating Bax-mediated apoptosis in a cell by administering to the cell a SNAP25 polypeptide, or an anti-apoptotic derivative thereof, or a polynucleotide that encodes said polypeptide or derivative.

To the best of the inventor's knowledge, SNAP25 has not been associated with any neurodegenerative disease or condition. Indeed, according to OMIM Reference No. 600322, SNAP25 has not been associated with any disease state and, as far as the inventor is aware, SNAP25 has not been used therapeutically.

Thus the invention includes a SNAP25 polypeptide, or an anti-apoptotic derivative thereof, or a polynucleotide that encodes said polypeptide or derivative, for use in medicine.

The invention also includes combating a neurodegenerative disease or condition in a patient by administering to the patient a SNAP25 polypeptide, or an anti-apoptotic derivative thereof, or a polynucleotide that encodes said polypeptide or derivative.

By SNAP25 (synaptosomal-associated protein 25 kDa) we include the meaning of a gene product of the human SNAP25 gene, including naturally occurring variants thereof. Two alternative transcript variants encoding different protein isoforms have been described for the human SNAP25 gene. A cDNA sequence corresponding to a human SNAP25 mRNA (variant 1) is found in Genbank Accession No NM_{—}003081. Variant 1 contains 8 exons and encodes isoform SNAP25A. A cDNA sequence corresponding to human SNAP25 mRNA (variant 2) is found in Genbank Accession No NM_{—}130811 and contains an alternate exon 5 as compared to transcript variant 1. This results in an isoform SNAP25B that is the same length, and contains the same N- and C-termini as isoform SNAP25A, but has 9 amino acid residue differences internally. The amino acid sequence of human SNAP25A and SNAP25B polypeptides includes the sequences found in Genbank Accession Nos. NP_{—}003072 and NP_{—}570824, respectively, and naturally occurring variants thereof. The SNAP25 sequence identified in the screening experiments described herein was from isoform B.

Suitable “anti-apoptotic derivatives” of SNAP25 include anti-apoptotic fragments of SNAP25. By “an anti-apoptotic fragment” of SNAP25 we mean any portion of the SNAP25A or SNAP25B polypeptide that has the ability to inhibit Bax-mediated apoptosis in a cell. Typically, the fragment has at least 30% of the anti-apoptotic activity of the SNAP25 polypeptide fragment encoded by the polynucleotide of SEQ ID No. 9. It is more preferred if the fragment has at least 50%, preferably at least 70% and more preferably at least 90% of the anti-apoptotic activity of the SNAP25 polypeptide encoded by the polynucleotide SEQ ID No. 9. Most preferably, the fragment has 100% or more of the anti-apoptotic activity of the SNAP25 polypeptide encoded by the polynucleotide SEQ ID No. 9.

Suitable “anti-apoptotic derivatives” of SNAP25 also include modifications of full-length SNAP25A or SNAP25B, or modifications of the fragment thereof, that have the ability to inhibit Bax-mediated apoptosis in a cell. Preferably, the modified SNAP25A, SNAP25B, or modified fragment thereof retains at least 90% sequence identity with the full-length SNAP25A, SNAP25B, or the respective fragment thereof. More preferably, the modified SNAP25A, SNAP25B, or modified fragment thereof has at least 91%, 92%, 93%, 94% or 95% sequence identity, and yet more preferably at least 96%, 97%, 98% or 99% sequence identity with the full-length SNAP25A or SNAP25B, or the respective fragment. Preferably, the modified SNAP25 or modified fragment thereof retains at least 30% of the anti-apoptotic activity of the SNAP25 polypeptide encoded by the polynucleotide SEQ ID No. 9. It is more preferred if the modified SNAP25 or modified derivative has at least 50%, preferably at least 70% and more preferably at least 90% of the anti-apoptotic activity of the SNAP25 polypeptide encoded by the polynucleotide SEQ ID No. 9. Most preferably, the modified SNAP25 or modified fragment thereof has 100% or more of the anti-apoptotic activity of the SNAP25 polypeptide encoded by the polynucleotide SEQ ID No. 9.

By SNAP25 we also include a homologous gene product from SNAP25 genes from other species. By “homologous gene product” we include an SNAP25 polypeptide having at least 90% sequence identity with the human SNAP25 amino acid sequences in Genbank Accession Nos. NP_{—}003072 and NP_{—}570824. More preferably, a homologous gene product includes an SNAP25 polypeptide having at least 91%, or at least 92%, or at least 93%, or at least 94%, or at least 95%, or at least 96%, or at least 97%, or at least 98% sequence identity with human SNAP25A or SNAP25B. Most preferably, a homologous gene product includes an SNAP25 polypeptide having at least 99% sequence identity with the human SNAP25A or SNAP25B amino acid sequence. It is appreciated that for applications in which SNAP25 is administered to a non-human subject, the SNAP25 is preferably from the same species as the subject. If the SNAP25 is administered to a human subject, the SNAP25 is preferably a human SNAP25 isoform, or an anti-apoptotic fragment or variant thereof.

By a polynucleotide encoding SNAP25 we include the cDNA encoding the human SNAP25 polypeptide and naturally occurring variants thereof. cDNA sequences encoding human SNAP25 are found in Genbank Accession Nos. NM_{—}003081 and NM_{—}130811. We also include other sequences which, by virtue of the degeneracy of the genetic code, also encode a SNAP25 polypeptide. An example of a polynucleotide encoding an anti-apoptotic fragment of SNAP25 is listed in SEQ ID No: 9, which is a near full-length fragment, missing the nucleotides coding for the first few amino acids.

### RIMS3

RIMS3 is described in Wang et al (2000) and Wang & Sudhof (2003).

RIM1 is a putative effector protein for Rab3s, synaptic GTP-binding proteins. RIM1 is localized close to the active zone at the synapse, where it interacts in a GTP-dependent manner with Rab3 located on synaptic vesicles. RIM2, is highly homologous to RIM1 and also expressed primarily in brain. Like RIM1, RIM2 contains an N-terminal zinc finger domain that binds to Rab3 as a function of GTP, a central PDZ domain, and two C-terminal C(2) domains that are separated by long alternatively spliced sequences. The 3′-end of the RIM2 gene produces an independent mRNA that encodes a smaller protein referred as NIM2. NIM2 is composed of a unique N-terminal sequence followed by the C-terminal part of RIM2. Data bank searches identified a third RIM/NIM-related gene, which encodes a NIM isoform referred to as NIM3 (RIMS3). NIMs, like RIMs, regulate exocytosis. The combination of conserved and variable sequences in RIMs and NIMs indicates that the individual domains of these proteins provide binding sites for interacting molecules during exocytosis.

To the best of the inventor's knowledge, RIMS3 has not been associated with apoptosis.

Thus the invention includes combating Bax-mediated apoptosis in a cell by administering to the cell a RIMS3 polypeptide, or an anti-apoptotic derivative thereof, or a polynucleotide that encodes said polypeptide or derivative.

To the best of the inventor's knowledge, RIMS3 has not been associated with any neurodegenerative disease or condition. Indeed, as far as the inventor is aware, RIMS3 has not been associated with any disease state and has not previously been used therapeutically.

Thus the invention includes a RIMS3 polypeptide, or an anti-apoptotic derivative thereof, or a polynucleotide that encodes said polypeptide or derivative, for use in medicine.

Thus the invention also includes combating a neurodegenerative disease or condition in a patient by administering to the patient a RIMS3 polypeptide, or an anti-apoptotic derivative thereof, or a polynucleotide that encodes said polypeptide or derivative.

By “RIMS3 polypeptide” (regulating synaptic membrane exocytosis 3; also known as Rab-3 interacting molecule 3 and Nim3) we include the meaning of a gene product of the human RIMS3 gene, including naturally occurring variants thereof. A cDNA sequence corresponding to a human RIMS3 mRNA is found in Genbank Accession No NM_{—}014747. Human RIMS3 polypeptide includes the amino acid sequence found in Genbank Accession No NP_{—}055562, and naturally occurring variants thereof.

Suitable “anti-apoptotic derivatives” of RIMS3 include anti-apoptotic fragments of RIMS3. By “an anti-apoptotic fragment” of RIMS3 we mean any portion of the RIMS3 polypeptide that has the ability to inhibit Bax-mediated apoptosis in a cell. Typically, the fragment has at least 30% of the anti-apoptotic activity of the RIMS3 polypeptide fragment encoded by the polynucleotide of SEQ ID No. 10. It is more preferred if the fragment has at least 50%, preferably at least 70% and more preferably at least 90% of the anti-apoptotic activity of the RIMS3 polypeptide encoded by the polynucleotide SEQ ID No. 10. Most preferably, the fragment has 100% or more of the anti-apoptotic activity of the RIMS3 polypeptide encoded by the polynucleotide SEQ ID No. 10.

Suitable “anti-apoptotic derivatives” of RIMS3 also include modifications of full-length RIMS3, or modifications of the fragment thereof, that have the ability to inhibit Bax-mediated apoptosis in a cell. Preferably, the modified RIMS3 or modified fragment of RIMS3 retains at least 90% sequence identity with the full-length RIMS3, or the respective fragment thereof. More preferably, the modified RIMS3, or modified fragment thereof has at least 91%, 92%, 93%, 94% or 95% sequence identity, and yet more preferably at least 96%, 97%, 98% or 99% sequence identity with the full-length RIMS3, or the respective RIMS3 fragment. Preferably, the modified RIMS3 or modified fragment thereof retains at least 30% of the anti-apoptotic activity of the RIMS3 polypeptide encoded by the polynucleotide SEQ ID No. 10. It is more preferred if the modified RIMS3 or modified derivative has at least 50%, preferably at least 70% and more preferably at least 90% of the anti-apoptotic activity of the RIMS3 polypeptide encoded by the polynucleotide SEQ ID No. 10. Most preferably, the modified RIMS3 or modified fragment thereof has 100% or more of the anti-apoptotic activity of the RIMS3 polypeptide encoded by the polynucleotide SEQ ID No. 10.

By RIMS3 we also include a homologous gene product from RIMS3 genes from other species. By “homologous gene product” we include an RIMS3 polypeptide having at least 80% sequence identity with the human RIMS3 amino acid sequence in Genbank Accession No NP_{—}055562. More preferably, a homologous gene product includes an RIMS3 polypeptide having at least 85% or at least 90% sequence identity with human RIMS3. Yet more preferably, a homologous gene product includes a RIMS3 polypeptide having at least 91%, or at least 92%, or at least 93%, or at least 94%, or at least 95%, or at least 96%, or at least 97%, or at least 98% sequence identity with human RIMS3. Most preferably, a homologous gene product includes an RIMS3 polypeptide having at least 99% sequence identity with the human RIMS3 amino acid sequence. It is appreciated that for applications in which RIMS3 is administered to a non-human subject, the RIMS3 is preferably from the same species as the subject. If the RIMS3 is administered to a human subject, the RIMS3 is preferably human RIMS3, or an anti-apoptotic fragment or variant thereof.

By a polynucleotide encoding RIMS3 we include the cDNA encoding the human RIMS3 polypeptide and naturally occurring variants thereof. A cDNA sequence encoding human RIMS3 is found in Genbank Accession No NM_{—}014747. We also include other sequences which, by virtue of the degeneracy of the genetic code, also encode a RIMS3 polypeptide.

The polynucleotide (SEQ ID No: 10) encoding RIMS3 that was isolated in the yeast screen was full length.

### RAB40B

RAB40B is a member of the RAS oncogene family (also known as RAR or SEC4L). Many members of the Ras superfamily of GTPases have been implicated in the regulation of haematopoietic cells, with roles in growth, survival, differentiation, cytokine production, chemotaxis, vesicle-trafficking, and phagocytosis. The Ras superfamily of proteins now includes over 150 small GTPases (distinguished from the large, heterotrimeric GTPases, the G-proteins). It comprises six subfamilies, the Ras, Rho, Ran, Rab, Arf, and Kir/Rem/Rad subfamilies. They exhibit remarkable overall amino acid identities, especially in the regions interacting with the guanine nucleotide exchange factors that catalyze their activation. The evolution of the Rab family of small GTP-binding proteins has been described by Pereira-Leal & Seabra (2001).

Regulation of the endocytic and exocytic vesicle transport pathways is essential for maintaining the structural and functional organization of oligodendrocytes. Vesicle transport pathways are regulated by the concerted control of: (1) the formation of the carrier vesicle in the donor compartment; (2) the movement of the vesicle along elements of the cytoskeleton; and (3) the targeting and fusion of the vesicle into the acceptor compartment. The mechanisms that regulate these events are conserved among eukaryotic cells. Rab proteins are key components in mechanisms that regulate vesicle formation, vesicle movement, and vesicle targeting. Each member of the Rab protein family regulates a specific vesicle trafficking pathway.

To the best of the inventor's knowledge, RAB40B has not been associated with apoptosis.

Thus the invention includes combating Bax-mediated apoptosis in a cell by administering to the cell a RAB40B polypeptide, or an anti-apoptotic derivative thereof, or a polynucleotide that encodes said polypeptide or derivative.

To the best of the inventor's knowledge, RAB40B has not been associated with any neurodegenerative disease or condition. Indeed, as far as the inventor is aware, RAB40B has not been associated with any disease state and has not previously been used therapeutically.

Thus the invention includes a RAB40B polypeptide, or an anti-apoptotic derivative thereof, or a polynucleotide that encodes said polypeptide or derivative, for use in medicine.

The invention also includes combating a neurodegenerative disease or condition in a patient by administering to the patient a RAB40B polypeptide, or an anti-apoptotic derivative thereof, or a polynucleotide that encodes said polypeptide or derivative.

By “RAB40B polypeptide” we include the meaning of a gene product of the human RAB40B gene, including naturally occurring variants thereof. A cDNA sequence corresponding to a human RAB40B mRNA is found in Genbank Accession No NM_{—}006822. Human RAB40B polypeptide includes the amino acid sequence found in Genbank Accession No NP_{—}006813, and naturally occurring variants thereof.

Suitable “anti-apoptotic derivatives” of RAB40B include anti-apoptotic fragments of RAB40B. By “an anti-apoptotic fragment” of RAB40B we mean any portion of the RAB40B polypeptide that has the ability to inhibit Bax-mediated apoptosis in a cell. Typically, the fragment has at least 30% of the anti-apoptotic activity of the RAB40B polypeptide fragment encoded by the polynucleotide of SEQ ID No. 11. It is more preferred if the fragment has at least 50%, preferably at least 70% and more preferably at least 90% of the anti-apoptotic activity of the RAB40B polypeptide encoded by the polynucleotide SEQ ID No. 11. Most preferably, the fragment has 100% or more of the anti-apoptotic activity of the RAB40B polypeptide encoded by the polynucleotide SEQ ID No. 11.

Suitable “anti-apoptotic derivatives” of RAB40B also include modifications of full-length RAB40B, or modifications of the fragment thereof, that have the ability to inhibit Bax-mediated apoptosis in a cell. Preferably, the modified RAB40B or modified fragment of RAB40B retains at least 90% sequence identity with the full-length RAB40B, or the respective fragment thereof. More preferably, the modified RAB40B, or modified fragment thereof has at least 91%, 92%, 93%, 94% or 95% sequence identity, and yet more preferably at least 96%, 97%, 98% or 99% sequence identity with the full-length RAB40B, or the respective RAB40B fragment. Preferably, the modified RAB40B or modified fragment thereof retains at least 30% of the anti-apoptotic activity of the RAB40B polypeptide encoded by the polynucleotide SEQ ID No. 11. It is more preferred if the modified RAB40B or modified derivative has at least 50%, preferably at least 70% and more preferably at least 90% of the anti-apoptotic activity of the RAB40B polypeptide encoded by the polynucleotide SEQ ID No. 11. Most preferably, the modified RAB40B or modified fragment thereof has 100% or more of the anti-apoptotic activity of the RAB40B polypeptide encoded by the polynucleotide SEQ ID No. 11.

By RAB40B we also include a homologous gene product from RAB40B genes from other species. By “homologous gene product” we include an RAB40B polypeptide having at least 90% sequence identity with the human RAB40B amino acid sequence in Genbank Accession No NP_{—}006813. More preferably, a homologous gene product includes an RAB40B polypeptide having at least 91%, or at least 92%, or at least 93%, or at least 94%, or at least 95%, or at least 96%, or at least 97%, or at least 98% sequence identity with human RAB40B. Most preferably, a homologous gene product includes an RAB40B polypeptide having at least 99% sequence identity with the human RAB40B amino acid sequence. It is appreciated that for applications in which RAB40B is administered to a non-human subject, the RAB40B is preferably from the same species as the subject. If the RAB40B is administered to a human subject, the RAB40B is preferably human RAB40B, or an anti-apoptotic fragment or variant thereof.

By a polynucleotide encoding RAB40B we include the cDNA encoding the human RAB40B polypeptide and naturally occurring variants thereof. A cDNA sequence encoding human RAB40B is found in Genbank Accession No NM_{—}006822. We also include other sequences which, by virtue of the degeneracy of the genetic code, also encode a RAB40B polypeptide. An example of a polynucleotide encoding an anti-apoptotic fragment of RAB40B is listed in SEQ ID No: 11. However, the full-length RAB40B is more effective at rescuing yeast cells from Bax-mediated apoptosis than this fragment of SEQ ID No: 11.

### HMGCS1

HMGCS1 (3-hydroxy-3-methylglutaryl-coenzyme A synthase 1) is a cytoplasmic enzyme that condenses acetyl-CoA with acetoacetyl-CoA to form HMG-CoA, which is the substrate for HMG-CoA reductase.

Acetyl-CoA+H₂O+acetoacetyl-CoA [symbol] (S)-3-hydroxy-3-methylglutaryl-CoA+CoA.

HMG coA synthase is also involved in the pathway of production of mevalonate from HMG-CoA prior to the synthesis of sterols such as cholesterol and isoprenoids. The gene for the mitochondrial HMG coA synthase is a target for PPAR (peroxisome proliferator-activated receptor) and this receptor mediates the induction of this gene by fatty acids. Human cytoplasmic 3-hydroxy-3-methylglutaryl coenzyme A synthase has been described by Rokosz (1994).

To the best of the inventor's knowledge, HMGCS1 has not been associated with apoptosis.

Thus the invention includes combating Bax-mediated apoptosis in a cell by administering to the cell a HMGCS1 polypeptide, or an anti-apoptotic derivative thereof, or a polynucleotide that encodes said polypeptide or derivative.

Although there is a link between HMG CoA reductase and AD, there is no known link between HMG CoA synthase and AD. To the best of the inventor's knowledge, HMGCS1 has not been associated with any neurodegenerative disease or condition. To the best of the inventor's knowledge, HMGCS1 has not been associated with any neurodegenerative disease or condition. Indeed, according to OMIM Reference No 142940, HMGCS1 has not been associated with any disease state and, as far as the inventor is aware, HMGCS1 has not been used therapeutically.

Thus the invention includes a HMGCS1 polypeptide, or an anti-apoptotic derivative thereof, or a polynucleotide that encodes said polypeptide or derivative, for use in medicine.

The invention also includes combating a neurodegenerative disease or condition in a patient by administering to the patient a HMGCS1 polypeptide, or an anti-apoptotic derivative thereof, or a polynucleotide that encodes said polypeptide or derivative.

By HMGCS1 polypeptide we include the meaning of a gene product of the human HMGCS1 gene, including naturally occurring variants thereof. A cDNA sequence corresponding to a human HMGCS1 mRNA is found in Genbank Accession No NM_{—}002130. Human HMGCS1 polypeptide includes the amino acid sequence found in Genbank Accession No NP_{—}002121, and naturally occurring variants thereof.

Suitable “anti-apoptotic derivatives” of HMGCS1 include anti-apoptotic fragments of HMGCS1. By “an anti-apoptotic fragment” of HMGCS1 we mean any portion of the HMGCS1 polypeptide that has the ability to inhibit Bax-mediated apoptosis in a cell. Typically, the fragment has at least 30% of the anti-apoptotic activity of the HMGCS1 polypeptide fragment encoded by the polynucleotide of SEQ ID No. 12. It is more preferred if the fragment has at least 50%, preferably at least 70% and more preferably at least 90% of the anti-apoptotic activity of the HMGCS1 polypeptide encoded by the polynucleotide SEQ ID No. 12. Most preferably, the fragment has 100% or more of the anti-apoptotic activity of the HMGCS1 polypeptide encoded by the polynucleotide SEQ ID No. 12.

Suitable “anti-apoptotic derivatives” of HMGCS1 also include modifications of full-length HMGCS1, or modifications of the fragment thereof, that have the ability to inhibit Bax-mediated apoptosis in a cell. Preferably, the modified HMGCS1 or modified fragment of HMGCS1 retains at least 90% sequence identity with the full-length human HMGCS1 (as defined in NP_{—}002121), or the respective fragment thereof. More preferably, the modified HMGCS1, or modified fragment thereof has at least 91%, 92%, 93%, 94% or 95% sequence identity, and yet more preferably at least 96%, 97%, 98% or 99% sequence identity with the full-length HMGCS1, or the respective HMGCS1 fragment. Preferably, the modified HMGCS1 or modified fragment thereof retains at least 30% of the anti-apoptotic activity of the HMGCS1 polypeptide encoded by the polynucleotide SEQ ID No. 12. It is more preferred if the modified HMGCS1 or modified derivative has at least 50%, preferably at least 70% and more preferably at least 90% of the anti-apoptotic activity of the HMGCS1 polypeptide encoded by the polynucleotide SEQ ID No. 12. Most preferably, the modified HMGCS1 or modified fragment thereof has 100% or more of the anti-apoptotic activity of the HMGCS1 polypeptide encoded by the polynucleotide SEQ ID No. 12.

By HMGCS1 we also include a homologous gene product from HMGCS1 genes from other species. By “homologous gene product” we include an HMGCS1 polypeptide having at least 80% sequence identity with the human HMGCS1 amino acid sequence in Genbank Accession No NP_{—}002121. More preferably, a homologous gene product includes an HMGCS1 polypeptide having at least 85% or at least 90% sequence identity with human HMGCS1. Yet more preferably, a homologous gene product includes a HMGCS1 polypeptide having at least 91%, or at least 92%, or at least 93%, or at least 94%, or at least 95%, or at least 96%, or at least 97%, or at least 98% sequence identity with human HMGCS1. Most preferably, a homologous gene product includes an HMGCS1 polypeptide having at least 99% sequence identity with the human HMGCS1 amino acid sequence. It is appreciated that for applications in which HMGCS1 is administered to a non-human subject, the HMGCS1 is preferably from the same species as the subject. If the HMGCS1 is administered to a human subject, the HMGCS1 is preferably human HMGCS1, or an anti-apoptotic fragment or variant thereof.

By a polynucleotide encoding HMGCS1 we include the cDNA encoding the human HMGCS1 polypeptide and naturally occurring variants thereof. A cDNA sequence encoding human HMGCS1 is found in Genbank Accession No NM_{—}002130. We also include other sequences which, by virtue of the degeneracy of the genetic code, also encode a HMGCS1 polypeptide. An example of a polynucleotide encoding an anti-apoptotic fragment of HMGCS1 is listed in SEQ ID No: 12, which is a partial 3′ sequence of HMGCS1.

### SCD5

SCD5 (stearoyl-CoA desaturase 5; previously known as Acyl-CoA Desaturase 4 isoform a; ACOD4), as with other stearoyl-CoA desaturases (SCD; EC 1.14.99.5) catalyses the committed step in the biosynthesis of monounsaturated fatty acids from saturated fatty acids. This reaction involves the introduction of a cis-double bond between carbons 9 and 10 in a spectrum of methylene-interrupted fatty acyl-CoAs.

To the best of the inventor's knowledge, SCD5 has not been associated with apoptosis.

Thus the invention includes combating Bax-mediated apoptosis in a cell by administering to the cell a SCD5 polypeptide, or an anti-apoptotic derivative thereof, or a polynucleotide that encodes said polypeptide or derivative.

To the best of the inventor's knowledge, SCD5 has not been associated with any neurodegenerative disease or condition. Indeed, according to OMIM Reference No 608370, SCD5 has not been associated with any disease state and, as far as the inventor is aware, SCD5 has not been used therapeutically.

Thus the invention includes a SCD5 polypeptide, or an anti-apoptotic derivative thereof, or a polynucleotide that encodes said polypeptide or derivative, for use in medicine.

The invention also includes combating a neurodegenerative disease or condition in a patient by administering to the patient a SCD5 polypeptide, or an anti-apoptotic derivative thereof, or a polynucleotide that encodes said polypeptide or derivative.

By “SCD5 polypeptide” we include the meaning of a gene product of the human SCD5 gene, including naturally occurring variants thereof. A cDNA sequence corresponding to a human SCD5 mRNA is found in Genbank Accession No. BC048971.1. Human SCD5 polypeptide includes the amino acid sequence found in Genbank Accession No. AAH48971, and naturally occurring variants thereof.

Suitable “anti-apoptotic derivatives” of SCD5 include anti-apoptotic fragments of SCD5. By “an anti-apoptotic fragment” of SCD5 we mean any portion of the SCD5 polypeptide that has the ability to inhibit Bax-mediated apoptosis in a cell. Typically, the fragment has at least 30% of the anti-apoptotic activity of the SCD5 polypeptide fragment encoded by the polynucleotide of SEQ ID No. 13. It is more preferred if the fragment has at least 50%, preferably at least 70% and more preferably at least 90% of the anti-apoptotic activity of the SCD5 polypeptide encoded by the polynucleotide SEQ ID No. 13. Most preferably, the fragment has 100% or more of the anti-apoptotic activity of the SCD5 polypeptide encoded by the polynucleotide SEQ ID No. 13.

Suitable “anti-apoptotic derivatives” of SCD5 also include modifications of full-length SCD5, or modifications of the fragment thereof, that have the ability to inhibit Bax-mediated apoptosis in a cell. Preferably, the modified SCD5 or modified fragment of SCD5 retains at least 90% sequence identity with the full-length human SCD5 (as defined in AAH48971), or the respective fragment thereof. More preferably, the modified SCD5, or modified fragment thereof has at least 91%, 92%, 93%, 94% or 95% sequence identity, and yet more preferably at least 96%, 97%, 98% or 99% sequence identity with the full-length SCD5, or the respective SCD5 fragment. Preferably, the modified SCD5 or modified fragment thereof retains at least 30% of the anti-apoptotic activity of the SCD5 polypeptide encoded by the polynucleotide SEQ ID No. 13. It is more preferred if the modified SCD5 or modified derivative has at least 50%, preferably at least 70% and more preferably at least 90% of the anti-apoptotic activity of the SCD5 polypeptide encoded by the polynucleotide SEQ ID No. 13. Most preferably, the modified SCD5 or modified fragment thereof has 100% or more of the anti-apoptotic activity of the SCD5 polypeptide encoded by the polynucleotide SEQ ID No. 13.

By SCD5 we also include a homologous gene product from SCD5 genes from other species. By “homologous gene product” we include an SCD5 polypeptide having at least 80% sequence identity with the human SCD5 amino acid sequence in Genbank Accession No. AAH48971. More preferably, a homologous gene product includes an SCD5 polypeptide having at least 85% or at least 90% sequence identity with human SCD5. Yet more preferably, a homologous gene product includes a SCD5 polypeptide having at least 91%, or at least 92%, or at least 93%, or at least 94%, or at least 95%, or at least 96%, or at least 97%, or at least 98% sequence identity with human SCD5. Most preferably, a homologous gene product includes an SCD5 polypeptide having at least 99% sequence identity with the human SCD5 amino acid sequence. It is appreciated that for applications in which SCD5 is administered to a non-human subject, the SCD5 is preferably from the same species as the subject. If the SCD5 is administered to a human subject, the SCD5 is preferably human SCD5, or an anti-apoptotic fragment or variant thereof.

By a polynucleotide encoding SCD5 we include the cDNA encoding the human SCD5 polypeptide and naturally occurring variants thereof. A cDNA sequence encoding human SCD5 is found in Genbank Accession No. BC048971.1. We also include other sequences which, by virtue of the degeneracy of the genetic code, also encode a SCD5 polypeptide. An example of a polynucleotide encoding an anti-apoptotic fragment of SCD5 is listed in SEQ ID No: 13.

### ATP2A2

ATP2A2 (also known as ATP2B and SERCA2) encodes one of the SERCA Ca²⁺-ATPases (EC 3.6.3.8), which are intracellular pumps located in the sarcoplasmic or endoplasmic reticula of muscle cells. This enzyme catalyzes the hydrolysis of ATP coupled with the translocation of calcium from the cytosol to the sarcoplasmic reticulum lumen, and is involved in regulation of the contraction/relaxation cycle.

According to Shull et al (2003), plasma membrane Ca²⁺-transporting ATPases (PMCAs) extrude Ca²⁺ from the cell, and sarco(endo)plasmic reticulum Ca²⁺-ATPases (SERCAs) and secretory pathway Ca²⁺-ATPases (SPCAs) sequester Ca²⁺ in intracellular organelles. However, the specific physiological functions of individual isoforms are less well understood. This information is beginning to emerge from studies of mice and humans carrying null mutations in the corresponding genes. Mice with targeted or spontaneous mutations in plasma membrane Ca²⁺-ATPase isoform 2 (PMCA2) are profoundly deaf and have a balance defect due to the loss of PMCA2 in sensory hair cells of the inner ear. In humans, mutations in SERCA1 (ATP2A1) cause Brody disease, an impairment of skeletal muscle relaxation; loss of one copy of the SERCA2 (ATP2A2) gene causes Darier disease, a skin disorder; and loss of one copy of the SPCA1 (ATP2C1) gene causes Hailey-Hailey disease, another skin disorder. In the mouse, SERCA2 null mutants do not survive to birth, and heterozygous SERCA2 mutants have impaired cardiac performance and a high incidence of squamous cell cancers. SERCA3 null mutants survive and appear healthy, but endothelium-dependent relaxation of vascular smooth muscle is impaired and Ca²⁺ signalling is altered in pancreatic beta cells. The diversity of phenotypes indicates that the various Ca²⁺-transporting ATPase isoforms serve very different physiological functions.

Darier's disease is a rare cutaneous disease with an autosomal dominant mode of inheritance. Greasy papules and plaques arise on the seborrheic areas and in the flexures and almost all patients have nail abnormalities. It is characterised by loss of adhesion between epidermal cells and abnormal keratinisation, and acantholysis and dyskeratosis are the typical histological findings. The underlying defect is a result of mutations in the ATP2A2 gene on chromosome 12q23-24 that encodes for a sarco/endoplasmic reticulum calcium ATPase (SERCA 2). Acantholysis is thought to result from desmosome breakdown. Darier's disease (also known as Darier-White disease and keratosis follicularis) is an example of a dominantly inherited disease caused by haplo-insufficiency. Oral retinoids are the most effective treatment but their adverse effects are troublesome. Topical retinoids, topical corticosteroids, surgery, and laser surgery have their advocates but evidence for efficacy is sparse (Cooper & Burge, 2003). Darier's disease is sometimes co-morbid with mood disorders such as bipolar disorder (Kato, 2001). The role of ATP2A2 in Darier's disease is reviewed by Foggia & Hovnanian (2004).

According to Dhitavat et al (2004), in Darier's disease, loss of desmosomal adhesion triggers anoikis, a type of apoptosis characterized by cell detachment in keratinocytes. The apoptosis was considered to be secondary to changes in Ca²⁺ transport activity. In support of their hypothesis, Dhitavat et al referred to the finding by Jackisch et al (2000) that inhibition of SERCA pumps with thapsigargin triggers apoptosis in a variety of epithelial cells.

Prasad et al (2005) describes that haploinsufficiency of ATP2A2 predisposes mice to squamous cell tumours. However, in clear contrast to the findings of Dhitavat et al, Prasad et al refer to Qu et al (2004) and state that “ER stress induced by thapsigargin, a SERCA2 inhibitor, has been shown to prevent p53-mediated apoptosis”.

Thus, to the best of the inventor's knowledge, ATP2A2 has not been directly associated with Bax-mediated apoptosis.

Accordingly, the invention includes combating Bax-mediated apoptosis in a cell by administering to the cell an ATP2A2 polypeptide, or an anti-apoptotic derivative thereof, or a polynucleotide that encodes said polypeptide or derivative.

Despite the co-morbidity of Darier's disease with bipolar disorder, to the best of the inventor's knowledge, ATP2A2 has not been directly associated with any neurodegenerative disease or condition. Indeed, in the context of this invention, bipolar disorder is not considered to be a neurodegenerative disease or condition.

Thus the invention also includes combating a neurodegenerative disease or condition in a patient by administering to the patient a ATP2A2 polypeptide, or an anti-apoptotic derivative thereof, or a polynucleotide that encodes said polypeptide or derivative.

By “ATP2A2 polypeptide” we include the meaning of a gene product of the human ATP2A2 gene, including naturally occurring variants thereof. Two alternative transcript variants encoding different protein isoforms have been described for the human ATP2A2 gene. A cDNA sequence corresponding to a human ATP2A2 mRNA (variant 1) is found in Genbank Accession No NM_{—}170665. A cDNA sequence corresponding to a human ATP2A2 mRNA (variant 2) is found in Genbank Accession No NM_{—}001681. Transcript variant 1 (NM_{—}170665) encodes the longer isoform 1 of the ATP2A2 protein. Transcript variant 2 (NM_{—}001681) uses alternative splice sites in the 3′ end of the coding region resulting in an earlier termination codon than variant 1. Isoform 2 has a shorter and distinct C-terminus compared to isoform 1. The amino acid sequence of human ATP2A2 variant 1 and ATP2A variant 2 polypeptides includes the sequences found in Genbank Accession Nos. NP_{—}733765 and NP_{—}001672, respectively, and naturally occurring variants thereof.

Suitable “anti-apoptotic derivatives” of ATP2A2 include anti-apoptotic fragments of ATP2A2. By “an anti-apoptotic fragment” of ATP2A2 we mean any portion of the ATP2A2 polypeptide, isoform 1 or isoform 2, that has the ability to inhibit Bax-mediated apoptosis in a cell. Typically, the fragment has at least 30% of the anti-apoptotic activity of the ATP2A2 polypeptide fragment encoded by the polynucleotide of SEQ ID No. 14. It is more preferred if the fragment has at least 50%, preferably at least 70% and more preferably at least 90% of the anti-apoptotic activity of the ATP2A2 polypeptide encoded by the polynucleotide SEQ ID No. 14. Most preferably, the fragment has 100% or more of the anti-apoptotic activity of the ATP2A2 polypeptide encoded by the polynucleotide SEQ ID No. 14.

Suitable “anti-apoptotic derivatives” of ATP2A2 also include modifications of full-length ATP2A2 (whether isoform 1 or isoform 2), or modifications of the fragment thereof, that have the ability to inhibit Bax-mediated apoptosis in a cell. Preferably, the modified ATP2A2 or modified fragment of ATP2A2 retains at least 90% sequence identity with the full-length human ATP2A2 (as defined in NP_{—}733765 or NP_{—}001672), or the respective fragment thereof. More preferably, the modified ATP2A2, or modified fragment thereof has at least 91%, 92%, 93%, 94% or 95% sequence identity, and yet more preferably at least 96%, 97%, 98% or 99% sequence identity with the full-length ATP2A2, or the respective ATP2A2 fragment. Preferably, the modified ATP2A2 or modified fragment thereof retains at least 30% of the anti-apoptotic activity of the ATP2A2 polypeptide encoded by the polynucleotide SEQ ID No. 14. It is more preferred if the modified ATP2A2 or modified derivative has at least 50%, preferably at least 70% and more preferably at least 90% of the anti-apoptotic activity of the ATP2A2 polypeptide encoded by the polynucleotide SEQ ID No. 14. Most preferably, the modified ATP2A2 or modified fragment thereof has 100% or more of the anti-apoptotic activity of the ATP2A2 polypeptide encoded by the polynucleotide SEQ ID No. 14.

By ATP2A2 we also include a homologous gene product from ATP2A2 genes from other species. By “homologous gene product” we include an ATP2A2 polypeptide having at least 90% sequence identity with the human ATP2A2 amino acid sequence in Genbank Accession Nos. NP_{—}733765 or NP_{—}001672. More preferably, a homologous gene product includes an ATP2A2 polypeptide having at least 91%, or at least 92%, or at least 93%, or at least 94%, or at least 95%, or at least 96%, or at least 97%, or at least 98% sequence identity with human ATP2A2. Most preferably, a homologous gene product includes an ATP2A2 polypeptide having at least 99% sequence identity with the human ATP2A2 amino acid sequence. It is appreciated that for applications in which ATP2A2 is administered to a non-human subject, the ATP2A2 is preferably from the same species as the subject. If the ATP2A2 is administered to a human subject, the ATP2A2 is preferably human ATP2A2, or an anti-apoptotic fragment or variant thereof.

By a polynucleotide encoding ATP2A2 we include the cDNA encoding the human ATP2A2 polypeptide and naturally occurring variants thereof. A cDNA sequence encoding human ATP2A2 is found in Genbank Accession Nos. NM_{—}170665 and NM_{—}001681. We also include other sequences which, by virtue of the degeneracy of the genetic code, also encode a ATP2A2 polypeptide. An example of a polynucleotide encoding an anti-apoptotic fragment of ATP2A2 is listed in SEQ ID No: 14.

### HRMT1L1

HRMT1L1 (heterogeneous nuclear ribonucleoprotein methyltransferase-like 1; also known as hmt1-like 1; protein arginine n-methyltransferase 2; prmt2) is an arginine methyltransferase which may act on RNA-binding proteins such as heterogeneous nuclear ribonucleoproteins.

To the best of the inventor's knowledge, HRMT1L1 has not been associated with apoptosis.

Thus the invention includes combating Bax-mediated apoptosis in a cell by administering to the cell an HRMT1L1 polypeptide, or an anti-apoptotic derivative thereof, or a polynucleotide that encodes said polypeptide or derivative.

To the best of the inventor's knowledge, HRMT1L1 has not been associated with any neurodegenerative disease or condition. Indeed, according to OMIM Reference No 601961, HRMT1L1 has not been associated with any disease state and, as far as the inventor is aware, has not previously been used therapeutically.

Thus the invention includes a HRMT1L1 polypeptide, or an anti-apoptotic derivative thereof, or a polynucleotide that encodes said polypeptide or derivative, for use in medicine.

The invention also includes combating a neurodegenerative disease or condition in a patient by administering to the patient an HRMT1L1 polypeptide, or an anti-apoptotic derivative thereof, or a polynucleotide that encodes said polypeptide or derivative.

By “HRMT1L1 polypeptide” we include the meaning of a gene product of the human HRMT1L1 gene, including naturally occurring variants thereof. Two alternative transcript variants encoding the same protein isoform have been described for the human HRMT1L1 gene. A cDNA sequence corresponding to a human HRMT1L1 mRNA (variant 1), which is the longer transcript, is found in Genbank Accession No NM_{—}206962. A cDNA sequence corresponding to a human HRMT1L1 mRNA (variant 2) is found in Genbank Accession No NM_{—}001535. Variant 2 differs in the 5′ UTR compared to variant 1. The amino acid sequence of human HRMT1L1 polypeptide includes the sequence found in Genbank Accession Nos. NP_{—}996845 and NP_{—}001526, and naturally occurring variants thereof.

Suitable “anti-apoptotic derivatives” of HRMT1L1 include anti-apoptotic fragments of HRMT1L1. By “an anti-apoptotic fragment” of HRMT1L1 we mean any portion of the HRMT1L1 polypeptide that has the ability to inhibit Bax-mediated apoptosis in a cell. Typically, the fragment has at least 30% of the anti-apoptotic activity of the HRMT1L1 polypeptide fragment encoded by the polynucleotide of SEQ ID No. 15. It is more preferred if the fragment has at least 50%, preferably at least 70% and more preferably at least 90% of the anti-apoptotic activity of the HRMT1L1 polypeptide encoded by the polynucleotide of SEQ ID No. 15. Most preferably, the fragment has 100% or more of the anti-apoptotic activity of the HRMT1L1 polypeptide encoded by the polynucleotide of SEQ ID No. 15.

Suitable “anti-apoptotic derivatives” of HRMT1L1 also include modifications of full-length HRMT1L1, or modifications of the fragment thereof, that have the ability to inhibit Bax-mediated apoptosis in a cell. Preferably, the modified HRMT1L1 or modified fragment of HRMT1L1 retains at least 90% sequence identity with the full-length human HRMT1L1 (as defined in NP_{—}996845), or the respective fragment thereof. More preferably, the modified HRMT1L1, or modified fragment thereof has at least 91%, 92%, 93%, 94% or 95% sequence identity, and yet more preferably at least 96%, 97%, 98% or 99% sequence identity with the full-length HRMT1L1, or the respective HRMT1L1 fragment. Preferably, the modified HRMT1L1 or modified fragment thereof retains at least 30% of the anti-apoptotic activity of the HRMT1L1 polypeptide encoded by the polynucleotide of SEQ ID No. 15. It is more preferred if the modified HRMT1L1 or modified derivative has at least 50%, preferably at least 70% and more preferably at least 90% of the anti-apoptotic activity of the HRMT1L1 polypeptide encoded by the polynucleotide SEQ ID No. 15. Most preferably, the modified HRMT1L1 or modified fragment thereof has 100% or more of the anti-apoptotic activity of the HRMT1L1 polypeptide encoded by the polynucleotide SEQ ID No. 15.

By HRMT1L1 we also include a homologous gene product from HRMT1L1 genes from other species. By “homologous gene product” we include an HRMT1L1 polypeptide having at least 80% sequence identity with the human HRMT1L1 amino acid sequence in Genbank Accession No NP_{—}996845. More preferably, a homologous gene product includes an HRMT1L1 polypeptide having at least 85% or at least 90% sequence identity with human HRMT1L1. Yet more preferably, a homologous gene product includes a HRMT1L1 polypeptide having at least 91%, or at least 92%, or at least 93%, or at least 94%, or at least 95%, or at least 96%, or at least 97%, or at least 98% sequence identity with human HRMT1L1. Most preferably, a homologous gene product includes an HRMT1L1 polypeptide having at least 99% sequence identity with the human HRMT1L1 amino acid sequence. It is appreciated that for applications in which HRMT1L1 is administered to a non-human subject, the HRMT1L1 is preferably from the same species as the subject. If the HRMT1L1 is administered to a human subject, the HRMT1L1 is preferably human HRMT1L1, or an anti-apoptotic fragment or variant thereof.

By a polynucleotide encoding HRMT1L1 we include the cDNA encoding the human HRMT1L1 polypeptide and naturally occurring variants thereof. A cDNA sequence encoding human HRMT1L1 is found in Genbank Accession Nos. NM_{—}206962 and NM_{—}001535. We also include other sequences which, by virtue of the degeneracy of the genetic code, also encode a HRMT1L1 polypeptide. An example of a polynucleotide encoding an anti-apoptotic fragment of HRMT1L1 is listed in SEQ ID No: 15, which is a nearly full-length HRMT1L1 polynucleotide.

The Sequence from Human Chromosome 3p of Previously Unknown Function

The polynucleotide from human chromosome 3p as defined in SEQ ID No: 16 (see FIG. 63) is present on clone RP11-605M1 map 3p (Genbank Accession No AC087091) and on clone Homo sapiens 3 BAC RP11-114K9 (Genbank Accession No AC011609). The length of the complete sequence of clone RP11-605M1 is 153013 nucleotides, and SEQ ID No: 16 is from nucleotides 91339-91958. The length of the complete sequence of clone RP11-114K9 is 184680 nucleotides, and SEQ ID No: 16 is from nucleotides 79443-80063. Two open reading frames are present within SEQ ID No: 16 (indicated by the arrows in FIG. 63). ORF-2 is found at position 98-217 bp of SEQ ID No: 16, and ORF-3 is found at position 260-361 bp of SEQ ID No: 16, and these ORFs would encode the following two short polypeptides (SEQ ID Nos: 20 and 21, respectively):

### Protein-ORF-2: MLRNLGLVAF VLEKLFLEDE SAVHVILQVM WHLFFKLKM

### Protein-ORF-3: MCQEFKNVSY WCVCALFQES TFSVFQLTAP FPS

To the best of the inventor's knowledge, neither the polynucleotide from human chromosome 3p as defined in SEQ ID No: 16, nor a polypeptide encoded by the polynucleotide of SEQ ID No: 16, nor a naturally-occurring polypeptide comprising the polypeptide encoded by the polynucleotide of SEQ ID No: 16, have previously been associated with apoptosis.

Thus the invention includes combating Bax-mediated apoptosis in a cell by administering to the cell a polypeptide encoded by the polynucleotide of SEQ ID No: 16, or a naturally-occurring polypeptide comprising the polypeptide encoded by the polynucleotide of SEQ ID No: 16, or an anti-apoptotic derivative thereof, or a polynucleotide that encodes said polypeptide or derivative.

To the best of the inventor's knowledge, neither the polynucleotide from human chromosome 3p as defined in SEQ ID No: 16, nor a polypeptide encoded by the polynucleotide of SEQ ID No: 16, nor a naturally-occurring polypeptide comprising the polypeptide encoded by the polynucleotide of SEQ ID No: 16, have previously been associated with any neurodegenerative disease or condition. Indeed, as far as the inventor is aware, such polypeptides have not been associated with any disease state and have not previously been used therapeutically.

Thus the invention includes a polypeptide encoded by the polynucleotide of SEQ ID No: 16, or a naturally-occurring polypeptide comprising the polypeptide encoded by the polynucleotide of SEQ ID No: 16, or an anti-apoptotic derivative thereof, or a polynucleotide that encodes said polypeptide or derivative, for use in medicine.

The invention includes combating a neurodegenerative disease or condition in a patient by administering to the cell a polypeptide encoded by the polynucleotide of SEQ ID No: 16, or a naturally-occurring polypeptide comprising the polypeptide encoded by the polynucleotide of SEQ ID No: 16, or an anti-apoptotic derivative thereof, or a polynucleotide that encodes said polypeptide or derivative.

Suitable “anti-apoptotic derivatives” include anti-apoptotic fragments of a polypeptide encoded by the polynucleotide of SEQ ID No: 16, or anti-apoptotic fragments of a naturally-occurring polypeptide comprising the polypeptide encoded by the polynucleotide of SEQ ID No: 16, or anti-apoptotic fragments of a polypeptide encoded by a naturally-occurring polynucleotide transcript comprising SEQ ID No: 16. By “an anti-apoptotic fragment” we mean any portion of the polypeptide that has the ability to inhibit Bax-mediated apoptosis in a cell. Typically, the fragment has at least 30% of the anti-apoptotic activity of the polypeptide fragment encoded by the polynucleotide of SEQ ID No. 16. It is more preferred if the fragment has at least 50%, preferably at least 70% and more preferably at least 90% of the anti-apoptotic activity of the polypeptide fragment encoded by the polynucleotide of SEQ ID No. 16. Most preferably, the fragment has 100% or more of the anti-apoptotic activity of the polypeptide fragment encoded by the polynucleotide of SEQ ID No. 16.

Suitable “anti-apoptotic derivatives” also include modifications of a polypeptide encoded by the polynucleotide of SEQ ID No: 16, or of a full-length naturally-occurring polypeptide comprising the polypeptide encoded by the polynucleotide of SEQ ID No: 16, or of a polypeptide encoded by a naturally-occurring polynucleotide transcript comprising SEQ ID No: 16, or modifications of the fragment thereof, that have the ability to inhibit Bax-mediated apoptosis in a cell. Preferably, the corresponding section of the modified derivative retains at least 90% sequence identity with the polypeptide encoded by the polynucleotide of SEQ ID No: 16, or the respective fragment thereof. More preferably, the corresponding section of the modified derivative, or modified fragment thereof, has at least 91%, 92%, 93%, 94% or 95% sequence identity, and yet more preferably at least 96%, 97%, 98% or 99% sequence identity with the polypeptide encoded by the polynucleotide of SEQ ID No: 16, or the respective fragment thereof. Preferably, the modified polypeptide or modified fragment thereof retains at least 30% of the anti-apoptotic activity of the polypeptide encoded by the polynucleotide of SEQ ID No. 16. It is more preferred if the modified polypeptide or modified fragment has at least 50%, preferably at least 70% and more preferably at least 90% of the anti-apoptotic activity of the polypeptide encoded by the polynucleotide SEQ ID No. 16. Most preferably, the modified polypeptide or modified fragment thereof has 100% or more of the anti-apoptotic activity of the polypeptide encoded by the polynucleotide SEQ ID No. 16.

It is appreciated that for applications in which this gene/polypeptide is administered to a subject, it is preferably from the same species as the subject, particularly if the subject is a human subject.

The Sequence from Human Chromosome 22q11.22-12.2 of Previously Unknown Function

The polynucleotide from human chromosome 22q11.22-12.2 as defined in SEQ ID No: 17 (see FIG. 64) is present on clone CTA-373H7 (Genbank Accession No Z99774). The length of the complete sequence of clone CTA-373H7 is 73239 nucleotides, and SEQ ID No: 17 is from nucleotides 41798-42499. Four open reading frames are present within SEQ ID No: 17 (indicated by the arrows in FIG. 64), two of which encode polypeptides of 45 residues, while the other two encode polypeptides of 30 amino acid residues. These four ORFs are located at the following positions within SEQ ID No: 17: ORF-8: 149-241; ORF-4: 153-290; ORF-9: 431-523; and ORF-5: 565-702.

SEQ ID No: 17 has strong homology to Homo sapiens cDNA FLJ45323 fis, clone BRHIP3006390 (Genbank Accession No AK127256), at nucleotides 4445-5146 of 5292.

To the best of the inventor's knowledge, neither the polynucleotide from human chromosome 22q11.22-12.2 as defined in SEQ ID No: 17, nor a polypeptide encoded by the polynucleotide of SEQ ID No: 17, nor a naturally-occurring polypeptide comprising the polypeptide encoded by the polynucleotide of SEQ ID No: 17, have previously been associated with apoptosis.

Thus the invention includes combating Bax-mediated apoptosis in a cell by administering to the cell a polypeptide encoded by the polynucleotide of SEQ ID No: 17, or a naturally-occurring polypeptide comprising the polypeptide encoded by the polynucleotide of SEQ ID No: 17, or an anti-apoptotic derivative thereof, or a polynucleotide that encodes said polypeptide or derivative.

Also, to the best of the inventor's knowledge, neither the polynucleotide from human chromosome 22q11.22-12.2 as defined in SEQ ID No: 17, nor a polypeptide encoded by the polynucleotide of SEQ ID No: 17, nor a naturally-occurring polypeptide comprising the polypeptide encoded by the polynucleotide of SEQ ID No: 17, have previously been associated with any neurodegenerative disease or condition. Indeed, as far as the inventor is aware, such polypeptides have not been associated with any disease state and have not previously been used therapeutically.

Thus the invention includes a polypeptide encoded by the polynucleotide of SEQ ID No: 17, or a naturally-occurring polypeptide comprising the polypeptide encoded by the polynucleotide of SEQ ID No: 17, or an anti-apoptotic derivative thereof, or a polynucleotide that encodes said polypeptide or derivative, for use in medicine.

The invention includes combating a neurodegenerative disease or condition in a patient by administering to the cell a polypeptide encoded by the polynucleotide of SEQ ID No: 17, or a naturally-occurring polypeptide comprising the polypeptide encoded by the polynucleotide of SEQ ID No: 17, or an anti-apoptotic derivative thereof, or a polynucleotide that encodes said polypeptide or derivative.

Suitable “anti-apoptotic derivatives” include anti-apoptotic fragments of a polypeptide encoded by the polynucleotide of SEQ ID No: 17, or anti-apoptotic fragments of a naturally-occurring polypeptide comprising the polypeptide encoded by the polynucleotide of SEQ ID No: 17, or anti-apoptotic fragments of a polypeptide encoded by a naturally-occurring polynucleotide transcript comprising SEQ ID No: 17. By “an anti-apoptotic fragment” we mean any portion of the polypeptide that has the ability to inhibit Bax-mediated apoptosis in a cell. Typically, the fragment has at least 30% of the anti-apoptotic activity of the polypeptide fragment encoded by the polynucleotide of SEQ ID No. 17. It is more preferred if the fragment has at least 50%, preferably at least 70% and more preferably at least 90% of the anti-apoptotic activity of the polypeptide fragment encoded by the polynucleotide of SEQ ID No. 17. Most preferably, the fragment has 100% or more of the anti-apoptotic activity of the polypeptide fragment encoded by the polynucleotide of SEQ ID No. 17.

Suitable “anti-apoptotic derivatives” also include modifications of a polypeptide encoded by the polynucleotide of SEQ ID No: 17, or of a full-length naturally-occurring polypeptide comprising the polypeptide encoded by the polynucleotide of SEQ ID No: 17, or of a polypeptide encoded by a naturally-occurring polynucleotide transcript comprising SEQ ID No: 17, or modifications of the fragment thereof, that have the ability to inhibit Bax-mediated apoptosis in a cell. Preferably, the corresponding section of the modified derivative retains at least 90% sequence identity with the polypeptide encoded by the polynucleotide of SEQ ID No: 16, or the respective fragment thereof. More preferably, the corresponding section of the modified derivative, or modified fragment thereof, has at least 91%, 92%, 93%, 94% or 95% sequence identity, and yet more preferably at least 96%, 97%, 98% or 99% sequence identity with the polypeptide encoded by the polynucleotide of SEQ ID No: 17, or the respective fragment thereof. Preferably, the modified polypeptide or modified fragment thereof retains at least 30% of the anti-apoptotic activity of the polypeptide encoded by the polynucleotide of SEQ ID No. 17. It is more preferred if the modified polypeptide or modified fragment has at least 50%, preferably at least 70% and more preferably at least 90% of the anti-apoptotic activity of the polypeptide encoded by the polynucleotide SEQ ID No. 17. Most preferably, the modified polypeptide or modified fragment thereof has 100% or more of the anti-apoptotic activity of the polypeptide encoded by the polynucleotide SEQ ID No. 17.

It is appreciated that for applications in which this gene/polypeptide is administered to a subject, it is preferably from the same species as the subject, particularly if the subject is a human subject.

The Sequence from the Genome of Human Mitochondrial Isolate WH6967 of Previously Unknown Function

Homo sapiens mitochondrion isolate WH6967 (Genbank Accession No AY255145), has a complete genome length of 16558 nucleotides. The polynucleotide as defined in SEQ ID No: 18 (see FIG. 65) is from nucleotides 8492-9143. Two open reading frames are present within SEQ ID No: 18 (indicated by the arrows in FIG. 65) at the following locations: ORF-3: 25-168, and ORF-5: 535-651.

To the best of the inventor's knowledge, neither the polynucleotide from the genome of human mitochondrial isolate WH6967 as defined in SEQ ID No: 18, nor a polypeptide encoded by the polynucleotide of SEQ ID No: 18, nor a naturally-occurring polypeptide comprising the polypeptide encoded by the polynucleotide of SEQ ID No: 18, have previously been associated with apoptosis.

Thus the invention includes combating Bax-mediated apoptosis in a cell by administering to the cell a polypeptide encoded by the polynucleotide of SEQ ID No: 18, or a naturally-occurring polypeptide comprising the a polypeptide encoded by the polynucleotide of SEQ ID No: 18, or an anti-apoptotic derivative thereof, or a polynucleotide that encodes said polypeptide or derivative.

Although certain mitochondrial proteins are known to be associated with disease states, to the best of the inventor's knowledge, neither the polynucleotide from the genome of human mitochondrial isolate WH6967 as defined in SEQ ID No: 18, nor a polypeptide encoded by the polynucleotide of SEQ ID No: 18, nor a naturally-occurring polypeptide comprising the polypeptide encoded by the polynucleotide of SEQ ID No: 18, have previously been associated with any neurodegenerative disease or condition. Indeed, as far as the inventor is aware, such polypeptides have not been associated with any disease state and have not previously been used therapeutically.

Thus the invention includes a polypeptide encoded by the polynucleotide of SEQ ID No: 18, or a naturally-occurring polypeptide comprising the a polypeptide encoded by the polynucleotide of SEQ ID No: 18, or an anti-apoptotic derivative thereof, or a polynucleotide that encodes said polypeptide or derivative, for use in medicine.

Thus the invention includes combating a neurodegenerative disease or condition in a patient by administering to the cell a polypeptide encoded by the polynucleotide of SEQ ID No: 18, or a naturally-occurring polypeptide comprising the a polypeptide encoded by the polynucleotide of SEQ ID No: 18, or an anti-apoptotic derivative thereof, or a polynucleotide that encodes said polypeptide or derivative.

Suitable “anti-apoptotic derivatives” include anti-apoptotic fragments of a polypeptide encoded by the polynucleotide of SEQ ID No: 18, or anti-apoptotic fragments of a naturally-occurring polypeptide comprising the polypeptide encoded by the polynucleotide of SEQ ID No: 18, or anti-apoptotic fragments of a polypeptide encoded by a naturally-occurring polynucleotide transcript comprising SEQ ID No: 18. By “an anti-apoptotic fragment” we mean any portion of the polypeptide that has the ability to inhibit Bax-mediated apoptosis in a cell. Typically, the fragment has at least 30% of the anti-apoptotic activity of the polypeptide fragment encoded by the polynucleotide of SEQ ID No. 18. It is more preferred if the fragment has at least 50%, preferably at least 70% and more preferably at least 90% of the anti-apoptotic activity of the polypeptide fragment encoded by the polynucleotide of SEQ ID No. 18. Most preferably, the fragment has 100% or more of the anti-apoptotic activity of the polypeptide fragment encoded by the polynucleotide of SEQ ID No. 18.

Suitable “anti-apoptotic derivatives” also include modifications of a polypeptide encoded by the polynucleotide of SEQ ID No: 18, or of a full-length naturally-occurring polypeptide comprising the a polypeptide encoded by the polynucleotide of SEQ ID No: 18, or of a polypeptide encoded by a naturally-occurring polynucleotide transcript comprising SEQ ID No: 18, or modifications of the fragment thereof, that have the ability to inhibit Bax-mediated apoptosis in a cell. Preferably, the corresponding section of the modified derivative retains at least 90% sequence identity with the polypeptide encoded by the polynucleotide of SEQ ID No: 186, or the respective fragment thereof. More preferably, the corresponding section of the modified derivative, or modified fragment thereof, has at least 91%, 92%, 93%, 94% or 95% sequence identity, and yet more preferably at least 96%, 97%, 98% or 99% sequence identity with the polypeptide encoded by the polynucleotide of SEQ ID No: 18, or the respective fragment thereof. Preferably, the modified polypeptide or modified fragment thereof retains at least 30% of the anti-apoptotic activity of the polypeptide encoded by the polynucleotide of SEQ ID No. 18. It is more preferred if the modified polypeptide or modified fragment has at least 50%, preferably at least 70% and more preferably at least 90% of the anti-apoptotic activity of the polypeptide encoded by the polynucleotide SEQ ID No. 18. Most preferably, the modified polypeptide or modified fragment thereof has 100% or more of the anti-apoptotic activity of the polypeptide encoded by the polynucleotide SEQ ID No. 18.

It is appreciated that for applications in which this gene/polypeptide is administered to a subject, it is preferably from the same species as the subject, particularly if the subject is a human subject.

The Sequence from the Genome of Human Mitochondrial Isolate S1216 of Previously Unknown Function

Homo sapiens mitochondrion isolate S1216 (Genbank Accession No AY289093), has a complete genome length of 16569 nucleotides. The polynucleotide as defined in SEQ ID No: 19 (see FIG. 66) is from nucleotides 7717-8283. Two open reading frames are present within SEQ ID No: 19 (indicated by the arrows in FIG. 66) at the following locations: ORF-2: 178-279, and ORF-1: 355-462.

To the best of the inventor's knowledge, neither the polynucleotide from the genome of human mitochondrial isolate S1216 as defined in SEQ ID No: 19, nor a polypeptide encoded by the polynucleotide of SEQ ID No: 19, nor a naturally-occurring polypeptide comprising the polypeptide encoded by the polynucleotide of SEQ ID No: 19, have previously been associated with apoptosis.

Thus the invention includes combating Bax-mediated apoptosis in a cell by administering to the cell a polypeptide encoded by the polynucleotide of SEQ ID No: 19, or a naturally-occurring polypeptide comprising the a polypeptide encoded by the polynucleotide of SEQ ID No: 19, or an anti-apoptotic derivative thereof, or a polynucleotide that encodes said polypeptide or derivative.

Although the mitochondrial genome is known to be affected in certain disease states, to the best of the inventor's knowledge, neither the polynucleotide from the genome of human mitochondrial isolate S1216 as defined in SEQ ID No: 19, nor a polypeptide encoded by the polynucleotide of SEQ ID No: 19, nor a naturally-occurring polypeptide comprising the a polypeptide encoded by the polynucleotide of SEQ ID No: 19, have previously been associated with any neurodegenerative disease or condition. Indeed, as far as the inventor is aware, such polypeptides have not been associated with any disease state and have not previously been used therapeutically.

Thus the invention includes a polypeptide encoded by the polynucleotide of SEQ ID No: 19, or a naturally-occurring polypeptide comprising the polypeptide encoded by the polynucleotide of SEQ ID No: 19, or an anti-apoptotic derivative thereof, or a polynucleotide that encodes said polypeptide or derivative, for use in medicine.

The invention includes combating a neurodegenerative disease or condition in a patient by administering to the cell a polypeptide encoded by the polynucleotide of SEQ ID No: 19, or a naturally-occurring polypeptide comprising the a polypeptide encoded by the polynucleotide of SEQ ID No: 19, or an anti-apoptotic derivative thereof, or a polynucleotide that encodes said polypeptide or derivative.

To the best of the inventor's knowledge, neither the polynucleotide from the genome of human mitochondrial isolate S1216 as defined in SEQ ID No: 19, nor a polypeptide encoded by the polynucleotide of SEQ ID No: 19, nor a naturally-occurring polypeptide comprising the a polypeptide encoded by the polynucleotide of SEQ ID No: 19, have previously been associated with apoptosis or with any neurodegenerative disorder.

Suitable “anti-apoptotic derivatives” include anti-apoptotic fragments of a polypeptide encoded by the polynucleotide of SEQ ID No: 19, or anti-apoptotic fragments of a naturally-occurring polypeptide comprising the a polypeptide encoded by the polynucleotide of SEQ ID No: 19, or anti-apoptotic fragments of a polypeptide encoded by a naturally-occurring polynucleotide transcript comprising SEQ ID No: 19. By “an anti-apoptotic fragment” we mean any portion of the polypeptide that has the ability to inhibit Bax-mediated apoptosis in a cell. Typically, the fragment has at least 30% of the anti-apoptotic activity of the polypeptide fragment encoded by the polynucleotide of SEQ ID No. 19. It is more preferred if the fragment has at least 50%, preferably at least 70% and more preferably at least 90% of the anti-apoptotic activity of the polypeptide fragment encoded by the polynucleotide of SEQ ID No. 19. Most preferably, the fragment has 100% or more of the anti-apoptotic activity of the polypeptide fragment encoded by the polynucleotide of SEQ ID No. 19.

Suitable “anti-apoptotic derivatives” also include modifications of a polypeptide encoded by the polynucleotide of SEQ ID No: 19, or of a full-length naturally-occurring polypeptide comprising the a polypeptide encoded by the polynucleotide of SEQ ID No: 19, or of a polypeptide encoded by a naturally-occurring polynucleotide transcript comprising SEQ ID No: 19, or modifications of the fragment thereof, that have the ability to inhibit Bax-mediated apoptosis in a cell. Preferably, the corresponding section of the modified derivative retains at least 90% sequence identity with the polypeptide encoded by the polynucleotide of SEQ ID No: 19, or the respective fragment thereof. More preferably, the corresponding section of the modified derivative, or modified fragment thereof, has at least 91%, 92%, 93%, 94% or 95% sequence identity, and yet more preferably at least 96%, 97%, 98% or 99% sequence identity with the polypeptide encoded by the polynucleotide of SEQ ID No: 19, or the respective fragment thereof. Preferably, the modified polypeptide or modified fragment thereof retains at least 30% of the anti-apoptotic activity of the polypeptide encoded by the polynucleotide of SEQ ID No. 19. It is more preferred if the modified polypeptide or modified fragment has at least 50%, preferably at least 70% and more preferably at least 90% of the anti-apoptotic activity of the polypeptide encoded by the polynucleotide SEQ ID No. 19. Most preferably, the modified polypeptide or modified fragment thereof has 100% or more of the anti-apoptotic activity of the polypeptide encoded by the polynucleotide SEQ ID No. 19.

It is appreciated that for applications in which this gene/polypeptide is administered to a subject, it is preferably from the same species as the subject, particularly if the subject is a human subject.

It is appreciated that further modifications of the above polypeptides useful in the fifth to tenth aspects of the invention may include a compound comprising a polypeptide or a fragment thereof, or the polynucleotide encoding the polypeptide or a fragment thereof, as described above, together with another agent. Preferably, the compound retains at least 30% of the anti-apoptotic activity of the respective human polypeptide as described above. It is more preferred if the compound has at least 50%, preferably at least 70% and more preferably at least 90%, or 100% or more, of the anti-apoptotic activity of the respective human polypeptide as described above. The agent may be fused to, combined with, bound to, connected to, or otherwise associated with the said polypeptide or polynucleotide, as is well known in the art.

Agents that may be useful include targeting moieties that can target the polypeptides or the polynucleotides to a target tissue, such as, for example, the hippocampus. Suitable targeting moieties include protein transduction domains such as HIV-Tat, antennapedia, PTD-5 and lysine homopolymers.

An eleventh aspect of the invention provides a method of increasing Bax-mediated apoptosis in a cell, the method comprising contacting the cell with an inhibitor or an antagonist of a polypeptide selected from FKBP2, SNCA, EEF1A1, VAMP3, SNAP25, RIMS3, RAB40B, HMGCS1, SCD5, ATP2A2, HRMT1L1, and a polypeptide encoded by a polynucleotide comprising SEQ ID No: 16, SEQ ID No: 17, SEQ ID No: 18 or SEQ ID No: 19.

Typically, the method is performed in vitro.

Typically, the cell is a mammalian cell, such as a human cell, although as is clear from the examples, the cell may from other species such as yeast. The cell may be from an established cell line, a primary cell culture, or a cell which is present in a tissue ex vivo.

Alternatively, the method may be performed in vivo.

A twelfth aspect of the invention provides the use of an inhibitor or an antagonist of a polypeptide selected from FKBP2, SNCA, EEF1A1, VAMP3, SNAP25, RIMS3, RAB40B, HMGCS1, SCD5, ATP2A2, HRMT1L1, and a polypeptide encoded by a polynucleotide comprising SEQ ID No: 16, SEQ ID No: 17, SEQ ID No: 18 or SEQ ID No: 19, in the preparation of a medicament for increasing Bax-mediated apoptosis in a cell of a patient.

A thirteenth aspect of the invention provides an inhibitor or an antagonist of a polypeptide selected from FKBP2, SNCA, EEF1A1, VAMP3, SNAP25, RIMS3, RAB40B, HMGCS1, SCD5, ATP2A2, HRMT1L1, and a polypeptide encoded by a polynucleotide comprising SEQ ID No: 16, SEQ ID No: 17, SEQ ID No: 18 or SEQ ID No: 19, or a polynucleotide that encodes any of said inhibitors or antagonists, for use in medicine.

A fourteenth aspect of the invention provides a pharmaceutical composition comprising an inhibitor or an antagonist of a polypeptide selected from FKBP2, SNCA, EEF1A1, VAMP3, SNAP25, RIMS3, RAB40B, HMGCS1, SCD5, ATP2A2, HRMT1L1, and a polypeptide encoded by a polynucleotide comprising SEQ ID No: 16, SEQ ID No: 17, SEQ ID No: 18 or SEQ ID No: 19, or a polynucleotide that encodes any of said inhibitors or antagonists, and a pharmaceutically acceptable carrier or excipient.

Increasing Bax-mediated apoptosis may be useful in combating cancers, in particular brain tumours. Cancer cells are immortal because normal cells fail to undergo apoptosis when they ought to. By finding apoptosis inhibitors from specific tissues, one would be able to treat cancer by down-regulating expression of proteins that inhibit the process of apoptosis.

A fifteenth aspect of the invention provides a method of combating cancer in a patient, the method comprising administering to the patient an inhibitor or an antagonist of a polypeptide selected from FKBP2, SNCA, EEF1A1, VAMP3, SNAP25, RIMS3, RAB40B, HMGCS1, SCD5, ATP2A2, HRMT1L1, and a polypeptide encoded by a polynucleotide comprising SEQ ID No: 16, SEQ ID No: 17, SEQ ID No: 18 or SEQ ID No: 19, or a polynucleotide that encodes any of said inhibitors or antagonists.

A sixteenth aspect of the invention provides the use of an inhibitor or an antagonist of a polypeptide selected from FKBP2, SNCA, EEF1A1, VAMP3, SNAP25, RIMS3, RAB40B, HMGCS1, SCD5, ATP2A2, HRMT1L1, and a polypeptide encoded by a polynucleotide comprising SEQ ID No: 16, SEQ ID No: 17, SEQ ID No: 18 or SEQ ID No: 19, or a polynucleotide that encodes any of said inhibitors or antagonists, in the preparation of a medicament for combating cancer in a patient.

Suitable inhibitors or antagonists of the above-listed polypeptides include antibodies that selectively bind to the said polypeptide. Suitable antibodies can be made by the skilled person using technology long-established in the art. Antibodies which inhibit the anti-apoptotic activity of the above-listed polypeptides, are especially preferred for the anti-cancer therapeutics, and they can be selected for this activity using methods well known in the art and described herein.

By an antibody that selectively binds a specified polypeptide we mean that the antibody molecule binds that polypeptide with a greater affinity than for an irrelevant polypeptide, such as human serum albumin (HSA). Preferably, the antibody binds the specified polypeptide with 5, or at least 10 or at least 50 times greater affinity than for an irrelevant polypeptide. More preferably, the antibody molecule binds the specified polypeptide with at least 100, or at least 1,000, or at least 10,000 times greater affinity than for the an irrelevant polypeptide. Such binding may be determined by methods well known in the art, such as one of the Biacore® systems. Preferably, the antibody molecule selectively binds the specified polypeptide without significantly binding other polypeptides in the body. It is preferred if the antibodies have an affinity for their target epitope on the specified polypeptide of at least 10⁻⁸ M, although antibodies with higher affinities may be even more preferred.

By “antibody” or “antibody molecule” we include not only whole immunoglobulin molecules as is well known in the art but also fragments thereof such as Fab, F(ab′)₂, Fv and other fragments thereof that retain the antigen-binding site. Similarly the term antibody includes genetically engineered derivatives of antibodies such as single chain Fv molecules (scFv) and single domain antibodies (dAbs) comprising isolated V domains. The term also includes antibody-like molecules which may be produced using phage-display techniques or other random selection techniques for molecules which bind to the specified polypeptide or to particular regions of it. Thus, the term antibody includes all molecules which contain a structure, preferably a peptide structure, which is part of the recognition site (i.e. the part of the antibody that binds or combines with the epitope or antigen) of a natural antibody. A general review of the techniques involved in the synthesis of antibody fragments which retain their specific binding sites is to be found in Winter & Milstein (1991) Nature 349, 293-299.

By “ScFv molecules” we mean molecules wherein the V_{H} and V_{L} partner domains are linked via a flexible oligopeptide. Engineered antibodies, such as ScFv antibodies, can be made using the techniques and approaches described in Huston et al (1988) “Protein engineering of antibody binding sites: recovery of specific activity in an anti-digoxin single chain Fv analogue produced in E. coli”, Proc. Natl. Acad. Sci. USA, 85, pp. 5879-5883, and in A. Pluckthun, (1991) “Antibody engineering; Advances from use of E. coli expression systems”, Bio/technology 9(6): 545-51.

The advantages of using antibody fragments, rather than whole antibodies, are several-fold. The smaller size of the fragments may lead to improved pharmacological properties, such as better penetration to the target site. Effector functions of whole antibodies, such as complement binding, are removed. Fab, Fv, ScFv and dAb antibody fragments can all be expressed in and secreted from E. coli, thus allowing the facile production of large amounts of the fragments. Whole antibodies, and F(ab′)₂ fragments are “bivalent”. By “bivalent” we mean that the antibodies and F(ab′)₂ fragments have two antigen combining sites. In contrast, Fab, Fv, ScFv and dAb fragments are monovalent, having only one antigen combining site.

It is preferred if the antibody is a monoclonal antibody. In some circumstance, particularly if the antibody is going to be administered repeatedly to a human patient, it is preferred if the monoclonal antibody is a human monoclonal antibody or a humanised monoclonal antibody. Suitable monoclonal antibodies which are reactive as described herein may be prepared by known techniques, for example those disclosed in “Monoclonal Antibodies; A manual of techniques”, H Zola (CRC Press, 1988) and in “Monoclonal Hybridoma Antibodies: Techniques and Application”, SGR Hurrell (CRC Press, 1982).

It is preferred if the antibody is a humanised antibody. Suitably prepared non-human antibodies can be “humanised” in known ways, for example by inserting the CDR regions of mouse antibodies into the framework of human antibodies. Humanised antibodies can be made using the techniques and approaches described in Verhoeyen et al (1988) Science, 239, 1534-1536, and in Kettleborough et al, (1991) Protein Engineering, 14(7), 773-783. Completely human antibodies may be produced using recombinant technologies. Typically large libraries comprising billions of different antibodies are used. In contrast to the previous technologies employing chimerisation or humanisation of e.g. murine antibodies this technology does not rely on immunisation of animals to generate the specific antibody. Instead the recombinant libraries comprise a huge number of pre-made antibody variants wherein it is likely that the library will have at least one antibody specific for any antigen. Thus, using such libraries, an existing antibody having the desired binding characteristics can be identified. In order to find the good binder in a library in an efficient manner, various systems where phenotype i.e. the antibody or antibody fragment is linked to its genotype i.e. the encoding gene have been devised. The most commonly used such system is the so called phage display system where antibody fragments are expressed, displayed, as fusions with phage coat proteins on the surface of filamentous phage particles, while simultaneously carrying the genetic information encoding the displayed molecule (McCafferty et al, 1990, Nature 348: 552-554). Phage displaying antibody fragments specific for a particular antigen may be selected through binding to the antigen in question. Isolated phage may then be amplified and the gene encoding the selected antibody variable domains may optionally be transferred to other antibody formats, such as e.g. full-length immunoglobulin, and expressed in high amounts using appropriate vectors and host cells well known in the art.

The format of displayed antibody specificities on phage particles may differ. The most commonly used formats are Fab (Griffiths et al, 1994. EMBO J. 13: 3245-3260) and single chain (scFv) (Hoogenboom et al, 1992, J Mol. Biol. 227: 381-388) both comprising the variable antigen binding domains of antibodies. The single chain format is composed of a variable heavy domain (V_{H}) linked to a variable light domain (V_{L}) via a flexible linker (U.S. Pat. No. 4,946,778). Before use as a therapeutic agent, the antibody may be transferred to a soluble format e.g. Fab or scFv and analysed as such. In later steps the antibody fragment identified to have desirable characteristics may be transferred into yet other formats such as full-length antibodies.

WO 98/32845 and Soderlind et al (2000) Nature BioTechnol. 18:852-856 describe technology for the generation of variability in antibody libraries. Antibody fragments derived from this library all have the same framework regions and only differ in their CDRs. Since the framework regions are of germline sequence the immunogenicity of antibodies derived from the library, or similar libraries produced using the same technology, are expected to be particularly low (Soderlind et al, 2000). This property is of great value for therapeutic antibodies, reducing the risk that the patient forms antibodies to the administered antibody, thereby reducing risks for allergic reactions, the occurrence of blocking antibodies, and allowing a long plasma half-life of the antibody. Thus, when developing therapeutic antibodies to be used in humans, modern recombinant library technology (Soderlind et al, 2001, Comb. Chem. & High Throughput Screen. 4: 409-416) is now used in preference to the earlier hybridoma technology.

Other suitable inhibitors or antagonists of the above-listed polypeptides include siRNA, antisense polynucleotides and ribozyme molecules that are specific for the polynucleotides encoding the above listed polypeptides, and which prevent their expression.

Small interfering RNAs are described by Hannon et al. Nature, 418 (6894): 244-51 (2002); Brummelkamp et al., Science 21, 21 (2002); and Sui et al., Proc. Natl. Acad. Sci. USA 99, 5515-5520 (2002). RNA interference (RNAi) is the process of sequence-specific post-transcriptional gene silencing in animals initiated by double-stranded (dsRNA) that is homologous in sequence to the silenced gene. The mediators of sequence-specific mRNA degradation are typically 21- and 22-nucleotide small interfering RNAs (siRNAs) which, in vivo, may be generated by ribonuclease III cleavage from longer dsRNAs. 21-nucleotide siRNA duplexes have been shown to specifically suppress expression of both endogenous and heterologous genes (Elbashir et al (2001) Nature 411: 494-498). In mammalian cells it is considered that the siRNA has to be comprised of two complementary 21mers as described below since longer double-stranded (ds) RNAs will activate PKR (dsRNA-dependent protein kinase) and inhibit overall protein synthesis.

Duplex siRNA molecules selective for a polynucleotide encoding any of the above listed polypeptides can readily be designed by reference to its cDNA sequence. For example, they can be designed by reference to the cDNA sequences in the Genbank Accession Nos. listed above, or to the cDNA sequences identified as a result of the screen described in the Examples (SEQ ID Nos: 5-19 in FIGS. 62-66). Typically, the first 21-mer sequence that begins with an AA dinucleotide which is at least 120 nucleotides downstream from the initiator methionine codon is selected. The RNA sequence perfectly complementary to this becomes the first RNA oligonucleotide. The second RNA sequence should be perfectly complementary to the first 19 residues of the first, with an additional UU dinucleotide at its 3′ end. Once designed, the synthetic RNA molecules can be synthesised using methods well known in the art.

siRNAs may be introduced into cells in the patient using any suitable method, such as those described herein. Typically, the RNA is protected from the extracellular environment, for example by being contained within a suitable carrier or vehicle. Liposome-mediated transfer is preferred. It is particularly preferred if the oligofectamine method is used.

Antisense nucleic acid molecules selective for a polynucleotide encoding any of the above listed polypeptides can readily be designed by reference to its cDNA or gene sequence, as is known in the art. Antisense nucleic acids, such as oligonucleotides, are single-stranded nucleic acids, which can specifically bind to a complementary nucleic acid sequence. By binding to the appropriate target sequence, an RNA-RNA, a DNA-DNA, or RNA-DNA duplex is formed. These nucleic acids are often termed “antisense” because they are complementary to the sense or coding strand of the gene. Recently, formation of a triple helix has proven possible where the oligonucleotide is bound to a DNA duplex. It was found that oligonucleotides could recognise sequences in the major groove of the DNA double helix. A triple helix was formed thereby. This suggests that it is possible to synthesise a sequence-specific molecules which specifically bind double-stranded DNA via recognition of major groove hydrogen binding sites. By binding to the target nucleic acid, the above oligonucleotides can inhibit the function of the target nucleic acid. This could, for example, be a result of blocking the transcription, processing, poly(A) addition, replication, translation, or promoting inhibitory mechanisms of the cells, such as promoting RNA degradations.

Antisense oligonucleotides are prepared in the laboratory and then introduced into cells, for example by microinjection or uptake from the cell culture medium into the cells, or they are expressed in cells after transfection with plasmids or retroviruses or other vectors carrying an antisense gene. Antisense oligonucleotides were first discovered to inhibit viral replication or expression in cell culture for Rous sarcoma virus, vesicular stomatitis virus, herpes simplex virus type 1, simian virus and influenza virus. Since then, inhibition of mRNA translation by antisense oligonucleotides has been studied extensively in cell-free systems including rabbit reticulocyte lysates and wheat germ extracts. Inhibition of viral function by antisense oligonucleotides has been demonstrated ex vivo using oligonucleotides which were complementary to the AIDS HIV retrovirus RNA (Goodchild, J. 1988 “Inhibition of Human Immunodeficiency Virus Replication by Antisense Oligodeoxynucleotides”, Proc. Natl. Acad. Sci. (USA) 85(15), 5507-11). The Goodchild study showed that oligonucleotides that were most effective were complementary to the poly(A) signal; also effective were those targeted at the 5′ end of the RNA, particularly the cap and 5N untranslated region, next to the primer binding site and at the primer binding site. The cap, 5′ untranslated region, and poly(A) signal lie within the sequence repeated at the ends of retrovirus RNA (R region) and the oligonucleotides complementary to these may bind twice to the RNA.

Typically, antisense oligonucleotides are 15 to 35 bases in length. For example, 20-mer oligonucleotides have been shown to inhibit the expression of the epidermal growth factor receptor mRNA (Witters et al., Breast Cancer Res Treat 53:41-50 (1999)) and 25-mer oligonucleotides have been shown to decrease the expression of adrenocorticotropic hormone by greater than 90% (Frankel et al, J Neurosurg 91:261-7 (1999)). However, it is appreciated that it may be desirable to use oligonucleotides with lengths outside this range, for example 10, 11, 12, 13, or 14 bases, or 36, 37, 38, 39 or 40 bases.

Antisense polynucleotides may be administered systemically. Alternatively, and preferably, the inherent binding specificity of polynucleotides characteristic of base pairing is enhanced by limiting the availability of the polynucleotide to its intended locus in vivo, permitting lower dosages to be used and minimising systemic effects. Thus, polynucleotides may be applied locally to the cancer achieve the desired effect. The concentration of the polynucleotides at the desired locus is much higher than if the polynucleotides were administered systemically, and the therapeutic effect can be achieved using a significantly lower total amount. The local high concentration of polynucleotides enhances penetration of the targeted cells and effectively blocks translation of the target nucleic acid sequences.

It will be appreciated that antisense agents also include larger molecules which bind to polynucleotides (mRNA or genes) encoding any of the above listed polypeptides and substantially prevent expression of the protein. Thus, antisense molecules which are substantially complementary to the respective mRNA is also envisaged.

The larger molecules may be expressed from any suitable genetic construct and delivered to the patient. Typically, the genetic construct which expresses the antisense molecule comprises at least a portion of the cDNA or gene operatively linked to a promoter which can express the antisense molecule in the cell. Preferably, the genetic construct is adapted for delivery to a human cell.

Ribozymes are RNA or RNA-protein complexes that cleave nucleic acids in a site-specific fashion. Ribozymes have specific catalytic domains that possess endonuclease activity. For example, a large number of ribozymes accelerate phosphoester transfer reactions with a high degree of specificity, often cleaving only one of several phosphoesters in an oligonucleotide substrate. This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids. For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications, and may be designed by reference to the cDNA sequences listed in the Genbank Accession Nos. given above, or by reference to SEQ ID Nos: 5-19.

Further agents that inhibit transcription of the genes encoding any of the above listed polypeptides can also be designed, for example using an engineered transcription repressor described in Isalan et al (Nat Biotechnol, 19(7): 656-60 (2001)) and in Urnov (Biochem Pharmacol, 64 (5-6): 919 (2002)). Additionally, they can be selected, for example using the screening methods described in later aspects of the invention.

The therapy (treatment) may be on humans or animals. Preferably, the methods of the inventions are used to treat humans.

The various therapeutic agents described above in the fifth to the sixteenth aspects of the invention are typically formulated for administration to an individual as a pharmaceutical composition, i.e. together with a pharmaceutically acceptable carrier or excipient.

By “pharmaceutically acceptable” is included that the formulation is sterile and pyrogen free. Suitable pharmaceutical carriers are well known in the art of pharmacy.

The carrier(s) must be “acceptable” in the sense of being compatible with the therapeutic agent and not deleterious to the recipients thereof. Typically, the carriers will be water or saline which will be sterile and pyrogen free; however, other acceptable carriers may be used.

In an embodiment, the pharmaceutical compositions or formulations of the invention are for parenteral administration, more particularly for intravenous administration, for example by injection. Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

In an alternative preferred embodiment, the pharmaceutical composition is suitable for topical administration to a patient.

Preferably, the formulation is a unit dosage containing a daily dose or unit, daily sub-dose or an appropriate fraction thereof, of the active ingredient.

The therapeutic agent may be administered orally or by any parenteral route, in the form of a pharmaceutical formulation comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

As used herein, the term “therapeutic agent” includes polypeptides, and polynucleotides and other molecules, except where the context demands otherwise. For example, in some circumstances, the term “therapeutic agent” refers to only polypeptides or refers only to polynucleotides, and this will be clear from the context.

In human therapy, the therapeutic agent can be administered alone but will generally be administered in admixture with a suitable pharmaceutical excipient, diluent or carrier selected with regard to the intended route of administration and standard pharmaceutical practice.

For example, the therapeutic agent can be administered orally, buccally or sublingually in the form of tablets, capsules, ovules, elixirs, solutions or suspensions, which may contain flavouring or colouring agents, for immediate-, delayed- or controlled-release applications. The compound of invention may also be administered via intracavernosal injection.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the therapeutic agents may be combined with various sweetening or flavouring agents, colouring matter or dyes, with emulsifying and/or suspending agents and with diluents such as water, ethanol, propylene glycol and glycerin, and combinations thereof.

The therapeutic agent can also be administered parenterally, for example, intravenously, intra-arterially, intraperitoneally, intrathecally, intraventricularly, intrasternally, intracranially, intramuscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water fore injections, immediately prior to use.

Extemporaneous injection solutions and suspensions may be prepared from sterile powders, granules and tablets of the kind previously described.

For oral and parenteral administration to human patients, the daily dosage level of the therapeutic agent will usually be from 1 to 1000 mg per adult (i.e. from about 0.015 to 15 mg/kg), administered in single or divided doses.

Thus, for example, the tablets or capsules of the therapeutic agent may contain from 1 mg to 1000 mg of active agent for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

The therapeutic agent may be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoro-ethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a therapeutic agent and a suitable powder base such as lactose or starch.

Aerosol or dry powder formulations are preferably arranged so that each metered dose or “puff” contains at least 1 mg of the therapeutic agent for delivery to the patient. It will be appreciated that the overall daily dose with an aerosol will vary from patient to patient, and may be administered in a single dose or, more usually, in divided doses throughout the day.

Alternatively, the therapeutic agent can be administered in the form of a suppository or pessary, or they may be applied topically in the form of a lotion, solution, cream, ointment or dusting powder. The therapeutic agents may also be transdermally administered, for example, by the use of a skin patch. They may also be administered by the ocular route, particularly for treating diseases of the eye. Eye diseases that may be treated according to the invention include glaucoma, retinitis pigmentosa, cataract formation, retinoblastoma, retinal ischemia, and diabetic retinopathy.

For ophthalmic use, the therapeutic agent can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the therapeutic agent can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier.

For veterinary use, a therapeutic agent is administered as a suitably acceptable formulation in accordance with normal veterinary practice and the veterinary surgeon will determine the dosing regimen and route of administration which will be most appropriate for a particular animal.

In an embodiment, polypeptides may be delivered using an injectable sustained-release drug delivery system. These are designed specifically to reduce the frequency of injections. An example of such a system is Nutropin Depot which encapsulates recombinant human growth hormone (rhGH) in biodegradable microspheres that, once injected, release rhGH slowly over a sustained period.

The polypeptide can be administered by a surgically implanted device that releases the drug directly to the required site. For example, Vitrasert releases ganciclovir directly into the eye to treat CMV retinitis. The direct application of this toxic agent to the site of disease achieves effective therapy without the drug's significant systemic side-effects.

Electroporation therapy (EPT) systems can also be employed for the administration of polypeptides. A device which delivers a pulsed electric field to cells increases the permeability of the cell membranes to the drug, resulting in a significant enhancement of intracellular drug delivery.

Polypeptides can also be delivered by electroincorporation (EI). EI occurs when small particles of up to 30 microns in diameter on the surface of the skin experience electrical pulses identical or similar to those used in electroporation. In EI, these particles are driven through the stratum corneum and into deeper layers of the skin. The particles can be loaded or coated with drugs or genes or can simply act as “bullets” that generate pores in the skin through which the drugs can enter.

An alternative method of polypeptide delivery is the ReGel injectable system that is thermo-sensitive. Below body temperature, ReGel is an injectable liquid while at body temperature it immediately forms a gel reservoir that slowly erodes and dissolves into known, safe, biodegradable polymers. The active drug is delivered over time as the biopolymers dissolve.

Polypeptide pharmaceuticals can also be delivered orally. The process employs a natural process for oral uptake of vitamin B₁₂ in the body to co-deliver proteins and peptides. By riding the vitamin B₁₂ uptake system, the protein or peptide can move through the intestinal wall. Complexes are synthesised between vitamin B₁₂ analogues and the drug that retain both significant affinity for intrinsic factor (IF) in the vitamin B₁₂ portion of the complex and significant bioactivity of the drug portion of the complex.

Polynucleotides may be administered by any effective method, for example, parenterally (e.g. intravenously, subcutaneously, intramuscularly) or by oral, nasal or other means which permit the oligonucleotides to access and circulate in the patient's bloodstream. Polynucleotides administered systemically preferably are given in addition to locally administered polynucleotides, but also have utility in the absence of local administration. A dosage in the range of from about 0.1 to about 10 grams per administration to an adult human generally will be effective for this purpose.

The polynucleotide may be administered as a suitable genetic construct as is described below and delivered to the patient where it is expressed. Typically, the polynucleotide in the genetic construct is operatively linked to a promoter which can express the compound in the cell. The genetic constructs of the invention can be prepared using methods well known in the art, for example in Sambrook et al (2001).

Although genetic constructs for delivery of polynucleotides can be DNA or RNA, it is preferred if they are DNA.

Preferably, the genetic construct is adapted for delivery to a human cell.

Means and methods of introducing a genetic construct into a cell in an animal body are known in the art. For example, the constructs of the invention may be introduced into cells by any convenient method, for example methods involving retroviruses, so that the construct is inserted into the genome of the cell. For example, in Kuriyama et al (1991, Cell Struc. and Func. 16, 503-510) purified retroviruses are administered. Retroviral DNA constructs comprising a polynucleotide as described above may be made using methods well known in the art. To produce active retrovirus from such a construct it is usual to use an ecotropic psi2 packaging cell line grown in Dulbecco's modified Eagle's medium (DMEM) containing 10% foetal calf serum (FCS). Transfection of the cell line is conveniently by calcium phosphate co-precipitation, and stable transformants are selected by addition of G418 to a final concentration of 1 mg/ml (assuming the retroviral construct contains a neo^{R} gene). Independent colonies are isolated and expanded and the culture supernatant removed, filtered through a 0.45 μm pore-size filter and stored at −70° C. For the introduction of the retrovirus into tumour cells, for example, it is convenient to inject directly retroviral supernatant to which 10 μg/ml Polybrene has been added. For tumours exceeding 10 mm in diameter it is appropriate to inject between 0.1 ml and 1 ml of retroviral supernatant; preferably 0.5 ml.

Alternatively, as described in Culver et al (1992, Science 256, 1550-1552), cells which produce retroviruses are injected. The retrovirus-producing cells so introduced are engineered to actively produce retroviral vector particles so that continuous productions of the vector occurred within the tumour mass in situ. Thus, proliferating epidermal cells can be successfully transduced in vivo if mixed with retroviral vector-producing cells.

Targeted retroviruses are also available for use in the invention; for example, sequences conferring specific binding affinities may be engineered into pre-existing viral env genes (see Miller & Vile (1995) Faseb J. 9, 190-199, for a review of this and other targeted vectors for gene therapy).

Other methods involve simple delivery of the construct into the cell for expression therein either for a limited time or, following integration into the genome, for a longer time. An example of the latter approach includes liposomes (Nässander et al (1992) Cancer Res. 52, 646-653).

Other methods of delivery include adenoviruses carrying external DNA via an antibody-polylysine bridge (see Curiel (1993) Prog. Med. Virol. 40, 1-18) and transferrin-polycation conjugates as carriers (Wagner et al (1990) Proc. Natl. Acad. Sci. USA 87, 3410-3414). In the first of these methods a polycation-antibody complex is formed with the DNA construct or other genetic construct of the invention, wherein the antibody is specific for either wild-type adenovirus or a variant adenovirus in which a new epitope has been introduced which binds the antibody. The polycation moiety binds the DNA via electrostatic interactions with the phosphate backbone. The adenovirus, because it contains unaltered fibre and penton proteins, is internalised into the cell and carries into the cell with it the DNA construct of the invention. It is preferred if the polycation is polylysine.

In an alternative method, a high-efficiency nucleic acid delivery system that uses receptor-mediated endocytosis to carry DNA macromolecules into cells is employed. This is accomplished by conjugating the iron-transport protein transferrin to polycations that bind nucleic acids. Human transferrin, or the chicken homologue conalbumin, or combinations thereof is covalently linked to the small DNA-binding protein protamine or to polylysines of various sizes through a disulphide linkage. These modified transferrin molecules maintain their ability to bind their cognate receptor and to mediate efficient iron transport into the cell. The transferrin-polycation molecules form electrophoretically stable complexes with DNA constructs or other genetic constructs of the invention independent of nucleic acid size (from short oligonucleotides to DNA of 21 kilobase pairs). When complexes of transferrin-polycation and the DNA constructs or other genetic constructs of the invention are supplied to the tumour cells, a high level of expression from the construct in the cells is expected.

High-efficiency receptor-mediated delivery of the DNA constructs or other genetic constructs of the invention using the endosome-disruption activity of defective or chemically inactivated adenovirus particles produced by the methods of Cotten et al (1992) Proc. Natl. Acad. Sci. USA 89, 6094-6098 may also be used. This approach appears to rely on the fact that adenoviruses are adapted to allow release of their DNA from an endosome without passage through the lysosome, and in the presence of, for example transferrin linked to the DNA construct or other genetic construct of the invention, the construct is taken up by the cell by the same route as the adenovirus particle. This approach has the advantages that there is no need to use complex retroviral constructs; there is no permanent modification of the genome as occurs with retroviral infection; and the targeted expression system is coupled with a targeted delivery system, thus reducing toxicity to other cell types.

It will be appreciated that “naked DNA” and DNA complexed with cationic and neutral lipids may also be useful in introducing the DNA of the invention into cells of the individual to be treated. Non-viral approaches to gene therapy are described in Ledley (1995, Human Gene Therapy 6, 1129-1144).

Alternative targeted delivery systems are also known such as the modified adenovirus system described in WO 94/10323 wherein, typically, the DNA is carried within the adenovirus, or adenovirus-like, particle. Michael et al (1995) Gene Therapy 2, 660-668 describes modification of adenovirus to add a cell-selective moiety into a fibre protein. Mutant adenoviruses which replicate selectively in p53-deficient human tumour cells, such as those described in Bischoff et al (1996) Science 274, 373-376 are also useful for delivering the genetic construct of the invention to a cell. Thus, it will be appreciated that a further aspect of the invention provides a virus or virus-like particle comprising a genetic construct of the invention. Other suitable viruses, viral vectors or virus-like particles include lentivirus and lentiviral vectors, HSV, adeno-assisted virus (AAV) and AAV-based vectors, vaccinia and parvovirus.

Methods of delivering polynucleotides to a patient are well known to a person of skill in the art and include the use of immunoliposomes, viral vectors (including vaccinia, modified vaccinia, and adenovirus), and by direct delivery of DNA, e.g. using a gene-gun and electroporation. Furthermore, methods of delivering polynucleotides to a target tissue of a patient for treatment are also well known in the art.

For applications involving therapeutic treatment of neurodegenerative disease, it may be preferred to administer the therapeutic agent or formulation directly to the brain of the patient, or to a specific region of the brain such as the hippocampus.

Methods of targeting and delivering therapeutic agents directly to specific regions of the body, including the brain, are well known to a person of skill in the art. For example, U.S. Pat. No. 6,503,242 describes an implanted catheter apparatus for delivering therapeutic agents directly to the hippocampus

In one embodiment, therapeutic agents including vectors can be distributed throughout a wide region of the CNS by injection into the cerebrospinal fluid, e.g., by lumbar puncture (See e.g., Kapadia et al (1996) Neurosurg 10: 585-587). Alternatively, precise delivery of the therapeutic agent into specific sites of the brain, can be conducted using stereotactic microinjection techniques. For example, the subject being treated can be placed within a stereotactic frame base (MRI-compatible) and then imaged using high resolution MRI to determine the three-dimensional positioning of the particular region to be treated. The MRI images can then be transferred to a computer having the appropriate stereotactic software, and a number of images are used to determine a target site and trajectory for microinjection of the therapeutic agent. The software translates the trajectory into three-dimensional coordinates that are precisely registered for the stereotactic frame. In the case of intracranial delivery, the skull will be exposed, burr holes will be drilled above the entry site, and the stereotactic apparatus used to position the needle and ensure implantation at a predetermined depth. The therapeutic agent can be delivered to regions of the CNS such as the hippocampus, cells of the spinal cord, brainstem, (medulla, pons, and midbrain), cerebellum, diencephalon (thalamus, hypothalamus), telencephalon (corpus stratium, cerebral cortex, or within the cortex, the occipital, temporal, parietal or frontal lobes), or combinations, thereof. In another embodiment, the therapeutic agent is delivered using other delivery methods suitable for localised delivery, such as localised permeation of the blood-brain barrier.

Viral and non-viral vectors can be delivered to cells of the central nervous system. In an embodiment, the invention uses adeno-associated viral (AAV) vectors for delivery of a polynucleotide that is or that encodes a therapeutic agent. Methods of producing viral vectors such as AAV vectors are well known in the art (see, Sambrook et al, 2001, supra).

AAV vectors can be constructed using known techniques to provide at least the operatively-linked components of control elements including a transcriptional initiation region, a polynucleotide that is, or that encodes, a therapeutic agent, a transcriptional termination region, and at least one post-transcriptional regulatory sequence. The control elements are selected to be functional in the targeted cell.

US Patent Application No 20050025746 describes delivery systems for localised delivery of an adeno-associated virus vector (AAV) vector encoding a therapeutic agent to a specific region of the brain that is associated with neurodegenerative diseases, such as the hippocampus, subthalamic nucleus of the basal ganglia, mesaphilia and thalamus.

Central nervous system (CNS) specific promoters such as, neuron-specific promoters (e.g., the neurofilament promoter (Byrne and Ruddle, 1989) and glial specific promoters (Morii et al, 1991) are preferably used for directing expression of a polynucleotide preferentially in cells of the CNS. Preferably, the promoter is tissue specific and is essentially not active outside the central nervous system, or the activity of the promoter is higher in the central nervous system than in other cells or tissues. For example, a promoter specific for the spinal cord, brainstem, (medulla, pons, and midbrain), cerebellum, diencephalon (thalamus, hypothalamus), telencephalon (corpus stratium, cerebral cortex, or within the cortex, the occipital, temporal, parietal or frontal lobes), or combinations, thereof. The promoter may be specific for particular cell types, such as neurons or glial cells in the CNS. If it is active in glial cells, it may be specific for astrocytes, oligodentrocytes, ependymal cells, Schwami cells, or microglia. If it is active in neurons, it may be specific for particular types of neurons, e.g., motor neurons, sensory neurons, or interneurons. Preferably, the promoter is specific for cells in particular regions of the brain, for example, the cortex, stratium, nigra and hippocampus.

Suitable neuronal specific promoters include, but are not limited to, neuron specific enolase (NSE; Olivia et al (1991); GenBank Accession No: X51956), and human neurofilament light chain promoter (NEFL; Rogaev et al (1992); GenBank Accession No: L04147). Glial specific promoters include, but are not limited to, glial fibrillary acidic protein (GFAP) promoter (Morii et al (1991); GenBank Accession No:M65210), S100 promoter (Morii et al (1991); GenBank Accession No: M65210) and glutamine synthase promoter (Van den et al (1991); GenBank Accession No: X59834). In a preferred embodiment, the gene is flanked upstream (i.e., 5′) by the neuron specific enolase (NSE) promoter. In another preferred embodiment, the gene of interest is flanked upstream (i.e., 5′) by the elongation factor 1 alpha (EF) promoter. A hippocampus specific promoter that might be used is the hippocampus specific glucocorticoid receptor (GR) gene promoter.

Alternatively, for treatment of cardiovascular disease, Svensson et al (1999) describes the delivery of recombinant genes to cardiomyocytes by intramyocardial injection or intracoronary infusion of cardiotropic vectors, such as recombinant adeno-associated virus vectors, resulting in transgene expression in murine cardiomyocytes in vivo. Melo et al (2004) review gene and cell-based therapies for heart disease. An alternative preferred route of administration is via a catheter or stent. Stents represent an attractive alternative for localized gene delivery, as they provide a platform for prolonged gene elution and efficient transduction of opposed arterial walls. This gene delivery strategy has the potential to decrease the systemic spread of the viral vectors and hence a reduced host immune response. Both synthetic and naturally occurring stent coatings have shown potential to allow prolonged gene elution with no significant adverse reaction. (Sharif et al, 2004). Preferably, the polynucleotide encoding the antibody molecule is operatively linked to targeting and/or regulatory sequences that direct expression of the antibody to the arteries, and preferably the arterial walls. Thus the polynucleotide allows generation of the specific antibodies within the affected individual. Suitable targeting and regulatory sequences are known to the skilled person.

It may be desirable to be able to temporally regulate expression of the polynucleotide in the cell. Thus, it may be desirable that expression of the polynucleotide is directly or indirectly (see below) under the control of a promoter that may be regulated, for example by the concentration of a small molecule that may be administered to the patient when it is desired to activate or, more likely, repress (depending upon whether the small molecule effects activation or repression of the said promoter) expression of the antibody from the polynucleotide. This may be of particular benefit if the expression construct is stable, i.e. capable of expressing the antibody (in the presence of any necessary regulatory molecules), in the cell for a period of at least one week, one, two, three, four, five, six, eight months or one or more years. Thus the polynucleotide may be operatively linked to a regulatable promoter. Examples of regulatable promoters include those referred to in the following papers: Rivera et al (1999) Proc Natl Acad Sci USA 96(15), 8657-62 (control by rapamycin, an orally bioavailable drug, using two separate adenovirus or adeno-associated virus (AAV) vectors, one encoding an inducible human growth hormone (hGH) target gene, and the other a bipartite rapamycin-regulated transcription factor); Magari et al (1997) J Clin Invest 100(11), 2865-72 (control by rapamycin); Bueler (1999) Biol Chem 380(6), 613-22 (review of adeno-associated viral vectors); Bohl et al (1998) Blood 92(5), 1512-7 (control by doxycycline in adeno-associated vector); Abruzzese et al (1996) J Mol Med 74(7), 379-92 (review of induction factors, e.g. hormones, growth factors, cytokines, cytostatics, irradiation, heat shock and associated responsive elements).

Another method of targeting therapeutic agents to cells of the pancreas for treating diabetes, to synovial cells for treating rheumatoid arthritis, or to specific cancers, is to conjugate the apoptosis inhibitory polypeptides to antibodies raised against antigens that are highly produced by the relevant cells in these disease states.

A seventeenth aspect of the invention provides a method of identifying a compound that is able to modulate expression of a gene selected from FKBP2, SNCA, EEF1A1, VAMP3, SNAP25, RIMS3, RAB40B, HMGCS1, SCD5, ATP2A2, HRMT1L1, and a gene whose transcribed message has a cDNA sequence comprising SEQ ID No: 16, SEQ ID No: 17, SEQ ID No: 18 or SEQ ID No: 19, the method comprising:
providing a cell comprising a reporter gene operably linked to a promoter and/or regulatory portion from a gene selected from FKBP2, EEF1A1, SNCA, VAMP3, SNAP25, RIMS3, RAB40B, HMGCS1, SCD5, ATP2A2, HRMT1L1, and a gene whose transcribed message has a cDNA sequence comprising SEQ ID No: 16, SEQ ID No: 17, SEQ ID No: 18 or SEQ ID No: 19.
contacting the cell with a candidate compound; and
measuring expression of the reporter gene,
   wherein a change in the expression of the reporter gene in response to the candidate compound identifies a compound that is able to modulate expression of the gene selected from FKBP2, SNCA, EEF1A1, VAMP3, SNAP25, RIMS3, RAB40B, HMGCS1, SCD5, ATP2A2, HRMT1L1, and a gene whose transcribed message has a cDNA sequence comprising SEQ ID No: 16, SEQ ID No: 17, SEQ ID No: 18 or SEQ ID No: 19.

An increase in the expression of the reporter gene indicates the identified compound is able to increase expression of the respective polypeptide from its naturally-occurring promoter in vivo.

A decrease in the expression of the reporter gene indicates the identified compound is able to decrease expression of the respective polypeptide from its naturally-occurring promoter in vivo.

Many reporter genes are known in the art and include β-GAL, GFP, and Aequorin (which luminescent upon increase of intracellular calcium).

By “a gene selected from FKBP2, SNCA, EEF1A1, VAMP3, SNAP25, RIMS3, RAB40B, HMGCS1, SCD5, ATP2A2, HRMT1L1, and a gene whose transcribed message has a cDNA sequence comprising SEQ ID No: 16, SEQ ID No: 17, SEQ ID No: 18 or SEQ ID No: 19” we mean the natural genomic sequence which when transcribed is capable of encoding the polypeptide. The natural genomic sequence of the genes may contain introns.

A promoter is an expression control element formed by a DNA sequence that permits binding of RNA polymerase and transcription to occur. Methods for the determination of the sequence of the promoter region of a gene are well known in the art. The presence of a promoter region may be determined by identification of known motifs, and confirmed by mutational analysis of the identified sequence. Preferably, the promoter sequence is located in the region between the transcription start site and 5 kb upstream (5′) of the transcription start site of the gene selected from FKBP2, SNCA, EEF1A1, VAMP3, SNAP25, RIMS3, RAB40B, HMGCS1, SCD5, ATP2A2, HRMT1L1, and a gene whose transcribed message has a cDNA sequence comprising SEQ ID No: 16, SEQ ID No: 17, SEQ ID No: 18 or SEQ ID No: 19. More preferably, it is located in the region between the transcription start site and 3 kb or 2 kb or 1 kb or 500 bp upstream (5′) of the start site, and still more preferably it is located within the 250 bp upstream (5′) of the transcription start site.

Regulatory regions, or transcriptional elements such as enhancers, are less predictable than promoters in their location relative to a gene. However, many motifs indicative of regulatory regions are well characterised and such regions affecting the level of transcription of the relevant gene can usually be identified on the basis of these motifs. The function of such a region can be demonstrated by well-known methods such as mutational analysis and in vitro DNA-binding assays including DNA footprinting and gel mobility shift assays.

Regulatory regions influencing the transcription of a gene selected from FKBP2, SNCA, EEF1A1, VAMP3, SNAP25, RIMS3, RAB40B, HMGCS1, SCD5, ATP2A2, HRMT1L1, and a gene whose transcribed message has a cDNA sequence comprising SEQ ID No: 16, SEQ ID No: 17, SEQ ID No: 18 or SEQ ID No: 19, are likely to be located within the region between the transcription start site and 20 kb or 10 kb or 71b 5 kb or 3 kb, or more preferably 1 kb 5′ upstream of the relevant transcription start site, or can be located within introns of the gene.

The genomic sequence of the human SNCA gene can be found in human PAC 27M07, and the 10.7-kb DNA fragment upstream of the α-synuclein translation start site (positions 19040-29776) can be used as a functional promoter sequence (Touchman et al, 2001). The SNCA Gene Accession No is AF163864, and the transcription start site is at position 29777 within this PAC. Additional suitable promoter and/or regulatory portions from the human SNCA gene can be readily identified by reference to the sequence data and the above positional information regarding the location of promoter and regulatory regions within the genomic sequence.

The genomic sequence of the human FKBP2 gene (Homo sapiens chromosome 11 genomic contig) can be found in Genbank Accession No NT_{—}033903, and the complete genomic sequence (exons and introns) begins at position 9314208 within this genomic sequence. Thus suitable promoter and/or regulatory portions from the FKBP2 gene can be readily identified by reference to the sequence data and the above positional information regarding the location of promoter and regulatory regions within the genomic sequence.

The genomic sequence of the human EEF1A1 gene can be found in Genbank Accession No AL603910 (human DNA sequence from clone RP11-505P4 on chromosome 6), and the complete genomic sequence (exons and introns) begins at position 113397 (complement) within this genomic sequence. Thus suitable promoter and/or regulatory portions from the human EEF1A1 gene can be readily identified by reference to the sequence data and the above positional information regarding the location of promoter and regulatory regions within the genomic sequence.

The genomic sequence of the human VAMP3 gene can be found in Genbank Accession No Z98884 (human DNA sequence from clone RP3-467L1 on chromosome 1p36.21-36.33), and the transcription start site is at position 50661 within this genomic sequence. Thus suitable promoter and/or regulatory portions from the human VAMP3 gene can be readily identified by reference to the sequence data and the above positional information regarding the location of promoter and regulatory regions within the genomic sequence.

The genomic sequence of the human SNAP25 gene can be found in Genbank Accession No NT 011520 (Homo sapiens chromosome 22 genomic contig), and the complete genomic sequence (exons and introns) begins at position 9347813 within this genomic sequence. Thus suitable promoter and/or regulatory portions from the human SNAP25 gene can be readily identified by reference to the sequence data and the above positional information regarding the location of promoter and regulatory regions within the genomic sequence.

The genomic sequence of the human RIMS3 gene can be found in Genbank Accession No AL031289 (human DNA sequence from clone RP4-739H11 on chromosome 1p33-34.2) and the complete genomic sequence (exons and introns) begins at position 56606 within this genomic sequence. Thus suitable promoter and/or regulatory portions from the human RIMS3 gene can be readily identified by reference to the sequence data and the above positional information regarding the location of promoter and regulatory regions within the genomic sequence.

The genomic sequence of the human RAB40B gene can be found in Genbank Accession No NT_{—}086886 (Homo sapiens chromosome 17 genomic contig), and the complete genomic sequence (exons and introns) begins at position 17680884 within this genomic sequence. Thus suitable promoter and/or regulatory portions from the human RAB40B gene can be readily identified by reference to the sequence data and the above positional information regarding the location of promoter and regulatory regions within the genomic sequence.

Additional sequence upstream of the human HMGCS1 coding region can be found in Genbank Accession No BC000297, which is an extension of the mRNA sequence. The coding region begins at position 106 within this sequence. Suitable promoter and/or regulatory portions from the human HMGCS1 gene may be located in the first 105 nucleotides of the sequence in Genbank Accession No BC000297. In any event, additional information on the human HMGCS1 promoter regions can readily be identified by the skilled person by reference to the sequence data and the above positional information regarding the location of promoter and regulatory regions within the genomic sequence.

Additional sequence upstream of the human SCD5 coding region can be found in Genbank Accession No NM_{—}001037582, which is an extension of the mRNA sequence. The coding region begins at position 236 within this sequence. Suitable promoter and/or regulatory portions from the human SCD5 gene may be located in the first 235 nucleotides of the sequence in Genbank Accession No NM_{—}001037582. In any event, additional information on the human SCD5 promoter regions can readily be identified by the skilled person by reference to the sequence data and the above positional information regarding the location of promoter and regulatory regions within the genomic sequence.

The genomic sequence of the human ATP2A2 gene can be found in Genbank Accession No NT_{—}009775 (Hoino sapiens chromosome 12 genomic contig). Suitable promoter and/or regulatory portions from the human ATP2A2 gene can be readily identified by reference to the sequence data and the above positional information regarding the location of promoter and regulatory regions within the genomic sequence.

The genomic sequence of the human HRMT1L1 gene can be found in Genbank Accession No AP001761 (Homo sapiens genomic DNA, chromosome 21q), and the complete genomic sequence (exons and introns) begins at position 558 within this genomic sequence. Thus suitable promoter and/or regulatory portions from the human HRMT1L1 gene can be readily identified by reference to the sequence data and the above positional information regarding the location of promoter and regulatory regions within the genomic sequence.

Typically, an increase in the expression of the reporter gene indicates the compound is an inhibitor of Bax-mediated apoptosis, while a decrease in the expression of the reporter gene indicates the compound is a promoter of Bax-mediated apoptosis.

The invention thus includes a method of identifying a compound that is able to modulate Bax-mediated apoptosis in a cell, the method comprising identifying a compound that modulates expression of a gene selected from FKBP2, SNCA, EEF1A1, VAMP3, SNAP25, RIMS3, RAB40B, HMGCS1, SCD5, ATP2A2, HRMT1L1, and a gene whose transcribed message has a cDNA sequence comprising SEQ ID No: 16, SEQ ID No: 17, SEQ ID No: 18 or SEQ ID No: 19, as described above in the seventeenth aspect of the invention, and testing the identified compound in an assay for Bax-mediated apoptosis. Suitable cell-based and in vivo assays of apoptosis are described above and in the Examples. In an eighteenth aspect, the invention provides a further method of identifying a compound that is able to modulate Bax-mediated apoptosis, the method comprising:
providing a cell that expresses a functional Bax polypeptide under the control of an inducible promoter, and that expresses a polypeptide selected from FKBP2, SNCA, EEF1A1, VAMP3, SNAP25, RIMS3, RAB40B, HMGCS1, SCD5, ATP2A2, HRMT1L1, and a polypeptide encoded by a polynucleotide comprising SEQ ID No: 16, SEQ ID No: 17, SEQ ID No: 18 or SEQ ID No: 19 under the control of its naturally-occurring promoter and/or regulatory sequence(s);
contacting the cell with a candidate compound under conditions that induce expression of the functional Bax polypeptide; and
performing an apoptosis assay,
   wherein a change in the level of apoptosis in comparison to a cell not contacted with the candidate compound indicates a compound that is able to modulate Bax-mediated apoptosis.

Suitable cell-based and in vivo assays of apoptosis are described above and in the Examples.

The inventor considers that an increase in the level of apoptosis indicates that the compound inhibits expression of the polypeptide from its naturally-occurring promoter, and hence is a pro-apoptotic compound. A decrease in the level of apoptosis indicates that the compound enhances or promotes expression of the polypeptide from its naturally-occurring promoter, and hence is an inhibitor of Bax-mediated apoptosis.

In this aspect of the invention, the cell is typically a mammalian cell, preferably a human cell. The cell is preferably from a cell line, that has been transfected with a construct that comprising a polynucleotide that encodes a functional Bax polypeptide under the control of an inducible or constitutive promoter. It is preferred if the cell is a brain cell, and more preferably a hippocampus cell. Suitable cell lines include SH-SY5Y and PC3 cells. In addition, rat and mouse hippocampus derived primary cells are commercially available.

It is preferred if the cell expresses the polypeptide selected from FKBP2, SNCA, EEF1A1, VAMP3, SNAP25, RIMS3, RAB40B, HMGCS1, SCD5, ATP2A2, HRMT1L1, and a polypeptide encoded by a polynucleotide comprising SEQ ID No: 16, SEQ ID No: 17, SEQ ID No: 18 or SEQ ID No: 19 under the control of its naturally-occurring promoter and/or regulatory sequence(s), in the absence of the candidate compound. In this way, expression of the polypeptide can be upregulated or downregulated by the candidate compound, leading to a decrease or an increase in the levels of Bax-mediated apoptosis, respectively.

Alternatively, the cell may not express the polypeptide selected from FKBP2, SNCA, EEF1A1, VAMP3, SNAP25, RIMS3, RAB40B, HMGCS1, SCD5, ATP2A2, HRMT1L1, and a polypeptide encoded by a polynucleotide comprising SEQ ID No: 16, SEQ ID No: 17, SEQ ID No: 18 or SEQ ID No: 19 under the control of its naturally-occurring promoter and/or regulatory sequence(s), in the absence of the candidate compound. In this way, only upregulation of the expression of the polypeptide by the candidate compound can detected, thus identifying a compound that inhibits Bax-mediated apoptosis.

A nineteenth aspect of the invention provides a method of screening for a compound that binds to a polypeptide selected from FKBP2, SNCA, EEF1A1, VAMP3, SNAP25, RIMS3, RAB40B, HMGCS1, SCD5, ATP2A2, HRMT1L1, and a polypeptide encoded by a polynucleotide comprising SEQ ID No: 16, SEQ ID No: 17, SEQ ID No: 18 or SEQ ID No: 19, or a suitable anti-apoptotic derivative thereof, the method comprising:
contacting the polypeptide with a candidate compound;
detecting the presence of a complex containing the polypeptide and the candidate compound; and
optionally, identifying any compound bound to the polypeptide.

Preferably the FKBP2, SNCA, EEF1A1, VAMP3, SNAP25, RIMS3, RAB40B, HMGCS1, SCD5, ATP2A2 and HRMT1L1 polypeptides, and the polypeptide encoded by a polynucleotide comprising SEQ ID No: 16, SEQ ID No: 17, SEQ ID No: 18 or SEQ ID No: 19, are human polypeptides as described above. Suitable anti-apoptotic derivatives of these polypeptides are also as described above

It is appreciated that a compound which binds to a polypeptide selected from FKBP2, SNCA, EEF1A1, VAMP3, SNAP25, RIMS3, RAB40B, HMGCS1, SCD5, ATP2A2, HRMT1L1, and a polypeptide encoded by a polynucleotide comprising SEQ ID No: 16, SEQ ID No: 17, SEQ ID No: 18 or SEQ ID No: 19, or a suitable anti-apoptotic derivative thereof, may modulate the activity of the polypeptide. Typically, the compound which binds to the polypeptide will inhibit or antagonise the activity of the polypeptide. However, in some case, the compound which binds to the polypeptide will enhance or promote the activity of the polypeptide.

In a preferred embodiment, the candidate compound is itself a peptide or polypeptide.

Suitable peptides or polypeptides that bind to these polypeptides may be identified using methods known in the art. One method, disclosed by Scott and Smith (1990) Science 249, 386-390 and Cwirla et al (1990) Proc. Natl. Acad. Sci. USA 87, 6378-6382, involves the screening of a vast library of filamentous bacteriophages, such as M13 or fd, each member of the library having a different peptide fused to a protein on the surface of the bacteriophage. Those members of the library that bind to the polypeptide are selected using an iterative binding protocol, and once the phages that bind most tightly have been purified, the sequence of the peptide may be determined simply by sequencing the DNA encoding the surface protein fusion. Another method that can be used is the NovaTope™ system commercially available from Novagen, Inc., 597 Science Drive, Madison, Wis. 53711. The method is based on the creation of a library of bacterial clones, each of which stably expresses a small peptide derived from a candidate protein in which the binding peptide is believed to reside. The library is screened by standard lift methods using the antibody or other binding agent as a probe. Positive clones can be analysed directly by DNA sequencing to determine the precise amino acid sequence of the binding peptide.

Further methods using libraries of beads conjugated to individual species of peptides as disclosed by Lam et al (1991) Nature 354, 82-84 or synthetic peptide combinatorial libraries as disclosed by Houghten et al (1991) Nature 354, 84-86 or matrices of individual synthetic peptide sequences on a solid support as disclosed by Pirrung et al in U.S. Pat. No. 5,143,854 may also be used to identify peptides that bind.

It will be appreciated that screening assays which are capable of high throughput operation will be particularly preferred. Examples may include cell based assays and protein-protein binding assays. An SPA-based (Scintillation Proximity Assay; Amersham International) system may be used. For example, an assay for identifying a compound capable of modulating the activity of a protein kinase may be performed as follows. Beads comprising scintillant and a polypeptide that may be phosphorylated may be prepared. The beads may be mixed with a sample comprising the protein kinase and ³²P-ATP or ³³P-ATP and with the test compound. Conveniently this is done in a 96-well format. The plate is then counted using a suitable scintillation counter, using known parameters for ³²P or ³³P SPA assays. Only ³²P or ³³P that is in proximity to the scintillant, i.e. only that bound to the polypeptide, is detected. Variants of such an assay, for example in which the polypeptide is immobilised on the scintillant beads via binding to an antibody, may also be used.

Other methods of detecting polypeptide/polypeptide interactions include ultrafiltration with ion spray mass spectroscopy/HPLC methods or other physical and analytical methods. Fluorescence Energy Resonance Transfer (FRET) methods, for example, well known to those skilled in the art, may be used, in which binding of two fluorescent labelled entities may be measured by measuring the interaction of the fluorescent labels when in close proximity to each other. Alternative methods of detecting binding of a polypeptide to macromolecules, for example DNA, RNA, proteins and phospholipids, include a surface plasmon resonance assay, for example as described in Plant et al (1995) Analyt Biochem 226(2), 342-348. Methods may make use of a polypeptide that is labelled, for example with a radioactive or fluorescent label.

A further method of identifying a compound that is capable of binding to a polypeptide selected from FKBP2, SNCA, EEF1A1, VAMP3, SNAP25, RIMS3, RAB40B, HMGCS1, SCD5, ATP2A2, HRMT1L1, and a polypeptide encoded by a polynucleotide comprising SEQ ID No: 16, SEQ ID No: 17, SEQ ID No: 18 or SEQ ID No: 19, or a suitable anti-apoptotic derivative thereof, is one where the polypeptide is exposed to the compound and any binding of the compound to the said polypeptide is detected and/or measured. The binding constant for the binding of the compound to the polypeptide may be determined. Suitable methods for detecting and/or measuring (quantifying) the binding of a compound to a polypeptide are well known to those skilled in the art and may be performed, for example, using a method capable of high throughput operation, for example a chip-based method. Newer VLSIPS™ technology has enabled the production of extremely small chips that contain hundreds of thousands or more of different molecular probes. These biological chips or arrays have probes arranged in arrays, each probe assigned a specific location. Biological chips have been produced in which each location has a scale of, for example, ten microns. The chips can be used to determine whether candidate compounds interact with any of the probes on the chip. After exposing the array to candidate compounds under selected test conditions, scanning devices can examine each location in the array and determine whether a candidate compound has interacted with the probe at that location.

Biological chips or arrays are useful in a variety of screening techniques for obtaining information about either the probes or the candidate compounds. For example, a library of peptides can be used as probes to screen for drugs. The peptides can be exposed to a receptor, and those probes that bind to the receptor can be identified (U.S. Pat. No. 5,874,219).

Another method of targeting proteins that bind and modulate the activity of these polypeptides is the yeast two-hybrid system (Fields & Song (1989) Nature 340: 245-246), where the polypeptides of the invention can be used to “capture” binding proteins.

It is desirable to identify compounds that may modulate the activity of a polypeptide selected from FKBP2, SNCA, EEF1A1, VAMP3, SNAP25, RIMS3, RAB40B, HMGCS1, SCD5, ATP2A2, HRMT1L1, and a polypeptide encoded by a polynucleotide comprising SEQ ID No: 16, SEQ ID No: 17, SEQ ID No: 18 or SEQ ID No: 19, in vivo. Thus it will be understood that reagents and conditions used in the method may be chosen such that the interactions between a polypeptide selected from FKBP2, SNCA, EEF1A1, VAMP3, SNAP25, RIMS3, RAB40B, HMGCS1, SCD5, ATP2A2, HRMT1L1, and a polypeptide encoded by a polynucleotide comprising SEQ ID No: 16, SEQ ID No: 17, SEQ ID No: 18 or SEQ ID No: 19, or a suitable anti-apoptotic derivative thereof, and the interacting polypeptide are substantially the same as between a naturally occurring polypeptide and a naturally occurring interacting polypeptide in vivo.

Alternatively, the methods may be used as “library screening” methods, a term well known to those skilled in the art. Thus, for example, the screening methods of the invention may be used to detect (and optionally identify) a polynucleotide capable of expressing a polypeptide activator of a polypeptide selected from FKBP2, SNCA, EEF1A1, VAMP3, SNAP25, RIMS3, RAB40B, HMGCS1, SCD5, ATP2A2, HRMT1L1, and a polypeptide encoded by a polynucleotide comprising SEQ ID No: 16, SEQ ID No: 17, SEQ ID No: 18 or SEQ ID No: 19, in vivo. Aliquots of an expression library in a suitable vector may be tested for the ability to give the required result.

It will be appreciated that in the screening methods described herein, the identified compound may be a drug-like compound or lead compound for the development of a drug-like compound.

The term “drug-like compound” is well known to those skilled in the art, and may include the meaning of a compound that has characteristics that may make it suitable for use in medicine, for example as the active ingredient in a medicament. Thus, for example, a drug-like compound may be a molecule that may be synthesised by the techniques of organic chemistry, less preferably by techniques of molecular biology or biochemistry, and is preferably a small molecule, which may be of less than 5000 daltons and which may be water-soluble. A drug-like compound may additionally exhibit features of selective interaction with a particular protein or proteins and be bioavailable and/or able to penetrate target cellular membranes, but it will be appreciated that these features are not essential.

The term “lead compound” is similarly well known to those skilled in the art, and may include the meaning that the compound, whilst not itself suitable for use as a drug (for example because it is only weakly potent against its intended target, non-selective in its action, unstable, poorly soluble, difficult to synthesise or has poor bioavailability) may provide a starting-point for the design of other compounds that may have more desirable characteristics.

Hence, an embodiment of the screening methods of the invention provides a method of identifying a drug-like compound or lead compound for the development of a drug-like compound that modulates the activity of a polypeptide selected from FKBP2, SNCA, EEF1A1, VAMP3, SNAP25, RIMS3, RAB40B, HMGCS1, SCD5, ATP2A2, HRMT1L1, and a polypeptide encoded by a polynucleotide comprising SEQ ID No: 16, SEQ ID No: 17, SEQ ID No: 18 or SEQ ID No: 19, in vivo, the method comprising contacting a test compound with the polypeptide or a suitable anti-apoptotic variant thereof, and determining whether an activity of the polypeptide is changed compared to the activity of the polypeptide or the anti-apoptotic derivative thereof in the absence of the test compound.

The screening methods of the seventeenth, eighteenth and nineteenth aspects of the invention may further comprise testing identified compounds for the ability to inhibit Bax-mediated apoptosis in a model of apoptosis, or in a suitable disease model.

Additionally or alternatively, the screening methods of the seventeenth, eighteenth and nineteenth aspects of the invention may further comprise modifying the identified compounds and testing the modified compounds in a model of apoptosis, or in a suitable disease model.

Suitable models of apoptosis are described-above and in the Examples.

Cell cultures from animal models of Alzheimer's disease that can be used for screening and testing drug efficacy are described in US Patent Application No 20050172344. An animal model simulating neurologic disease, particularly AD, is described in US Patent Application No 20050102708. Further animal models of neurodegenerative diseases, including animal models of Alzheimer's disease, animal models of Huntington's disease, rodent and primate models of Parkinson's disease, and genetic/non-genetic Dysmyelination models, are described by Boulton, Baker & Butterworth (1992) in Animal Models of Neurological Disease, I: Neurodegenerative Diseases (Human Press, ISBN: 0-89603-208-6).

Suitable animal models for diabetes, cancer and cardiovascular disease are well known in the art and include chemically-induced diabetes models; nude mice and SCID mouse models for cancer where mouse or human cancer cells are injected into the animals; and ventricular hypertrophy pressure overload models for cardiovascular disease.

As would be appreciated by the person skilled in the art, the method may further comprise formulating a compound which has the ability to inhibit Bax-mediated apoptosis in a model of apoptosis, and especially in an in vivo model, into a pharmaceutically acceptable composition.

Similarly, the method may further comprise formulating a compound which has the therapeutic utility in a disease model, and especially in an in vivo model, into a pharmaceutically acceptable composition.

All of the documents referred to herein are incorporated herein, in their entirety, by reference.

The listing or discussion of a prior-published document in this specification should not necessarily be taken as an acknowledgement that the document is part of the state of the art or is common general knowledge.

The invention will now be described in more detail with the aid of the following Figures and Examples.

### FIGURE LEGENDS

FIG. 1 is a schematic illustration of construct pBGAL1Ms.

FIG. 2 is a schematic illustration of construct yIPLEUG1.

FIG. 3 is a schematic illustration of construct yIPADEG1.

FIG. 4 is a schematic illustration of construct yIPTRPG1.

FIG. 5 is a schematic illustration of construct yIPHISG1.

FIG. 6 is a schematic illustration of construct iLEUBAX.

FIG. 7 is a schematic illustration of construct iADEBAX.

FIG. 8 is a schematic illustration of construct iTRPBAX.

FIG. 9 is a schematic illustration of construct iHISBAX.

FIG. 10 is a schematic illustration of construct pcDNA3.1/Bax.

FIG. 11 is a schematic illustration of construct pIRESEGFP/BAX.

FIG. 12 is a schematic illustration of construct pSYI214.

FIG. 13 is a schematic illustration of construct pRS426/GAL1MS.

FIG. 14 is a schematic illustration of construct pRS426/PGKMS.

FIG. 15 is a schematic illustration of construct pRS416/GAL1MS.

FIG. 16 is a schematic illustration of construct pRS416/PGKMS.

FIG. 17 is a schematic illustration of construct 426/GALBcl2.

FIG. 18 is a schematic illustration of construct 426/GALBcl-xL.

FIG. 19 is a schematic illustration of construct 426/PGKBcl2.

FIG. 20 is a schematic illustration of construct 426/PGKBcl-xL.

FIG. 21 is a schematic illustration of construct 416GAL/Bcl2.

FIG. 22 is a schematic illustration of construct 416GAL/Bcl-xL.

FIG. 23 is a schematic illustration of construct 416PGK/Bcl2.

FIG. 24 is a schematic illustration of construct 416PGK/Bcl-xL.

FIG. 25. Comparison of rescue of Bax-mediated inhibition of cell growth by Bcl-2 and Bcl-xL borne on 2-micron and centromere plasmids (FIGS. 17 to 24). The Bcl-2 and Bcl-xL genes are introduced into different Bax strains (see Table 4) by transformation or via mating. Different observations are made for different Bax strains, plasmids, and their combinations.

Panel A: Spot tests on SD or SG.

Panel B: The score for “growth” is obtained via (background time−growth time)/growth time, in which “growth time” is the days all the positive spots are observed on SG medium. “Background” implies strain transformed with the empty plasmids (FIGS. 13 to 16). The “background time” is scored by the days which the first false-positive colony is observed.

Compared with the other strains, W303baxleu has low background and high growth. The two different promoters, GAL1 and PGK1, do not make any significant difference in any of these strains, P>0.05.

FIG. 26 is a schematic illustration of construct BluKS/p21-HA.

FIG. 27 is a schematic illustration of construct Blu/Bcl2A1-HA.

FIG. 28 is a schematic illustration of construct Blu/FKBP2-HA.

FIG. 29 is a schematic illustration of construct Blu/SNCA-HA.

FIG. 30 is a schematic illustration of construct Blu/VAMP3-HA.

FIG. 31 is a schematic illustration of construct Blu/EEF1A1-HA.

FIG. 32 is a schematic illustration of construct Blu/Bclxl-HA.

FIG. 33 is a schematic illustration of construct pcD/Bcl2A1-HA.

FIG. 34 is a schematic illustration of construct pcD/FKBP2-HA.

FIG. 35 is a schematic illustration of construct pcD/SNCA-HA.

FIG. 36 is a schematic illustration of construct pcD/VAMP3-HA.

FIG. 37 is a schematic illustration of construct pcD/EEF1A1-HA.

FIG. 38 is a schematic illustration of construct pcD/BclxL-HA.

FIG. 39 (A) is a schematic illustration of construct pSYE224.

FIG. 39 (B) is a schematic illustration of construct pYES2 (Invitrogen).

FIG. 40 is a schematic illustration of construct pSYE239.

FIG. 41 is a schematic illustration of construct 224/Bcl2A1-HA.

FIG. 42 is a schematic illustration of construct 224/FKBP2-HA.

FIG. 43 is a schematic illustration of construct 224/SNCA-HA.

FIG. 44 is a schematic illustration of construct 224/VAMP3-HA.

FIG. 45 is a schematic illustration of construct 224/EEF1A1-HA.

FIG. 46 is a schematic illustration of construct 224/BclxL-HA.

FIG. 47 is a schematic illustration of construct 239/Bcl2A1-HA.

FIG. 48 is a schematic illustration of construct 239/FKBP2-HA.

FIG. 49 is a schematic illustration of construct 239/SNCA-HA.

FIG. 50 is a schematic illustration of construct 239/VAMP3-HA.

FIG. 51 is a schematic illustration of construct 239/EEF1A1-HA.

FIG. 52 is a schematic illustration of construct 239/BclxL-HA.

FIG. 53. After transformation into the Bax-strain W303baxleu, the rescuing ability of these genes is tested. Spot tests are done by spotting 5 μl aliquots (i.e. identical number of cells) onto SD and SG plates. The cells that co-express the novel genes and BAX grow to different degrees on SG plates. This is in contrast to the cells that harbour only BAX but contain the empty vector. As expected, these cells do not grow at all. Similar results are obtained with the novel genes being expressed under the control of the GAL1 and PGK1 promoters.

FIG. 54 is a representative picture of immunocytochemistry for the presence of HA-tagged proteins.

FIG. 55. Panel A shows the percentage of dead cells for the indicated times after Bax-transfection. Panel B shows the relative percentages of viable cells on the third day after Bax or vector transfection.

FIG. 56. Ability to rescue from Bax-mediated death by the novel proteins. Viable cells (trypan blue negative cells) with or without the rescuing proteins are counted under a light microscope after Bax-transfection, using a haemocytometer. Bax-mediated death is calculated according to the formula shown above. Compared to cell lines which contain the vector alone, the cells that express the newly found anti-apoptotic genes show a significant decrease in Bax-mediated death (P<0.01). The percentage of cells in each cell line undergoing Bax-mediated death, in the absence or presence of the newly found anti-apoptotic genes, is annotated in the figure.

FIG. 57. Quantitative TUNEL-FACS analysis of apoptosis in HEK293 cells expressing human Bcl-2A1, FKBP2, SNCA, VAMP3, EEF1A1 and Bcl-xL stably after Bax-transfection. Cells are cultured and assayed as described in Example 6 and DNA fragmentation (that results in apoptosis) is detected by flow cytometry. The percentages of cells that have undergone apoptosis, in the absence and presence of the newly found anti-apoptotic proteins, are annotated in the figure. H₂O₂-treated cells are taken as a positive control since H₂O₂ is known to induce profound apoptosis in all eukaryotic cells. The y-axis stands for fluorescence intensity of FITC-dUTP, whereas the x-axis represents DNA content.

FIG. 58. The novel genes inhibit Bax-induced Cytochrome c release. On the third day post-transfection, the cells are labelled with an anti-cytochrome c monoclonal antibody followed by the Alexa 488-conjugated goat anti-mouse IgG. The labelled cells are visualised by fluorescence microscopy using FITC-specific filters. The punctate cytochrome c labelling pattern represents the mitochrondrial localisation of cytochrome c while the diffuse labelling pattern represents a cytosolic localisation of it. An intense labelling of cytosolic cytochrome c is observed with cells after Bax-transfection but not containing the Bax-rescuing proteins. On the other hand, the cell lines which co-express Bcl-2A1, FKBP2, SNCA, VAMP3, EEF1A1 or Bcl-xL together with Bax display a decrease of cytosolic cytochrome c localization pattern.

FIG. 59. The novel Bax-rescuing genes inhibit Bax-induced ΔΨₘ loss. Bax is transfected into HEK293 cell lines with or without novel gene expression. The transfectants are incubated for 72 h as described. ΔΨₘ is assessed by flow-cytometry with DiOC₆(3) staining. HEK293 treated with H₂O₂ triggers the negative or positive control. In the flow-cytometric profiles, the y-axis stand for the events analyzed (10,000); the x-axis refer to the log of fluorescence intensity; the percentages refer to ΔΨₘ loss.

FIG. 60. The novel genes decrease Bax-induced intracellular ROS. The expression of Bcl-2A1, FKBP2, SNCA, VAMP3, EEF1A1 and Bcl-xL leads to decreased fluorescence-intensity compared to cells which express Bax alone with the empty vector (Vector). The parent HEK293 cells transfected with pcDNA3.1(+) is the negative control and is represented as 100% (CTL), while H₂O₂-treated HEK293 is the positive control.

FIG. 61. Polynucleotide sequences of GAL1MS construct, XhoI-SacI fragment (SEQ ID No: 1); and the human Bax alpha BglII-XbaI fragment synthesised using yeast-biased codons (SEQ ID No: 2); and the polypeptide sequence encoded by human Bax alpha BglII-XbaI fragment (SEQ ID No: 3).

FIG. 62. Polynucleotide sequences of “hits” obtained from the yeast screen for inhibitors of Bax-mediated apoptosis. The sequences listed are SCNA (SEQ ID No: 5), FKBP2 (SEQ ID No: 6), SNAP25 (SEQ ID No: 9), EEF1A1 (SEQ ID No: 7), VAMP3 (SEQ ID No: 8), RIMS3 (SEQ ID NO: 10), RAB40B (SEQ ID No: 11), HMGCS1 (SEQ ID NO: 12), SCD5 (SEQ ID No: 13), ATP2A2 (SEQ ID No: 14) and HRMT1L1 (SEQ ID No: 15). For ATP2A2, the fragment isolated was 3200 bp which has been only partially sequenced. The sequences that were obtained using 5′ and 3′ primers match the reference database sequence in the regions shown.

FIG. 63. The sequence of the polynucleotide from human chromosome 3p of previously unknown function as defined in SEQ ID No: 16. As indicated by the arrows, two open reading frames are present within SEQ ID No: 16. Restriction enzyme sites are also indicated.

FIG. 64. The sequence of the polynucleotide from human chromosome 22q11.22-12.2 of previously unknown function as defined in SEQ ID No: 17. As indicated by the arrows, four open reading frames are present within SEQ ID No: 17. Restriction enzyme sites are also indicated.

FIG. 65. The sequence of the polynucleotide from human mitochondrial isolate WH6967 of previously unknown function as defined in SEQ ID No: 18. As indicated by the arrows, two open reading frames are present within SEQ ID No: 18. Restriction enzyme sites are also indicated.

FIG. 66. The sequence of the polynucleotide from human mitochondrial isolate S1216 of previously unknown function as defined in SEQ ID No: 19. As indicated by the arrows, two open reading frames are present within SEQ ID No: 19. Restriction enzyme sites are also indicated.

FIG. 67. A comparison of the amino acid sequences of human EEF1A1 (SEQ ID No: 22) and human EEF1A2, showing 92.6% sequence identity (SEQ ID No: 23).

### EXAMPLE 1

### Generation of DNA Constructs

Generation of DNA Constructs that Allow Expression of Pro-Apoptotic Bax
Constructs for Inducible Expression of Bax in Yeast from Different Chromosomal Loci

Yeast integrative vectors pRS305 (with the Saccharomyces cerevisiae LEU2 gene as selective marker), pRS402 (with the S. cerevisiae ADE2 gene as selective marker) and pRS404 (with the S. cerevisiae TRP1 gene as selective marker) are purchased from ATCC (LGC Promochem, Middlesex, UK).

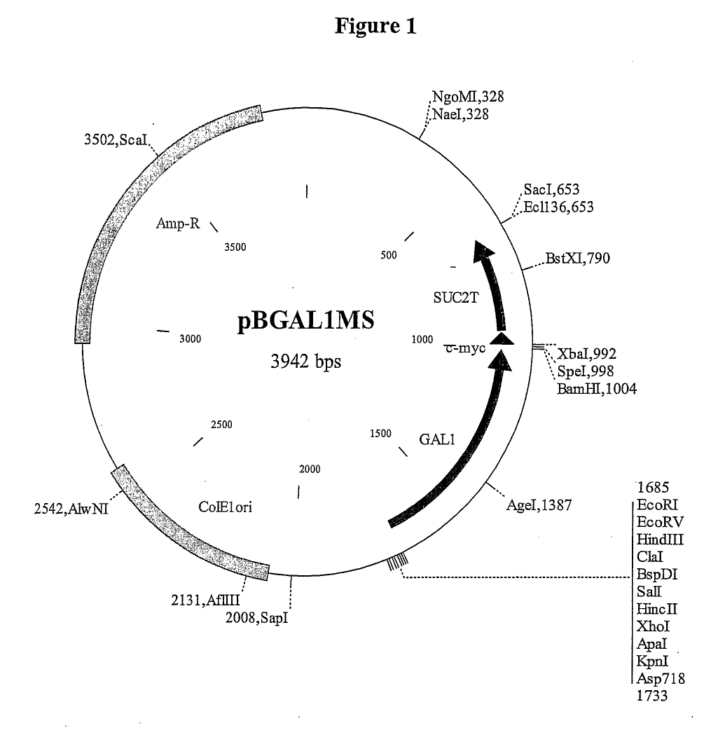

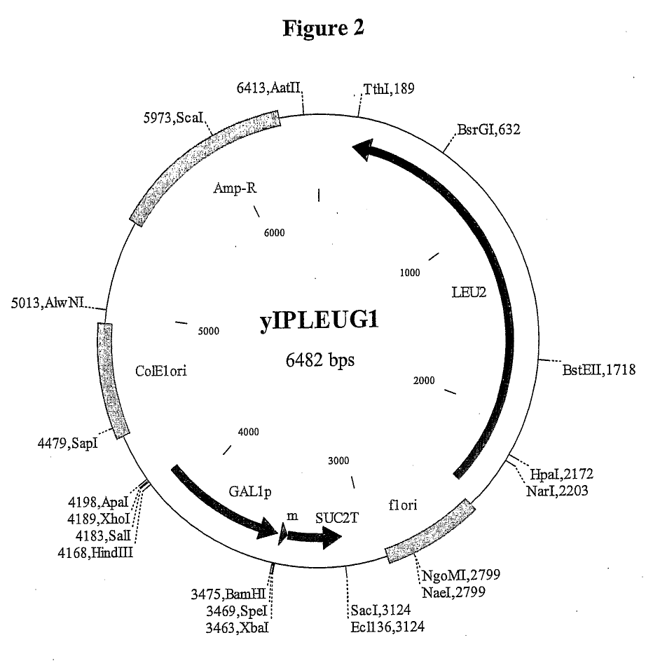

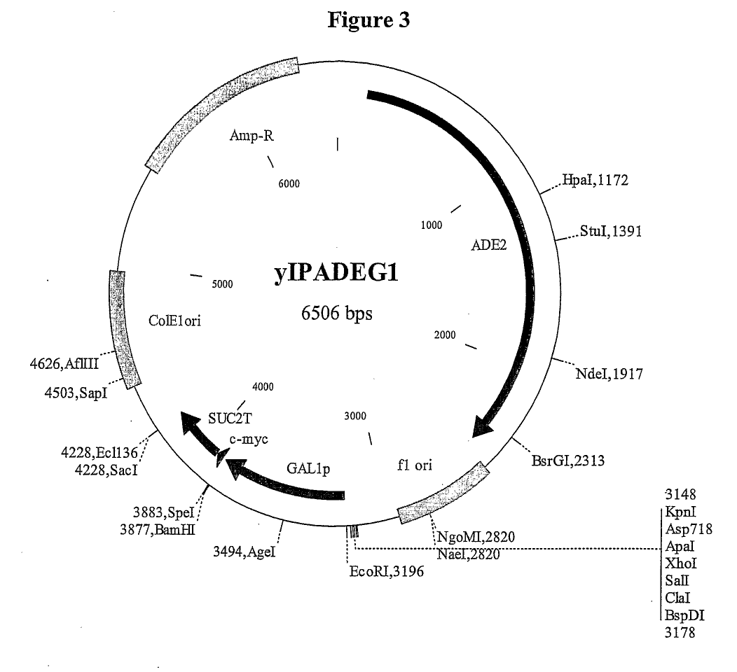

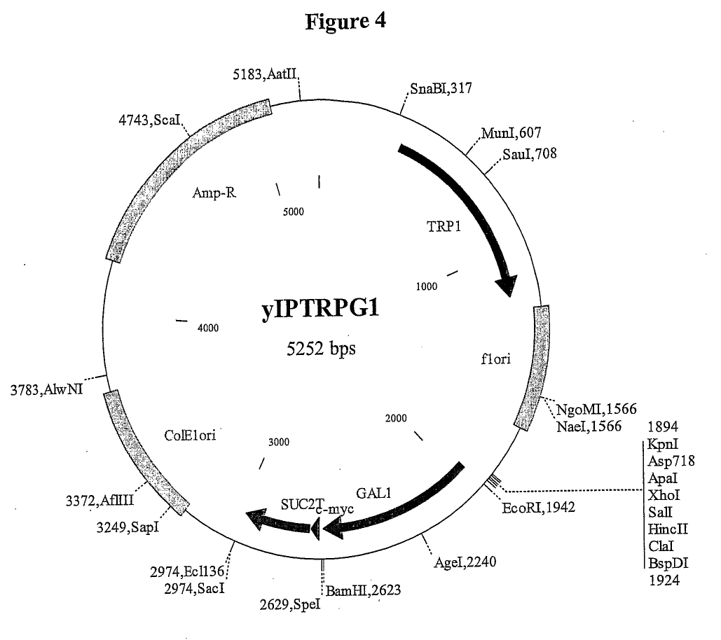

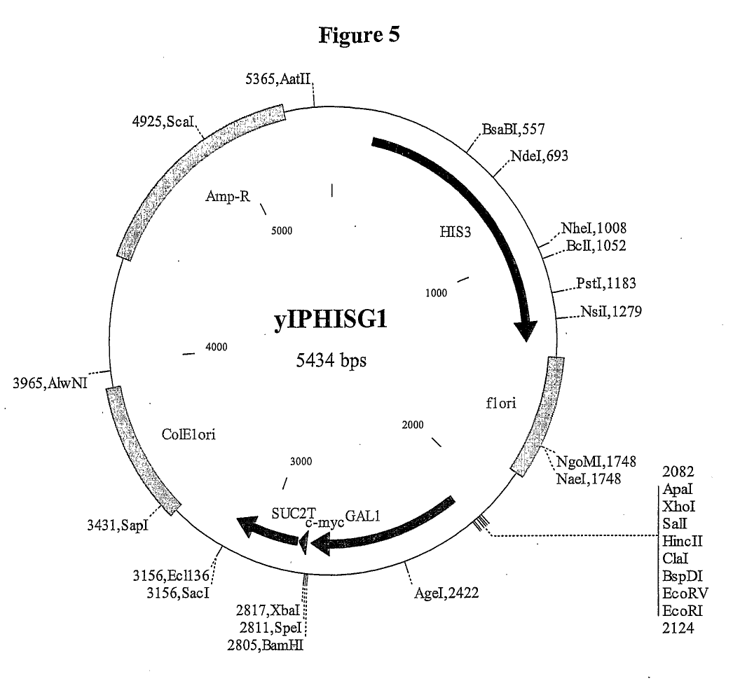

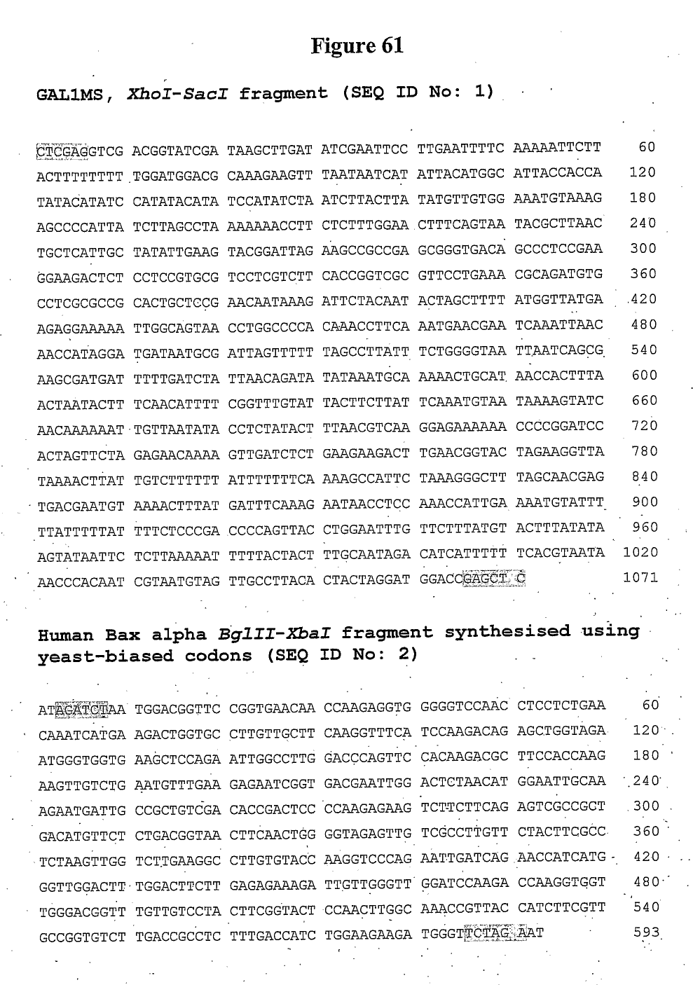

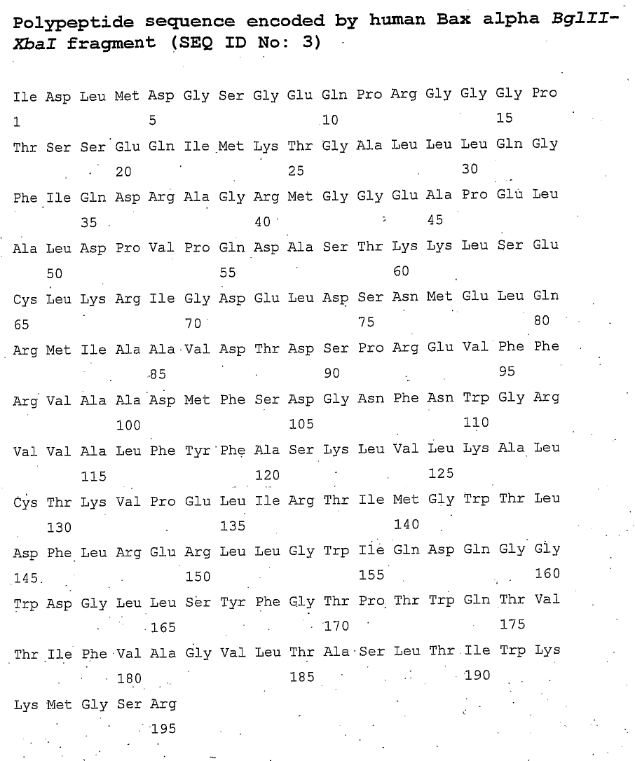

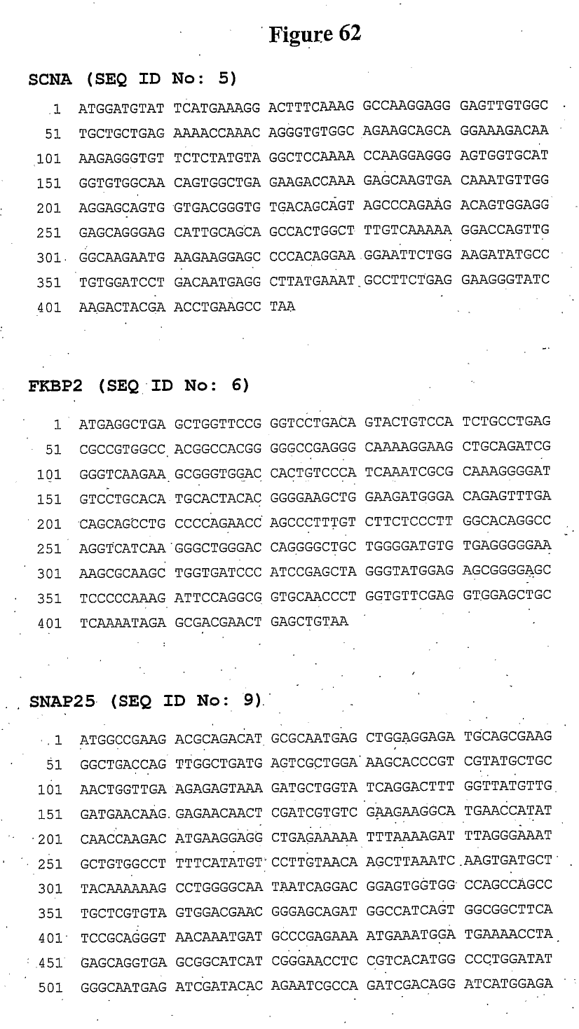

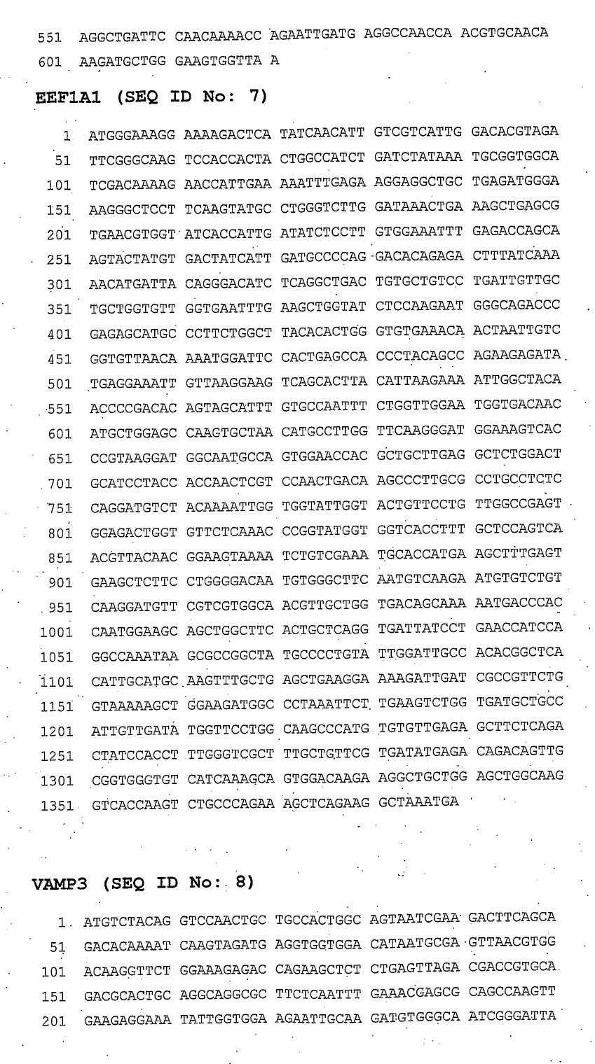

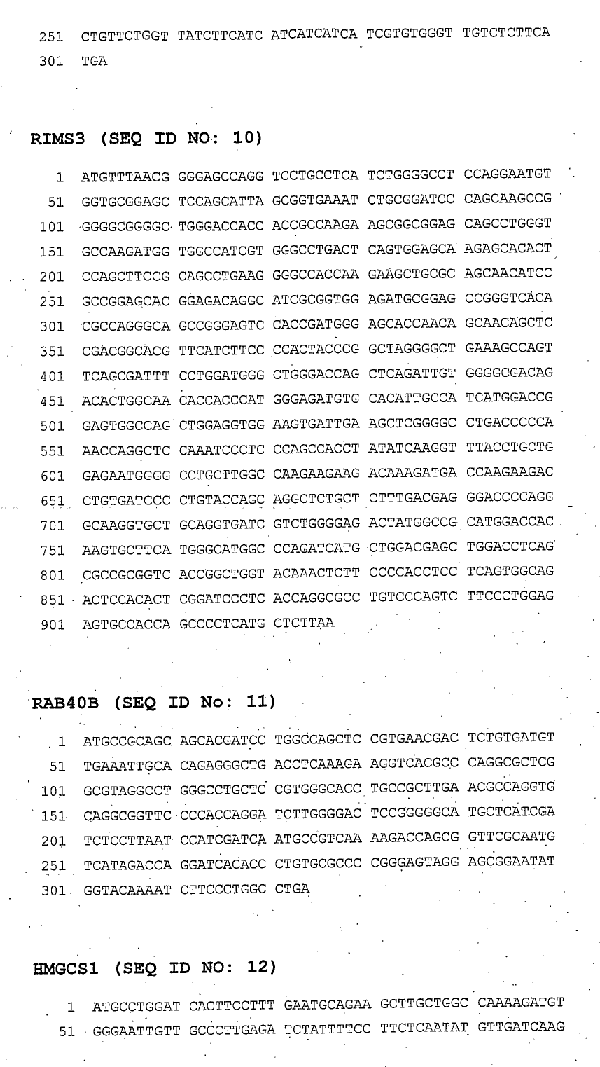

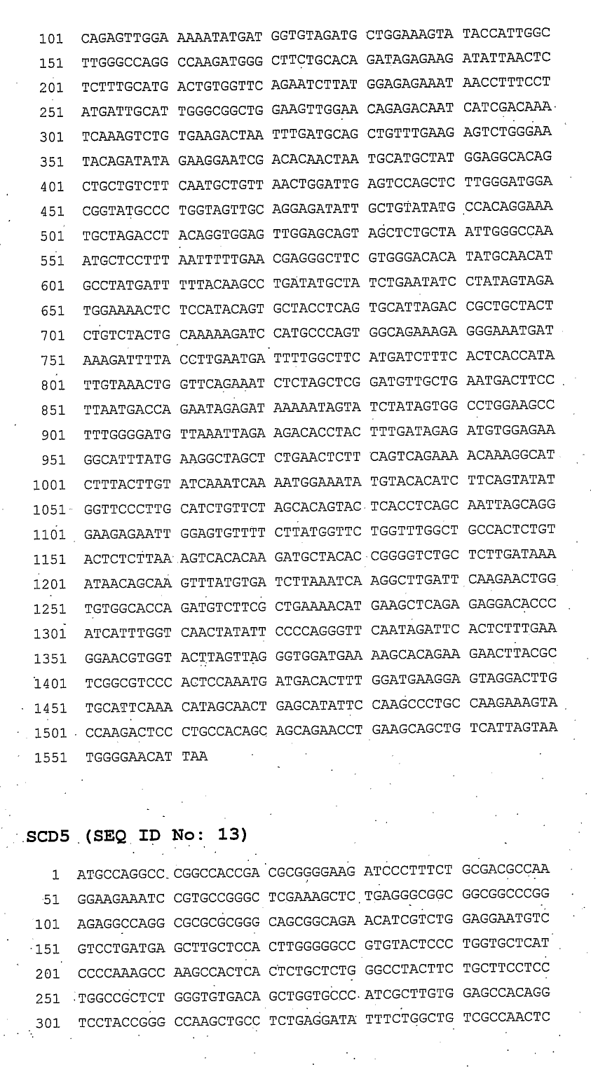

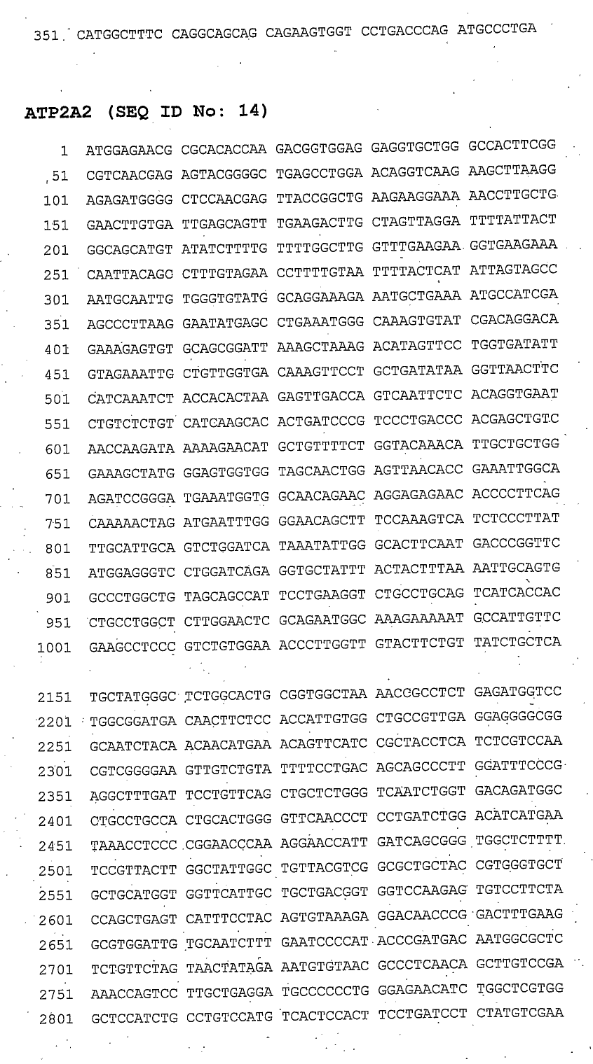

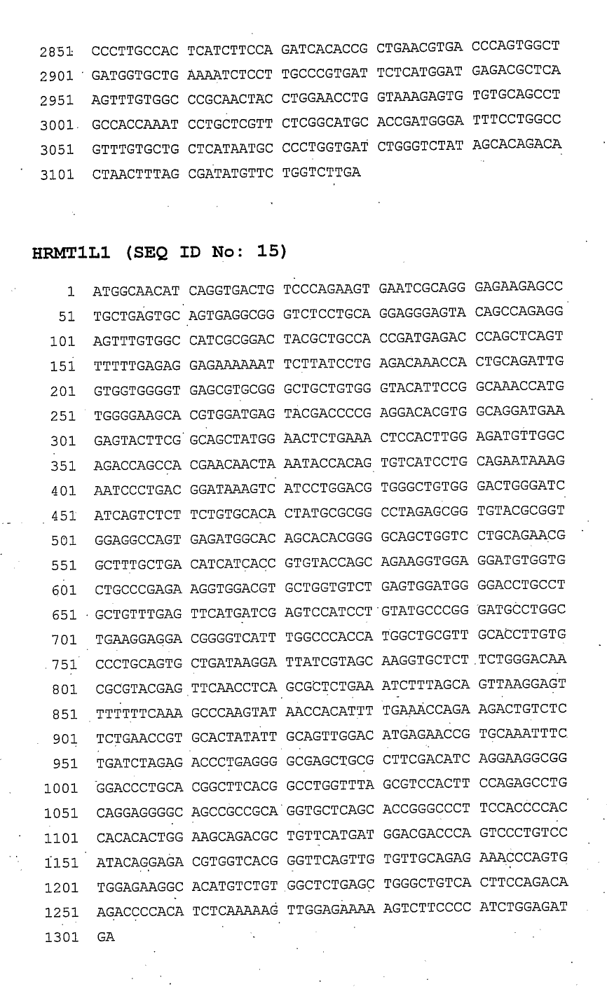

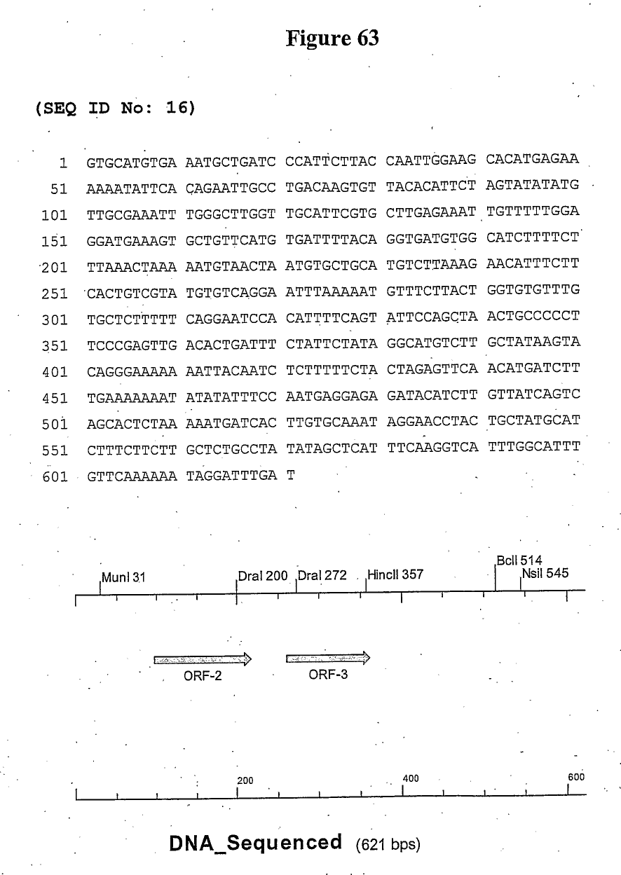

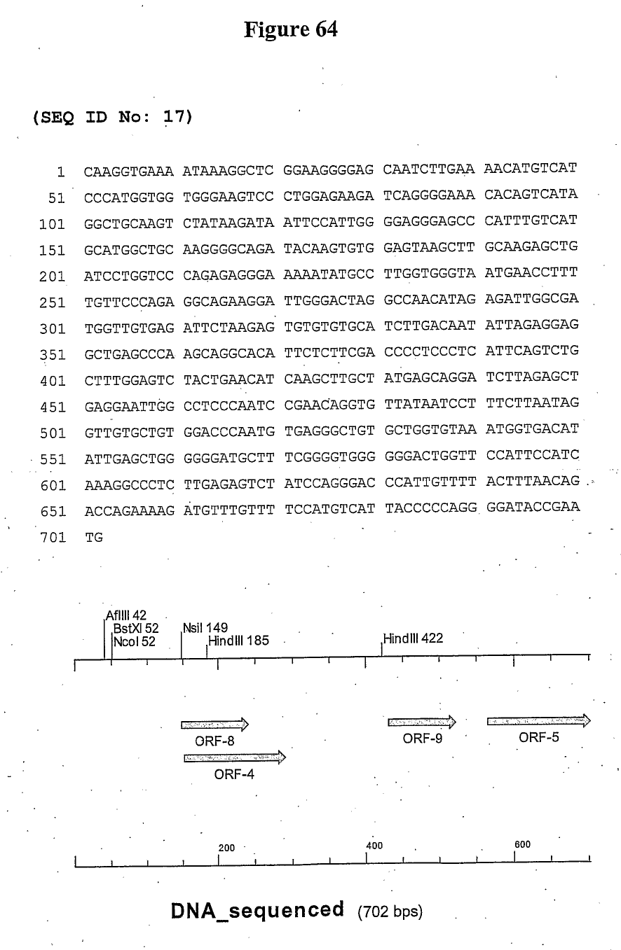

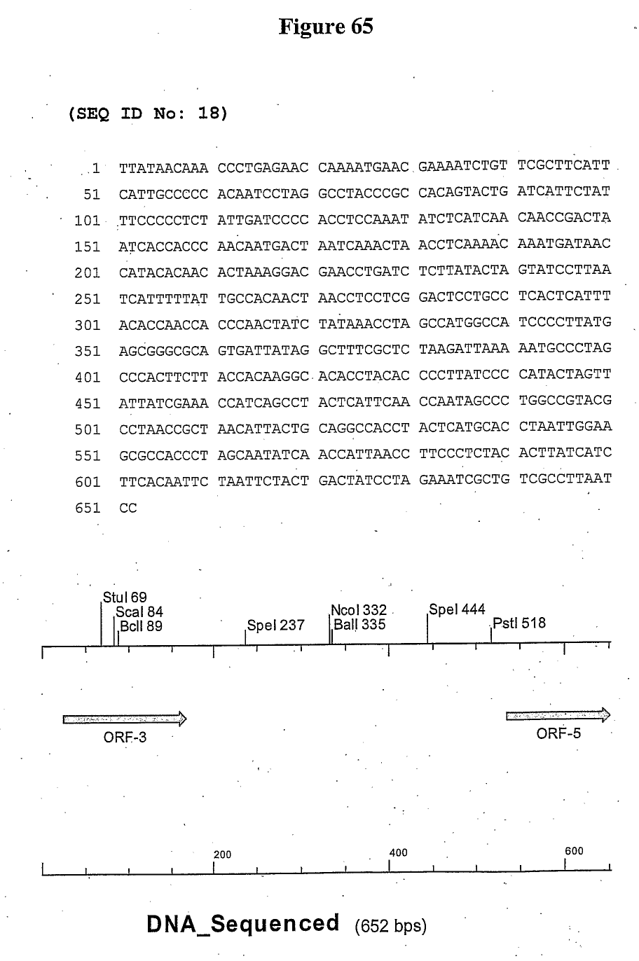

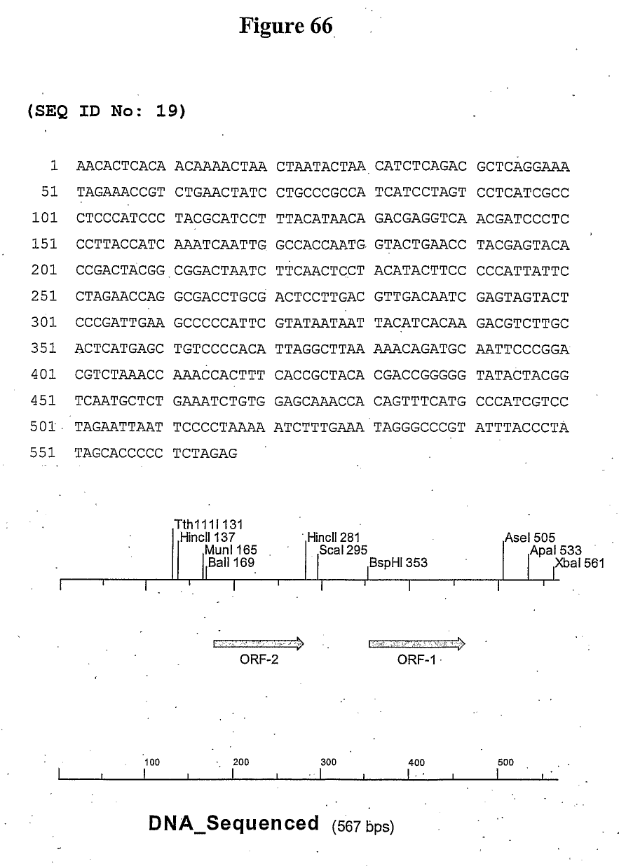

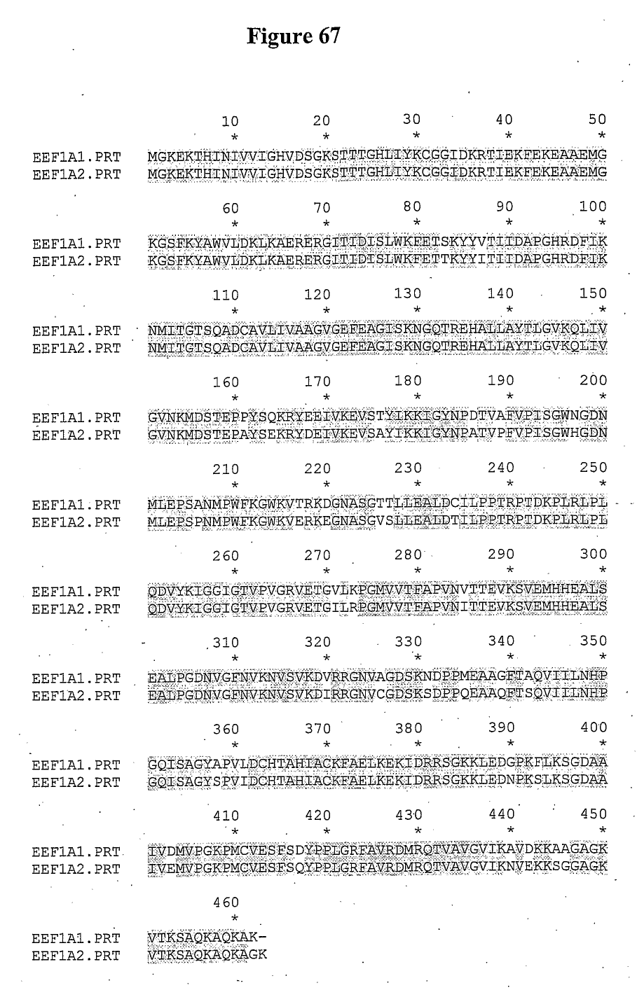

The XhoI-SacI GAL1p-MS fragment, containing the S. cerevisiae GAL1 gene promoter fragment and the SUC2 gene terminator fragment (S) which is preceded by a c-myc Tag fragment (M), is isolated from the construct pBGAL1MS (a pBlueScript KS(+) derivative; FIG. 1; Sequence of GAL1MS shown as SEQ ID No: 1 in FIG. 61) and is ligated to the XhoI-SacI digested pRS305, pRS402 and pRS404 vectors that allows integration at the LEU2, ADE2, TRP1 and HIS3 chromosomal loci. The four resultant plasmids yIPLEUG1 (FIG. 2), yIPADEG1 (FIG. 3), yIPTRPG1 (FIG. 4) and yIPHISG1 (FIG. 5) have unique BamHI, SpeI and/or XbaI restriction sites in between the 3′-end of the GAL1 promoter and the 5′-end of the c-myc tag linked to the SUC2 terminator.

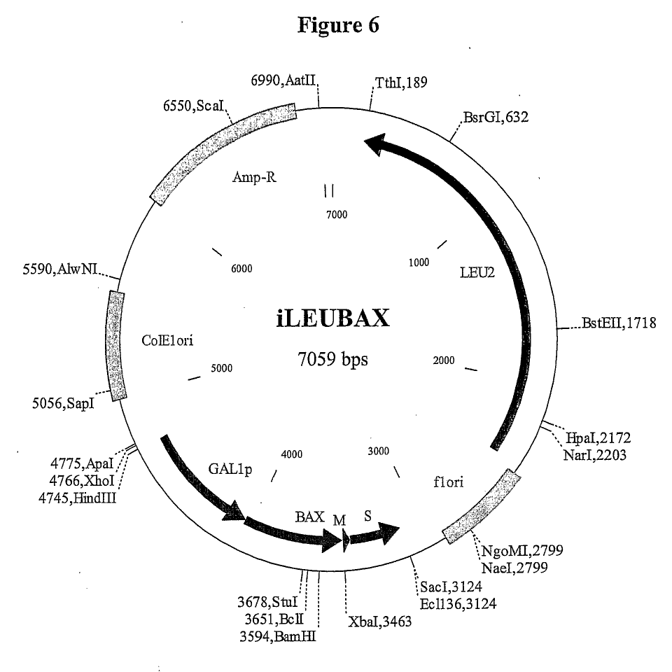

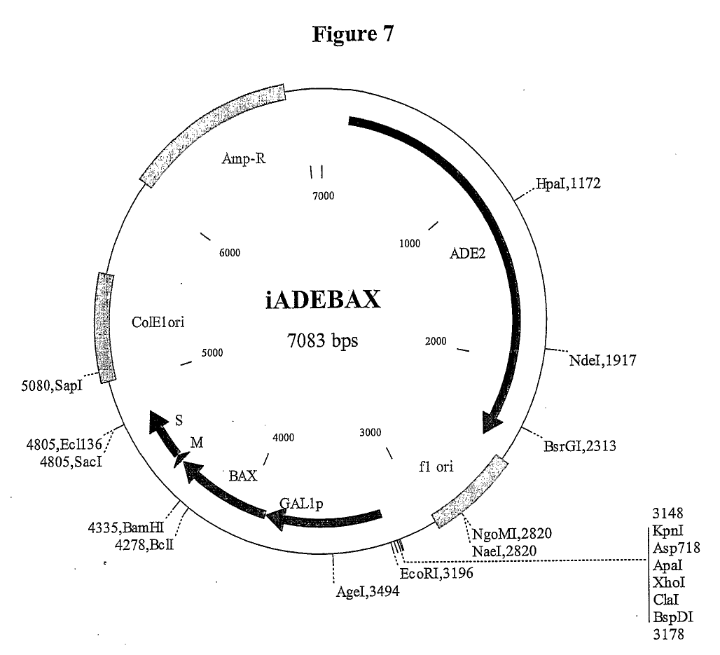

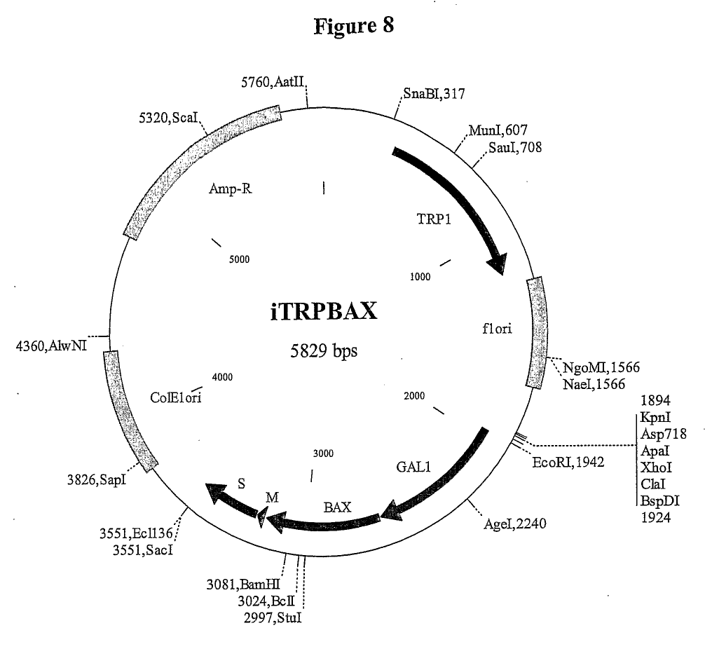

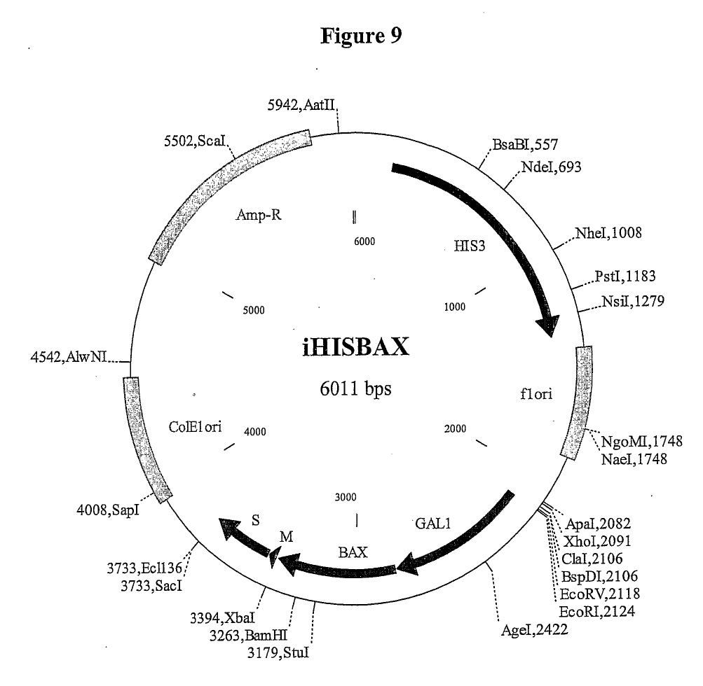

The plasmids yIPLEUG1 (FIG. 2), yIPADEG1 (FIG. 3), yIPTRPG1 (FIG. 4) and yIPHISG1 (FIG. 5) are digested with BamHI and SpeI and are individually ligated to the BglII-XbaI fragment of the Bax gene which is isolated from the plasmid pUC19A/Bax-MS (where the BAX gene, SEQ ID No: 2, has been synthesised using yeast-biased codons (Bennetzen & Hall, 1982)). The resultant BAX-harbouring plasmids are named iLEUBAX (FIG. 6) iADEBAX (FIG. 7), iTRPBAX (FIG. 8), and iHISBAX (FIG. 9). All four plasmids have unique restriction sites in their yeast selection markers that allow linearisation of these plasmids for integration into the respective chromosomal loci.

### Constructs for Constitutive Expression of Bax in Mammalian Cells

Subcloning of the BAX Gene Isolated from SHSY-5Y Neuroblastoma Cells in pcDNA3.1(+)

An EcoRI-XbaI fragment of the BAX gene is cloned from the total mRNA, isolated from the human neuroblastoma cell line SHSY-5Y, using reverse-transcriptase mediated (RT) PCR. The sequence of the isolated BAX gene upon translation corresponds exactly to the Bax-alpha protein, reported in the NCBI database. This fragment is used for further subcloning in pBluescriptKS(+) (Stratagene) digested with EcoRI and XbaI to obtain the plasmid pKS(+)/EcoRI-XbaI/Bax.

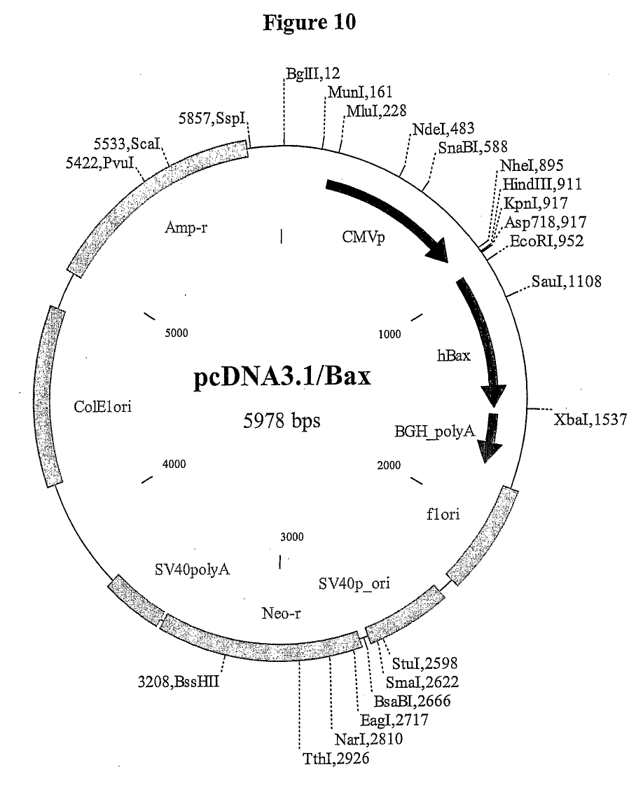

The EcoRI-XbaI Bax gene insert, encoding full-length BAX gene that includes the Start and Stop codons, is isolated from the construct pKS(+)/EcoRI-XbaI/Bax and is ligated to EcoRI-XbaI-digested vector pcDNA3.1(+) (Invitrogen, Paisley, UK) to obtain pcDNA3.1/Bax (FIG. 10).

Subcloning of the BAX Gene Isolated from SHSY-5Y Neuroblastoma Cells in pIRES2-GFP

The plasmid pIRES2-EGFP (Clontech Laboratories, Inc., Oxford, UK) is a bicistronic vector which allows rapid and efficient selection of positive clones that express human Bax protein together with the selection marker, enhanced green fluorescent protein (EGFP), so that virtually all transfected cells that express EGFP (monitored by fluorescence microscopy) also express the Bax protein. The pIRES2-EGFP vector contains the internal ribosome entry site (IRES) of the encephalomyocarditis virus (ECMV), which permits the translation of two open reading frames from one messenger RNA (Clontech Laboratories). Ribosomes can enter the bicistronic mRNA either at the 5′ end to translate BAX or at the ECMV IRES to translate the selection marker EGFP.

The EcoRI-XbaI BAX gene insert, encoding full-length BAX gene isolated from SHSY-5Y (see 1.1.2.1) and which includes Start (ATG) and Stop (TAG) codons, is isolated from the construct pKS(+)/EcoRI-XbaI/Bax and is ligated to EcoRI-XbaI-digested vector pSP73 (Promega) to obtain the plasmid pSP73/EcoRI-XbaI/Bax.

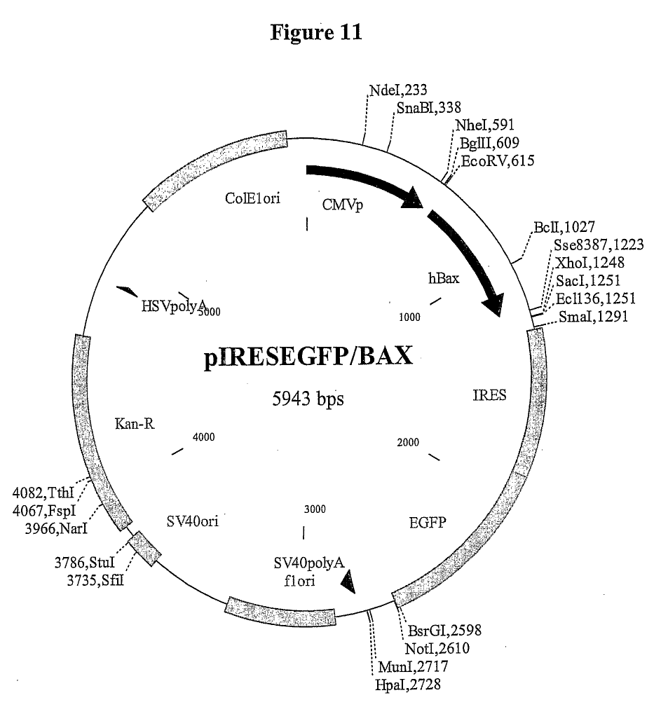

A BglII-XhoI BAX gene fragment is isolated from pSP73/EcoRI-XbaI/Bax (the BglII site is upstream of EcoRI and XhoI is downstream of XbaI in pSP73) and is ligated to the BglII-SalI-digested pIRES2-EGFP vector. The resultant plasmid pIRES2-EGFP/Bax (FIG. 11) allows the simultaneous expression of Bax and EGFP.

Generation of Other DNA Constructs for the Yeast Apoptosis Screen (YAS) that Allow Expression of Anti-Apoptotic Proteins

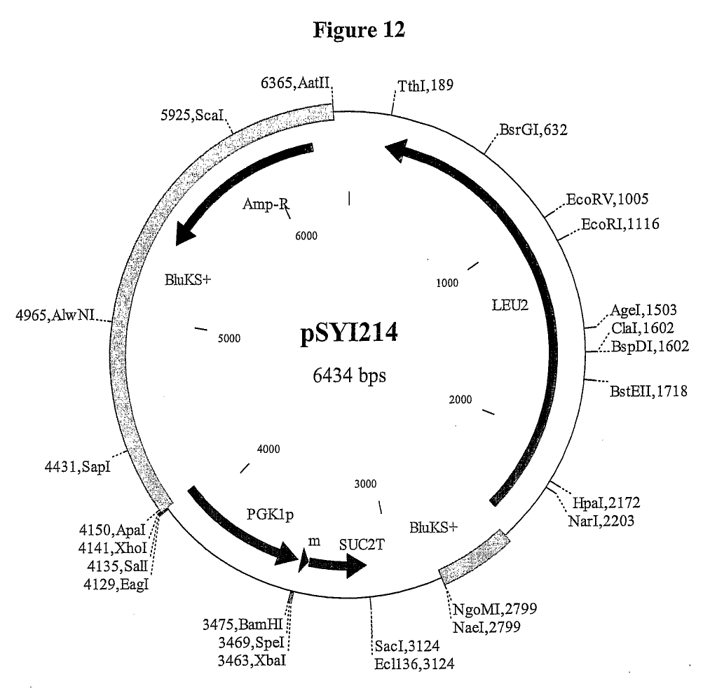

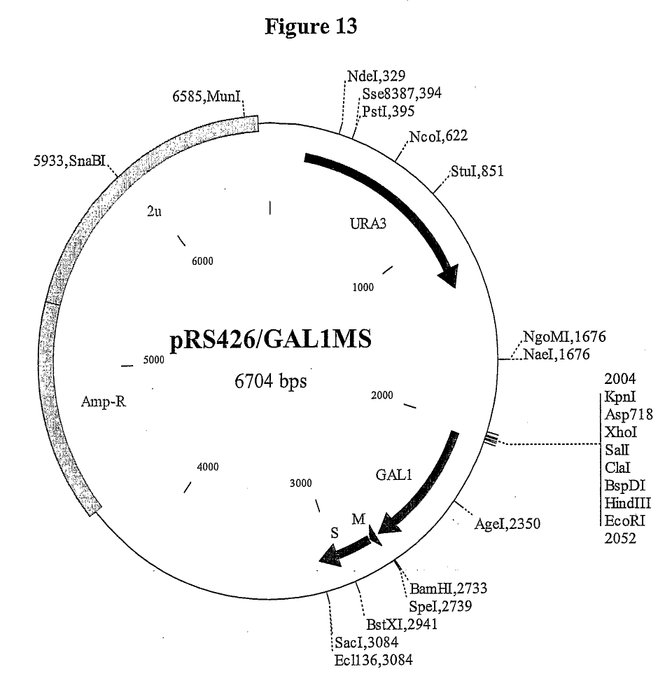

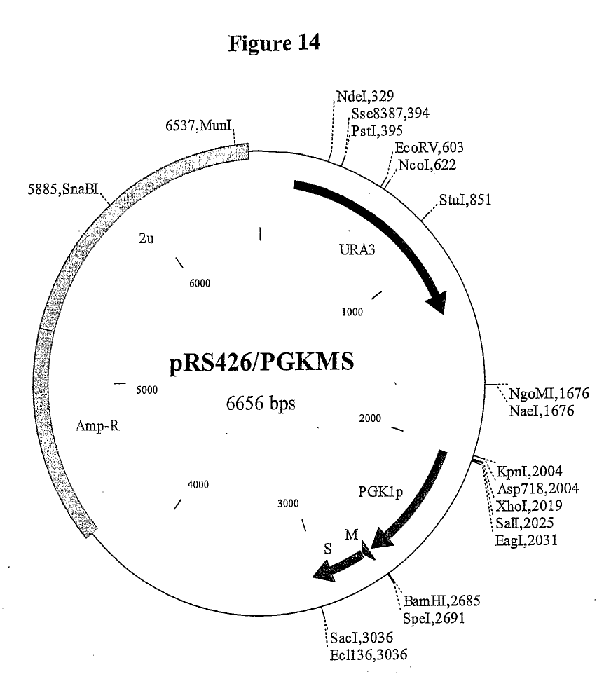

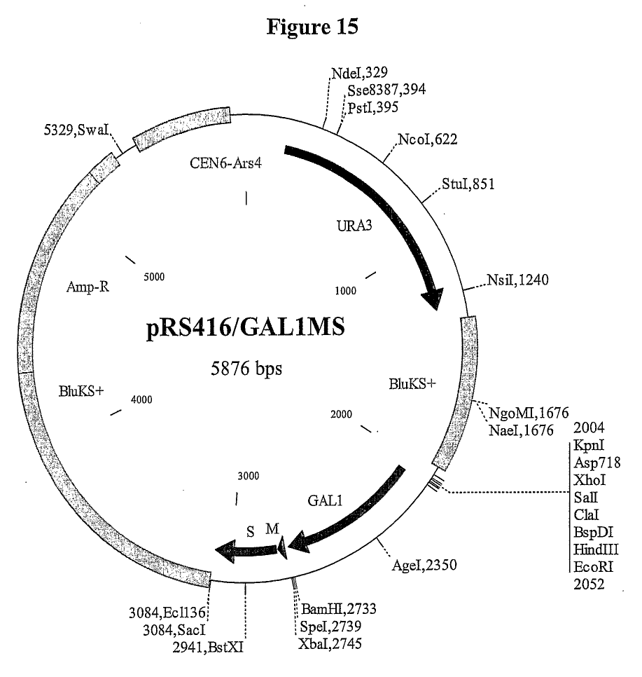

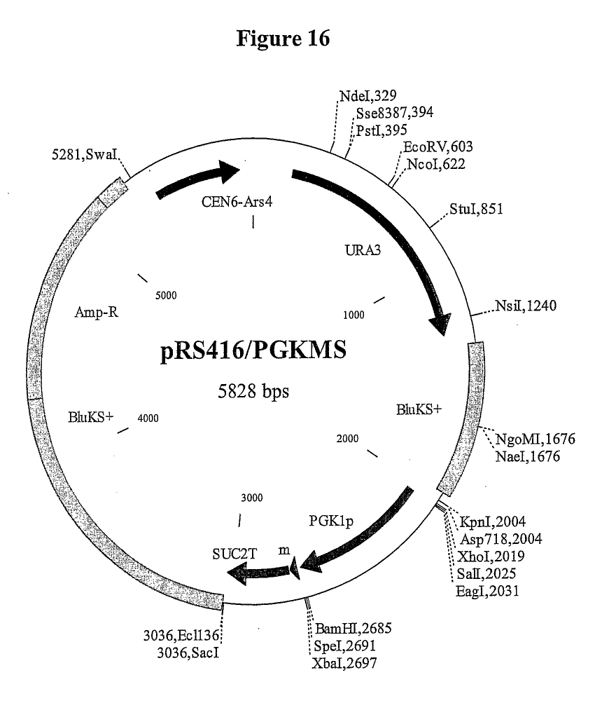

XhoI-SacI-fragments of GAL1p-MS fragment (i.e. GAL1 promoter together with a SUC2 terminator fragment preceded by a c-myc tag sequence that ends with a Stop codon) and PGKp-MS fragment (i.e. PGK1 promoter together with a SUC2 terminator fragment preceded by a c-myc tag sequence that ends with a Stop codon) are isolated from the constructs pBGAL1MS (FIG. 1) and pSYI214 (FIG. 12). The fragments are ligated to XhoI-SacI digested vectors pRS416 and pRS426 (both plasmids are obtained from ATCC) to obtain the plasmids pRS426/GAL1MS (FIG. 13), pRS426/PGKMS (FIG. 14), pRS416/GAL1MS (FIG. 15) and pRS416/PGKMS (FIG. 16). The plasmid pRS416 is a single-copy centromeric vector whereas pRS426 is a multi-copy 2-micron vector.

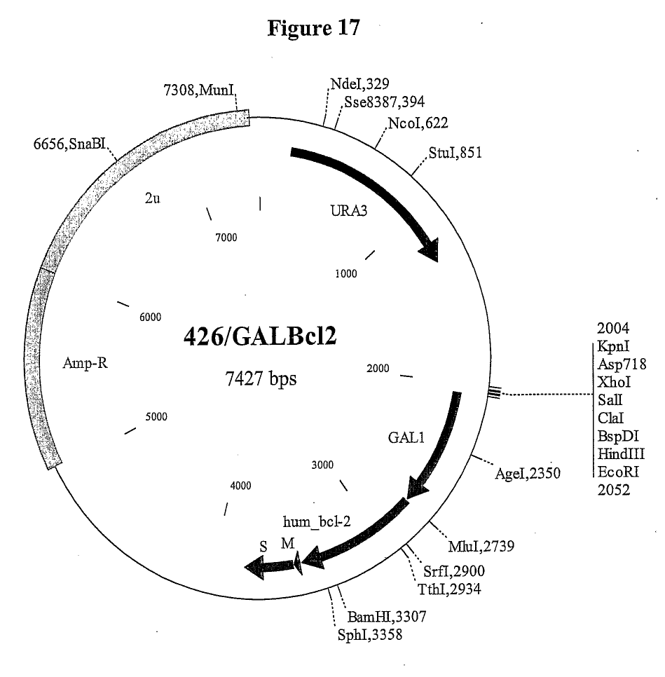

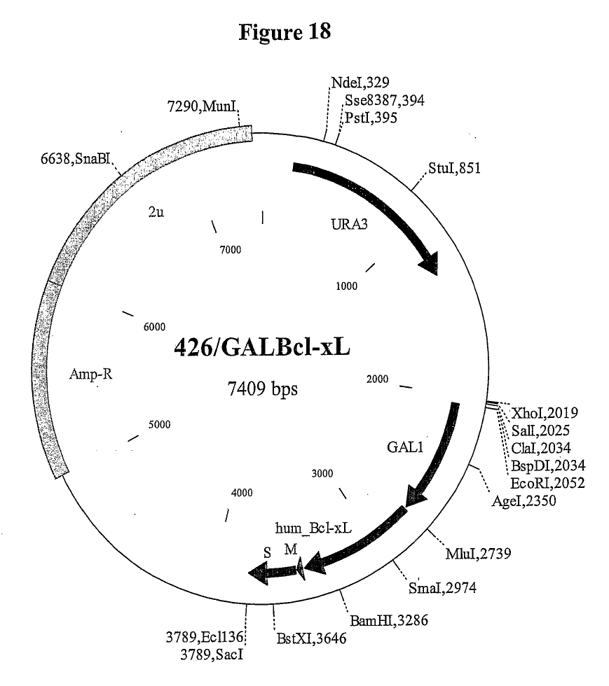

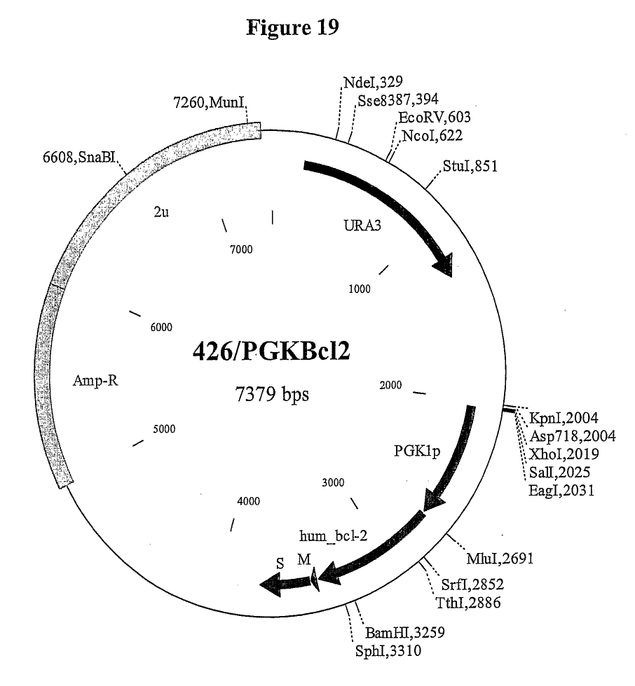

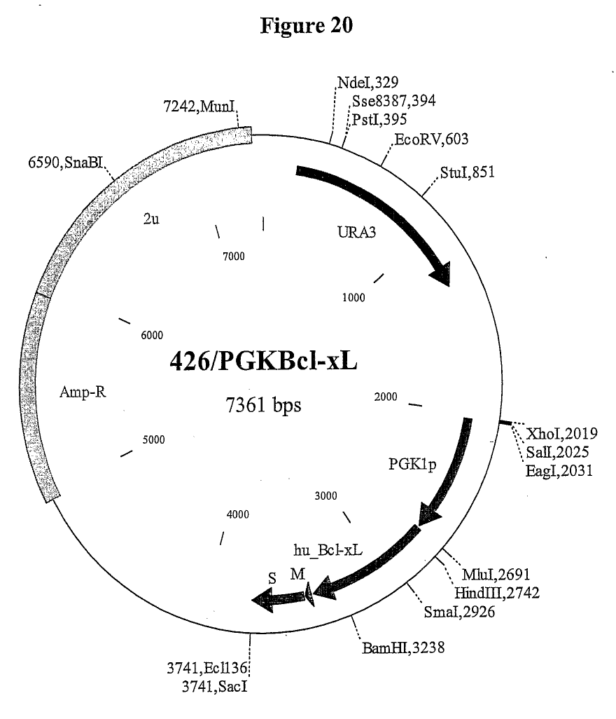

BglII-XbaI-digested fragments encoding human Bcl-2 and human Bcl-xL genes, isolated from plasmids pSP73/BglII-XbaI/Bcl2 and pSP73/BglII-XbaI/Bcl-xL, are ligated into BamHI-SpeI-digested pRS426/GAL1pMS or pRS426/PGKpMS to obtain the plasmids 426GAL/Bcl2 (FIG. 17), 426GAL/Bcl-xL (FIG. 18), 426PGK/Bcl2 (FIG. 19), 426PGK/Bcl-xL (FIG. 20).

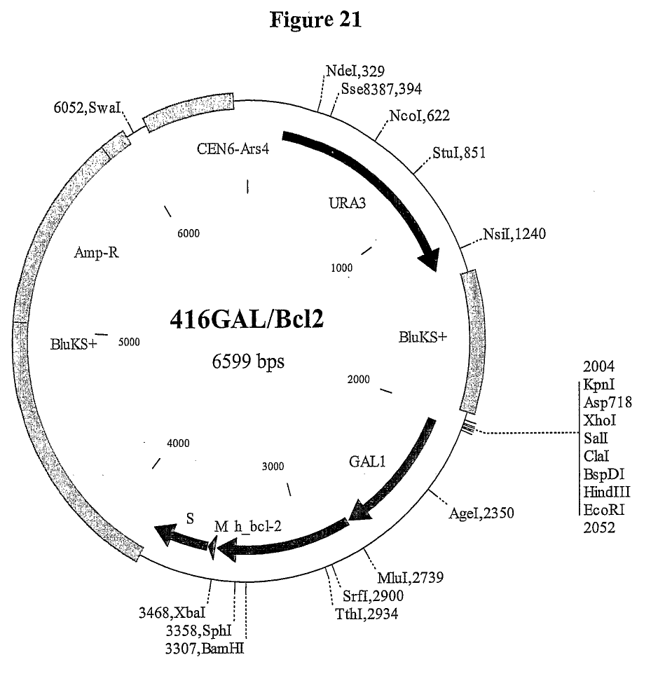

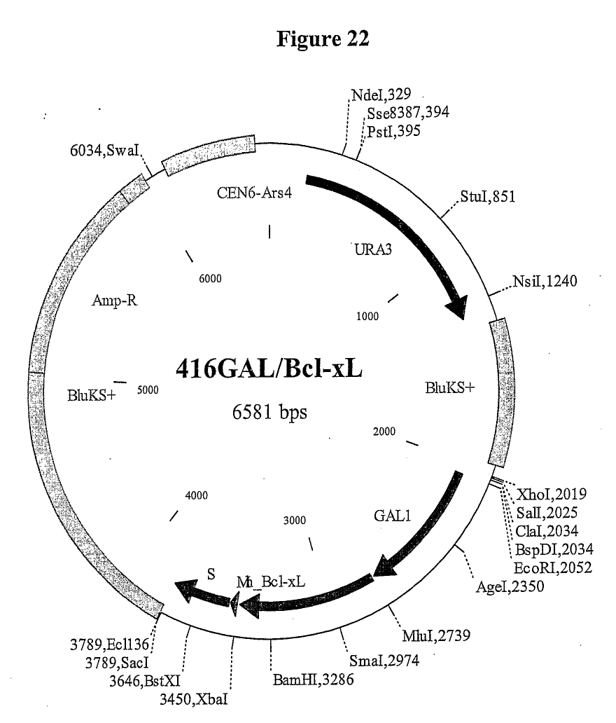

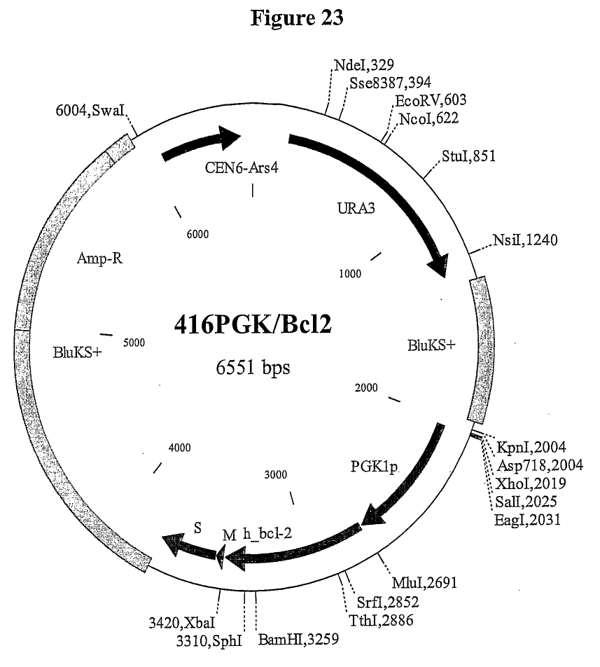

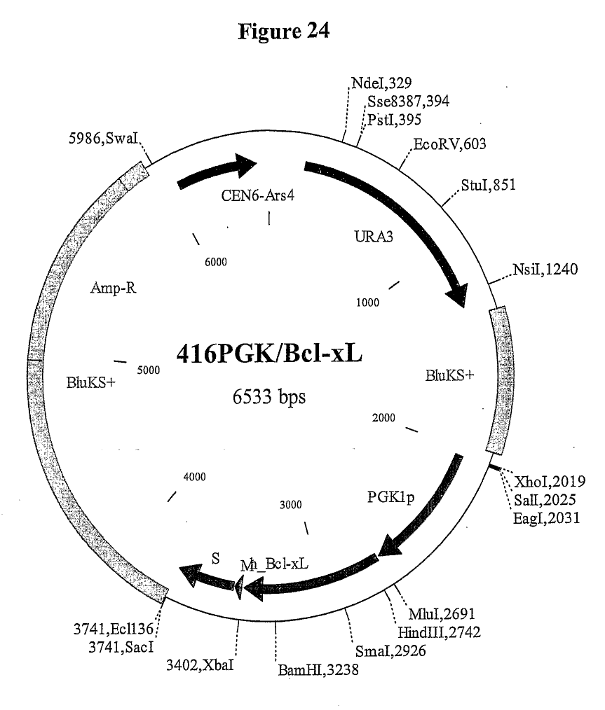

Similarly, BamHI-XbaI-digested fragments encoding human Bcl-2 and human Bcl-xL genes are ligated into BamHI-SpeI-digested pRS416/GAL1pMS or pRS416/PGKpMS to obtain the plasmids 416GAL/Bcl2 (FIG. 21), 416GAL/Bcl-xL (FIG. 22), 416PGK/Bcl2 (FIG. 23), 416PGK/Bcl-xL (FIG. 24).

### EXAMPLE 2

### Optimisation of the Yeast Screening System YAS) for the Selection of Novel Anti-Apoptotic Genes from the Human Hippocampus

### Yeast Growth Conditions

YPD (1% Bacto yeast extract, 2% Bacto peptone and 2% glucose) is a rich medium. Synthetic minimal medium (SC) consists of 0.67% yeast nitrogen base (Difco) and 2% glucose (SD) or 2% galactose (SG). Appropriate growth supplements (adenine, histidine, lysine, leucine, uracil or tryptophan) are added to SD or SG for growth of the specific strains depending on the plasmids carried by the strains. The supplements are kept as stock solutions and are added to the medium in appropriate volumes. Given below are the concentrations of the stock solutions and the volume of stock solutions necessary for preparing a litre of medium. Final concentration for adenine uracil, tryptophan, histidine is 20 mg/l, lysine, tyrosine is 30 mg/l and leucine is 100 mg/l.

### Establishment of Yeast Anti-Apoptotic Screening System

A number of parallel experiments have been performed to select the best yeast strain for the yeast apoptosis screen (YAS).

### Bax Integration Strains

Four yeast Saccharomyces cerevisiae strains are used for integrating the human BAX gene (SEQ ID No: 2), chemically synthesised using yeast-biased codons, at different chromosomal loci of the yeast genome. The yeast strains are:
(1) HT444 (MAT-a, leu2-3, leu2-2, 112 his4-519, ura3, lys2),
(2) W303a (MAT-a ade2-1, trp1-1, leu2-3, leu2-112, his3-11, his3-15, ura3-1),
(3) W303alpha (MAT-alpha, ade2-1, trp1-1, leu2-3, leu2-112, his3-11, h is 3-15, ura3-1) and
(4) JL20 (MAT-a leu2-3, leu2-2, leu2-112, his4-519, ade1-100, ura3-52).

The plasmids used for integration at different chromosomal loci are:

(1) iLEUBAX (FIG. 6)
(2) iADEBAX (FIG. 7)
(3) iTRPBAX (FIG. 8)
(4) iHISBAX (FIG. 9)

The integrations yield single copies of the BAX expression cassette, under the control of the inducible GAL1 promoter, at yeast chromosomal loci where the LEU2, ADE2, TRP1 and HIS3 genes are naturally situated.

The resultant yeast strains obtained after integration are listed in Table 2.

TABLE 2
Yeast Strains bearing a BAX expression cassette or an empty
plasmid in different chromosomal loci of the yeast genome

| | Plasmid used for | Resultant Yeast |
|---|---|---|
| Parent Yeast Strain | Integration | Strain Name |
| HT444 | iLEUBAX (FIG. 6) | HT444baxleu |
| HT444 | yIPLEUG1 (FIG. 2) | HT444leu |
| HT444 | iADEBAX (FIG. 7) | HT444baxade |
| HT444 | yIPADEG1 (FIG. 3) | HT444ade |
| HT444 | iTRPBAX (FIG. 8) | HT444baxtrp |
| HT444 | yIPTRPG1 (FIG. 4) | HT444trp |
| HT444 | iHISBAX (FIG. 9) | HT444baxhis |
| HT444 | yIPHISG1 (FIG. 5) | HT444his |
| W303a | iLEUBAX (FIG. 6) | W303baxleu |
| W303a | yIPLEUG1 (FIG. 2) | W303leu |
| W303a | iADEBAX (FIG. 7) | W303baxade |
| W303a | yIPADEG1 (FIG. 3) | W303ade |
| W303a | iTRPBAX (FIG. 8) | W303baxtrp |
| W303a | yIPTRPG1 (FIG. 4) | W303trp |
| W303a | iHISBAX (FIG. 9) | W303baxhis |
| W303a | yIPHISG1 (FIG. 5) | W303lhis |
| W303alpha | iLEUBAX (FIG. 6) | W303alpbaxleu |
| W303alpha | yIPLEUG1 (FIG. 2) | W303alpleu |
| W303alpha | iADEBAX (FIG. 7) | W303alpbaxade |
| W303alpha | yIPADEG1 (FIG. 3) | W303alpade |
| W303alpha | iTRPBAX (FIG. 8) | W303alpbaxtrp |
| W303alpha | yIPTRPG1 (FIG. 4) | W303alptrp |
| W303alpha | iHISBAX (FIG. 9) | W303alpbaxhis |
| W303alpha | yIPHISG1 (FIG. 5) | W303alphis |
| JL20 | iLEUBAX (FIG. 6) | JL20baxleu |
| JL20 | yIPLEUG1 (FIG. 2) | JL20leu |
| JL20 | iADEBAX (FIG. 7) | JL20baxade |
| JL20 | yIPADEG1 (FIG. 3) | JL20ade |
| JL20 | iTRPBAX (FIG. 8) | JL20baxtrp |
| JL20 | yIPTRPG1 (FIG. 4) | JL20trp |
| JL20 | iHISBAX (FIG. 9) | JL20baxhis |
| JL20 | yIPHISG1 (FIG. 5) | JL20his |

### Screen ing for an Optimal BAX Bearing Strain for Screening

Ideally, all strains should grow in SD (i.e. minimal growth medium containing glucose together with appropriate growth supplements) but should not grow in SG (minimal growth medium containing galactose together with appropriate growth supplements) since galactose will induce Bax protein expression and thereby stop cell growth and kill yeast cells. All strains listed in Table 2 fail to grow as much as control strains (that bear the empty plasmid and no BAX) in galactose under normal growth conditions (72 h at 30° C.) when ˜10⁶ cells are plated out on a 10 cm diameter plate. However, in order to have a stringent selection system for screening potential anti-apoptotic genes from a cDNA library made from any human tissue of choice, one ought to have a strain that does not show any growth when relatively large number of cells are plated (i.e. >10⁸ cells are plated on a 10 cm diameter plate) and incubated for 72 h at 30° C., as would be done during the screening procedure. Unexpectedly, the strain W303baxleu is found to be ideally suited for screening as indicated from the results in Table 3. It shows no growth in galactose even after 7 days of incubating >10⁸ cells at 30° C.

TABLE 3
Selection of a Yeast Strain for YAS

| Yeast Strains | Growth in | | |
|---|---|---|---|
| after | SD, 72 h, | Growth in SG: 10⁶ | Growth in SG: >10⁸ |
| Integration | 30° C. | cells, 72 h, 30° C. | cells, 72 h, 30° C. |
| HT444baxleu | ++++ | − | + |
| HT444leu | ++++ | ++++ | +++++ |
| HT444baxade | ++++ | −/+ | + |
| HT444ade | ++++ | ++++ | +++++ |
| HT444baxtrp | ++++ | −/+ | + |
| HT444trp | ++++ | ++++ | +++++ |
| HT444baxhis | ++++ | −/+ | + |
| HT444his | ++++ | ++++ | +++++ |
| W303baxleu | ++++ | − | − |
| W303leu | ++++ | ++++ | +++++ |
| W303baxade | ++++ | − | −/+ |
| W303ade | ++++ | ++++ | +++++ |
| W303baxtrp | ++++ | −/+ | + |
| W303trp | ++++ | ++++ | +++++ |
| W303baxhis | ++++ | − | + |
| W303lhis | ++++ | ++++ | +++++ |
| W303alpbaxleu | ++++ | − | −/+ |
| W303alpleu | ++++ | ++++ | +++++ |
| W303alpbaxade | ++++ | −/+ | + |
| W303alpade | ++++ | ++++ | +++++ |
| W303alpbaxtrp | ++++ | −/+ | + |
| W303alptrp | ++++ | ++++ | +++++ |
| W303alpbaxhis | ++++ | −/+ | + |
| W303alphis | ++++ | ++++ | +++++ |
| JL20baxleu | ++++ | − | + |
| JL20leu | ++++ | ++++ | +++++ |
| JL20baxade | ++++ | −/+ | + |
| JL20ade | ++++ | ++++ | +++++ |
| JL20baxtrp | ++++ | −/+ | + |
| JL20trp | ++++ | ++++ | +++++ |
| JL20baxhis | ++++ | −/+ | + |
| JL20his | ++++ | ++++ | +++++ |

### Rescue of Bax-Mediated Yeast Cell Killing

Human Bcl-xL and Bcl-2 are known anti-apoptotic proteins. The human Bcl-2 and Bcl-xL genes have been subcloned in centromeric (single-copy) and 2-micron (multi-copy) vectors, under the control of GAL1 (strong inducible) and PGK1 (strong constitutive) promoters plasmids depicted in FIG. 17 to FIG. 24). All plasmids carry the URA3 gene for selection in yeast. The plasmids are transformed in the Bax-bearing strain, W303baxleu and also W303alpha. The latter transformations are performed solely for the purpose of a mating experiment where the BAX gene is harboured on a strain with the Mat-a mating type whereas the rescuing anti-apoptotic genes Bcl-2 and Bcl-xL are borne by another strain of the opposite Mat-alpha mating type.

For transformation of anti-apoptotic gene bearing plasmids directly into a BAX-bearing yeast strain, 5×10⁷ cells of the W303baxleu strain is harvested and the cell pellet is washed to remove the YPD medium used for the overnight culture. 100 μg denatured salmon sperm DNA (Sigma) and 0.5 μg plasmid DNA (bearing the anti-apoptotic genes Bcl-2 or Bcl-xL; FIGS. 17 to 24) are added to the pelleted cells followed by 500%1 of PEG (polyethylene glycol)-3500 solution (40% PEG; 0.1M lithium acetate pH 7.5; 10 mM Tris-HCl pH 7.5; 1 mM EDTA pH 7.5) and DMSO is added to a final concentration of 5% (v/v). After the incubation on a Thermo-mixer (Eppendorf) for 15 min, 400 rpm at 25° C., cells are heat shocked for 15 min at 42° C. in 10% ethanol (final concentration). The cells are resuspended in 500 μl of TE pH7.5 after washing twice with the same volume of TE. 200 μl of resuspended cells are spread directly onto SD and SG plates carrying the necessary amino acid and nucleoside supplements. The plates are incubated at 30° C. for 3 days (SD) and around 20 days (SG).

The plasmids bearing human Bcl-2 and Bcl-xL genes (FIGS. 17 to 24) are transformed into the W303alpha (Mat-alpha) yeast strain so that transformants can be mated with the W303baxleu cells and thereby their rescuing ability of BAX after mating can be gauged. The transformants of Bcl-2 and Bcl-xL bearing plasmids (URA3⁺) are initially grown in SD medium that contained leucine, histidine, adenine and tryptophan for 3 days at 30° C. A tiny loopful of cells from 6 individual colonies from each transformation is mixed in 100 μl TE. Similar number of cells from W303baxleu (Mat-a, BAX⁺) are added to the Mat-alpha cell mix (Bcl-2⁺ or Bcl-xL⁺) and are mixed thoroughly. The mixture is plated onto a YPD plate, incubated for 24 h at 30° C., and then selected on SD plates that contain only histidine, adenine and tryptophan.

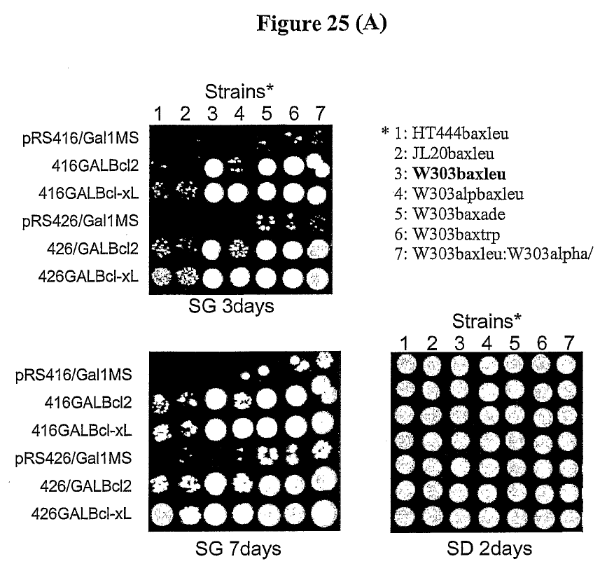

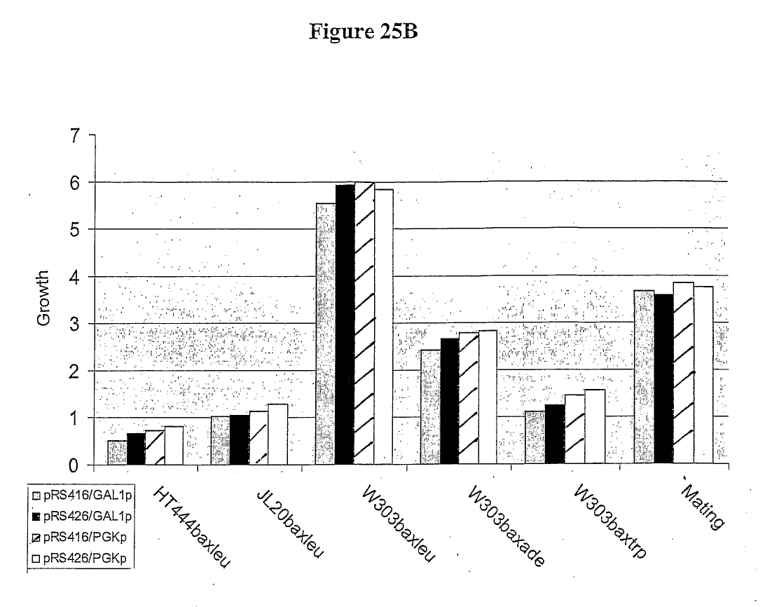

All transformants (directly or after mating) are tested for their ability to abrogate Bax-mediated inhibition of cell growth. The strains are compared with regard to (a) their rescue efficiency and (b) growth speed. The results are depicted in Table 4 and FIG. 25.

TABLE 4
Bax strains used to determine the best possible combination
for screening of cDNA libraries derived from any human
tissue, cell or a genome from any organism

| | | Supplements |
|---|---|---|
| | | added |
| Name | Description | to SD/SG* |
| HT444baxleu | HT444::pRS305/GAL1p-Bax (LEU2) | HKU |
| W303baxleu | W303a:: pRS305/GAL1p-Bax (LEU2) | HAWU |
| W303alpbaxleu | W303αlphα:: pRS305/GAL1p-Bax | HAWU |
| | (LEU2) | |
| JL20baxleu | JL20:: pRS305/GAL1p-Bax (LEU2) | HAU |
| W303baxade | W303a::pRS402/GAL1p-Bax (ADE2) | LHWU |
| W303baxtrp | W303a::pRS404/GAL1p-Bax (TRP1) | LHAU |
| W303alpha | W303αlphα | LHAWU |

*A = adenine, H = histidine, K = lysine, L = leucine, U = uracil, W = tryptophan

### EXAMPLE 3

### Screening of Anti-Apoptotic Genes in Human Hippocampus

Establishing and Amplifying the Human Hippocampal cDNA Library

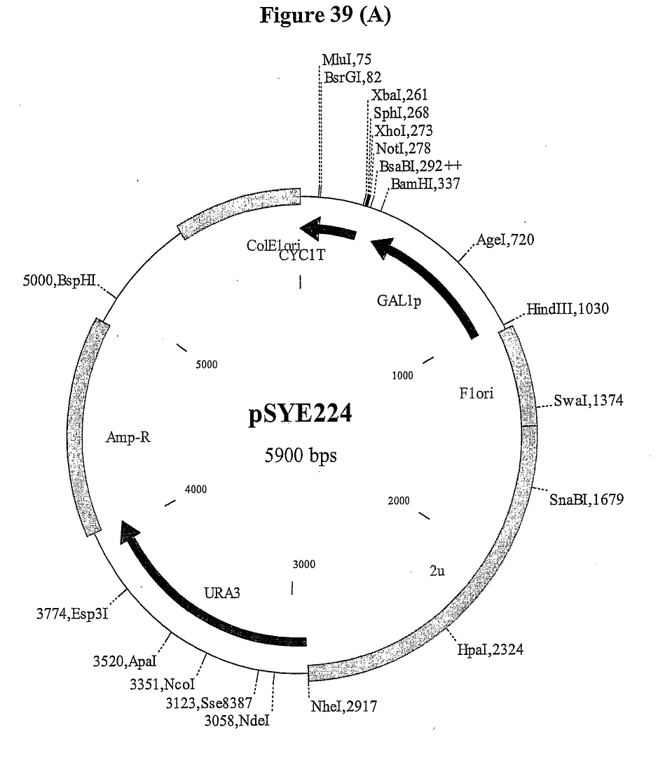

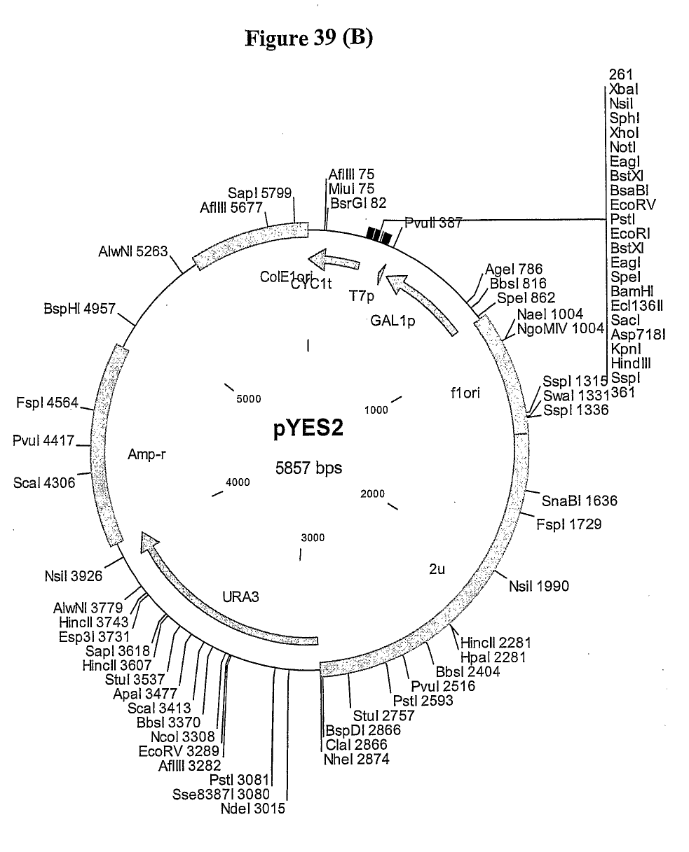

Human hippocampus cDNA Library was custom synthesised by BioCat GmbH (Heidelberg, Germany). The cDNA library is made from whole human adult normal brain hippocampus mRNA. First-strand cDNA synthesis is performed using an oligo(dT)-NotI primer and M-MLV-RNase H-reverse transcriptase. The second strand synthesis is carried out with a random linker primer and Klenow Exo-DNA polymerase. For directional cloning, the blunt-ended cDNA is first digested with Not I and then size-fractionated on a 1.3% agarose gel. Following elution of cDNAs greater than 0.6 kb the cDNA was ligated into the NotI and BsaBI digested yeast expression vector pSYE224 (FIG. 39). The plasmid cDNA library is obtained as glycerol stock. In order to maintain all the genetic information of this library, a method that uses semi-solid amplification is employed. 1000 ml of autoclaved 2×LB agarose (0.27% SeaPrep agarose (FMC), 2% Tryptone, 1% Yeast Extract, 1% NaCl in double-distilled H₂O) is cooled in a 37° C. water bath for 2 h. 1.22×10⁶ primary cDNA transformants (that represents the glycerol stock solution) with 0.2 g ampicillin is added and mixed thoroughly on a stirrer plate for 2 min. After incubation on ice for 1 h, the culture bottle is gently moved into a gravity flow incubator set at 30° C. and incubated for 45 h without disturbance. The cells are then harvested to prepare purified DNA using the QIAGEN plasmid Maxi-prep kit. Glycerol stock solutions are kept before DNA purification.

High Efficiency Transformation of the Hippocampal cDNA Library and Screening for Bax-Resistant Transformants

A culture of the yeast strain W303baxleu in SD (containing histidine, adenine, uracil and trytophan) liquid medium at mid-logarithmic phase is harvested and washed with 100 mM lithium acetate solution. To a pellet that contains 1×10⁹ cells, the transformation mix is added. The transformation mix contains 240 μl PEG-3500 (50% w/v, Sigma), 36 μl 1.0 M lithium acetate, 50 μl single-stranded DNA (freshly boiled, 2.0 mg/ml, Sigma), 33 μl sterile double-distilled H₂O and 1 μl (1 μg/μl) cDNA library DNA. The mixture is thoroughly mixed before use. The cell suspension is incubated with shaking at 30° C. for 30 min. The transformants are harvested and are selected directly on galactose-containing synthetic dropout medium that lacks leucine and uracil but contains histidine, adenine and trytophan (i.e. SG+HAW). An aliquot of the transformation mixture is also spread on a SD+HAW plate to determine transformation efficiency. The transformants on this SD+HAW plate are later screened on a SG+HAW by replica plating using sterile velvet cloth cut into square pieces. The transformation is repeated several times until individual transformants are obtained that are greater than 3 times the number of representative clones present in the original cDNA library. In this way, one can be sure that one has screened in the yeast apoptosis assay (YAS) all cDNAs present in the cDNA library. The Bax-resistant colonies from SG+HAW plates are collected individually.

Plasmid DNA Isolation from Bax-Resistant Transformants

Bax-resistant colonies from SG+HAW plates are picked and grown in 1 ml YPD liquid medium overnight at 30° C. The cells are harvested by centrifugation in a microfuge and are resuspended in 500 μl of 1 M sorbitol, 0.1 M Na₂EDTA (pH 7.5). After incubation with 20 μl of 2.5 mg/ml Zymolyase 100T (Fisher Scientific) for 1 h at 37° C., the cells are centrifuged and resuspended in 500 μl of 50 mM Tris-Cl (pH 7.5), 20 mM Na₂EDTA with 50 μl of 10% SDS. The mixture is incubated for 30 min at 65° C. with shaking at 300 rpm. After addition of 200 μl of 5 M potassium acetate, the cells are further incubation on ice for 1 h. The cells are then centrifuged at 13,000 rpm for 5 min and the supernatant is precipitated in one volume of 100% isopropanol. The air-dried pellet is resuspended in 300 μl of TE (pH 7.5) and incubated with 75 μg of RNase followed by addition of 30 μl of 3 M sodium acetate and 200 μl of 100% isopropanol. The pelleted DNA is resuspended in 30 μl of TE (pH 7.5) and transformed into competent DH5a bacterial cells. Thus the plasmid DNA from all yeast colonies, that bear the Bax-rescuing gene, is amplified via bacterial cells and purified using QIAGEN plasmid Mini-prep kit. These plasmids, in theory, should be bearing novel Bax-antagonists (i.e. novel anti-apoptotic genes) that are present in the human hippocampus.

Elimination of False-Positives from the Bax Rescue Screen

All plasmids isolated from Bax-resistant yeast cells are transformed back into W303baxleu as described in Example 2 above and are plated on SD+HAW plates. Transformants are tested for their genuine Bax-rescuing ability by plating 100 cells from 10 individual colonies from each transformation onto SD+HAW and SG+HAW plates. The plates are incubated at 30° C. for 2-5 days. The plasmid transformed cells which grow on both SD and SG plates are considered spot-test positive and therefore bear a plasmid that genuinely rescues Bax-mediated cell growth inhibition. Spot-test negative plasmids are treated as the false-positives. These plasmids bear genes that do not rescue Bax and are therefore eliminated from the experiment.

Yeast cells that survive on SG plates are checked for the presence of the Bax gene to be absolutely sure that the spot-test positive plasmids indeed do contain anti-apoptotic genes. PCR is performed in a 100 μl reaction volume containing 100 pmol of each primer, 0.8 mM dNTPs, 2 mM MgCl₂, and 2.5 U Taq Polymerase (ABG). After an initial denaturation period of 7 min at 99° C., 30 cycles are performed, consisting of denaturation at 95° C. for 1 min, annealing at 60° C. for 1 min 30 sec and extension at 72° C. for 3 min. 25 μl of the reactions are loaded on a 1% TAE agarose gel and visualized by ethidium bromide staining. The primers used to identify Bax are:

(SEQ ID No: 24)

| | |
|---|---|
| 5′primer: | 5′-GGAAGATCTATGGACGGTTCCGGT GAACAA-3′. |

(SEQ ID No: 25)

| | |
|---|---|
| 3′primer: | 5′-CTAGTCTAGAACCCATCTTCTTCCAGATGGTC-3′. |

### Sequencing the Anti-Apoptotic Genes

23 plasmids, that demonstrate Bax-rescuing function, are subjected to DNA sequencing reactions using ABI's BigDye terminator sequencing chemistry (PNACL DNA sequencing service, Leicester University). The primers used for sequencing the inserts, identified in pSYE224 (FIG. 39) clones, are

| | |
|---|---|
| 5′primer: | |
| 5′-CCTCTATACTTTAACGTCAAGGAG-3′. | (SEQ ID No: 26) |
| 3′primer: | |
| 5′-CGTGAATGTAAGCGTGACATAAC-3′. | (SEQ ID No: 27) |

The homology of the DNA sequence is determined using BLAST, a program supported by the National Center for Biotechnology Information (NCBI) at http://www.ncbi.nlm.nih.gov/BLAST/

### Results

In the YAS screen that identifies Bax-resistant colonies, 106 colonies out of 3.808×10⁶ transformants survive on SG plates. After re-testing to identify false positives, 23 plasmids (21.70%) remained that still demonstrate Bax-rescuing function. The genes are listed above in Table 1.

HA-tagged DNA sequences of the proteins #1 to #5 have been used further for expression in yeast and mammalian cells.

Bcl-2A1 is a known Bax antagonist. We are not aware of any report that any of the other genes listed in Table 1 have an anti-apoptotic function.

### EXAMPLE 4

### Expression Constructs for the Novel Anti-Apoptotic Genes Identified from the Human Hippocampal cDNA Library

### The Basic Plasmid Containing HA-tag

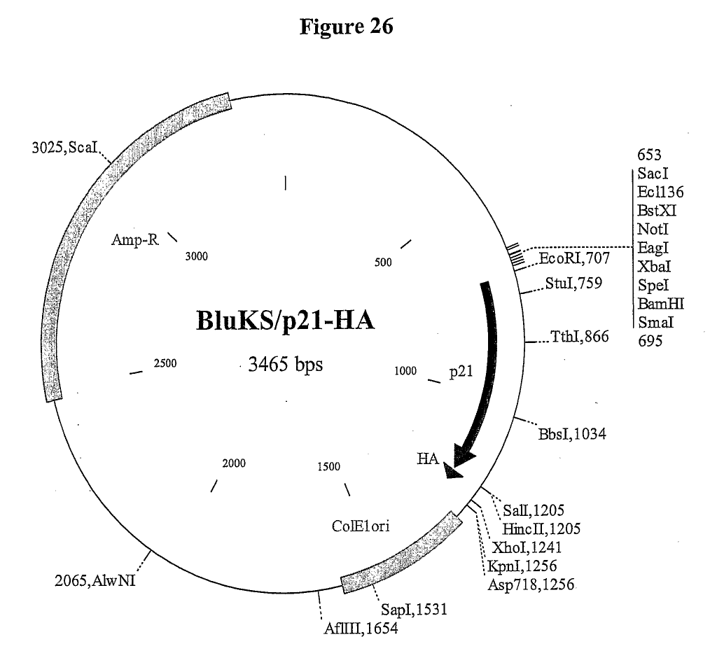

The basic plasmid BluKS/p21-HA (FIG. 26) is made by subcloning an EcoRI-XhoI fragment that contains p21^{wAF1/CIP1} gene (henceforth, p21) and the DNA sequence that codes for HA-tag in XhoI-EcoRI-digested pBlueScriptKS(+) vector (Stratagene). The p21^{wAF1/CIP1} gene is linked to the HA-tag sequence by the SalI restriction site. The HA-tag sequence is followed by a Stop codon.

The above plasmid BluKS/p21-HA is digested with SpeI-SalI or BamHI-SalI to subclone SpeI-SalI or BamHI-SalI anti-apoptotic gene fragments of interest (i.e. novel genes or the known gene Bcl-xL which is used as a control) that would allow them to be tagged with the HA DNA sequence at the 3′-end. The gene fragments do not contain a 3′-Stop codon. The Stop codon is at the 3′-end of the HA sequence.

### Mammalian/Yeast Expression Constructs

Full-length SpeI-YhoI fragments of human Bcl-2A1, FKBP2, SNCA (alpha-synuclein), VAMP3, and EEF1A1 are amplified by PCR from the same human hippocampus cDNA Library from which they were originally identified as anti-apoptotic gene sequences. The amplified fragments are cloned into the SpeI-SalI-digested BluKS/p21-HA vector, which is devoid of p21. The sense primers, used for PCR, contain 6 codons of complementarity to the 5′ end of the coding sequence, a SpeI restriction site and a consensus translational start sequence (i.e. the Kozak sequence, GCCACC). The antisense primers, used for PCR, contain 6 codons complementary to the 3′ end of the coding sequence and an XhoI restriction site. All primers were synthesised by Invitrogen (Paisley, UK) and are listed in Table 5.

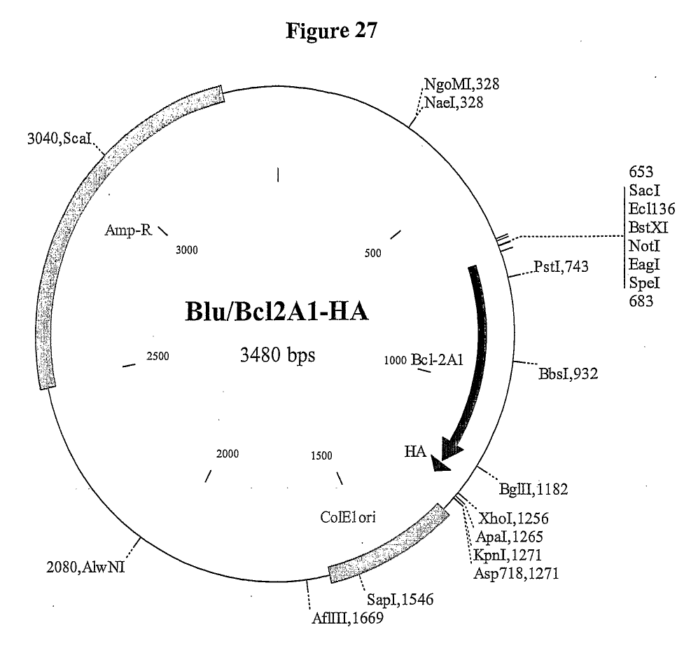

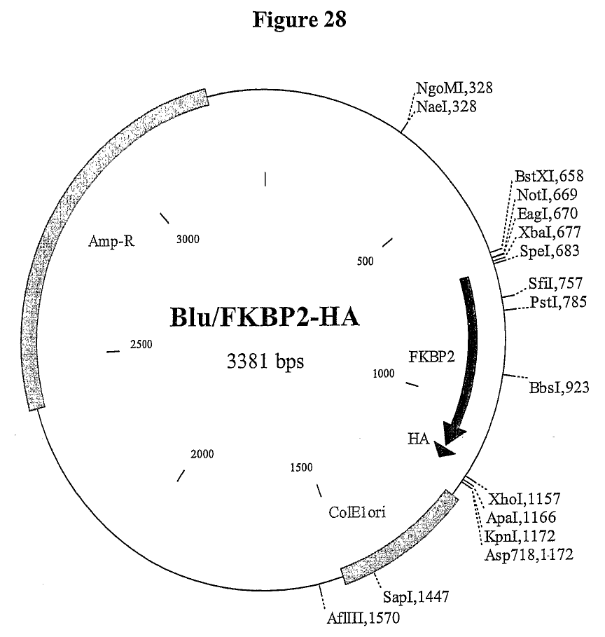

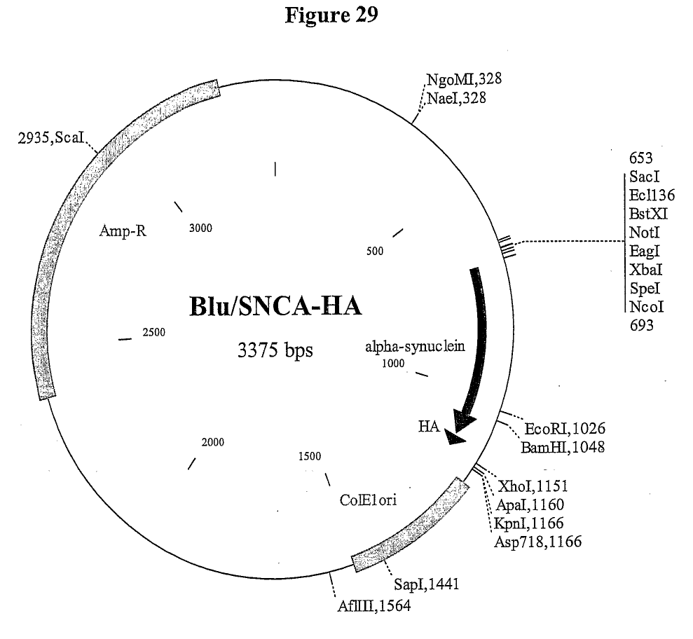

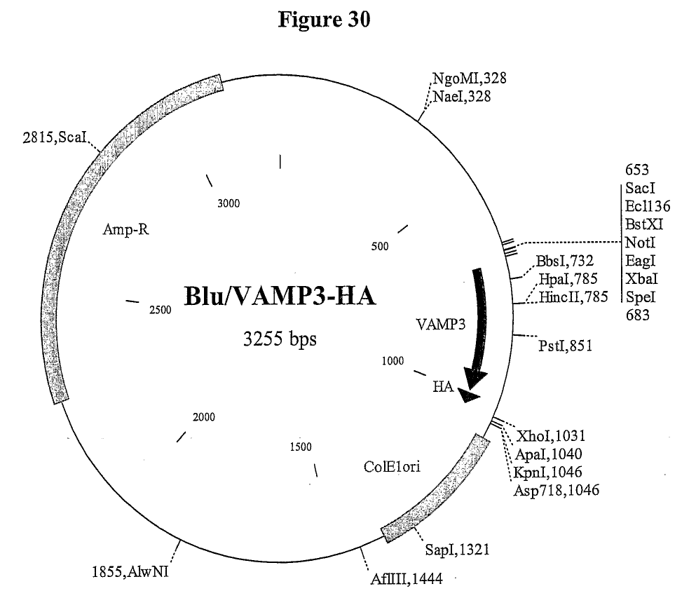

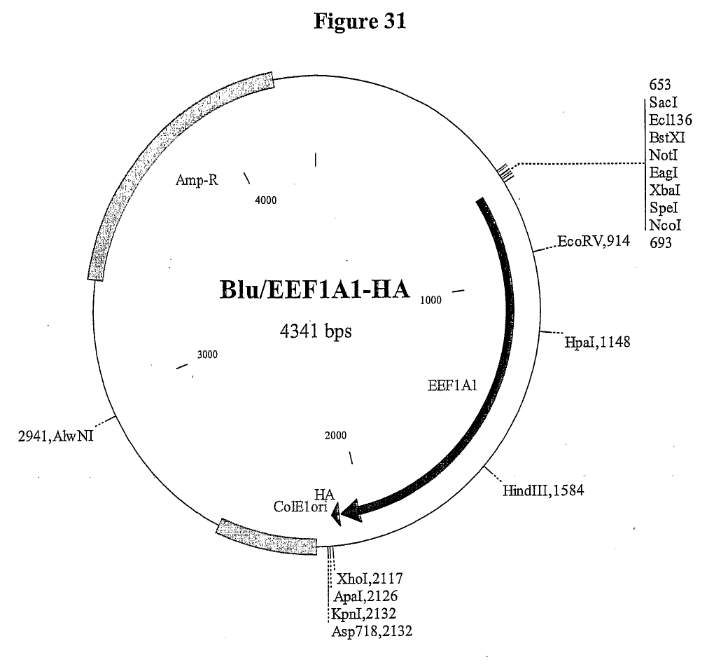

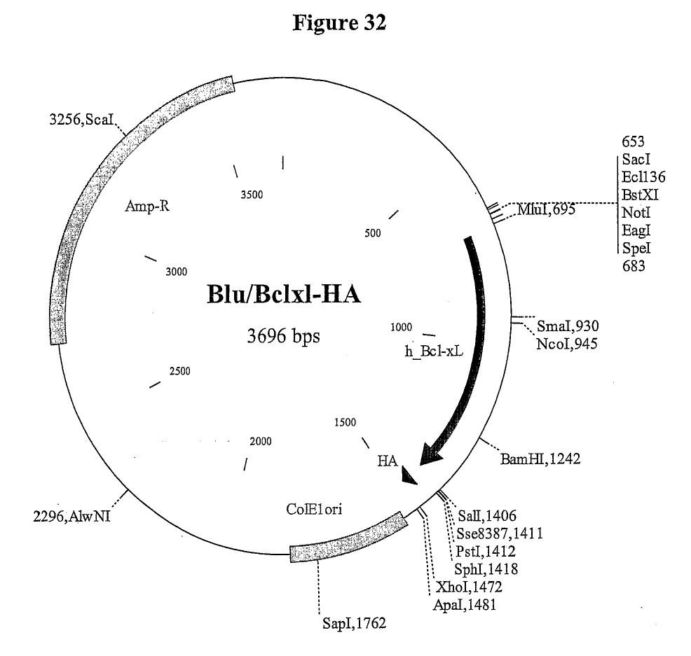

Following restriction enzyme digestion, the SpeI-XhoI PCR fragments are cloned into the BluKS/p21-HA vector which is first digested with SpeI and SalI and the vector devoid of p21 is then isolated. The resultant plasmids obtained are named Blu/Bcl2A1-HA (FIG. 27), Blu/FKBP2-HA (FIG. 28), Blu/SNCA-HA (FIG. 29), Blu/VAMP3-HA (FIG. 30) and Blu/EEF1A1-HA (FIG. 31). The BglII-XhoI digested insert encoding full-length human Bcl-xL is isolated from the construct pSP73/BglII-XbaI/Bcl-xL and ligated to BamHI-SalI-digested BluKS/p21-HA vector fragment to obtain the plasmid Blu/BclxL-HA (FIG. 32).

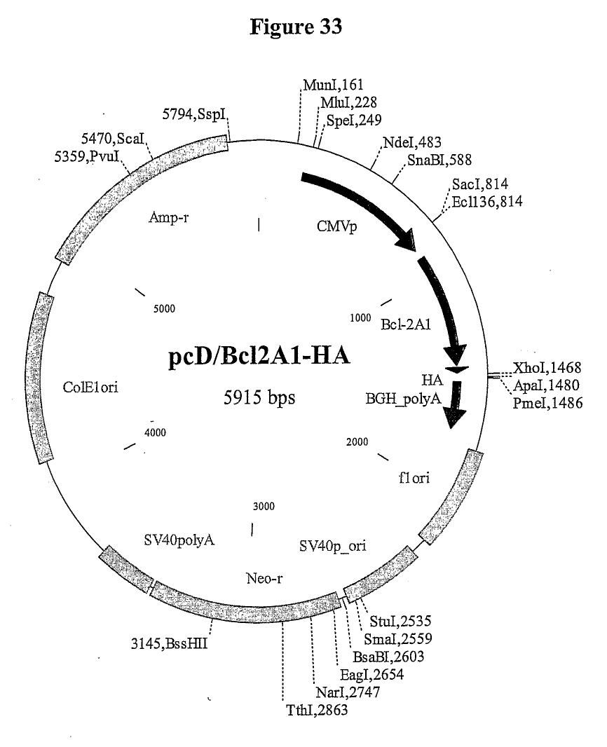

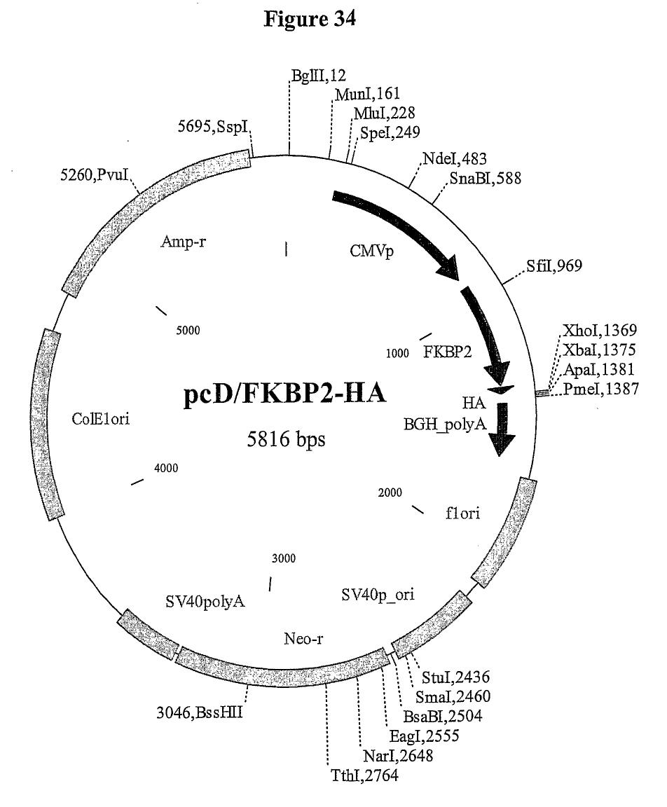

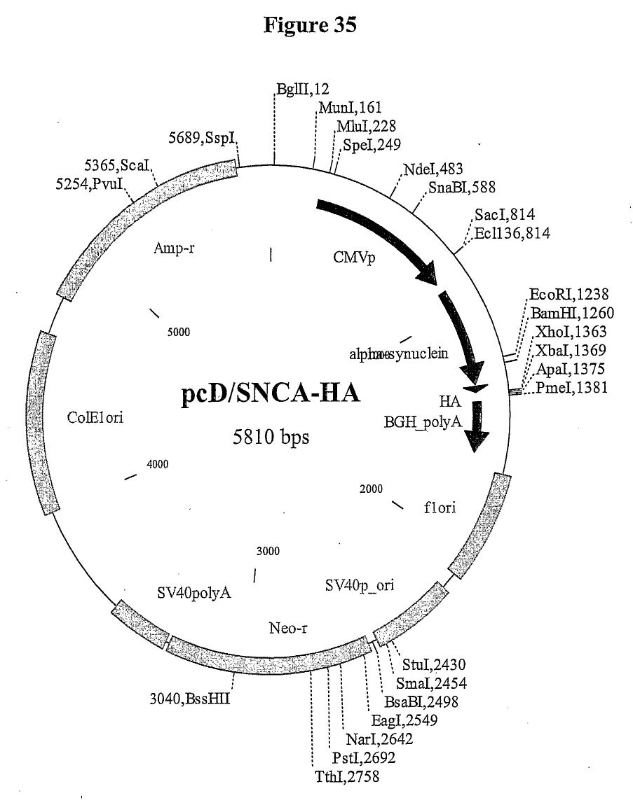

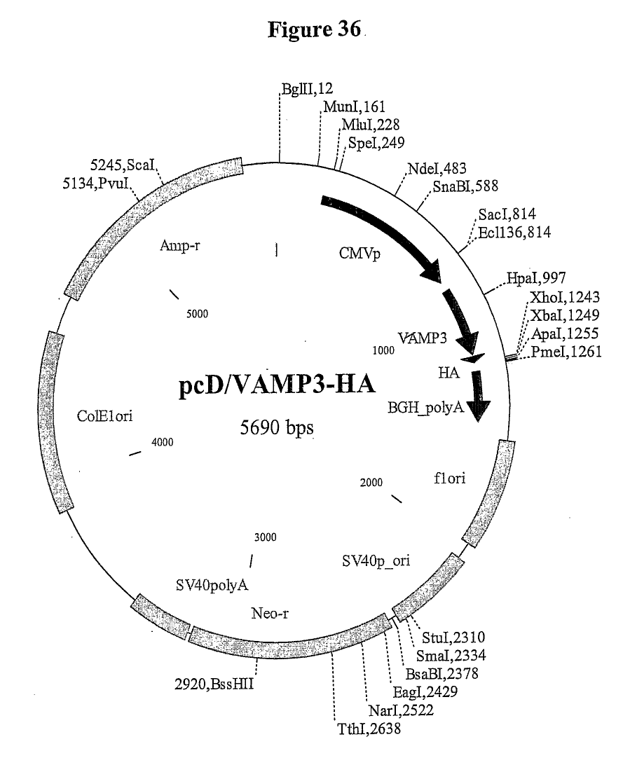

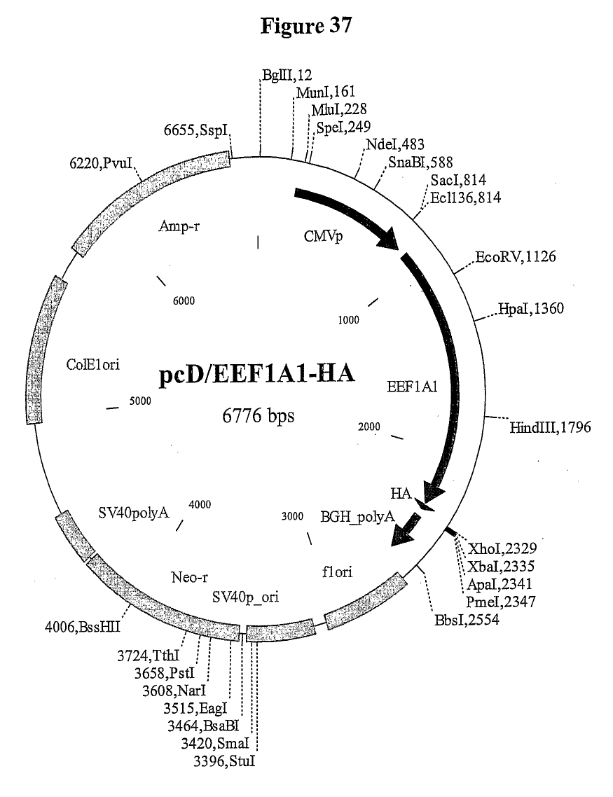

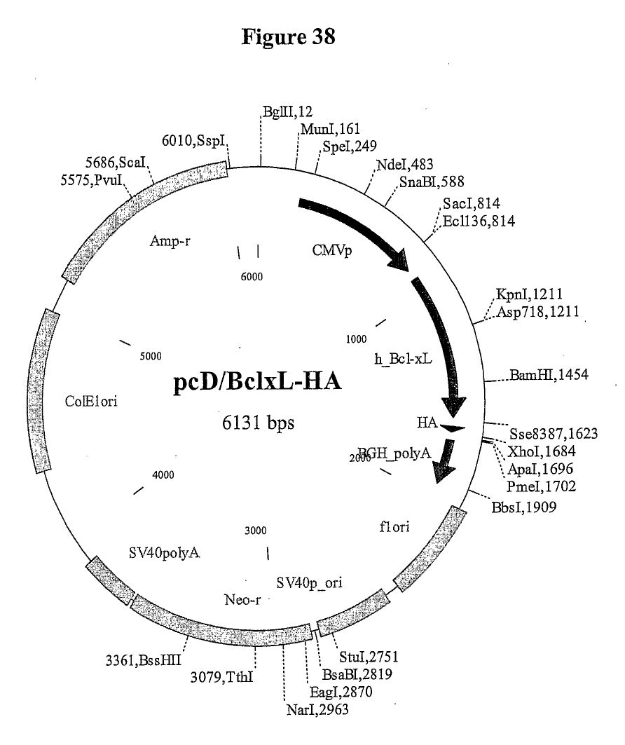

The above pBlueScript-derived plasmids (FIGS. 27 to 32) are digested with SpeI-XhoI and HA-tagged full-length Bcl-2A1, FKBP2, SNCA, VAMP3, EEF1A1 and Bcl-xL gene fragments are isolated. They are subcloned into the NheI-XhoI digested mammalian cell expression vector pcDNA3.1 (+) (Invitrogen, Paisley, UK). The resultant plasmids are named pcD/Bcl2A1-HA (FIG. 33), pcD/FKBP2-HA (FIG. 34), pcD/SNCA-HA (FIG. 35), pcD/VAMP3-HA (FIG. 36), pcD/EEF1A1-HA (FIG. 37) and pcD/BclxL-HA (FIG. 38).

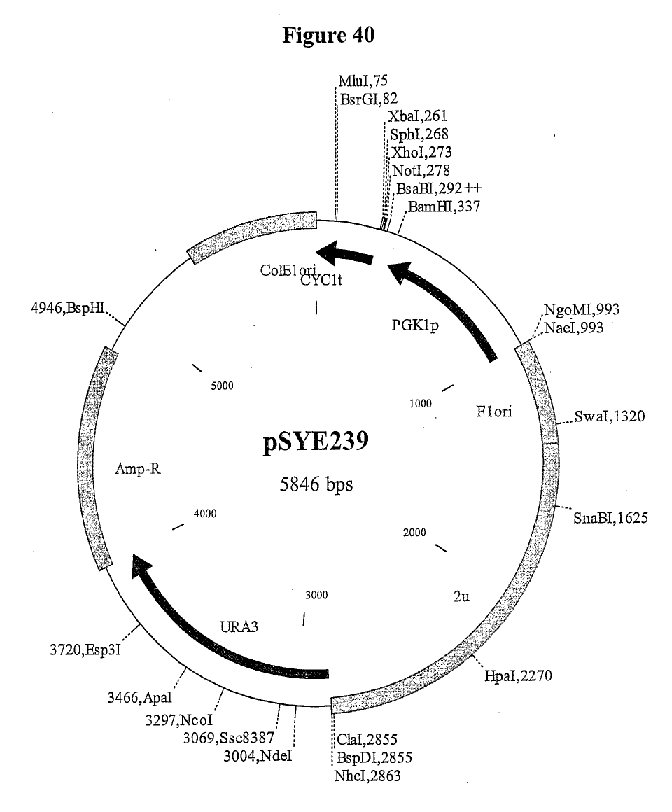

The pBlueScript-derived plasmids (FIGS. 27 to 32) are digested with SpeI-XhoI and HA-tagged full-length Bcl-2A1, FKBP2, SNCA, VAMP3, EEF1A1 and Bcl-xL gene fragments are isolated. They are also subcloned into the SpeI-XhoI digested 2-micron, multi-copy yeast expression vectors pSYE224 (FIG. 39), pSYE239 (FIG. 40). The plasmids pSYE224 and pSYE239 contain a GAL1 promoter (GAL1p) and a PGK1 promoter (PGK1p), respectively. Both plasmids contain the URA3 gene as an auxotrophic selection marker.

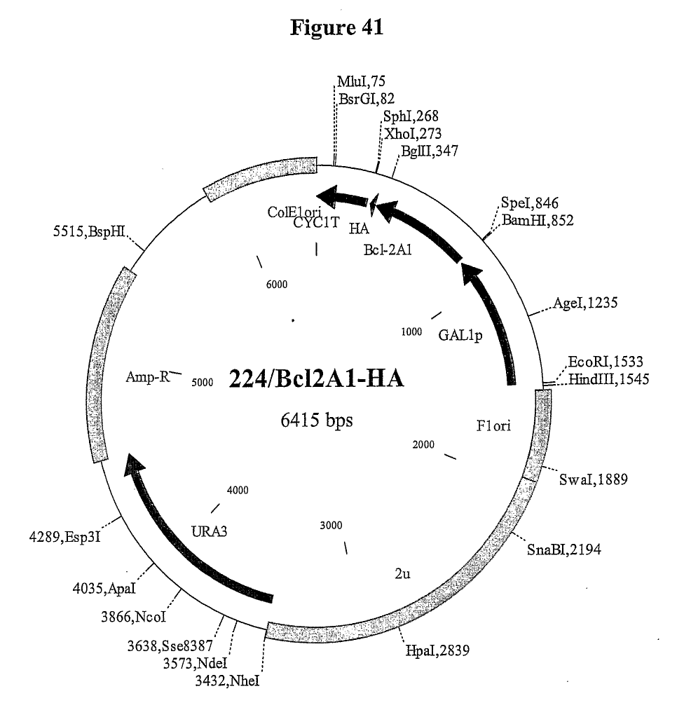

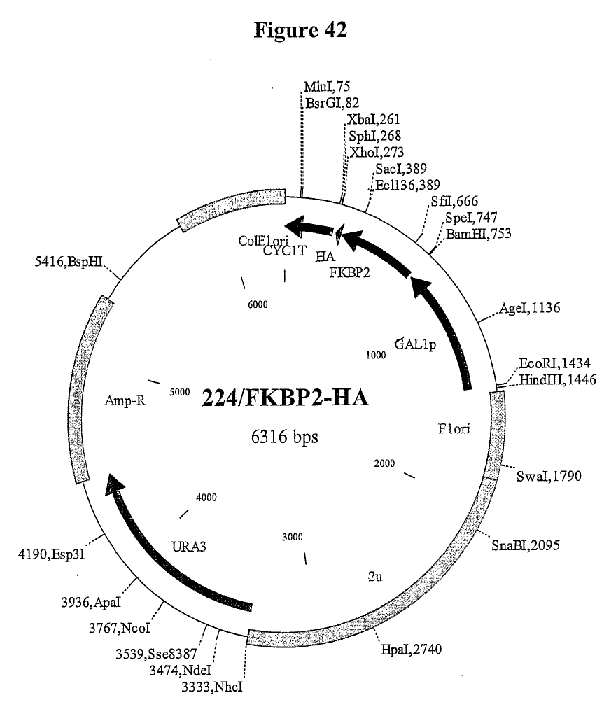

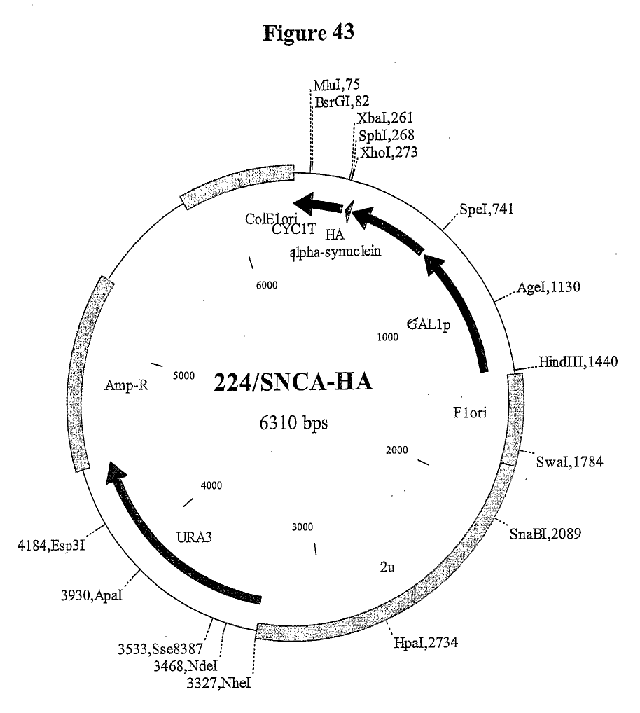

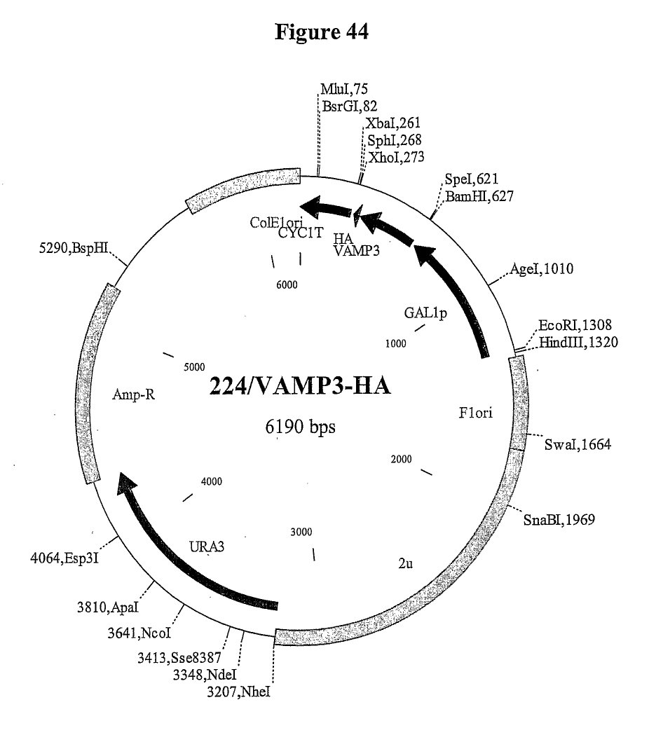

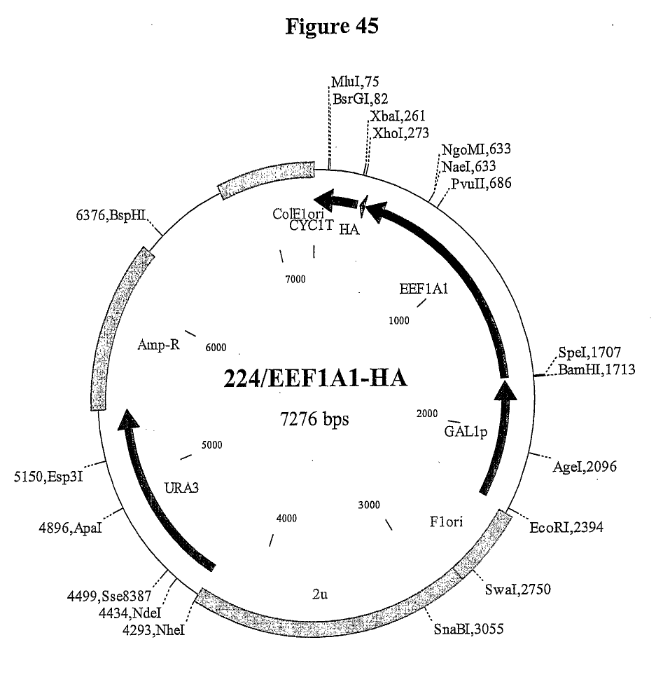

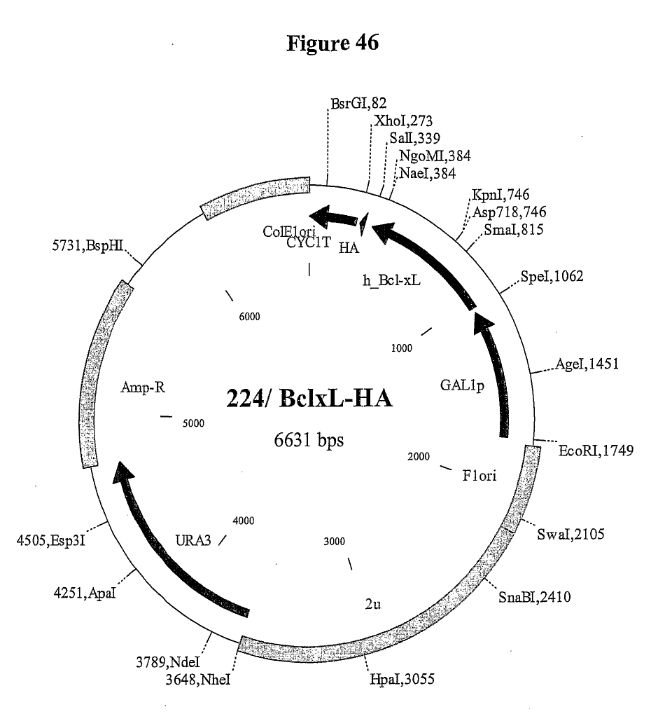

The plasmids derived from pSYE224 (FIG. 39A), bearing the HA-tagged anti-apoptotic genes under the control of the GAL1 promoter, are named as 224/Bcl2A1-HA (FIG. 41), 224/FKBP2-HA (FIG. 42), 224/SNCA-HA (FIG. 43), 224/VAMP3-HA (FIG. 44), 224/EEF1A1-HA (FIG. 45) and 224/BclxL-HA (FIG. 46).

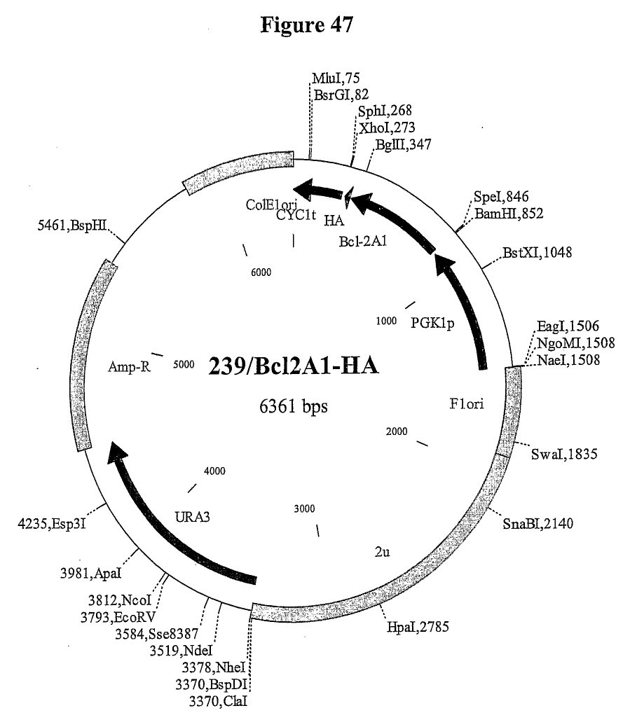

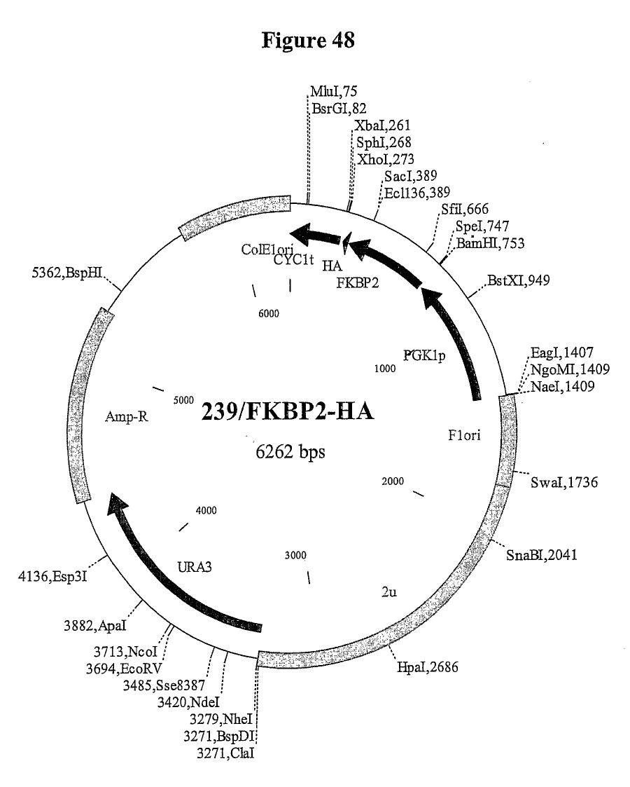

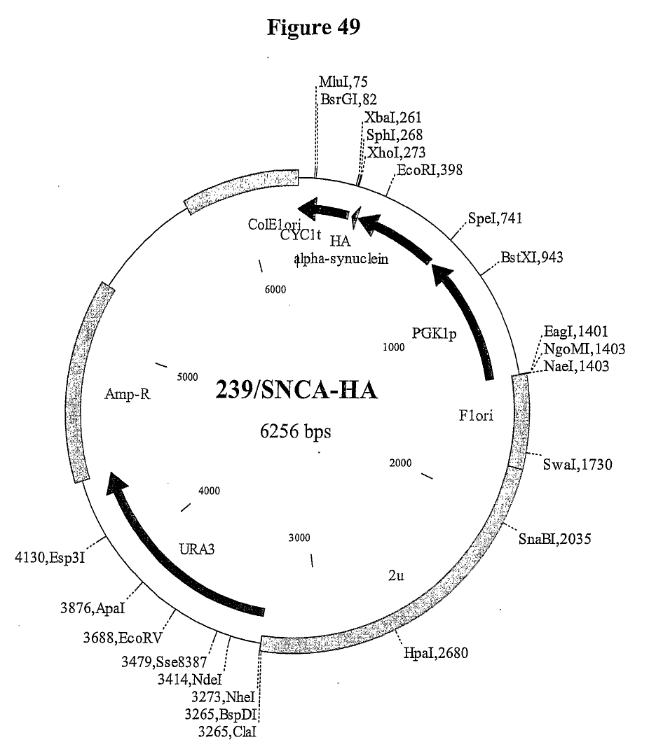

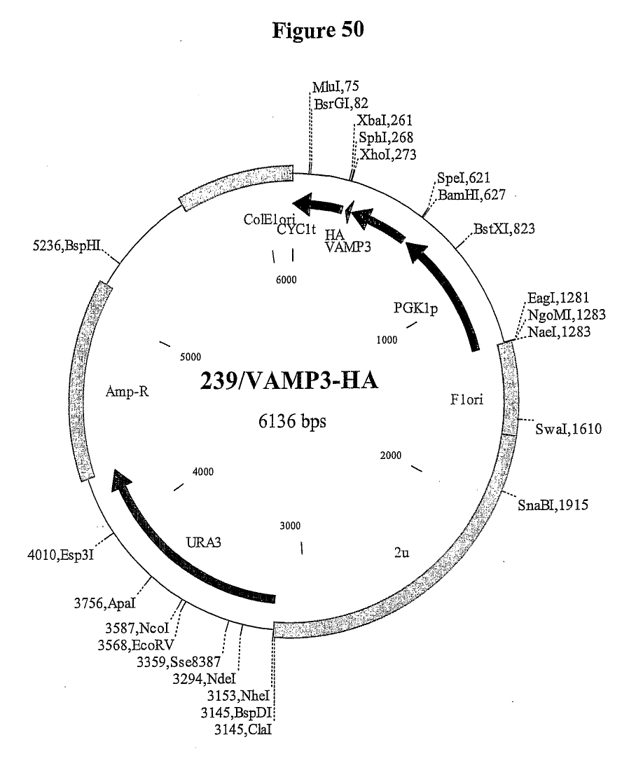

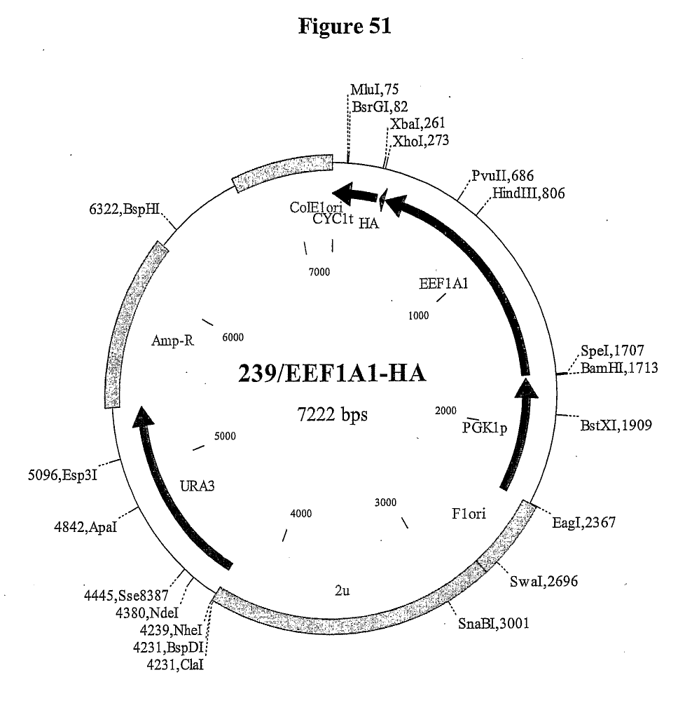

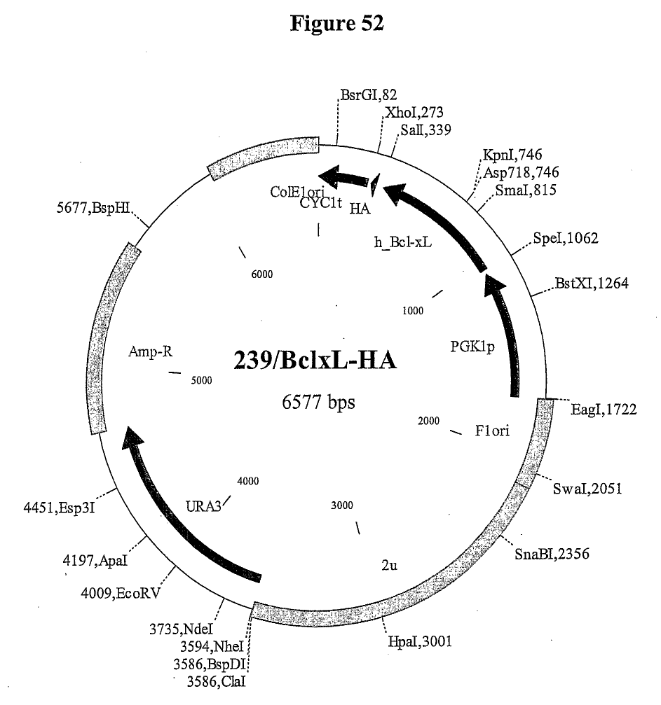

The plasmids derived from pSYE239 (FIG. 40), bearing the HA-tagged anti-apoptotic genes under the control of the PGK1 promoter, are named as 239/Bcl2A1-HA (FIG. 47), 239/FKBP2-HA (FIG. 48), 239/SNCA-HA (FIG. 49), 239/VAMP3-HA (FIG. 50), 239/EEF1A1-HA (FIG. 51) and 239/BclxL-HA (FIG. 52).

It is appreciated that the Invitrogen vector pYES2 (FIG. 39B) can be employed in place of pSYE224. We have used the SpeI-NotI sites for uni-directional cloning of our cDNA library into pSYE224. NotI is an 8-bp restriction site (with a 5′-overhang) and a rare cutter. We intend to introduce the 8-bp PacI restriction site (with a 3′-overhang), which is also a rare cutter like NotI, into pYES2 for uni-directional cloning purposes. Thereafter, the PacI site would be used for uni-directional cloning instead of the 6-bp restriction site SpeI.

TABLE 5

Primers used for cloning the five novel anti-apoptotic genes
obtained from screening the human hippocampal cDNA library
(SEQ ID Nos: 28-37, respectively).

| | |
|---|---|
| Name | Sequence |

| | | |
|---|---|---|
| YAN001 | 5′-Bc12A1 | 5′-GGACTAGTGCCACCATGACAGACTGTGAATTTGG-3′ |
| YAN002 | 3′-Bc12A1 | 5′-CCGCTCGAGACAGTATTGCTTCAGGAGAG-3′ |
| YAN003 | 5′-FKBP2 | 5′-GGACTAGTGCCACCATGAGGCTGAGCTGGTTCCGGGTCC-3′ |
| YAN004 | 3′-FKBP2 | 5′-CCGCTCGAGCAGCTCAGTTCGTCGCTCTATTTTGAGC-3′ |
| YAN005 | 5′-a-synuclein | 5′-GGACTAGTGCCACCATGGATGTATTCATGAAAGGACTTTC-3′ |
| YAN006 | 3′-a-synuclein | 5′-CCGCTCGAGGGCTTCAGGTTCGTAGTCTTGATACCC-3′ |
| YAN007 | 5′-VAMP3 | 5′-GGACTAGTGCCACCATGTCTACAGGTCCAACTGCTGCCAC-3′ |
| YAN008 | 3′-VAMP3 | 5′-CCGCTCGAGTGAAGAGACAACCCACACGATGATG-3′ |
| YAN009 | 5′-ETEF1A1 | 5′-GGACTAGTGCCACCATGGGAAAGGAAAAGACTCATATCAA |
| | | CATTG-3′ |
| YAN010 | 3′-ETEF1A1 | 5′-CCGCTCGAGTTTAGCCTTCTGAGCTTTCTGGGCAG-3′ |

### EXAMPLE 5

### The Anti-Apoptotic Function of Selected Genes in Yeast

Full-length human Bcl-2A1, FKBP2, SNCA, VAMP3, and EEF1A1 are amplified by PCR from the same human hippocampus cDNA Library to construct the plasmids represented in FIG. 41 to FIG. 45 and FIG. 47 to FIG. 51 which contain all the genes as a HA-Tag either under the control of the GAL1 promoter (FIG. 41 to FIG. 45) or under the control of the PGK1 promoter (FIG. 47 to FIG. 51). These plasmids are transformed into W303baxleu as described in Example 2 above to analyse their anti-apoptotic function in yeast.

### Bax-Rescuing Ability of the Five Novel Genes

### Spot Tests

Small scoopful of cells from individual colonies (i.e. transformants), that harbour the novel genes, are resuspended in 200 μl water and 5 μl aliquots of these transformants are spotted onto SD and SG plates that contain the appropriate supplements.

### Increased Colony-Forming Ability Against Bax by Novel Genes

Novel Anti-Apoptotic Genes Allow Survival of Yeast from Bax-Mediated Death.

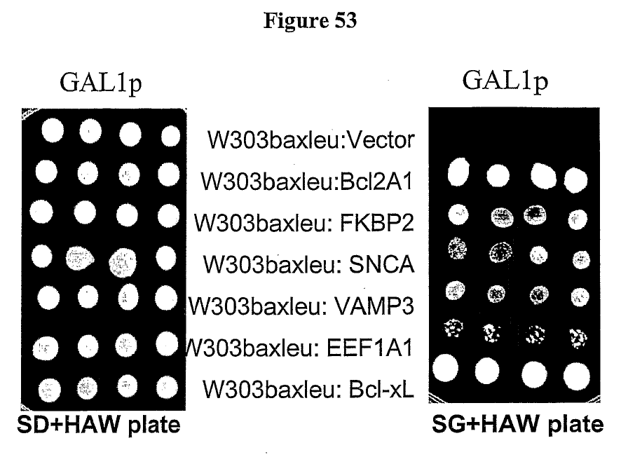

The full-length genes are amplified from the human hippocampal cDNA library and inserted downstream of the GAL1 and PGK1 promoters in yeast expression plasmids pSYE224 (FIG. 39) and pSY239 (FIG. 40). All genes have an HA-tag at their 3′-ends. Representative results for genes driven by the GAL1 promoter are shown (i.e. pSYE224 derivatives; FIG. 41 to FIG. 46) in FIG. 53. Similar results are obtained with pSYE239 derivatives (i.e. FIGS. 47 to 52).

### EXAMPLE 6

### The Anti-Apoptotic Function of Selected Genes in Mammalian Cells

### Mammalian Cell Culture Assay

### Cells

Human embryonic kidney cells (HEK293, ATCC CRL-1573, LGC Promochem, Middlesex, UK) are grown in Dulbecco's modified Eagle's medium (DMEM; Gibco BRL, Grand Island, N.Y., USA) supplemented with 10% Fetal Bovine Serum (FBS; Bio Whittaker, Walkersville, Md., USA) and 2 mM L-glutamine in a humified atmosphere of 5% CO₂ at 37° C. in 25 or 75 cm² tissue culture flasks (Corning, N.Y. 14831, USA). For the analysis of stable expression of protein, cells were cultured in the same medium containing G418 (GIBCO BRL) at a concentration of 1 mg/ml to select for transfected cells. For immunocytochemical analysis, the cells are grown on the cover-slips for 24-48 h.

### Trypsination

Cells are detached for subculture or Nucleofector-mediated transfection (Amaxa, Cologne, Germany) at 70-80% confluency with 0.05% trypsin, 0.04% EDTA in phosphate buffered saline (PBS) at 37° C. After 5 min, trypsination is stopped with a two-fold amount of growth medium and transferred into a new flask or 6-well plates with cover-slips.

### Transient Transfection

DNA for transfection was prepared using the QIAGEN Endotoxin-free plasmid Maxi kit (QIAGEN, C-12362, West Sussex, UK) according to the instruction of the manufacturer. For Nucleofector-mediated transfection, either parent HE 293 cells or its transfectants stably expressing genes of interest are washed with PBS and aspirated to discard PBS. The growth medium is aspirated, cultured adherent cells are washed once with phosphate buffered saline (PBS) and immediately detached with trypsin/EDTA, and centrifuged for 5 min at 170×g. 1×10⁶ cells are resuspended in 100 μl Nucleofector Solution V (Amaxa, Cologne, Germany). Afterwards, 5 μg of either pcDNA3.1 (+) (Invitrogen) or pIRES2-EGFP (Clontech, Palo Alto, Calif., USA) vector DNA or vectors containing the fragment of Bax (FIG. 10, FIG. 11) are added, the mixture is transferred into an electroporation cuvette and placed in the Nucleofector device (Amaxa). Program Q-01 is used for transfection. (Further details are proprietary information of Amaxa Biosystems, Cologne, Germany). Immediately after Nucleofection, the suspension is transferred into T-25 (Corning), containing 4 ml pre-warmed culture medium. Medium is changed after 15-20 h. After 24 h, the efficiency of transfected cells is determined by counting the number of cells positive for eGFP under a fluorescence microscope in 10 randomly selected fields.

### Establishment of Cell Lines Stably Expressing Human Bcl-2A1, FKBP2, SNCA, VAMP3, EEF1A1 and Bcl-xL

### Clone Selection by G418 Resistance

The plasmid DNA derived from pcDNA3.1 (+) and carrying HA-tagged full-length Bcl-2A1, FKBP2, SNCA, VAMP3, EEF1A1 and Bcl-xL gene fragments (FIGS. 33 to 38) and pcDNA3.1 vector are transfected into HEK293 cells (see Example 2). The transfectants are seeded onto a 100-mm tissue culture dish (Corning) containing 8 ml pre-warmed culture medium. After incubation for 2 days, the transfected cells are selected for G418 resistance in medium containing 1 mg/ml G418 (Gibco/BRL Life Technologies). After 10-15 days, the surviving single colonies (G418-resistant colonies) are picked up using cloning rings and transferred to 24-well dishes with further selection in medium containing 200 mg/ml G418. The expression of human Bcl-2A1, FKBP2, SNCA, VAMP3, EEF1A1 and Bcl-xL is determined using anti-HA-fluorescein (Roche, Penzberg, Germany) using immunocytochemistry. The homogeneity of the transfectants is assured by repeated subcloning.

### Isolation of Stable Clones

Transient transfection efficiencies are typically 90%, respectively.

Stable transformants are designated as HEK293/Bcl2A1, HE 293/FKBP2, HEK293/SNCA, HEK293/VAMP3, HEK293/EEF1A1 and HEK293/Bcl-xL, which express HA-tagged Bcl-2A1, FKBP2, SNCA, VAMP3, EEF1A1 and Bcl-xL, respectively. As a mock control, pcDNA3.1(+) is introduced into HEK93 and named HEK293pc0 (Table 6).

TABLE 6
Characteristics of transfectants

| | | No. of | Clones that are | |
|---|---|---|---|---|
| | | G418 | anti-HA | Clones that are |
| Name of | Protein | resistant | fluorescence | Western blot |
| transformant | Expressed | clones | positive | positive |
| HEK293pc0 | — | 3 | — | — |
| HEK293/Bcl2A1 | Bcl-2A1 | 14 | Clone 3, 4 | Clone 3, 4 |
| HEK293/FKBP2 | FKBP2 | 10 | Clone 3, 5 | Clone 3, 5 |
| HEK293/SNCA | SNCA | 6 | Clone 2, 3, 4, 5 | Clone 2, 3, 4, 5 |
| HEK293/VAMP3 | VAMP3 | 10 | Clone 2, 4 | Clone 2, 4 |
| HEK293/EEF1A1 | EEF1A1 | 10 | Clone 2, 5 | Clone 2, 5 |
| HEK293/Bcl-xL | Bcl-xL | 10 | Clone 1, 5 | Clone 1, 5 |

### Immunocytochemistry

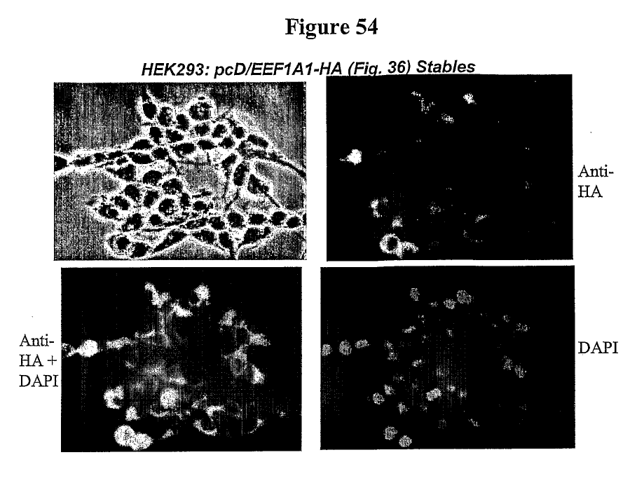

Immunocytochemistry (ICH) is an important method for identification of proteins in cells and in tissues. Approximately 24-48 h after the transfected cells are seeded into six-well plates containing sterilized coverslips, which are pre-coated with 0.001% poly-L-lysine (Sigma), the cells are washed with pre-warmed PBS twice and fixed with 4% paraformaldehyde (PFA) (Sigma)/PBS at 37° C. for 15 min. The cells are washed twice and permeabilised using 0.3% Triton X-100/10 mM sodium phosphate/0.5 M NaCl, pH 7.4 at room temperature for 5 min. Then the cells are incubated for 1 h with a fluorescein-conjugated monoclonal antibody against the HA-tagged recombinant proteins. (Roche, Penzberg, Germany (1:50 dilution). After washing in PBS containing 10 μg/100 ml DAPI for 5 min at RT, the cells on the cover-slips are mounted on glass slides using the anti-fade mounting medium (0.1% P-phenylenediamine (PPD) (Sigma) in glycerol. Slides are then analyzed by Olympus IX-70 fluorescence microscopy with Analysis 5.5 B software and the images are captured by an Optronics digital imaging camera. Cells are counterstained with DAPI to identify the nuclear DNA. A representative picture is shown in FIG. 54.

Table 6 summarises the results obtained from immunocytochemistry which confirms the presence of HA-tagged proteins.

### Western Blotting

20 μg of total protein from cell lysates are fractionated using 10% PAGE. A monoclonal antibody that recognises the HA-tag is used to illuminate the HA-tagged proteins.

Table 6 summarises the results obtained from Western blotting which confirms the presence of HA-tagged proteins.

### Bax-Mediated Cell Death in HEK293

After transfection of Bax, using plasmids pcDNA3.1/Bax (FIG. 10) and pIRESEGFP/Bax (FIG. 11), into HE 93, the cell viability is determined using trypan blue exclusion assay to detect the Bax-mediated cell death.

### Experiment

HEK293 cells are transfected with pcDNA3.1/Bax (FIG. 10), the control plasmid cDNA3.1 (Invitrogen), pIRESEGFP/Bax (FIG. 11) and the control plasmid pIRES2-EGFP (Clontech). Each transfection is plated equally on to 6 wells of a 6-well plate. Exactly the same amount of cells and plasmid DNA is utilized for each transfection. The transfectants are incubated as described in Example 6 above. The medium is changed after 15-20 h. For the first 5 days after transfection, on each day all 6 wells of the same transfectants are harvested individually. The cells are rinsed, gently scraped, pelleted by centrifugation, resuspended in 500 μl of culture medium containing 0.1% trypan blue, loaded into a haemocytometer, and examined by light microscopy. Viable and nonviable (blue cells) cells are then counted, and the scores obtained indicate dead cells as a percentage of total cells. The relative survival (i.e. viable) rate is also calculated. This experiment is repeated 3 times.

### Results

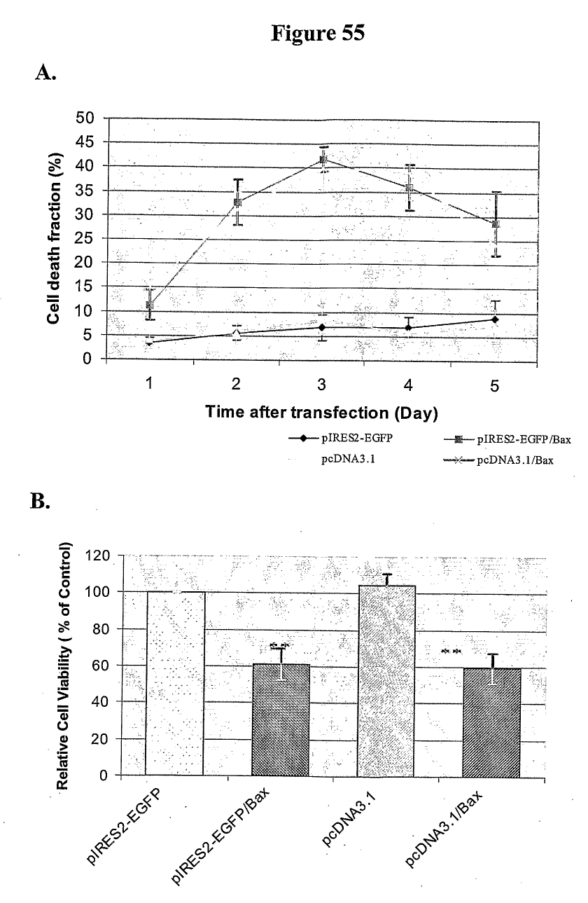

Cell death occurs in HEK293 after Bax-transfection. Exclusion of trypan blue dye by viable cells is evaluated under a light microscope using a haemocytometer after transient transfection of the Bax containing plasmids pcDNA3.1/Bax (FIG. 10) and pIRESEGFP/Bax (FIG. 11). The results clearly show a decrease in cell viability. The cultured cells are harvested and counted to determine the percentage of dead cells for the indicated times after Bax-transfection (FIG. 55, Panel A). Cells are counted from the first day to the fifth day. The results are compared with cells transfected with the empty vector (i.e. pcDNA3.1 and pIRES2-EGFP). Cells transfected with pcDNA3.1/Bax (FIG. 10) and pIRESEGFP/Bax (FIG. 11; labelled in FIG. 55 as pIRES2-EGFP/Bax) show an increase in the percentage of dead cells (P<0.001). It seems that cell death is independent of the vector used (P>0.05). Panel B (FIG. 55) shows the relative percentages of viable cells on the third day after Bax or vector transfection.

### Anti-Apoptotic Function in Human Cells of the Novel Genes Selected by YAS

Inhibition of Bax-Mediated Death by the Proteins Expressed from the Novel Genes

A similar trypan blue exclusion assay is used to determine the capacity of Bcl-2A1, FKBP2, SNCA, VAMP3, EEF1A1 and Bcl-xL proteins to protect HE 93 from Bax-mediated death.

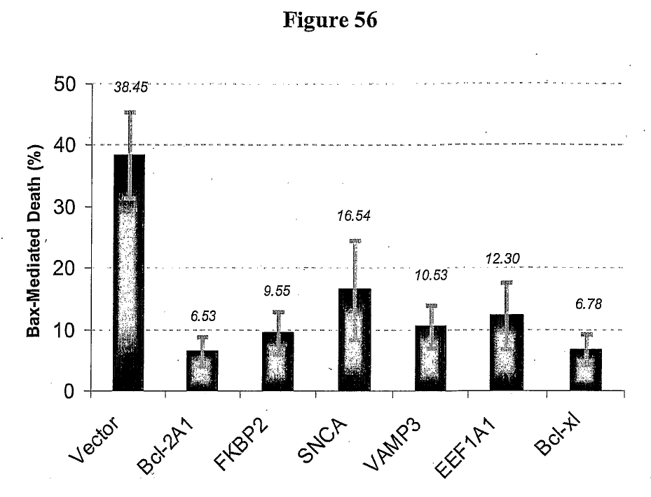

pIRES2-EGFP/Bax is transfected into Bcl-2A1, FKBP2, SNCA, VAMP3, EEF1A1 Bcl-xL and vector transfected stable cell lines. The vector-transfection is done as negative control. The exact same amount of cells and plasmid DNA is utilized for each transfection. The transfectants are incubated as described above in Example 6. The medium is changed after 15-20 h and the transfection efficiency is calculated. After incubation for 3 days, the viable cells are counted as described above. The score obtained yields the percentage of cells undergoing Bax-mediated death and is calculated according to the following formula. The experiments are repeated 6 times and the results are depicted in FIG. 56.

Bax-mediated cell death(%)=[(V₀−V_{Bax})/(Fₜ×V₀)]×100%

V₀ stands for the number of viable cells without Bax that is, after vector-transfection; V_{Bax} stands for the number of viable cells after transfection with Bax alone; Fₜ stands for the average transfection efficiency.

### Detection of DNA Fragmentation by TUNEL Assay

Bax-induced apoptosis is quantified by TUNEL (terminal deoxynucleotidyltransferase [TdT]-mediated dUTP nick end labelling) using FACS (fluorescence-activated cell sorting) analysis.

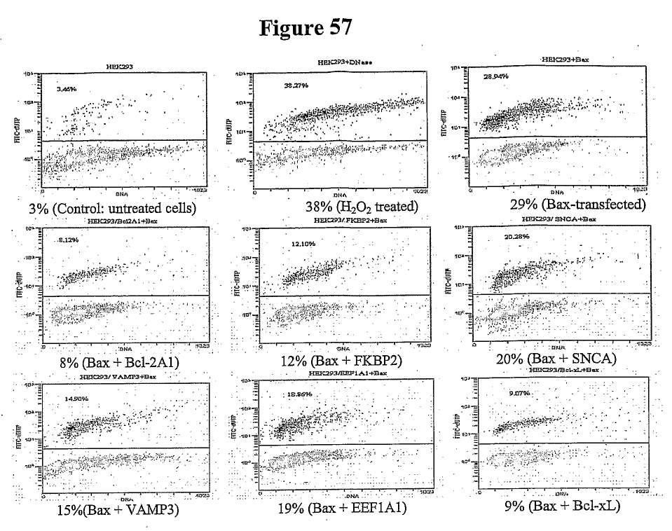

pIRES2-EGFP/Bax is transfected into the HEK293 cells which stably express human Bcl-2A1, FKBP2, SNCA, VAMP3, EEF1A1 and Bcl-xL. After incubation for 3 days, the transfectants are harvested and washed in PBS. Then the cells fixed with freshly prepared 2% paraformaldehyde in PBS for 60 min at 15-25° C. After washed again with PBS, the cells are permeabilised with 1% Triton X-100 in 1% sodium citrate for 2 min on ice, followed by washing twice with PBS. Cells are then incubated with an Apotag (Roche, East Sussex, UK) enzymatic reaction mixture containing terminal deoxynucleotidyl transferase and FITC-dUTP for 60 min at 37° C. in a humidified atmosphere in the dark according to the manufacturer's instructions before a final wash in PBS and resuspension in PI/RNase solution (0.001% Propidium iodide, 0.05% RNase A in PBS). FACS analysis is then performed using Beckmann-Coulter flow cytometer (Epic-Altra) equipped with a 488 nm Argon Laser as the light source. Propidium iodide (DNA) fluoresces at 623 nm (which determines the total number of cells) and FITC at 520 nm (which determines apoptotic cells). A total of 10,000 events of interest are analyzed. The results are depicted in FIG. 57.

Determination of Cytochrome c Release from Mitochondria by In Situ Immunofluorescence

It has been shown that the pro-apoptotic Bax releases cytochrome c from the mitochondria in order to induce apoptosis. The newly found anti-apoptotic proteins from the human hippocampus using the yeast screen should prevent cytochrome c release in human cells if they are truly anti-apoptotic.

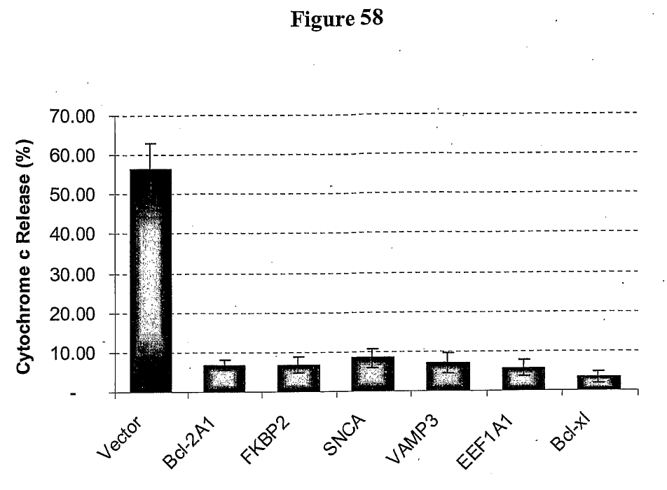

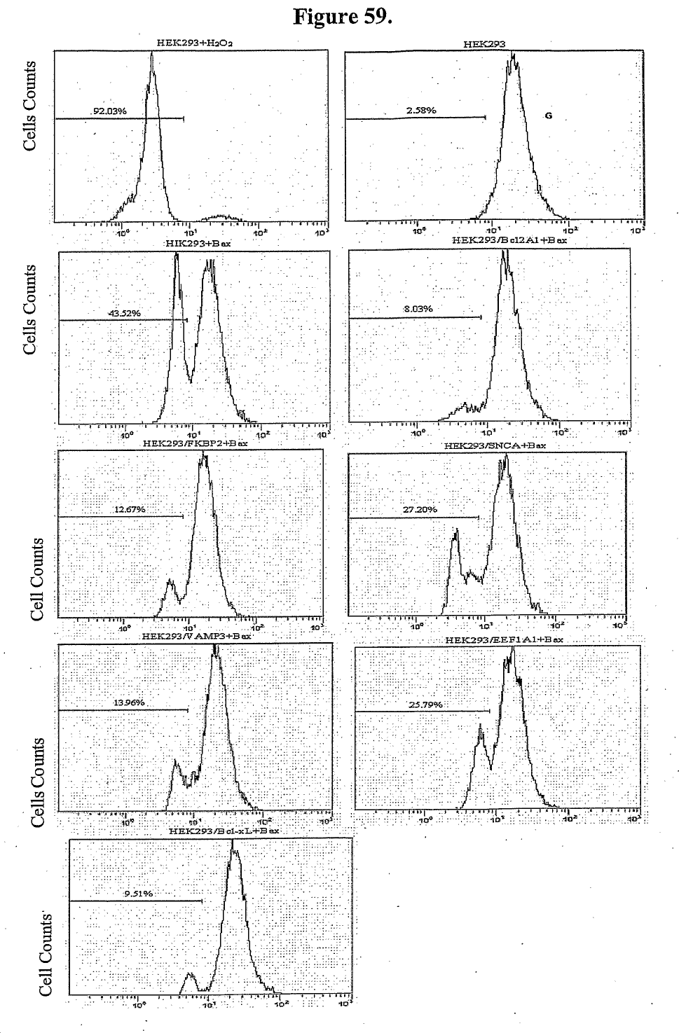

pcDNA3.1/Bax is transfected into the HEK293 cells which stably expresses human Bcl-2A1, FKBP2, SNCA, VAMP3, EEF1A1, Bcl-xL and empty vector (see above). A certain amount of transfected cells are directly seeded into six-well plates containing sterilized cover-slips, which are pre-coated with 0.001% poly-L-lysine (Sigma). After incubation for 3 days, cytochrome c is labelled using SelectFX™ Alexa Fluor® 488 Cytochrome c Apoptosis Detection Kit (Molecular Probes) following the instruction of the manufacturer. Briefly, the cells are grown on cover-slips and are washed with pre-warmed PBS twice and fixed with freshly prepared 4% paraformaldehyde (PFA) (Sigma)/PBS at 37° C. for 15 min. The cells are washed twice and permeabilised using 0.3% Triton X-100/10 mM sodium phosphate/0.5 M NaCl, pH 7.4 at room temperature for 5 min. Samples are blocked with the blocking solution (10% heat-inactivated goat serum in PBS after washed twice with PBS). Primary Anti-cytochrome c mouse IgG₁ is 500-fold diluted in this blocking solution and incubated with the cells on slides in a wet box for 1 h at room temperature. Samples are rinsed twice with the blocking solution and incubated with the Alexa 488 goat anti-mouse secondary antibody (Molecular Probes) in the dark for 30 min at room temperature. Samples are rinsed three times in PBS and mounted on glass slides using the anti-fade mounting medium (0.1% P-phenylenediamine (PPD) (Sigma) in glycerol). Slides are then analyzed by Olympus IX-70 fluorescence microscopy and the images are captured by an Optronics digital imaging camera. The results are shown in FIG. 58.

### Measurement of Mitochondrial Membrane Potential

The mitochondrial membrane potential (ΔΨₘ) is quantified by flow cytometric analysis using 3,3′dihexyloxacarbocyanine iodide [DiOC6(3)]-stained cells. The assay utilizes a lipophilic cationic fluorescent dye DiOC6(3) that is transported into the mitochondria by the negative mitochondrial membrane potential and thus is concentrated within the mitochondrial matrix. Decreased mitochondrial membrane potential leads to reduced cellular fluorescence.

pcDNA3.1/Bax is transfected into the HEK293 cells which stably expresses human Bcl-2A1, FKBP2, SNCA, VAMP3, EEF1A1, Bcl-xL and empty vector. A certain amount of transfected cells are directly seeded into six-well plates containing sterilized cover-slips, which are pre-coated with 0.001% poly-L-lysine (Sigma). Cells are stained with DiOC6(3) (40 nM in PBS; Molecular Probes, Inc.) for 15 min at 37° C. and washed once with PBS, followed by analysis on a flow-cytometer (Beckmann-Coulter Epic-Altra; excitation at 488 nm, emission at 525 nm). The results are shown in FIG. 59.

### Intracellular Generation of Reactive Oxygen Species (ROS)

Bax-mediated ROS production is monitored using a fluorescent dye, 2′,7′-dichlorodihydrofluorescein diacetate (H₂DCFDA; Molecular Probes, Paisley, UK). This dye is a stable nonpolar compound that readily diffuses into cells and yields DCFH. Intracellular H₂O₂ or other low-molecular-weight peroxides, in the presence of peroxidases oxidise DCFH to the highly fluorescent compound DCF. Thus the fluorescence emitted by DCF directly reflects the overall oxidative status of a cell (Wang & Joseph, 1999).

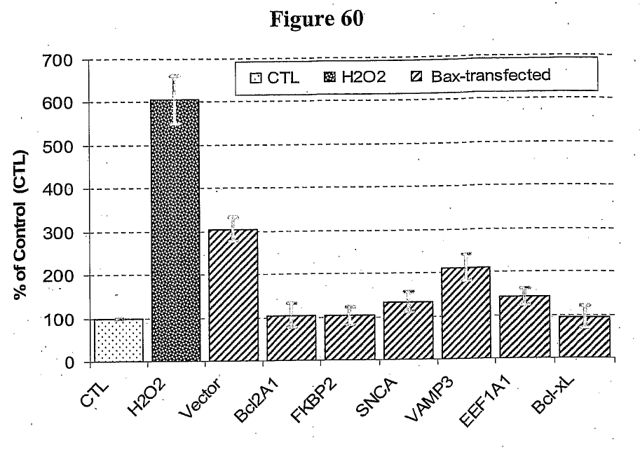

pcDNA3.1/Bax is transfected into the HEK293 cells which stably expresses human Bcl-2A1, FKBP2, SNCA, VAMP3, EEF1A1, Bcl-xL and empty vector. On the fourth day after Bax transfection, the medium is aspirated and the cultured cells are washed twice with physiological buffer (140 mM NaCl, 5 mM KCl, 1 mM CaCl₂, 1.2 mM NaHPO₄, 5 mM glucose and 20 mM Hepes, pH 7.4) and immediately detached with trypsin/EDTA, then incubated in 2 ml of cell culture medium without FBS but containing 5 μM H₂DCFDA for 30 min in 5% CO₂/95% air at 37° C. The cells are washed twice with PBS to remove the extra-cellular H₂DCFDA. Then 1×10⁶ cells are resuspended in 1 ml of PBS. 100 μl of the cells from each treatment are loaded into the 96-well plate with black sides. Cellular fluorescence is detected at wavelengths of 492 nm (excitation) and 520 nm (emission). The relative fluorescence-intensity is monitored using a Synergy™ HT Multi-Detection Microplate Reader (Bio-Tek Instruments, Winooski, Vt., USA). The values from 10 independent measurements are calculated using the Bio-Tek KC4 software to give a mean value. The results are depicted in FIG. 60.

In the above experiments, data are expressed as mean±SD unless otherwise indicated. We use SPSS12.0 software for all statistical calculations. In all tests, same experiment is repeated at least 3 times (n≧3) and the difference is considered to be statistically significant if the p value is <0.05.

### REFERENCES

Baier et al (2003) “Apoptosis in rheumatoid arthritis”. Curr Opin Rheumatol. 15(3): 274-9.
Bennetzen & Hall (1982) “Codon selection in yeast”. J Biol. Chem. 257(6): 3026-31.
Brands et al (1986) The primary structure of the alpha subunit of human elongation factor 1: structural aspects of guanine-nucleotide-binding sites. Europ. J. Biochem. 155: 167-171, 1986.
Budihardo et al (1999) “Biochemical pathways of caspase activation during apoptosis”. Annu Rev Cell Dev Biol. 15: 269-90.
Burtnick et al (2004) “Structure of the N-terminal half of gelsolin bound to actin: roles in severing, apoptosis and FAF”. EMBO J. 23(14): 2713-22.
Byrne and Ruddle (1989) Proc. Natl. Acad. Sci. USA 86: 5473-5477
Cardoso & Oliveira (2003) “Inhibition of NF-kB renders cells more vulnerable to apoptosis induced by amyloid beta peptides”. Free Radic Res. 37(9): 967-73.
Chong et al (2005) “Activating Akt and the brain's resources to drive cellular survival and prevent inflammatory injury”. Histol Histopathol. 20(1): 299-315.
Cnop et al (2005) “Mechanisms of Pancreatic {beta}-Cell Death in Type 1 and Type 2 Diabetes: Many Differences, Few Similarities”. Diabetes. 54 Suppl 2: S97-S107.
Cooper & Burge (2003) “Darier's disease: epidemiology, pathophysiology, and management.” Am J Clin Dermatol. 4(2): 97-105
Cory et al (2003) “The Bcl-2 family: roles in cell survival and oncogenesis. Oncogene 22(53): 8590-607.
Davies (2000) “Neurotrophins: more to NGF than just survival.” Curr Biol. 10(10): R374-6
Dhitavat et al (2004) Calcium pumps and keratinocytes: lessons from Darier's disease and Hailey-Hailey disease. British Journal of Dermatology 150: 821-828.
DiLella et al (1992) “Chromosomal band assignments of the genes encoding human FKBP12 and FKBP13”. Biochem. Biophys. Res. Commun. 189(2): 819-23.
Distelhorst & Shore (2004) “Bcl-2 and calcium: controversy beneath the surface”. Oncogene. 23(16): 2875-80.
Ditzel et al (2000) Cloning and expression of a novel human antibody—antigen pair associated with Felty's syndrome. Proc. Nat. Acad. Sci. USA 97: 9234-9239.
Fan et al (1996) Mutations in the RNA polymerase II transcription machinery suppress the hyperrecombination mutant hpr1 delta of Saccharomyces cerevisiae. Genetics. 142(3): 749-59.
Foggia & Hovnanian (2004) Calcium pump disorders of the skin. Am. J. Med Genet. C (Semin. Med. Genet) 131C: 20-31.
Greenhalf et al (1996) Role of mitochondria and C-terminal membrane anchor of Bcl-2 in Bax induced growth arrest and mortality in Saccharomyces cerevisiae. FEBS Lett. 380(1-2): 169-75.
Hajra & Liu (2004) “Apoptosome dysfunction in human cancer”. Apoptosis. 9(6): 691-704.
Halliwell & Whiteman (2004) “Measuring reactive species and oxidative damage in vivo and in cell culture: how should you do it and what do the results mean?” Br J Pharmacol. 142(2): 231-55.
Harms et al (2004) “Neuronal gelsolin prevents apoptosis by enhancing actin depolymerization”. Mol Cell Neurosci. 25(1): 69-82.
Heaton et al (2003) “Effects of ethanol on neurotrophic factors, apoptosis-related proteins, endogenous antioxidants, and reactive oxygen species in neonatal striatum: relationship to periods of vulnerability”. Brain Res Dev Brain Res. 140(2): 237-52.
Jackisch et al (2000) Delayed micromolar elevation in intracellular calcium precedes induction of apoptosis in thapsigargin-treated breast cancer cells. Clin Cancer Res 6: 2844-50.
Johnston (1987) “A model fungal gene regulatory mechanism: the GAL genes of Saccharomyces cerevisiae”. Microbiol Rev. 51(4): 458-76.
Johnston (2005) “Excitotoxicity in perinatal brain injury”. Brain Pathol. 15(3): 234-40.
Kalivendi et al (2004) Alpha-synuclein up-regulation and aggregation during MPP+ induced apoptosis in neuroblastoma cells: intermediacy of transferrin receptor iron and hydrogen peroxide. J Biol Chem 279(15): 15240-7.
Kato (2001) “Molecular genetics of bipolar disorder” Neurosci Res. 40(2): 105-13.
Kirkland et al (2002) “A Bax-induced pro-oxidant state is critical for cytochrome c release during programmed neuronal death”. J Neurosci. 22(15): 6480-90.
Lamberti et al (2004) The translation elongation factor 1A in tumorigenesis, signal transduction and apoptosis: Review article. Amino Acids 26: 443-448.
Leroy et al (2005) “Protein kinase C zeta associates with death inducing signalling complex and regulates Fas ligand-induced apoptosis”. Cell Signal. 17(9): 1149-57.
Li et al (2004) “Cyclophilin-D promotes the mitochondrial permeability transition but has opposite effects on apoptosis and necrosis”. Biochem J. 383(1): 101-9.
Ligr et al (1998) Mammalian Bax triggers apoptotic changes in yeast. FEBS Lett. 438(1-2): 61-65.
Manon et al (1997) Release of cytochrome c and decrease of cytochrome c oxidase in Bax-expressing yeast cells, and prevention of these effects by coexpression of Bcl-xL. FEBS Lett. 415(1): 29-32.
Matsuyama et al (1999) Yeast as a tool for apoptosis research. Curr Opin Microbiol. 2(6): 618-23.
Melo L G et al, (2004) Gene and cell-based therapies for heart disease. FASEB J. 18(6): 648-63.
Millet et al (2005) “Targeted expression of the anti-apoptotic gene CrmA to NOD pancreatic islets protects from autoimmune diabetes. Targeted expression of the anti-apoptotic gene CrmA to NOD pancreatic islets protects from autoimmune diabetes”. J Autoimmun. December 6; [Epub ahead of print]
Morii et al (1991) Biochem. Biophys Res. Commun. 175: 185-191
Mundle (2005) “Par-4: A common facilitator/enhancer of extrinsic and intrinsic pathways of apoptosis”. Leuk Res. November 10; [Epub ahead of print]
Olivia et al (1991) Genomics 10: 157-165
Partaledis & Berlin (1993) The FKB2 gene of Saccharomyces cerevisiae, encoding the immunosuppressant-binding protein FKBP-13, is regulated in response to accumulation of unfolded proteins in the endoplasmic reticulum. Proc Natl Acad Sci USA. 90(12): 5450-4.
Pereira-Leal & Seabra (2001) Evolution of the Rab family of small GTP-binding proteins. J Mol Biol. 313(4): 889-901.
Poirier, Ed. (1997) Apoptosis Techniques and Protocols, Humana Press, Totowa, N.J., USA.
Pong (2003) “Oxidative stress in neurodegenerative diseases: therapeutic implications for superoxide dismutase mimetics”. Expert Opin Biol Ther. 3(1): 127-39.
Poon et al (2004) “Free radicals and brain aging”. Clin Geriatr Med. 20(2): 329-59.
Prasad et al (2005) Haploinsufficiency of Atp2a2, Encoding the Sarco(endo)plasmic Reticulum Ca²⁺-ATPase Isoform 2 Ca²⁺ Pump, Predisposes Mice to Squamous Cell Tumors via a Novel Mode of Cancer Susceptibility. Cancer Res 65(19): 8655-61.
Priault et al (2003) Yeast as a tool to study Bax/mitochondrial interactions in cell death. FEMS Yeast Res. 4(1): 15-27.
Qu et al (2004) Endoplasmic reticulum stress induces p53 cytoplasmic localization and prevents p53-dependent apoptosis by a pathway involving glycogen synthase kinase-3h. Genes Dev 18: 261-77.
Reeder & Lang (1994) “The mechanism of transcription termination by RNA polymerase I”. Mol. Microbiol. 12(1): 11-5.
Reeve et al (2005) “Don't lose heart—therapeutic value of apoptosis prevention in the treatment of cardiovascular disease.” J Cell Mol Med. 9(3): 609-22.
Rogaev et al (1992) Hum. Mol. Genet. 1: 781
   Rokosz et al (1994) “Human cytoplasmic 3-hydroxy-3-methylglutaryl coenzyme A synthase: expression, purification, and characterization of recombinant wild-type and Cys129 mutant enzymes. Arch. Biochem. Biophys. 312 (1): 1-13.
Rothstein (1983) One-step gene disruption in yeast. Meth. Enzymol. 101: 202-11.
Rouaux et al (2004) “Targeting CREB-binding protein (CBP) loss of function as a therapeutic strategy in neurological disorders”. Biochem Pharmacol. 68(6): 1157-64.
Savolainen et al (1998) “Interactions of excitatory neurotransmitters and xenobiotics in excitotoxicity and oxidative stress: glutamate and lead”. Toxicol Lett. 102-103: 363-7.
Sellers et al (1994) J. Immmunol. Meth. 172, 255-264.
Sharif F, et al (2004) “Current status of catheter- and stent-based gene therapy.” Cardiovasc Res. 64(2): 208-16.
Shull et al (2003) Physiological functions of plasma membrane and intracellular Ca²⁺ pumps revealed by analysis of null mutants. Ann N Y Acad Sci. 986: 453-60.
Soane & Fiskum (2005) “Inhibition of mitochondrial neural cell death pathways by protein transduction of Bcl-2 family proteins”. J Bioenerg Biomembr. 37(3): 179-90.
Sreedhar & Csermely (2004) “Heat shock proteins in the regulation of apoptosis: new strategies in tumor therapy: a comprehensive review”. Pharmacol Ther. 101(3): 227-57.
Sullivan et al (2005) “Mitochondrial permeability transition in CNS trauma: cause or effect of neuronal cell death?” J Neurosci Res 79(1-2): 231-9.
Sung et al (2005) “The pleiotropy of telomerase against cell death”. Mol. Cells. 19(3): 303-9.
Svensson E C, et al. (1999) Efficient and stable transduction of cardiomyocytes after intramyocardial injection or intracoronary perfusion with recombinant adeno-associated virus vectors. Circulation. 99: 201-5.
Talapatra & Thompson (2001) Growth factor signalling in cell survival: implications for cancer treatment. J Pharmacol Exp Ther 298(3): 873-8.
Talapatra et al (2001) Elongation factor-1 alpha is a selective regulator of growth factor withdrawal and ER stress-induced apoptosis. Cell Death Differ 9: 856-861.
Telford et al (1994) J. Immunol. Meth. 172, 1-16,
Thomas & Rothstein (1989) Elevated recombination rates in transcriptionally active DNA. Cell 56: 619-630.
Thornton et al (2003) Not just for housekeeping: protein initiation and elongation factors in cell growth and tumorigenesis. J Mol Med 81: 536-548
Touchman et al (2001) Human and Mouse α-Synuclein Genes: Comparative Genomic Sequence Analysis and Identification of a Novel Gene Regulatory Element. Genome Research 11(1): 78-86
Valko et al (2005) “Metals, toxicity and oxidative stress”. Curr Med Chem. 12(10): 1161-208.
Van den et al (1991) Biochem. Biophys. Acta. 2: 249-251.
van Duijn et al (2001) PARK7, a novel locus for autosomal recessive early-onset parkinsonism, on chromosome 1p36. Am. J. Hum. Genet. 69: 629-634
Veal et al (2003) Ybp1 is required for the hydrogen peroxide-induced oxidation of the Yap1 transcription factor. J Biol Chem. 278(33): 30896-904. Erratum in: J Biol Chem. (2004) 279(47): 49562.
Voth et al (2005) ACE2, CBK1, and BUD4 in budding and cell separation. Eukaryot Cell. 4(6): 1018-28.
Wang & Joseph (1999) “Quantifying cellular oxidative stress by dichlorofluorescein assay using microplate reader.” Free Radic Biol Med. 27(5-6): 612-6.
Wang & Sudhof (2003) Genomic definition of RIM proteins: evolutionary amplification of a family of synaptic regulatory proteins. Genomics. 81(2):126-37.
Wang et al (2000) The RIM/NIM family of neuronal C2 domain proteins. Interactions with Rab3 and a new class of Src homology 3 domain proteins. J Biol Chem. 275(26): 20033-44.
Xu & Reed (1998) Bax inhibitor-1, a mammalian apoptosis suppressor identified by functional screening in yeast. Mol Cell. 1(3): 337-46.
Yamamoto & Behl (2001) “Human Nck-associated protein 1 and its binding protein affect the metabolism of beta-amyloid precursor protein with Swedish mutation”. Neurosci Lett. 316(1): 50-4.

Yano et al (2005) “Functional proteins involved in regulation of intracellular Ca(2+) for drug development: role of calcium/calmodulin-dependent protein kinases in ischemic neuronal death”. J Pharmacol Sci. 97(3): 351-4.
Zweifel et al (2005) “Functions and mechanisms of retrograde neurotrophin signalling”. Nat Rev Neurosci. 6(8): 615-25.

1. A Saccharomyces cerevisiae yeast cell which has the genotype MAT-a, ade2-1, trp1-1, leu2-3, leu2-112, his3-11, his3-15, ura3-1 and can1-100, and
(a) which contains a polynucleotide that encodes a functional Bax polypeptide under the control of a galactose-inducible promoter that is integrated at the LEU2 chromosomal locus, or
(b) which contains a yeast integrating plasmid which comprises a polynucleotide that encodes a functional Bax polypeptide under the control of a galactose-inducible promoter, and which is suitable for integration at the LEU2 chromosomal locus.

2. (canceled)

3. The cell of claim 1 wherein the yeast cell is strain W303-1A.

4. The cell of claim 1 wherein the functional Bax polypeptide comprises a human Bax polypeptide, or a pro-apoptotic fragment or variant thereof.

5. The cell of claim 1 wherein the codons of the polynucleotide encoding the functional Bax polypeptide have been optimised for yeast.

6. The cell of claim 1 wherein the polynucleotide encoding the functional Bax polypeptide comprises the sequence of SEQ ID No: 2.

7. The cell of claim 1 wherein the galactose-inducible promoter is GAL1 or GAL10.

8. The cell of claim 1 wherein the polynucleotide encoding the functional Bax polypeptide is terminated by a SUC2 transcription terminator sequence.

9. The cell of claim 1 wherein expression of the functional Bax polypeptide in the presence of galactose results in cell death.

10. The cell of claim 1 which is strain W303baxleu.

11. A kit comprising yeast cells of claim 1 and a yeast plasmid vector suitable for transforming a library of polynucleotides into the yeast cells.

12. The kit of claim 11 wherein the yeast plasmid vector is suitable for expressing a polynucleotide from the library of polynucleotides under the control of an inducible promoter.

13. The kit of claim 11 wherein the yeast plasmid vector is pYES2 (Stratagene).

14. The kit of claim 12 further comprising an agent that induces expression of the polynucleotide which is under the control of the inducible promoter in the yeast cell.

15. The kit of claim 14 wherein the agent that induces expression of the polynucleotides in the yeast cell is galactose.

16. The kit of claim 14 further comprising galactose.

17. The kit of claim 11 wherein the library of polynucleotides is a cDNA library.

18. The kit of claim 11 further comprising instructions for performing a method of screening the library of polynucleotides for an inhibitor of Bax-mediated apoptosis.

19. A method of screening for a polynucleotide that is or encodes an inhibitor of Bax-mediated apoptosis, which method comprises:
(a) providing a library of polynucleotides in yeast plasmid vectors;
(b) transforming the library of polynucleotides into yeast cells as defined in claim 1;
(c) plating the transformed yeast under conditions that allow expression of the functional Bax polypeptide and of the polynucleotides in the yeast plasmid vectors; and
(d) identifying a yeast colony that grows,
wherein growth of a yeast colony indicates that the polynucleotide in the yeast plasmid vector is, or encodes, an inhibitor of Bax-mediated apoptosis.

20. The method of claim 19 wherein the library of polynucleotides is a cDNA library generated from human brain tissue, a tissue that is involved in diabetes, a cancer tissue, heart tissue, a tissue that is involved in rheumatoid arthritis, a cell line, or a bacterial or viral genome.

21. The method of claim 19 wherein the library of polynucleotides in the yeast plasmid vectors are under the control of an inducible promoter.

22. The method of claim 21 wherein the inducible promoter is a tetracycline-inducible promoter, a methionine-inducible promoter, a galactose-inducible promoter, an ADH2 promoter or a metallothionein promoter.

23. The method of claim 22 wherein the galactose-inducible promoter is GAL1 or GAL10.

24. The method of claim 19 wherein the yeast plasmid vector is pYES2.

25. The method of claim 19 wherein the transforming step (b) is a high efficiency transformation.

26. The method of claim 22 wherein the plating step (c) comprises incubating the plated yeast cells at 30° C. in the presence of galactose for at least 72 hours.

27. The method of claim 19 further comprising obtaining the sequence of the polynucleotide in the yeast plasmid vector present in a yeast colony identified in step (d).

28. The method of claim 19 further comprising retesting the polynucleotide from the plasmid vector present in a yeast colony identified in step (d), or a polypeptide encoded by said polynucleotide, for the ability to inhibit Bax-mediated apoptosis in a model of apoptosis.

29. The method of claim 19 further comprising modifying the polynucleotide from the plasmid vector present in a yeast colony identified in step (d), and testing the modified polynucleotide, or a polypeptide encoded by said modified polynucleotide, for the ability to inhibit Bax-mediated apoptosis in a model of apoptosis.

30. The method of claim 27 further comprising identifying the polynucleotide based upon the obtained sequence data, and testing a polynucleotide that corresponds to the identified polynucleotide, or a polypeptide encoded by said corresponding polynucleotide, for the ability to inhibit Bax-mediated apoptosis in a model of apoptosis.

31. The method of claim 28 wherein the model of apoptosis is selected from a yeast cell model, a mammalian cell model, and an in vivo model of apoptosis.

32. The method of claim 19, further comprising the step of formulating a polynucleotide or polypeptide which has the ability to inhibit Bax-mediated apoptosis into a pharmaceutically acceptable composition.

33.-75. (canceled)
